SEQUENCE LISTING (29 sequences)

SEQ ID NO: 1 - RNA, 19 nt, synthetic construct
  source 1..19: mol_type = other RNA
  modified_base 1..2: mod_base = OTHER; note = 2'-methoxy and 5'-thiophosphate modified nucleotides
  modified_base 3..6: mod_base = OTHER; note = 2'-methoxy modified nucleotides
  modified_base 7..9: mod_base = OTHER; note = 2'-fluoro modified nucleotides
  modified_base 10..19: mod_base = OTHER; note = 2'-methoxy modified nucleotides
ttttaatcctcactctaaa

SEQ ID NO: 2 - RNA, 21 nt, synthetic construct
  source 1..21: mol_type = other RNA
  modified_base 1: mod_base = OTHER; note = 2'-methoxy and 5'-thiophosphate modified nucleotide
  modified_base 2: mod_base = OTHER; note = 2'-fluoro and 5'-thiophosphate modified nucleotide
  modified_base 3..5: mod_base = OTHER; note = 2'-methoxy modified nucleotides
  modified_base 6: mod_base = OTHER; note = 2'-fluoro modified nucleotide
  modified_base 7..13: mod_base = OTHER; note = 2'-methoxy modified nucleotides
  modified_base 14: mod_base = OTHER; note = 2'-fluoro modified nucleotide
  modified_base 15: mod_base = OTHER; note = 2'-methoxy modified nucleotide
  modified_base 16: mod_base = OTHER; note = 2'-fluoro modified nucleotide
  modified_base 17..18: mod_base = OTHER; note = 2'-methoxy modified nucleotides
  modified_base 19..20: mod_base = OTHER; note = 2'-methoxy and 5'-thiophosphate modified nucleotides
  modified_base 21: mod_base = OTHER; note = 2'-methoxy modified nucleotide
tttagagtgaggattaaaatg

SEQ ID NO: 3 - RNA, 19 nt, synthetic construct
  source 1..19: mol_type = other RNA
  modified_base 1..2: mod_base = OTHER; note = 2'-methoxy and 5'-thiophosphate modified nucleotides
  modified_base 3: mod_base = OTHER; note = 2'-methoxy modified nucleotide
  modified_base 4: mod_base = OTHER; note = n is the synthetic nucleotide analog NM031 as described in the disclosure (e.g., NM031-1 ((1-[(2R,3R,4S, 5R)-5-{[bis(4-methoxyphenyl)(phenyl)methoxy]methyl}-3,4-di hydroxyoxolan-2-yl]-3H-pyrimidin-2,4-dione)).
  modified_base 5..6: mod_base = OTHER; note = 2'-methoxy modified nucleotides
  modified_base 7..9: mod_base = OTHER; note = 2'-fluoro modified nucleotides
  modified_base 10..19: mod_base = OTHER; note = 2'-methoxy modified nucleotides
tttnaatcctcactctaaa

SEQ ID NO: 4 - RNA, 19 nt, synthetic construct
  source 1..19: mol_type = other RNA
  modified_base 1..2: mod_base = OTHER; note = 2'-methoxy and 5'-thiophosphate modified nucleotides
  modified_base 3: mod_base = OTHER; note = 2'-methoxy modified nucleotide
  modified_base 4: mod_base = OTHER; note = n is the synthetic nucleotide analog Uhd as described in the disclosure.
  modified_base 5..6: mod_base = OTHER; note = 2'-methoxy modified nucleotides
  modified_base 7..9: mod_base = OTHER; note = 2'-fluoro modified nucleotides
  modified_base 10..19: mod_base = OTHER; note = 2'-methoxy modified nucleotides
tttnaatcctcactctaaa

SEQ ID NO: 5 - RNA, 19 nt, synthetic construct
  source 1..19: mol_type = other RNA
  modified_base 1..2: mod_base = OTHER; note = 2'-methoxy and 5'-thiophosphate modified nucleotides
  modified_base 3..4: mod_base = OTHER; note = 2'-methoxy modified nucleotides
  modified_base 5: mod_base = OTHER; note = n is the synthetic nucleotide analog NM058 as described in the disclosure(e.g., NM058-1 (1N-(9-[(2S,5R)- 5-{[bis(4-methoxyphenyl)(phenyl)methoxy]methyl}-3,4-dihydr oxytetrahydrofuran-2-yl]-9H-purin-6-yl)benzamide)).
  modified_base 6: mod_base = OTHER; note = 2'-methoxy modified nucleotide
  modified_base 7..9: mod_base = OTHER; note = 2'-fluoro modified nucleotides
  modified_base 10..19: mod_base = OTHER; note = 2'-methoxy modified nucleotides
ttttnatcctcactctaaa

SEQ ID NO: 6 - RNA, 19 nt, synthetic construct
  source 1..19: mol_type = other RNA
  modified_base 1..2: mod_base = OTHER; note = 2'-methoxy and 5'-thiophosphate modified nucleotides
  modified_base 3..5: mod_base = OTHER; note = 2'-methoxy modified nucleotides
  modified_base 6: mod_base = OTHER; note = n is the synthetic nucleotide analog NM058 as described in the disclosure(e.g., NM058-1 (1N-(9-[(2S,5R)- 5-{[bis(4-methoxyphenyl)(phenyl)methoxy]methyl}-3,4-dihydr oxytetrahydrofuran-2-yl]-9H-purin-6-yl)benzamide)).
  modified_base 7..9: mod_base = OTHER; note = 2'-fluoro modified nucleotides
  modified_base 10..19: mod_base = OTHER; note = 2'-methoxy modified nucleotides
ttttantcctcactctaaa

SEQ ID NO: 7 - DNA, 20 bp, synthetic construct
  source 1..20: mol_type = other DNA
gggttccacgtccatcagta

SEQ ID NO: 8 - DNA, 20 bp, synthetic construct
  source 1..20: mol_type = other DNA
acaccgtcctttccagcagt

SEQ ID NO: 9 - DNA, 19 bp, synthetic construct
  source 1..19: mol_type = other DNA
tgcaccaccaactgcttag

SEQ ID NO: 10 - DNA, 19 bp, synthetic construct
  source 1..19: mol_type = other DNA
ggatgcagggatgatgttc

SEQ ID NO: 11 - RNA, 19 nt, synthetic construct
  source 1..19: mol_type = other RNA
ttttaatcctcactctaaa

SEQ ID NO: 12 - RNA, 21 nt, synthetic construct
  source 1..21: mol_type = other RNA
tttagagtgaggattaaaatg

SEQ ID NO: 13 - RNA, 19 nt, synthetic construct
  source 1..19: mol_type = other RNA
  modified_base 1..2: mod_base = OTHER; note = 2'-methoxy and 5'-thiophosphate modified nucleotides
  modified_base 3: mod_base = OTHER; note = 2'-methoxy modified nucleotide
  modified_base 4: mod_base = OTHER; note = n is the synthetic nucleotide analog Uhd as described in the disclosure.
  modified_base 5..6: mod_base = OTHER; note = 2'-methoxy modified nucleotides
  modified_base 7..10: mod_base = OTHER; note = 2'-fluoro modified nucleotides
  modified_base 11..19: mod_base = OTHER; note = 2'-methoxy modified nucleotides
tttnaatcctcactctaaa

SEQ ID NO: 14 - RNA, 19 nt, synthetic construct
  source 1..19: mol_type = other RNA
  modified_base 1..2: mod_base = OTHER; note = 2'-methoxy and 5'-thiophosphate modified nucleotides
  modified_base 3: mod_base = OTHER; note = 2'-methoxy modified nucleotide
  modified_base 4: mod_base = OTHER; note = n is the synthetic nucleotide analog NM031 as described in the disclosure (e.g., NM031-1 ((1-[(2R,3R,4S, 5R)-5-{[bis(4-methoxyphenyl)(phenyl)methoxy]methyl}-3,4-di hydroxyoxolan-2-yl]-3H-pyrimidin-2,4-dione)).
  modified_base 5..6: mod_base = OTHER; note = 2'-methoxy modified nucleotides
  modified_base 7..10: mod_base = OTHER; note = 2'-fluoro modified nucleotides
  modified_base 11..19: mod_base = OTHER; note = 2'-methoxy modified nucleotides
tttnaatcctcactctaaa

SEQ ID NO: 15 - RNA, 19 nt, synthetic construct
  source 1..19: mol_type = other RNA
  modified_base 1..2: mod_base = OTHER; note = 2'-methoxy and 5'-thiophosphate modified nucleotides
  modified_base 3: mod_base = OTHER; note = 2'-methoxy modified nucleotide
  modified_base 4: mod_base = OTHER; note = n is the nucleotide analog NM073 as described in the disclosure.
  modified_base 5..6: mod_base = OTHER; note = 2'-methoxy modified nucleotides
  modified_base 7..10: mod_base = OTHER; note = 2'-fluoro modified nucleotides
  modified_base 11..19: mod_base = OTHER; note = 2'-methoxy modified nucleotides
tttnaatcctcactctaaa

SEQ ID NO: 16 - RNA, 19 nt, synthetic construct
  source 1..19: mol_type = other RNA
  modified_base 1..2: mod_base = OTHER; note = 2'-methoxy and 5'-thiophosphate modified nucleotides
  modified_base 3: mod_base = OTHER; note = 2'-methoxy modified nucleotide
  modified_base 4: mod_base = OTHER; note = n is the synthetic nucleotide analog NM074 as described in the disclosure.
  modified_base 5..6: mod_base = OTHER; note = 2'-methoxy modified nucleotides
  modified_base 7..10: mod_base = OTHER; note = 2'-fluoro modified nucleotides
  modified_base 11..19: mod_base = OTHER; note = 2'-methoxy modified nucleotides
tttnaatcctcactctaaa

SEQ ID NO: 17 - RNA, 19 nt, synthetic construct
  source 1..19: mol_type = other RNA
  modified_base 1..2: mod_base = OTHER; note = 2'-methoxy and 5'-thiophosphate modified nucleotides
  modified_base 3..6: mod_base = OTHER; note = 2'-methoxy modified nucleotides
  modified_base 7..10: mod_base = OTHER; note = 2'-fluoro modified nucleotides
  modified_base 11..19: mod_base = OTHER; note = 2'-methoxy modified nucleotides
ttttaatcctcactctaaa

SEQ ID NO: 18 - RNA, 19 nt, synthetic construct
  source 1..19: mol_type = other RNA
  modified_base 1..2: mod_base = OTHER; note = 2'-methoxy and 5'-thiophosphate modified nucleotides
  modified_base 3: mod_base = OTHER; note = 2'-methoxy modified nucleotide
  modified_base 4: mod_base = OTHER; note = n is the synthetic nucleotide analog NM200 as described in the disclosure.
  modified_base 5..6: mod_base = OTHER; note = 2'-methoxy modified nucleotides
  modified_base 7..10: mod_base = OTHER; note = 2'-fluoro modified nucleotides
  modified_base 11..19: mod_base = OTHER; note = 2'-methoxy modified nucleotides
tttnaatcctcactctaaa

SEQ ID NO: 19 - RNA, 19 nt, synthetic construct
  source 1..19: mol_type = other RNA
  modified_base 1..2: mod_base = OTHER; note = 2'-methoxy and 5'-thiophosphate modified nucleotides
  modified_base 3: mod_base = OTHER; note = 2'-methoxy modified nucleotide
  modified_base 4: mod_base = OTHER; note = n is the synthetic nucleotide analog NM201 as described in the disclosure.
  modified_base 5..6: mod_base = OTHER; note = 2'-methoxy modified nucleotides
  modified_base 7..10: mod_base = OTHER; note = 2'-fluoro modified nucleotides
  modified_base 11..19: mod_base = OTHER; note = 2'-methoxy modified nucleotides
tttnaatcctcactctaaa

SEQ ID NO: 20 - RNA, 19 nt, synthetic construct
  source 1..19: mol_type = other RNA
  modified_base 1..2: mod_base = OTHER; note = 2'-methoxy and 5'-thiophosphate modified nucleotides
  modified_base 3..5: mod_base = OTHER; note = 2'-methoxy modified nucleotides
  modified_base 6: mod_base = OTHER; note = n is the synthetic nucleotide analog NM202 as described in the disclosure.
  modified_base 7..10: mod_base = OTHER; note = 2'-fluoro modified nucleotides
  modified_base 11..19: mod_base = OTHER; note = 2'-methoxy modified nucleotides
ttttantcctcactctaaa

SEQ ID NO: 21 - RNA, 19 nt, synthetic construct
  source 1..19: mol_type = other RNA
  modified_base 1..2: mod_base = OTHER; note = 2'-methoxy and 5'-thiophosphate modified nucleotides
  modified_base 3: mod_base = OTHER; note = 2'-methoxy modified nucleotide
  modified_base 4: mod_base = OTHER; note = n is the synthetic nucleotide analog NM087 as described in the disclosure.
  modified_base 5..6: mod_base = OTHER; note = 2'-methoxy modified nucleotides
  modified_base 7..10: mod_base = OTHER; note = 2'-fluoro modified nucleotides
  modified_base 11..19: mod_base = OTHER; note = 2'-methoxy modified nucleotides
tttnaatcctcactctaaa

SEQ ID NO: 22 - RNA, 21 nt, synthetic construct
  source 1..21: mol_type = other RNA
  modified_base 1: mod_base = OTHER; note = 2'-methoxy and 5'-thiovinylphosphonate modified nucleotide
  modified_base 2: mod_base = OTHER; note = 2'-fluoro and 5'-thiophosphate modified nucleotide
  modified_base 3..5: mod_base = OTHER; note = 2'-methoxy modified nucleotides
  modified_base 6: mod_base = OTHER; note = 2'-fluoro modified nucleotide
  modified_base 7..13: mod_base = OTHER; note = 2'-methoxy modified nucleotides
  modified_base 14: mod_base = OTHER; note = 2'-fluoro modified nucleotide
  modified_base 15: mod_base = OTHER; note = 2'-methoxy modified nucleotide
  modified_base 16: mod_base = OTHER; note = 2'-fluoro modified nucleotide
  modified_base 17..18: mod_base = OTHER; note = 2'-methoxy modified nucleotides
  modified_base 19..20: mod_base = OTHER; note = 2'-methoxy and 5'-thiophosphate modified nucleotides
  modified_base 21: mod_base = OTHER; note = 2'-methoxy modified nucleotide
tttagagtgaggattaaaatg

SEQ ID NO: 23 - RNA, 21 nt, synthetic construct
  source 1..21: mol_type = other RNA
  modified_base 1: mod_base = OTHER; note = 2'-methoxy and 5'-thiovinylphosphonate modified nucleotide
  modified_base 2: mod_base = OTHER; note = 2'-fluoro and 5'-thiophosphate modified nucleotide
  modified_base 3..5: mod_base = OTHER; note = 2'-methoxy modified nucleotides
  modified_base 6: mod_base = OTHER; note = 2'-fluoro modified nucleotide
  modified_base 7..11: mod_base = OTHER; note = 2'-methoxy modified nucleotides
  modified_base 14: mod_base = OTHER; note = 2'-fluoro modified nucleotide
  modified_base 15: mod_base = OTHER; note = 2'-methoxyethyl modified nucleotide
  modified_base 16: mod_base = OTHER; note = 2'-fluoro modified nucleotide
  modified_base 17..18: mod_base = OTHER; note = 2'-methoxy modified nucleotides
  modified_base 19..20: mod_base = OTHER; note = 2'-methoxy and 5'-thiophosphate modified nucleotides
  modified_base 21: mod_base = OTHER; note = 2'-methoxy modified nucleotide
  modified_base 12: mod_base = OTHER; note = 2'-fluoro modified nucleotide
  modified_base 13: mod_base = OTHER; note = 2'-methoxy modified nucleotide
tttagagtgaggattaaaatg

SEQ ID NO: 24 - RNA, 21 nt, synthetic construct
  source 1..21: mol_type = other RNA
  modified_base 1: mod_base = OTHER; note = 2'-methoxy and 5'-thiovinylphosphonate modified nucleotide
  modified_base 2: mod_base = OTHER; note = 2'-fluoro and 5'-thiophosphate modified nucleotide
  modified_base 3..5: mod_base = OTHER; note = 2'-methoxy modified nucleotides
  modified_base 6: mod_base = OTHER; note = 2'-fluoro modified nucleotide
  modified_base 7..8: mod_base = OTHER; note = 2'-methoxy modified nucleotides
  modified_base 14: mod_base = OTHER; note = 2'-fluoro modified nucleotide
  modified_base 15: mod_base = OTHER; note = 2'-methoxyethyl modified nucleotide
  modified_base 16: mod_base = OTHER; note = 2'-fluoro modified nucleotide
  modified_base 17..18: mod_base = OTHER; note = 2'-methoxy modified nucleotides
  modified_base 19..20: mod_base = OTHER; note = 2'-methoxy and 5'-thiophosphate modified nucleotides
  modified_base 21: mod_base = OTHER; note = 2'-methoxy modified nucleotide
  modified_base 13: mod_base = OTHER; note = 2'-methoxy modified nucleotide
  modified_base 9: mod_base = OTHER; note = 2'-fluoro modified nucleotide
  modified_base 10..12: mod_base = OTHER; note = 2'-methoxy modified nucleotides
tttagagtgaggattaaaatg

SEQ ID NO: 25 - RNA, 19 nt, synthetic construct
  source 1..19: mol_type = other RNA
  modified_base 1..2: mod_base = OTHER; note = 2'-methoxy and 5'-thiophosphate modified nucleotides
  modified_base 3..6: mod_base = OTHER; note = 2'-methoxy modified nucleotides
  modified_base 7..10: mod_base = OTHER; note = 2'-fluoro modified nucleotides
  modified_base 11..19: mod_base = OTHER; note = 2'-methoxy modified nucleotides
ttttaatcctcactctaaa

SEQ ID NO: 26 - DNA, 19 bp, synthetic construct
  source 1..19: mol_type = other DNA
ttaactgaaggcgagcatg

SEQ ID NO: 27 - DNA, 18 bp, synthetic construct
  source 1..18: mol_type = other DNA
tgcccaggtctccaacat

SEQ ID NO: 28 - DNA, 21 bp, synthetic construct
  source 1..21: mol_type = other DNA
ccgtgaaaagatgacccagat

SEQ ID NO: 29 - DNA, 18 bp, synthetic construct
  source 1..18: mol_type = other DNA
gccaggtccagacgcagg

(19) United States
(12) Patent Application Publication
(10) Pub. No.: US 2026/0132165 A1
(43) Pub. Date: May 14, 2026

(54) MODIFIED NUCLEOSIDE ANALOGUE AND USE THEREOF, AND NUCLEOSIDE ANALOGUE-CONTAINING DOUBLE-STRANDED OLIGONUCLEOTIDE AND USE THEREOF

(71) Applicant: RIGERNA THERAPEUTICS (BEIJING) CO., LTD., Beijing (CN)

(72) Inventors: Yuanyu Huang, Beijing (CN); Haitao Li, Beijing (CN); Yongxin Gao, Beijing (CN); Hao Song, Beijing (CN); Jing Liu, Beijing (CN)

(73) Assignee: RIGERNA THERAPEUTICS (BEIJING) CO., LTD., Beijing (CN)

(21) Appl. No.: 18/996,323

(22) PCT Filed: May 10, 2024
(86) PCT No.: PCT/CN2024/092369

§ 371 Date: Jan. 5, 2026

(30) Foreign Application Priority Data
May 12, 2023 (CN) 202310540505.6
Dec. 4, 2023 (CN) 202311644617.2

(51) Int. Cl. C07H 19/10; A61K 31/712; A61K 31/713; A61P 25/00; C07H 21/00

(52) U.S. Cl. CPC C07H 19/10; A61K 31/712; A61K 31/713; A61P 25/00; C07H 21/00

(57) ABSTRACT

The present disclosure relates to a nucleoside analogue and a use thereof. The nucleoside analogue is a compound shown in formula (Ia) or a prodrug thereof. The nucleoside analogue can enhance targeted delivery of oligonucleotide drugs to nervous system cells, thereby increasing the inhibition rate of the oligonucleotide drugs delivered in a targeted manner on expression of specific genes in the nervous system cells, or enabling the oligonucleotide drugs delivered in a targeted manner to achieve the purpose of preventing and/or treating pathological conditions or diseases caused by abnormal expression of the specific genes in the nervous system cells. The present disclosure also relates to a nucleotide analogue-containing double-stranded oligonucleotide and a use thereof. The double-stranded oligonucleotide comprises a sense strand and an antisense strand, each strand has 17-25 modified and/or unmodified nucleotides, and the antisense strand and the sense strand are complementary to form a duplex region; the sense strand and/or the antisense strand contains at least one nucleotide analogue represented by the structure of formula (100), or a tautomer, or stereoisomer, or pharmaceutically acceptable salt thereof; the double-stranded oligonucleotide can effectively treat and/or prevent pathological conditions or diseases caused by abnormal expression of specific genes in nervous system cells.

### CROSS-REFERENCE TO RELATED APPLICATIONS

The present disclosure claims the priority to the Chinese patent application with the filing No. 202310540505.6, filed on May 12, 2023 with the Chinese Patent Office and entitled “MODIFIED NUCLEOSIDE ANALOG AND USE THEREOF”, and the Chinese patent application with the filing No. 202311644617.2, filed on Dec. 4, 2023 with the Chinese Patent Office and entitled “NUCLEOTIDE ANALOG-CONTAINING DOUBLE-STRANDED OLIGONUCLEOTIDE AND USE THEREOF”, the contents of which are incorporated herein by reference in entirety.

### TECHNICAL FIELD

The present disclosure pertains to the technical field of bio-pharmaceuticals. Specifically, the present disclosure relates to a modified nucleoside analog and use thereof, and a nucleotide analog-containing oligonucleotide and use thereof. More specifically, the present disclosure relates to a nucleoside analog, a double-stranded oligonucleotide, a composition and uses thereof, a method for inhibiting expression of a specific gene in nervous system cells and a method for preventing and/or treating diseases caused by inhibiting abnormal expression of a specific gene in nervous system cells.

### BACKGROUND ART

RNA interference (RNAi) refers to a phenomenon of highly conserved, double-stranded RNA (dsRNA) induced, and highly efficient specific degradation of homologous mRNA during evolution. Oligonucleotide-containing RNAi agents currently can be delivered to cells in vivo for treatment of diseases caused by abnormal expression of a specific gene in the cells. With regard to treatment of liver-related disorders and diseases, oligonucleotides in RNAi agents can be targeted and delivered to hepatocytes to play a therapeutic role, and have a good application prospect. However, for treatment of extrahepatic tissue diseases, the effect of targeted delivery of oligonucleotides in RNAi agents to extrahepatic cells is poor. At present, oligonucleotides are difficult to be effectively targeted and delivered to the central nervous system (CNS) for treatment of diseases, because free oligonucleotides cannot cross the blood brain barrier (BBB), and a viable way is to deliver oligonucleotides into the CNS through intrathecal delivery. However, using liposome-forming complexes cannot effectively deliver oligonucleotides into target cells of the CNS, resulting in that the oligonucleotides cannot achieve the therapeutic effect in the nervous system cells.

Therefore, there is an urgent need to develop new drugs or methods for targeted delivery of oligonucleotide-containing RNAi agents to the nervous system cells.

### SUMMARY

The present disclosure aims at solving at least one of the technical problems existing in the prior art at least to some extent. In view of this, the present disclosure provides a nucleoside analog, wherein the nucleoside analog can improve effect of targeted delivery of oligonucleotide drugs on nervous system cells, so as to improve an inhibition rate of the targeted delivery of oligonucleotide drugs on expression of a specific gene in the nervous system cells, or to enable the targeted delivery of oligonucleotide drugs to achieve a purpose of preventing and/or treating diseases caused by abnormal expression of the specific gene in the nervous system cells.

The disclosure further provides a nucleotide analog, a nucleotide analog-containing double-stranded oligonucleotide and uses thereof. In the present disclosure, by introducing the nucleotide analog into the double-stranded oligonucleotide, modified oligonucleotide molecules can improve an effect of targeted delivery of oligonucleotide drugs on the nervous system cells, and can be used for treating and/or preventing pathological conditions or diseases caused by abnormal expression of a specific gene in the nervous system cells.

In the first aspect of the present disclosure, the present disclosure provides a nucleoside analog, which is a compound as represented by Formula (Ia) or a prodrug thereof:

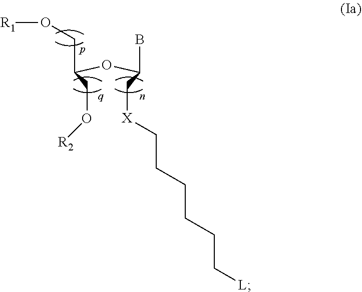

wherein R₁ is selected from the group consisting of H or a hydroxyl protecting group;
R₂ is selected from the group consisting of H or a phosphorus-containing leaving group;
B is selected from the group consisting of substituted or unsubstituted nucleobase, or substituted or unsubstituted nucleobase analog, each substituent in B is independently selected from the group consisting of halogen, C₁-C₃ alkyl, C₁-C₃ alkyloxy, or substituted amide group; and if an amino is present in the B, the amino is protected with an amino protecting group;
X is selected from the group consisting of O, S, NH or —NH—C(═O)—;
L is selected from the group consisting of substituted or unsubstituted C₄-C₁₂ hydrocarbyl, and each substituent in L is independently selected from the group consisting of halogen, C₁-C₃ alkyl or C₁-C₃ alkoxy;
p and q are each independently selected from 1, 2, 3 or 4; and
n is selected from 1, 2 or 3.

In the second aspect of the present disclosure, the present disclosure provides a nucleoside analog, which is a compound as represented by Formula (Ib), Formula (Ic) or Formula (Id) or a prodrug thereof:

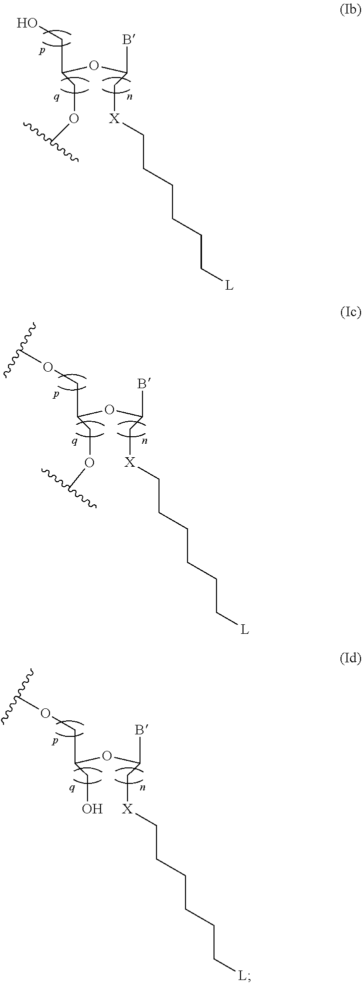

wherein R₁ is selected from the group consisting of H or a hydroxyl protecting group;
R₂ is selected from the group consisting of H or a phosphorus-containing leaving group;
B′ is selected from the group consisting of substituted or unsubstituted nucleobase or substituted or unsubstituted nucleobase analog; each substituent in B′ is independently selected from the group consisting of halogen, C₁-C₃ alkyl, C₁-C₃ alkyloxy, or substituted amide group; and if an amino is present in the B′, the amino is not protected;
X is selected from the group consisting of O, S, NH or —NH—C(═O)—;
L is selected from the group consisting of substituted or unsubstituted C₄-C₁₂ hydrocarbyl, and each substituent in L is independently selected from the group consisting of halogen, C₁-C₃ alkyl or C₁-C₃ alkoxy;
p and q are each independently selected from 1, 2, 3 or 4; and
n is selected from 1, 2 or 3.

In the third aspect of the present disclosure, the present disclosure provides use of the nucleoside analog according to the first aspect, and/or the nucleoside analog according to the second aspect in preparation of oligonucleotides.

In the fourth aspect of the present disclosure, the present disclosure provides a double-stranded oligonucleotide, wherein the double-stranded oligonucleotide comprises a sense strand and an antisense strand, the antisense strand and the sense strand have a complementary region with bases at least partially paired, and the sense strand and/or the antisense strand contain at least one nucleoside analog according to the second aspect.

In the fifth aspect of the present disclosure, the present disclosure provides a composition. According to embodiments of the present disclosure, the composition comprises the double-stranded oligonucleotide according to the fourth aspect.

In the sixth aspect of the present disclosure, the present disclosure provides use of the nucleoside analog according to the first aspect, the nucleoside analog according to the second aspect, the double-stranded oligonucleotide according to the fourth aspect or the composition according to the fifth aspect in preparation of medicaments for treating and/or preventing pathological conditions or diseases caused by abnormal expression of a specific gene in target cells.

In the seventh aspect of the present disclosure, the present disclosure provides a method for inhibiting expression of a specific gene in target cells, wherein the method comprises: contacting the double-stranded oligonucleotide according to the fourth aspect or the composition according to the fifth aspect with the target cells.

In the eighth aspect of the present disclosure, the present disclosure provides a method for preventing and/or treating pathological conditions or diseases caused by abnormal expression of a specific gene in target cells, wherein the method comprises: administering to a subject a pharmaceutically acceptable dose of the double-stranded oligonucleotide according to the fourth aspect or the composition according to the fifth aspect.

In the ninth aspect of the present disclosure, the present disclosure provides a nucleotide analog as represented by (100), or a tautomer thereof, or a stereoisomer thereof, or a pharmaceutically acceptable salt thereof:

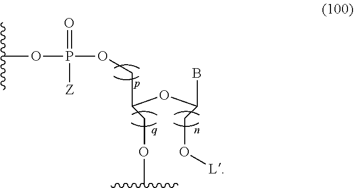

wherein [symbol] represents a covalently linking position of the nucleotide analog;
B is selected from the group consisting of substituted or unsubstituted nucleobase, or substituted or unsubstituted nucleobase analog; and if the B contains a substituent, the substituent is independently selected from the group consisting of halogen, C₁-C₃ alkyl, C₁-C₃ alkyloxy, or amide group;
L′ is selected from the group consisting of substituted or unsubstituted C₁₆-C₂₄ hydrocarbyl; and if the L′ contains a substituent, the substituent is independently selected from the group consisting of halogen, C₁-C₃ alkyl or C₁-C₃ alkoxy;
p is selected from 1, 2, 3 or 4;
q is selected from 1, 2, 3 or 4;
n is selected from 1, 2 or 3; and
Z is selected from the group consisting of hydroxyl or thiol.

In the tenth aspect of the present disclosure, the present disclosure provides a double-stranded oligonucleotide, wherein the double-stranded oligonucleotide comprises a sense strand and an antisense strand, each strand has 17-25 modified and/or unmodified nucleotides, the antisense strand and the sense strand are complementary to form a duplex region; and the sense strand and/or the antisense strand contain at least one nucleotide analog according to the ninth aspect of the present disclosure, or a tautomer thereof, or a stereoisomer thereof, or a pharmaceutically acceptable salt thereof:

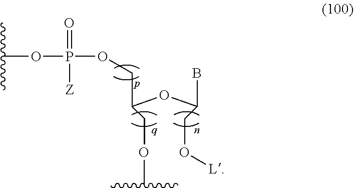

wherein [symbol] represents a covalently linking position between the nucleotide analog and a neighboring nucleotide;
B is selected from the group consisting of substituted or unsubstituted nucleobase, or substituted or unsubstituted nucleobase analog; and if the B contains a substituent, the substituent is independently selected from the group consisting of halogen, C₁-C₃ alkyl, C₁-C₃ alkyloxy, or amide group;
L′ is selected from the group consisting of substituted or unsubstituted C₁₆-C₂₄ hydrocarbyl; and if the L′ contains a substituent, the substituent is independently selected from the group consisting of halogen, C₁-C₃ alkyl or C₁-C₃ alkoxy;
p is selected from 1, 2, 3 or 4;
q is selected from 1, 2, 3 or 4;
n is selected from 1, 2 or 3; and
Z is selected from the group consisting of hydroxyl or thiol.

In some embodiments of the present disclosure, the sense stand contains one of the nucleotide analogs.

In some embodiments of the present disclosure, the nucleoside analog is located at Positions 2-8 of the sense strand counting from 5′-end.

In some embodiments of the present disclosure, the antisense strand does not contain the nucleotide analog, and the antisense strand is basically reversely complementary, substantially reversely complementary or fully reversely complementary to a nucleotide sequence in an mRNA expressed by the target gene.

In some optional embodiments of the present disclosure, nucleotides of the double-stranded oligonucleotide other than the nucleotide replaced with the nucleotide analog are all modified, and are each independently selected from the following modified nucleotides: 2′-O-methoxy modified nucleotides, 2′-fluoro modified nucleotides and 2′-O-methoxyethyl (2′-O-MOE) modified nucleotides.

In the eleventh aspect of the present disclosure, the present disclosure further provides a composition, which comprises the double-stranded oligonucleotide according to the tenth aspect of the present disclosure.

In the twelfth aspect of the present disclosure, the present disclosure further provides use of the nucleotide analog according to the ninth aspect of the present disclosure, and/or the double-stranded oligonucleotide according to the tenth aspect of the present disclosure, and/or the composition according to the eleventh aspect of the present disclosure in preparation of medicaments for treating and/or preventing pathological conditions or diseases caused by abnormal expression of a specific gene in nervous system cells.

In the thirteenth aspect of the present disclosure, the present disclosure further provides a pharmaceutical composition, which comprises the double-stranded oligonucleotide according to the tenth aspect of the present disclosure, and/or the composition according to the eleventh aspect of the present disclosure.

In some embodiments of the present disclosure, the composition further contains optionally one or more pharmaceutically acceptable carriers or adjuvants.

In the fourteenth aspect of the present disclosure, the present disclosure further provides a medicament, which comprises the double-stranded oligonucleotide according to the tenth aspect of the present disclosure, and/or the composition according to the eleventh aspect of the present disclosure, and/or the pharmaceutical composition according to the thirteenth aspect of the present disclosure.

In the fifteenth aspect of the present disclosure, the present disclosure further provides a method for reducing expression of a gene in target cells, wherein the method comprises: contacting the double-stranded oligonucleotide according to the tenth aspect of the present disclosure, and/or the composition according to the eleventh aspect of the present disclosure, and/or the pharmaceutical composition according to the thirteenth aspect of the present disclosure, and/or the medicament according to the fourteenth aspect of the present disclosure with the target cell.

In the sixteenth aspect of the present disclosure, the present disclosure further provides a method for reducing expression of a target gene in a subject, wherein the method comprises: administering to the subject the nucleotide analog according to the ninth aspect of the present disclosure, and/or the double-stranded oligonucleotide according to the tenth aspect of the present disclosure, and/or the composition according to the eleventh aspect of the present disclosure, and/or the pharmaceutical composition according to the thirteenth aspect of the present disclosure, and/or the medicament according to the fourteenth aspect of the present disclosure.

In the seventeenth aspect of the present disclosure, the present disclosure further provides a method for preventing and/or treating pathological conditions or diseases caused by abnormal expression of a specific gene in target cells, wherein the method comprises: administering to the subject a pharmaceutically acceptable dose of the double-stranded oligonucleotide according to the tenth aspect of the present disclosure, and/or the composition according to the eleventh aspect of the present disclosure, and/or the pharmaceutical composition according to the thirteenth aspect of the present disclosure, and/or the medicament according to the fourteenth aspect of the present disclosure.

In the eighteenth aspect of the present disclosure, the present disclosure further provides a method for treating a subject suffering from CNS disorder-associated diseases or conditions, wherein the method comprises:
administering to the subject a therapeutically effective amount of the double-stranded oligonucleotide according to the tenth aspect of the present disclosure, and/or the composition according to the eleventh aspect of the present disclosure, and/or the pharmaceutical composition according to the thirteenth aspect of the present disclosure, and/or the medicament according to the fourteenth aspect of the present disclosure, so as to treat the CNS disorder-associated diseases or conditions of the subject.

In the nineteenth aspect of the present disclosure, the present disclosure provides a kit, wherein the kit comprises the nucleotide analog according to the ninth aspect of the present disclosure, and/or the double-stranded oligonucleotide according to the tenth aspect of the present disclosure, and/or the composition according to the eleventh aspect of the present disclosure, and/or the pharmaceutical composition according to the thirteenth aspect of the present disclosure, and/or the medicament according to the fourteenth aspect of the present disclosure.

The double-stranded oligonucleotide and composition provided in the present disclosure can effectively treat and/or prevent pathological conditions or diseases caused by abnormal expression of a specific gene in the nervous system cells

In the twentieth aspect of the present disclosure, the present disclosure further provides a method for inhibiting expression of a target gene in skeletal muscle cells, ocular cells, myocardial cells or adipose cells, wherein the method comprises: administering to the subject a therapeutically effective amount of the double-stranded oligonucleotide according to the tenth aspect of the present disclosure, and/or the composition according to the eleventh aspect of the present disclosure, and/or the pharmaceutical composition according to the thirteenth aspect of the present disclosure.

In the twenty-first aspect of the present disclosure, the present disclosure further provides a method for preventing and/or treating diseases or conditions associated with skeletal muscles, cardiac muscles, eyes or adipose tissues, wherein the method comprises: administering to the subject a therapeutically effective amount of the double-stranded oligonucleotide according to the tenth aspect of the present disclosure, and/or the composition according to the eleventh aspect of the present disclosure, and/or the pharmaceutical composition according to the thirteenth aspect of the present disclosure.

Additional aspects and advantages of the present disclosure will be partially given in the following description, and partially become apparent from the following description, or be comprehended by practicing the present disclosure.

### BRIEF DESCRIPTION OF DRAWINGS

The above and/or additional aspects and advantages of the present disclosure will become apparent and easily understood from the description of embodiments in conjunction with the drawings attached below. In the drawings:

FIG. 1 is a diagram of relative expression level of target gene (SOD1) of interest in different parts of brain regions of mice in RZ899008 group and PBS group in Test Example 1 of the present disclosure;

FIG. 2 is a diagram of relative expression level of target gene (SOD1) of interest in lumbar spine samples of various groups of mices in Test Example 2 of the present disclosure;

FIG. 3 is a diagram of relative expression level of target gene (SOD1) of interest in thoracic spine samples of various groups of mices in Test Example 2 of the present disclosure;

FIG. 4 is a diagram of relative expression level of target gene (SOD1) of interest in cerebellum samples of various groups of mices in Test Example 2 of the present disclosure;

FIG. 5 shows relative remaining expression level of target gene of interest in mouse cerebellum after administration of the siRNAs in Test Example 3;

FIG. 6 shows relative remaining expression level of target gene of interest of mice after administration of the siRNAs in Test Example 4;

FIG. 7 shows relative remaining expression level of target gene of interest in rats after administration of the siRNAs in Test Example 5;

FIG. 8 shows relative expression level of target gene of interest in rats after administration of the siRNA conjugates in Test Example 6;

FIG. 9 shows relative expression level of target gene of interest in mouse heart tissues after administration of the siRNA conjugates in Test Example 7;

FIG. 10 shows relative expression level of target gene of interest in mouse adipose tissues after administration of the siRNA conjugates in Test Example 7;

FIG. 11 shows relative expression level of target gene of interest in mouse skeletal muscle tissues after administration of the siRNA conjugates in Test Example 7;

FIG. 12 shows relative expression level of target gene of interest in mouse liver tissues after administration of the siRNA conjugates in Test Example 7;

FIG. 13 shows relative expression level of target gene of interest in mouse kidney tissues after administration of the siRNA conjugates in Test Example 7;

FIG. 14 shows relative expression level of target gene of interest in mouse adipose tissues after administration of the siRNA conjugates in Test Example 8;

FIG. 15 shows relative expression level of target gene of interest in mice after administration of the siRNA conjugates in Test Example 9;

FIG. 16 shows relative expression level of target gene of interest in mouse heart tissues after administration of the siRNA conjugate in Test Example 10;

FIG. 17 shows relative expression level of target gene of interest in mouse adipose tissues after administration of the siRNA conjugate in Test Example 10;

FIG. 18 shows relative expression level of target gene of interest in mouse heart tissues after administration of the siRNA conjugate in Test Example 11;

FIG. 19 shows relative expression level of target gene of interest in mouse ocular tissues after administration of the siRNA conjugate in Test Example 11;

FIG. 20 shows relative expression level of target gene of interest in mouse adipose tissues after administration of the siRNA conjugate in Test Example 12.

### DETAILED DESCRIPTION OF EMBODIMENTS

Embodiments of the present disclosure will be described in detail below. The embodiments described below are illustrative, merely for explaining the present disclosure, but should not be construed as limitation to the present disclosure.

### Definitions and General Terms

It should be noted that the terms “first” and “second” are merely used for descriptive purpose, but should not be construed as indicating or implying importance in the relativity or implicitly indicating the number of a related technical feature. Thus, defining a feature with “first” or “second” may explicitly or implicitly mean that one or more such features are comprised. Further, in the description of the present disclosure, unless otherwise stated, “a plurality of” means two or more.

In the context of the present disclosure, unless otherwise stated, “nucleic acid analog” in the present disclosure can be present independently in a salt, mixed salt or non-salt (e.g., free acid or free base) form. When being present in the form of salt or mixed salt, it can be a pharmaceutically acceptable salt.

The term “pharmaceutically acceptable” herein means that a substance or composition must be chemically and/or toxicologically compatible with other ingredients containing a nucleic acid analog and/or mammals being treated therewith. Preferably, the “pharmaceutically acceptable” in the present disclosure means approved by a federal regulatory agency or a state government or listed in the U.S. Pharmacopeia or other generally recognized pharmacopeias for use in animals, particularly in humans.

In the context of the present disclosure, “pharmaceutically acceptable salts” comprises pharmaceutically acceptable acid addition salts and pharmaceutically acceptable base addition salts.

In the context of the present disclosure, “pharmaceutically acceptable acid addition salts” refers to those salts that can retain biological effectiveness of free bases without other side effects and that are formed with inorganic or organic acids. Inorganic acid salts comprise, but are not limited to, hydrochloride, hydrobromide, sulphate, nitrate, phosphate, and the like, and organic acid salts comprise, but are not limited to, formate, acetate, 2,2-dichloroacetate, trifluoroacetate, propionate, hexanoate, octanoate, decanoate, undecylenate, glycolate, gluconate, lactate, sebacate, adipate, glutarate, malonate, oxalate, maleate, succinate, fumarate, tartrate, citrate, palmitate, stearate, oleate, cinnamate, laurate, malate, glutamate, pyroglutamate, aspartate, benzoate, methanesulfonate, benzenesulfonate, p-toluenesulfonate, alginate, ascorbate, salicylate, 4-aminosalicylate, naphthalenedisulfonate, and the like. These salts can be prepared by methods known in the art.

In the context of the present disclosure, “pharmaceutically acceptable base addition salts” refers to those salts that can retain biological effectiveness of free acids without other side effects and that are formed with inorganic or organic bases. Salts derived from inorganic bases comprise, but are not limited to, sodium, potassium, lithium, ammonium, calcium, magnesium, iron, zinc, copper, manganese, aluminum salts, and the like. Preferred inorganic salts are ammonium, sodium, potassium, calcium and magnesium salts, preferably sodium salts. Salts derived from organic bases comprise, but are not limited to, salts of primary, secondary and tertiary amines, substituted amines including naturally occurring substituted amines, cyclic amines and basic ion exchange resins, such as ammonia, isopropylamine, trimethylamine, diethylamine, triethylamine, tripropylamine, ethanolamine, diethanolamine, triethanolamine, dimethylethanolamine, 2-dimethylaminoethanol, 2-diethylaminoethanol, dicyclohexylamine, lysine, arginine, histidine, caffeine, procaine, choline, betaine, ethylenediamine, glucosamine, methylglucamine, theobromine, purines, piperazine, piperidine, N-ethylpiperidine, polyamine resins, and the like. Preferred organic bases comprise isopropylamine, diethylamine, ethanolamine, trimethylamine, dicyclohexylamine, choline and caffeine. These salts can be prepared by methods known in the art.

The term “prodrug” as used in the present disclosure represents a compound that is converted in vivo to a compound as represented by Formula (I). Such conversion is affected by hydrolysis of a precursor drug in blood or by enzymatic conversion of the prodrug to a parent structure in blood or tissue. Precursor drug compounds in the present disclosure can be esters, and in existing inventions, esters that can be used as precursor drugs comprise phenyl esters, aliphatic (C₁-C₂₄) esters, acetyloxymethyl esters, carbonates, carbamates and amino acid esters. For example, a compound in the present disclosure that contains a hydroxyl group can be acylated to provide a compound in a precursor drug form. Other precursor drug forms comprise phosphates, for example, these phosphate compounds are obtained by phosphorylation of hydroxyl on the parent. A thorough discussion of precursor drugs can be found in the following documents: T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987, J. Rautio et al., Prodrugs: Design and Clinical Applications, Nature Review Drug Discovery, 2008, 7, 255-270, and S. J. Hecker et al., Prodrugs of Phosphates and Phosphonates, Journal of Medicinal Chemistry, 2008, 51, 2328-2345.

In the context of the present disclosure, “pharmaceutical composition” can be used for treatment for diseases, and can also be used for in vitro culture experiments of cells. For the treatment of diseases, the term “pharmaceutical composition” generally refers to a unit dosage form, and can be prepared by any of well-known methods in the pharmaceutical art. All the methods comprise a step of combining an active ingredient with an adjuvant constituting one or more accessory ingredients. Generally, the composition is prepared by uniformly and sufficiently combining active siRNA with a liquid adjuvant, a finely crushed solid adjuvant or both.

The term “comprising” or “including” herein is an open-ended expression, i.e., including what is specified in the present disclosure, but not excluding contents in other aspects.

The term “optionally” or “optional” herein generally mean that the event or case subsequently described can but may not occur, and that the description comprises situations where the event or case occurs, and situations where the event or case does not occur.

The term “optionally substituted” can be used interchangeably with the term “unsubstituted or substituted with . . . ”, that is, the structure is unsubstituted or substituted with one or more substituents described in the present disclosure, and the substituents described in the present disclosure comprise, but are not limited to, D, F, Cl, Br, I, N₃, —CN, —NO₂, —NH₂, —OH, —SH, —COOH, —CONH₂, —C(═O)NHCH₃, —C(═O)N(CH₃)₂, —C(═O)-alkyl, —C(═O)-alkoxy, alkyl, alkenyl, alkynyl, haloalkyl, alkoxy, haloalkoxy, alkylthio, alkylamino, hydroxy-substituted alkyl, cyano-substituted alkyl, amino-substituted alkyl, (alkoxy)-alkylene, (alkylamino)-alkylene, (cycloalkyl)-alkylene, (heterocyclyl)-alkylene, (aryl)-alkylene, (heteroaryl)-alkylene, cycloalkyl, heterocyclyl, aryl, heteroaryl, and the like.

Generally, the terms “substituted” means that any one or more hydrogen atoms in a given structure is replaced with a specific substituent (e.g., C₁₋₃ alkyl, C₁₋₃ alkoxy or halo) provided that a normal valence of the specified atom is not exceeded and substitution results in a stable compound. Unless otherwise indicated, a substituted group may have a substituent that performs substitution at various substitutable positions of the group. When more than one position in a given structure can be substituted with one or more substituents selected from specific groups, the substituents can be substitute at various substitutable positions in the same or different ways.

In the context of the present disclosure, the terms “each . . . independently selected from”, “ . . . each independently selected from“and” . . . independently selected from” are used interchangeably, and all should be interpreted in a broad sense, meaning that specific options expressed between the same symbol in different groups do not affect each other, and that specific options expressed between the same symbol in the same group do not affect each other. The term “hydrocarbyl” refers to a group formed by removing one or more hydrogen atoms from a hydrocarbon molecule (hydrocarbon), and it comprises monovalent saturated radical (also called as alkyl), monovalent unsaturated radical (also called as alkenyl and alkynyl), divalent radical (also called as subunit) or trivalent radical (also called as ylidyne).

The term “alkyl” has a general structural formula

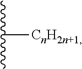

and alkyl can be linear alkyl or branched alkyl. Exemplarily, the term “C₁-C₃ alkyl” refers to a chained alkyl having 1 to 3 carbon atoms; and the term “C₄-C₁₂ alkyl” refers to a chained alkyl having 4 to 12 carbon atoms.

The term “alkylene” has a general structural formula

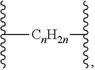

and alkylene can be linear alkyl or branched alkyl. Exemplarily, the term “methylene” has a general structural formula —CH₂—.

The term “alkyloxy” has a general structural formula

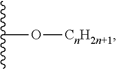

wherein alkyl can be linear alkyl or branched alkyl.

The term “alkanoyl” has a general structural formula

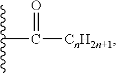

wherein alkyl can be linear alkyl or branched alkyl.

The term “aryl” represents monocyclic, bicyclic and tricyclic carbocycle system having 5-10 ring atoms, or 6-10 ring atoms, wherein at least one ring system is aromatic, wherein each ring system contains a ring consisting of 3-7 atoms. An aryl group is generally, but not necessarily, linked to a parent molecule via an aromatic ring of the aryl group. The term “aryl” can be used interchangeably with the term “aromatic ring” or “aryl ring”. The aryl group can comprise phenyl, indenyl, naphthyl and anthryl. The aryl group is optionally substituted with one or more substituents described in the present disclosure.

The term “aryloxy” is —O-aryl, wherein the term definition of aryl is given above.

The term “arylcarbonyl” is —CO-aryl, wherein the term definition of aryl is given above.

The term “NH” refers to imidogen, with a structural formula

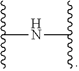 [SMILES: *N*]

The term “CO” or “C(═O)” refers to carbonyl, with a structural formula

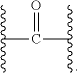 [SMILES: *C(*)=O]

The term “—NH—CO—” or “—NH—C(═O)—” has a structural formula

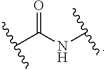 [SMILES: *NC(*)=O]

The terms “halogen” and “halo” are used interchangeably in the present disclosure, and refer to fluorine (F), chlorine (Cl), bromine (Br) or iodine (I).

The term “trityl” has a structural formula

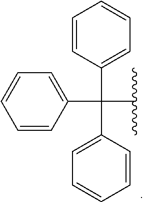 [SMILES: *C(c1ccccc1)(c1ccccc1)c1ccccc1]

The term “4-methoxytrityl” has a structural formula

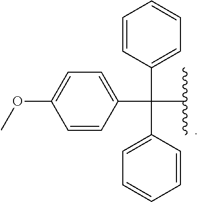 [SMILES: *C(c1ccccc1)(c1ccccc1)c1ccc(OC)cc1]

The term “4,4′-dimethoxytrityl” has a structural formula

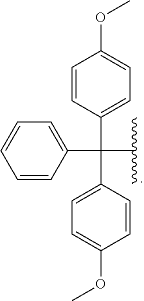 [SMILES: *C(c1ccccc1)(c1ccc(OC)cc1)c1ccc(OC)cc1]

The term “4,4′,4″-trimethoxytriphenyl” has a structural formula

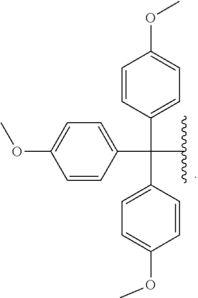 [SMILES: *C(c1ccc(OC)cc1)(c1ccc(OC)cc1)c1ccc(OC)cc1]

In the context of the present disclosure, the term “stereoisomers” refers to compounds that have the same chemical constitution, but differ in arrangement of atoms or groups in space. Stereoisomers comprise enantiomers, diastereomers, conformers (rotamers), geometric (cis/trans) isomers, atropisomers, etc.

In the context of the present disclosure, the term “chiral” means that a molecule has the property of being non-superimposable with a mirror image thereof; and “achiral” means that a molecule is superimposable with a mirror image thereof.

In the context of the present disclosure, the term “enantiomers” refers to two isomers of a compound that are non-superimposable but have a relationship of being mirror image to each other.

In the context of the present disclosure, the term “diastereomers” refers to stereoisomers that have two or more centers of chirality and molecules being not mirror images to one another. Diastereomers have different physical properties, such as melting points, boiling points, spectral properties and reactivities. Mixtures of diastereomers can be separated under high-resolution analytical procedures, such as electrophoresis and chromatography, e.g., HPLC.

In the context of the present disclosure, the term “ligand” generally refers to any compound or molecule capable of binding to a biologically active substance (such as an oligonucleotide) covalently or otherwise chemically. In certain embodiments, the ligand is capable of interacting directly or indirectly with another compound, such as a receptor, the receptor interacting with the ligand can be present on a cell surface, or alternatively can be an intracellular and/or an intercellular receptor, and interaction of the ligand with the receptor may cause a biochemical reaction, or may simply be a physical interaction or binding.

The term “ [symbol] ” represents a site at which a group is linked by a covalent bond.

In chemical structures of the ligand or compound of the present disclosure, bond “ [symbol] ” represents an unspecified configuration. If a chiral isomerism is present in a chemical structure, the bond “ [symbol] ” can be “ [symbol] ”, “ [symbol] ” or contain both “ [symbol] ” and “ [symbol] ” configurations. While all of the above structures are depicted in certain isomeric forms for simplicity, the present disclosure may comprise all isomers, such as, tautomers, rotamers, geometric isomers, diastereomers, racemates and enantiomers.

In chemical structures of the ligands or compounds in the present disclosure, bond “ [symbol] ” represents an unspecified configuration. If cis-trans isomerism is present in a chemical structure, configuration of the bond “ [symbol] ” can be E-configuration, Z-configuration, or contain both E and Z configurations.

In the context of the present disclosure, unless otherwise stated, “selected from . . . and . . . ” in the present disclosure means that at least one therein can be selected.

In the context of the present disclosure, unless otherwise stated, “is . . . or . . . ” in the present disclosure means that at least one therein can be selected.

In the context of the present disclosure, “oligonucleotide” is a deoxyribonucleic acid (DNA) or ribonucleic acid (RNA), generally composed of 10-50 nucleotides. Oligonucleotides can regulate gene expression through a series of processes such as ribonucleic acid interference, ribonuclease-mediated target degradation, splicing regulation, non-coding RNA suppression, gene activation and programmed gene editing.

In the context of the present disclosure, the term “small interfering RNA (siRNA)” is a double-stranded RNA having a length of 17 to 25 nucleotides, and comprises a sense strand and an antisense strand. siRNA mediates RNA transcript targeted cleavage of RISC pathway by forming a silencing complex (RNA-induced silencing complex, RISC). Specifically, siRNA guides specific degradation of mRNA sequence through a known RNA interference (RNAi) process, and inhibits translation of mRNA into amino acid and conversion to protein.

In the context of the present disclosure, the term “antisense strand (or guide strand)” comprises a region that is basically complementary to a target sequence. The term “sense strand (or passenger strand)” refers to a strand that contains an iRNA that is basically complementary to the antisense strand. The term “basically complementary” means fully complementary or at least partially complementary, for example, the antisense strand is fully complementary or at least partially complementary to the target sequence. In a partially complementary case, mispairing may occur within internal or terminal region of molecule, wherein the most tolerated mispairing occurs within the terminal region, for example, within 5, 4, 3 or 2 nucleotides at 5′- and/or 3′-end of the iRNA. It should be noted that the antisense strand is “at least partially basically complementary” to an mRNA means that the antisense strand has a polynucleotide that is basically complementary to a contiguous portion of the mRNA of interest.

In the context of the present disclosure, “antisense oligonucleotides (ASO)” is a single-stranded oligonucleotide molecule, generally composed of 10-50 nucleotides. After entering a cell, ASO binds to a complementary target mRNA under the action of ribonuclease H1 through the base complementary pairing principle, so as to inhibit expression of target gene.

In the context of the present disclosure, the terms “complementary” and “reversely complementary” can be used interchangeably, and have the meaning well known to those skilled in the art that, namely, in a double-stranded nucleic acid molecule, bases in one strand are paired complementarily with bases in the other strand. In DNA, a purine base adenine (A) is always paired with a pyrimidine base thymine (T) (or a uracil (U) in RNA); and a purine base guanine (G) is always paired with a pyrimidine base cytosine (C). Each base pair comprises a purine and a pyrimidine. When adenines in one strand are always paired with thymines (or uracils) in the other strand, and guanines are always paired with cytosines, the two strands are considered as being complementary to each other, and a sequence of a strand can be deduced from a sequence of its complementary strand. Correspondingly, “mispairing” in the art means that bases at corresponding positions are not present in a form of being complementarily paired in a double-stranded nucleic acid.

In the context of the present disclosure, unless otherwise stated, “basically reversely complementary” means that there are no more than 3 base mispairings between two nucleotide sequences involved; “substantially reversely complementary” means that there is no more than 1 base mispairing between two nucleotide sequences; and “fully complementary” means that there is no base mispairing between two nucleotide sequences.

In the context of the present disclosure, unless otherwise stated, “complementary region with bases at least partially paired” means there is a contiguous portion of basically complementary polynucleotides between the two nucleotide strands, for example, no less than 3, 5, 10, or 15 nucleotides being complementary.

In the context of the present disclosure, particularly when describing a preparation method for the double-stranded oligonucleotide or composition of the present disclosure, unless specifically stated, the nucleoside monomer refers to, according to a type and order of nucleotides in an oligonucleotide or composition to be prepared, a modified or unmodified nucleoside monomer used in phosphoramidite solid phase synthesis. Phosphoramidite solid phase synthesis is a method well known to those skilled in the art to be used in synthesis of RNA. The nucleoside monomers used in the present disclosure are all commercially available.

The term “pharmaceutically acceptable carrier” comprises any solvent, drug stabilizer, or combination thereof, and all of these carriers are known to those skilled in the art (for example, as described in Remington's Pharmaceutical Sciences, 18th Ed. Mack Printing Company, 1990, pp. 1289-1329). Except insofar as any conventional carrier is incompatible with an active ingredient, use thereof in a therapeutic or pharmaceutical composition is encompassed.

The term “pharmaceutically acceptable adjuvant” may comprise any solvent and the like, suitable for a particular dosage form of interest. Except insofar as any conventional adjuvant is incompatible with the nucleic acid analog of the present disclosure, for example, any undesirable biological effects produced or interaction with any other components of the pharmaceutically acceptable composition in a deleterious manner, their use is also within the scope contemplated by the present disclosure.

In the context of the present disclosure, the term “administering” generally refers to introducing a pharmaceutical formulation of the present disclosure into the body of subjects by any route of introduction or delivery. Any method known to those skilled in the art for contacting cells, organs or tissues with the drug can be employed. The administration may comprise, but is not limited to, intravenous, intraarterial, intranasal, intraperitoneal, intramuscular, subcutaneous or oral administration. A daily dosage can be divided into one, two or more suitable forms of dose to be administered at one, two or more time points in a certain period of time.

As used herein, “treating”, “alleviating” or “ameliorating” can be used interchangeably herein. These terms refer to an approach for obtaining beneficial or desired results, including but not limited to therapeutic benefits. By “therapeutic benefit” is meant eradication or amelioration of underlying disorder being treated. The therapeutic benefit herein is achieved by eradicating or ameliorating one or more physiological symptoms associated with the underlying disorder such that amelioration is observed in the subjects, notwithstanding that the subjects may still be afflicted with the underlying disorder.

The terms “preventing” and “avoiding”, as used herein, can be used interchangeably. These terms refer to an approach for obtaining beneficial or desired results, including but not limited to prophylactic benefit. For “prophylactic benefit, the double-stranded oligonucleotide or composition can be administered to subjects at risk of developing a particular disease, or to subjects reporting one or more physiological symptoms of a disease, even though a diagnosis of this disease may not have been made.

The term “subject”, as used herein, refers to any animal, for example, a mammal or a marsupial. The subject of the present disclosure comprises, but is not limited to, human, non-human primate (e.g., rhesus or other types of macaques), mouse, pig, horse, donkey, cow, sheep, rat or any kind of poultry.

In the present disclosure, the term “regulating (modulating) gene expression” means up-regulating or down-regulating expression of a gene, or a level of RNA molecule or equivalent RNA molecule encoding one or more proteins or protein subunits, so that expression, level or activity is greater or less than that observed in the absence of a regulator. For example, the term “regulating” can mean “inhibiting”, but use of the word “regulating” is not limited to this definition.

In the first aspect of the present disclosure, the present disclosure provides a nucleoside analog, which is a compound as represented by Formula (Ia) or a prodrug thereof:

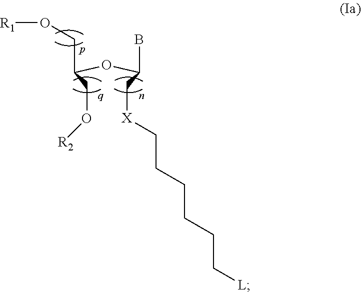

wherein R₁ is selected from the group consisting of H or a hydroxyl protecting group;
R₂ is selected from the group consisting of H or a phosphorus-containing leaving group;
B is selected from the group consisting of substituted or unsubstituted nucleobase, or substituted or unsubstituted nucleobase analog, each substituent in B is independently selected from the group consisting of halogen, C₁-C₃ alkyl, C₁-C₃ alkyloxy, or substituted amide group; and if an amino is present in the B, the amino is protected with an amino protecting group;
X is selected from the group consisting of O, S, NH or —NH—C(═O)—;
L is selected from the group consisting of substituted or unsubstituted C₄-C₁₂ hydrocarbyl, and each substituent in L is independently selected from the group consisting of halogen, C₁-C₃ alkyl or C₁-C₃ alkoxy;
p and q are each independently selected from 1, 2, 3 or 4; and
n is selected from 1, 2 or 3.

In the present disclosure, the substituted amide group has a structural formula

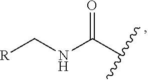

wherein R can be selected from the group consisting of

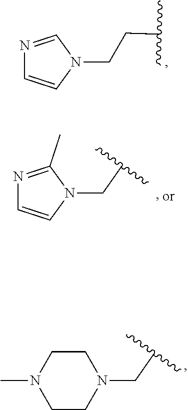

etc.

In some optional embodiments of the present disclosure, L is selected from the group consisting of substituted or unsubstituted C₄-C₁₂ hydrocarbyl, substituted or unsubstituted C₄-C₁₁ hydrocarbyl, substituted or unsubstituted C₄-C₁₀ hydrocarbyl, substituted or unsubstituted C₅-C₁₂ hydrocarbyl, substituted or unsubstituted C₅-C₁₁ hydrocarbyl, substituted or unsubstituted C₅-C₁₀ hydrocarbyl, substituted or unsubstituted C₆-C₁₂ hydrocarbyl, substituted or unsubstituted C₆-C₁₁ hydrocarbyl, substituted or unsubstituted C₆-C₁₀ hydrocarbyl, substituted or unsubstituted C₇-C₁₂ hydrocarbyl, substituted or unsubstituted C₇-C₁₁ hydrocarbyl, substituted or unsubstituted C₇-C₁₀ hydrocarbyl, substituted or unsubstituted C₈-C₁₂ hydrocarbyl, substituted or unsubstituted C₈-C₁₁ hydrocarbyl, substituted or unsubstituted C₈-C₁₀ hydrocarbyl, substituted or unsubstituted C₉-C₁₂ hydrocarbyl, substituted or unsubstituted C₉-C₁₁ hydrocarbyl, or substituted or unsubstituted C₉-C₁₀ hydrocarbyl; and each substituent in L is independently selected from the group consisting of halogen, C₁-C₃ alkyl or C₁-C₃ alkyloxy.

According to embodiments of the present disclosure, L is selected from the group consisting of substituted or unsubstituted linear or branched C₄-C₁₂ alkenyl, or substituted or unsubstituted linear or branched C₄-C₁₂ alkyl; and each substituent in L is independently selected from the group consisting of halogen, C₁-C₃ alkyl or C₁-C₃ alkyloxy.

According to embodiments of the present disclosure, L is selected from the group consisting of substituted or unsubstituted C₄-C₁₂ linear alkyl or substituted or unsubstituted C₄-C₁₂ linear alkenyl; and each substituent in L is independently selected from the group consisting of halogen, C₁-C₃ alkyl or C₁-C₃ alkyloxy.

In some optional embodiments of the present disclosure, L is selected from the group consisting of substituted or unsubstituted C₄-C₁₂ linear alkyl, substituted or unsubstituted C₄-C₁₁ linear alkyl, substituted or unsubstituted C₄-C₁₀ linear alkyl, substituted or unsubstituted C₅-C₁₂ linear alkyl, substituted or unsubstituted C₅-C₁₁ linear alkyl, substituted or unsubstituted C₅-C₁₀ linear alkyl, substituted or unsubstituted C₆-C₁₂ linear alkyl, substituted or unsubstituted C₆-C₁₁ linear alkyl, substituted or unsubstituted C₆-C₁₀ linear alkyl, substituted or unsubstituted C₇-C₁₂ linear alkyl, substituted or unsubstituted C₇-C₁₁ linear alkyl, substituted or unsubstituted C₇-C₁₀ linear alkyl, substituted or unsubstituted C₈-C₁₂ linear alkyl, substituted or unsubstituted C₈-C₁₁ linear alkyl, substituted or unsubstituted C₈-C₁₀ linear alkyl, substituted or unsubstituted C₉-C₁₂ linear alkyl, substituted or unsubstituted C₉-C₁₁ linear alkyl or substituted or unsubstituted C₉-C₁₀ linear alkyl; and each substituent in L is independently selected from the group consisting of halogen, C₁-C₃ alkyl or C₁-C₃ alkyloxy.

In some optional embodiments of the present disclosure, L is selected from the group consisting of substituted or unsubstituted C₄-C₁₂ linear alkenyl, substituted or unsubstituted C₄-C₁₁ linear alkenyl, substituted or unsubstituted C₄-C₁₀ linear alkenyl, substituted or unsubstituted C₅-C₁₂ linear alkenyl, substituted or unsubstituted C₅-C₁₁ linear alkenyl, substituted or unsubstituted C₅-C₁₀ linear alkenyl, substituted or unsubstituted C₆-C₁₂ linear alkenyl, substituted or unsubstituted C₆-C₁₁ linear alkenyl, substituted or unsubstituted C₆-C₁₀ linear alkenyl, substituted or unsubstituted C₇-C₁₂ linear alkenyl, substituted or unsubstituted C₇-C₁₁ linear alkenyl, substituted or unsubstituted C₇-C₁₀ linear alkenyl, substituted or unsubstituted C₈-C₁₂ linear alkenyl, substituted or unsubstituted C₈-C₁₁ linear alkenyl, substituted or unsubstituted C₈-C₁₀ linear alkenyl, substituted or unsubstituted C₉-C₁₂ linear alkenyl, substituted or unsubstituted C₉-C₁₁ linear alkenyl, or substituted or unsubstituted C₉-C₁₀ linear alkenyl; and each substituent in L is independently selected from the group consisting of halogen, C₁-C₃ alkyl or C₁-C₃ alkyloxy.

In some optional embodiments of the present disclosure, L is selected from the group consisting of substituted or unsubstituted linear or branched C₄-C₁₂ alkenyl, or substituted or unsubstituted linear or branched C₄-C₁₂ alkyl, and each substituent in L is independently selected from the group consisting of halogen, C₁-C₃ alkyl or C₁-C₃ alkoxy.

In some optional embodiments of the present disclosure, L is selected from the group consisting of substituted or unsubstituted C₄-C₁₂ linear alkyl, or substituted or unsubstituted C₄-C₁₂ linear alkenyl, and each substituent in L is independently selected from the group consisting of halogen, C₁-C₃ alkyl or C₁-C₃ alkyloxy.

In some optional embodiments of the present disclosure, L is selected from the group consisting of C₄-C₁₂ linear alkyl or C₄-C₁₂ linear alkenyl.

In some optional embodiments of the present disclosure, L is selected from the group consisting of C₉-C₁₂ linear alkyl or C₉-C₁₂ linear alkenyl.

In some optional embodiments of the present disclosure, L is selected from the group consisting of C₉-C₁₀ linear alkyl or C₁₂ linear alkenyl.

According to embodiments of the present disclosure, L has any structure selected from the group consisting of:

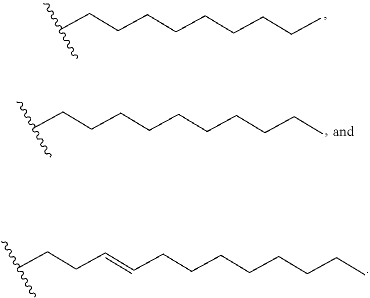

According to embodiments of the present disclosure, L is selected from

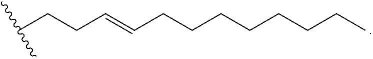 [SMILES: *CC/C=C/CCCCCCCC]

According to embodiments of the present disclosure, L is selected from

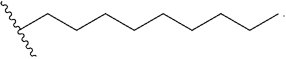 [SMILES: *CCCCCCCCC]

According to embodiments of the present disclosure, L is selected from

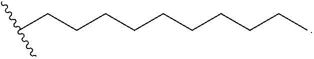 [SMILES: *CCCCCCCCCC]

According to embodiments of the present disclosure, X is selected from the group consisting of O or —NH—C(═O)—.

According to embodiments of the present disclosure, X is selected from O.

According to embodiments of the present disclosure, X is selected from —NH—C(═O)—.

According to embodiments of the present disclosure, X is selected from O, and L is selected from

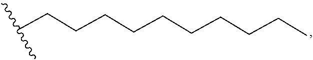 [SMILES: *CCCCCCCCCC]

According to embodiments of the present disclosure, X is selected from —NH—C(═O)—, and L is selected from

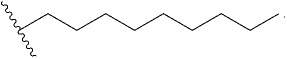 [SMILES: *CCCCCCCCC]

According to embodiments of the present disclosure, X is selected from —NH—C(═O)—, and L is selected from

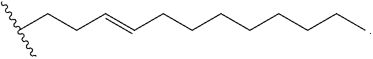 [SMILES: *CC/C=C/CCCCCCCC]

According to embodiments of the present disclosure, n=1.

In some optional embodiments of the present disclosure, p and q are each independently selected from 1 or 2.

According to embodiments of the present disclosure, p=1 and q=1.

According to embodiments of the present disclosure, p=1 and q=2.

According to embodiments of the present disclosure, p=2 and q=1.

According to embodiments of the present disclosure, p=2 and q=2.

In the present disclosure, “nucleotide” refers to compound composed of three substances, i.e., base such as purine base or pyrimidine base, ribose or deoxyribose, and phosphoric acid; “nucleoside” refers to compound composed of two substances, i.e., base such as purine base or pyrimidine base, and ribose or deoxyribose; “nucleobase” refers to base in nucleoside such as purine base (adenine, guanine) and pyrimidine base (cytosine, uracil, thymine); and “nucleobase analog” refers to compound that has similarity to a nucleobase in chemical structure.

In some optional embodiments of the present disclosure, B is selected from the group consisting of nucleobase or nucleobase analog, and if an amino is present in the B, the amino is protected with an amino protecting group.

In some optional embodiments of the present disclosure, the nucleobase is selected from the group consisting of cytosine, adenine, guanine, thymine, or uracil.

In some optional embodiments of the present disclosure, the nucleobase analog is selected from the group consisting of hypoxanthine-9-yl, purin-9-yl, 2-aminopurine-9-yl, 2,4-difluoro-5-methylphenyl, 5-nitroindol-1-yl, 3-nitropyrrole-1-yl, 4-fluoro-6-methylbenzimidazole-1-yl, or 4-methylbenzimidazole-1-yl.

In some optional embodiments of the present disclosure, the amino protecting group is selected from the group consisting of an alkoxycarbonyl amino protecting group, an acyl amino protecting group or an alkyl amino protecting group.

In some optional embodiments of the present disclosure, the alkoxycarbonyl amino protecting group comprises, but is not limited to, carbobenzoxy (Cbz), t-butyloxycarbonyl (Boc), fluorenylmethoxycarbonyl (Fmoc), allyloxycarbonyl (Alloc), trimethylsilyloxycarbonyl (Teoc), methyl (or ethyl) oxycarbonyl, and the like.

In some optional embodiments of the present disclosure, the acyl amino protecting group comprises, but is not limited to, phthaloyl (Pht), p-toluenesulfonyl (Tos), trifluoroacetyl (Tfa), o- (or p-)nitrobenzenesulfonyl (Ns), pivaloyl (Piv), acetyl (Ac), benzoyl (Bz), and the like.

In some optional embodiments of the present disclosure, the alkyl amino protecting group comprises, but is not limited to, trityl (Trt), 2,4-dimethoxybenzyl (Dmb), p-methoxybenzyl (PMB), benzyl (Bn), and the like.

According to embodiments of the present disclosure, the amino protecting group is selected from the acyl amino protecting group.

According to embodiments of the present disclosure, the amino protecting group is selected from benzoyl

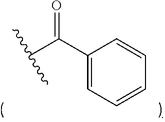

In some optional embodiments of the present disclosure, B has a structure selected from any one of

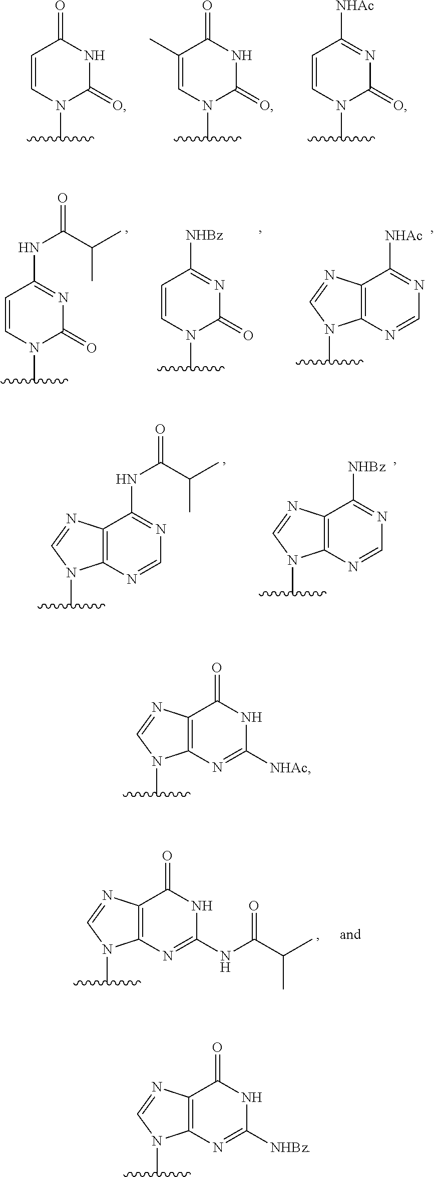

According to embodiments of the present disclosure, B is selected from

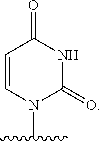 [SMILES: *n1ccc(=O)[nH]c1=O]

According to embodiments of the present disclosure, B is selected from

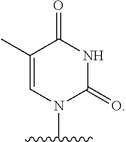 [SMILES: *n1cc(C)c(=O)[nH]c1=O]

According to embodiments of the present disclosure, B is selected from

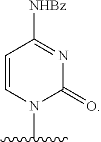

According to embodiments of the present disclosure, B is selected from

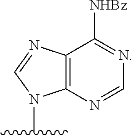

According to embodiments of the present disclosure, B is selected from

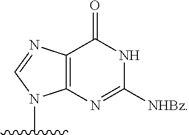

In the present disclosure, the hydroxyl protecting group can be various types of hydroxyl protecting groups, as long as it can protect hydroxyl, and specific types are not limited. In some embodiments, the hydroxyl protecting group is stable under basic conditions, but can be removed under acidic conditions.

In some optional embodiments of the present disclosure, the hydroxyl protecting group comprises, but is not limited to, monomethoxytrityl, dimethoxytrityl, trimethoxytrityl, 9-phenylxanthine-9-yl (Pixyl) and 9-(p-methoxyphenyl)xanthine-9-yl (Mox), trityl (Tr), 4-methoxytrityl (MMTr), 4,4′-dimethoxytrityl (DMTr) and 4,4′,4″-trimethoxytriphenyl (TMTr).

In some optional embodiments of the present disclosure, the hydroxyl protecting group is selected from the group consisting of trityl (Tr), 4-methoxytrityl (MMTr), 4,4′-dimethoxytrityl (DMTr) or 4,4′,4″-trimethoxytriphenyl (TMTr).

According to embodiments of the present disclosure, R₁ is selected from 4,4′-dimethoxytrityl.

According to embodiments of the present disclosure, R₁ is selected from H:

In the present disclosure, the term “phosphorus-containing leaving group” refers to a phosphorus-containing group that can be removed by reaction with other compounds.

In some optional embodiments of the present disclosure, the phosphorus-containing leaving group is selected from the group consisting of

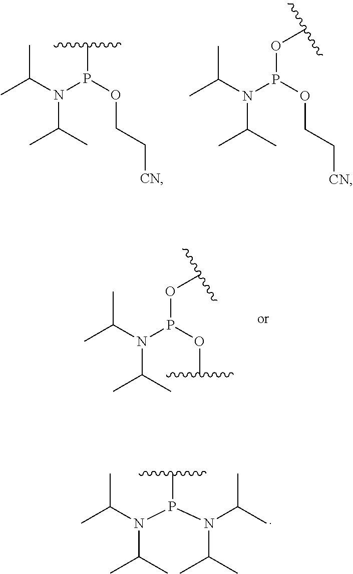 [SMILES: *P(OCCC#N)N(C(C)C)C(C)C ; *OP(OCCC#N)N(C(C)C)C(C)C ; *OP(O*)N(C(C)C)C(C)C ; *P(N(C(C)C)C(C)C)N(C(C)C)C(C)C]

In some optional embodiments of the present disclosure, the phosphorus-containing leaving group is selected from the group consisting of

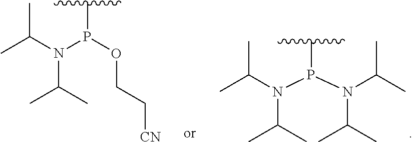 [SMILES: *P(OCCC#N)N(C(C)C)C(C)C ; *P(N(C(C)C)C(C)C)N(C(C)C)C(C)C]

According to embodiments of the present disclosure, R₂ is selected from

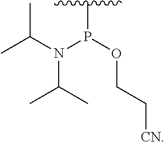 [SMILES: *P(OCCC#N)N(C(C)C)C(C)C]

According to embodiments of the present disclosure, R₂ is selected from H.

According to embodiments of the present disclosure, the nucleoside analog is a compound as represented by Formula (IIa) or a prodrug thereof:

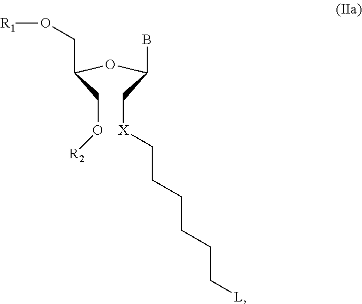

wherein groups of R₁, R₂, B, X and L are as shown in the preceding.

According to embodiments of the present disclosure, the nucleoside analog is a compound as represented by Formula (IIIa) or a prodrug thereof:

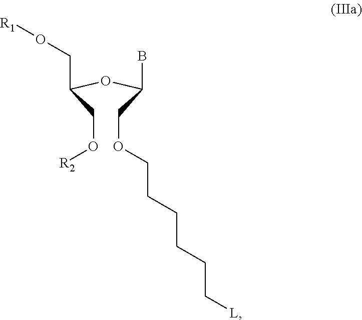

wherein groups of R₁, R₂, B and L are as shown in the preceding.

According to embodiments of the present disclosure, the nucleoside analog is a compound as represented by Formula (IVa) or a prodrug thereof:

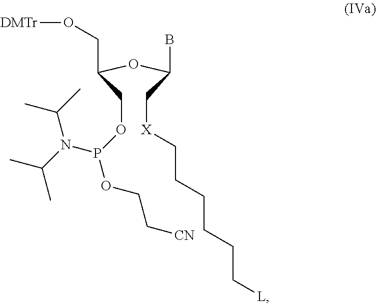

wherein groups of B, X and L are as shown in the preceding.

According to embodiments of the present disclosure, the nucleoside analog has a structure selected from any one of

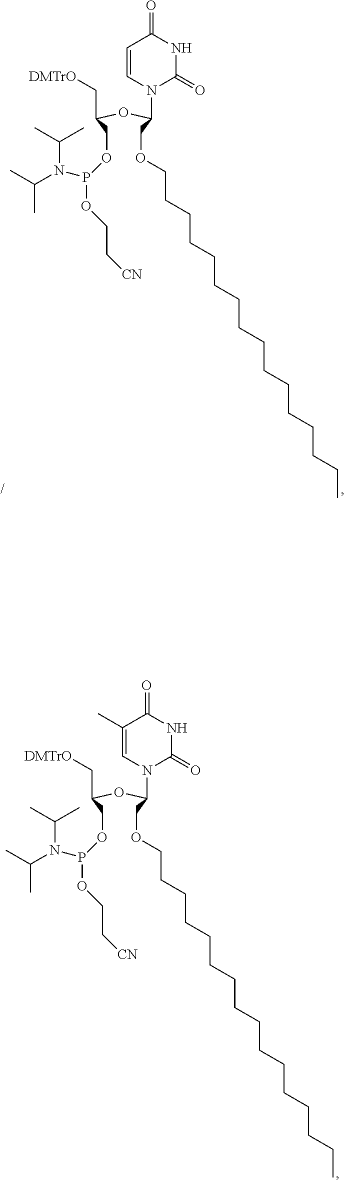

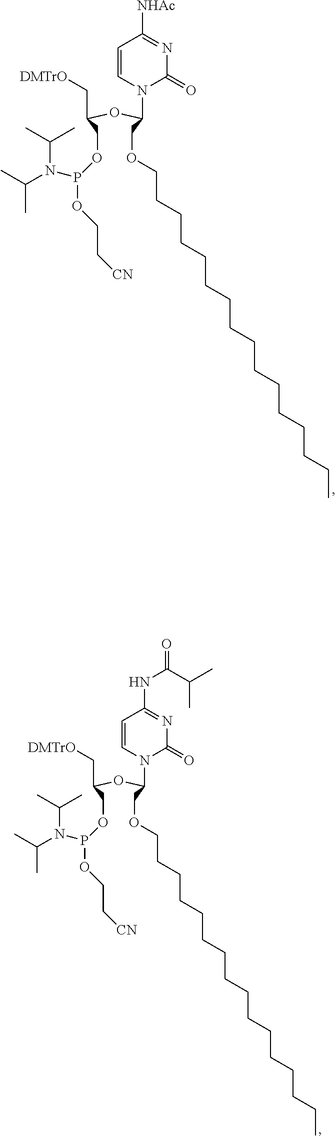

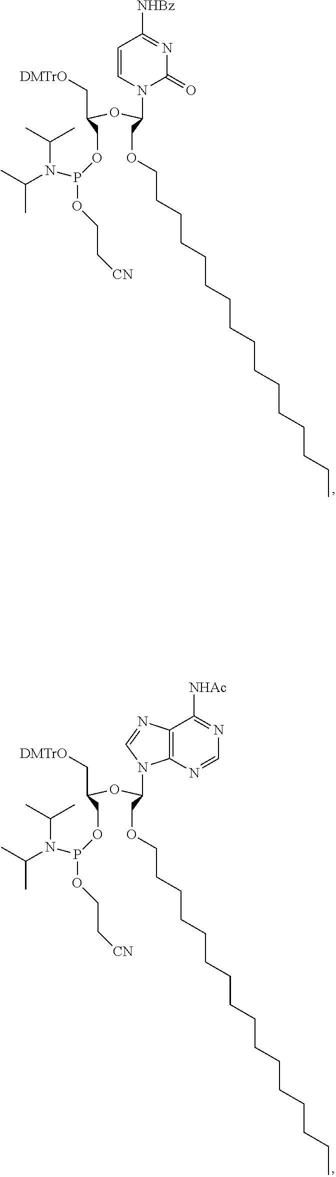

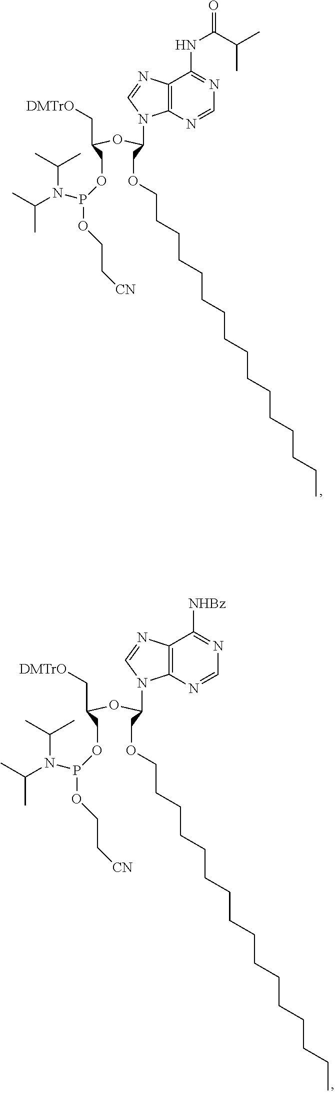

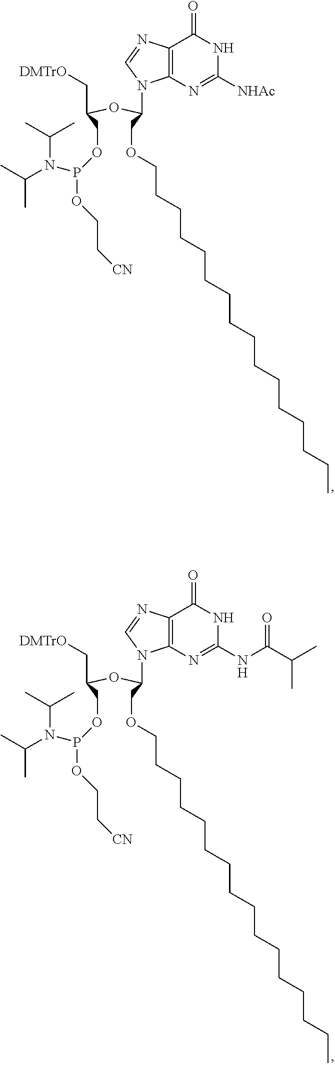

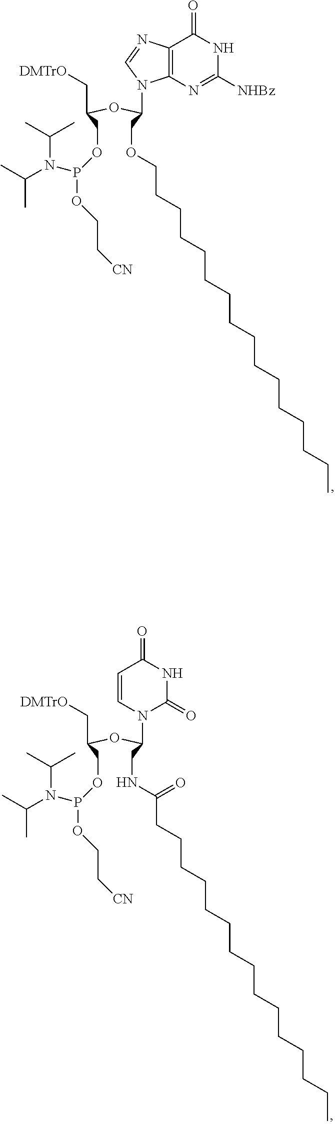

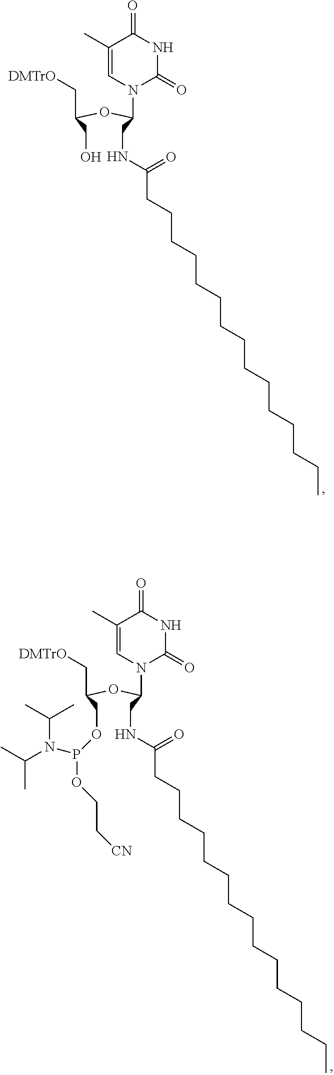

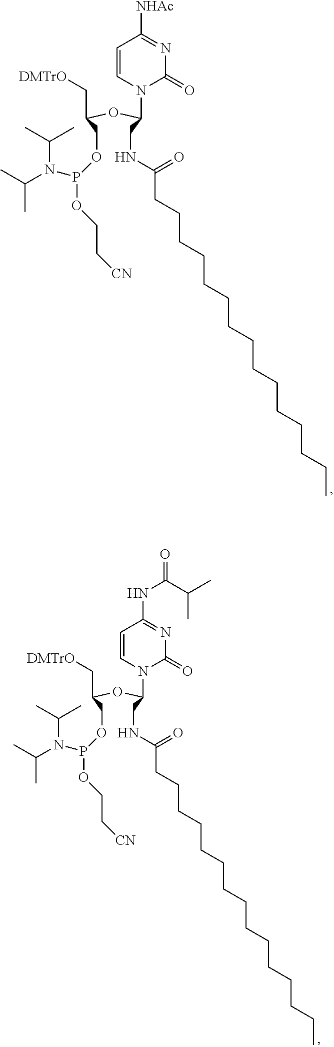

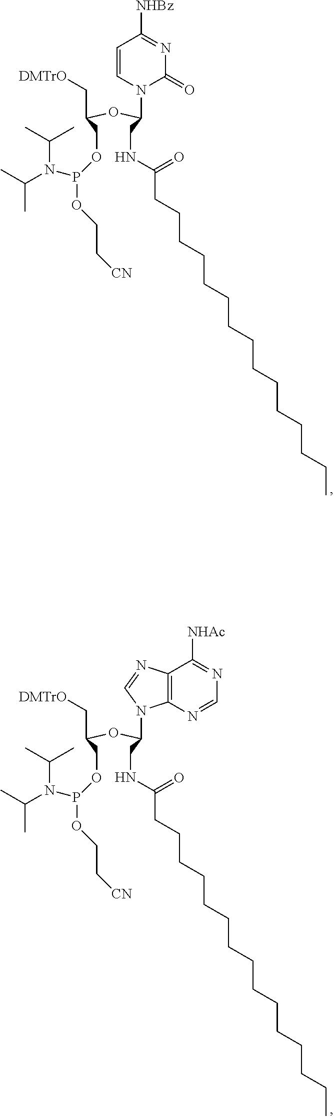

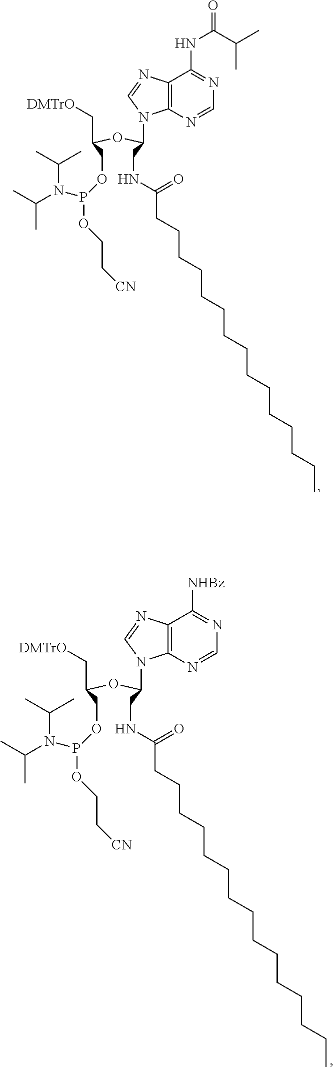

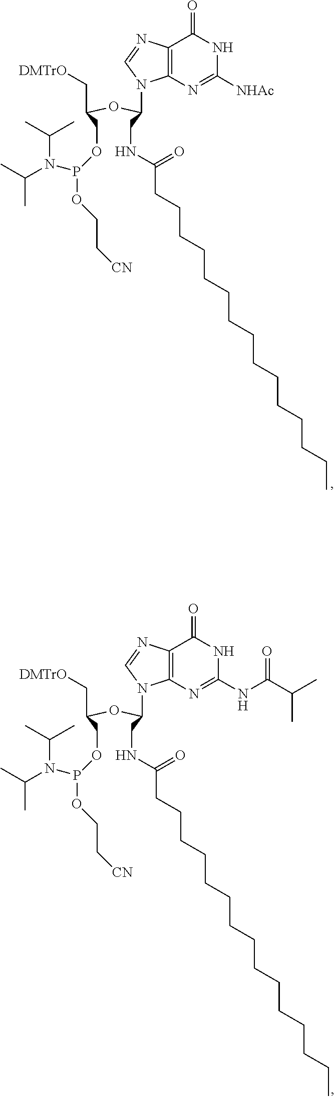

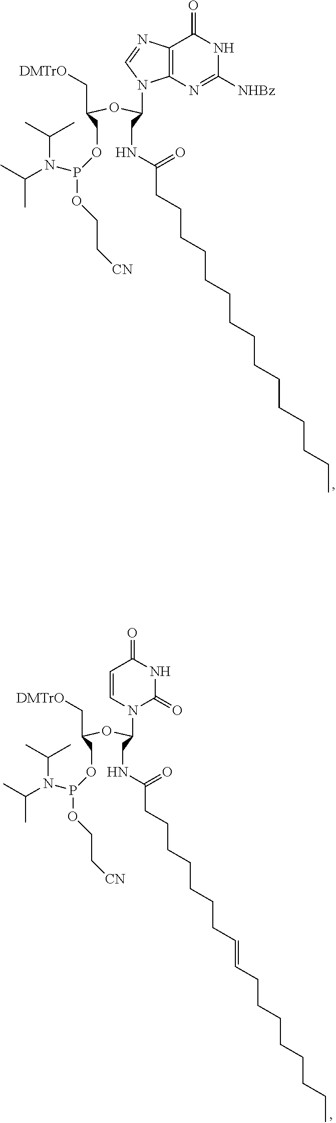

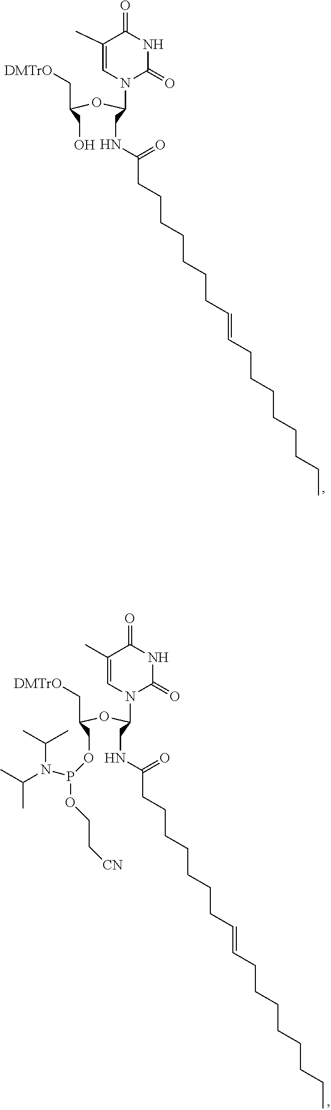

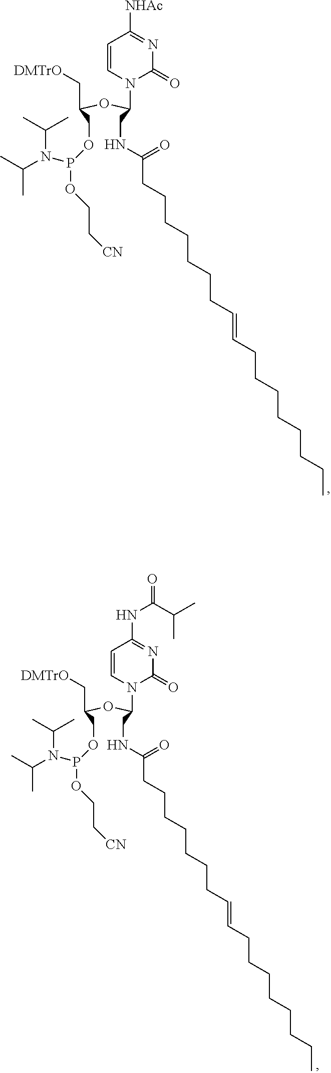

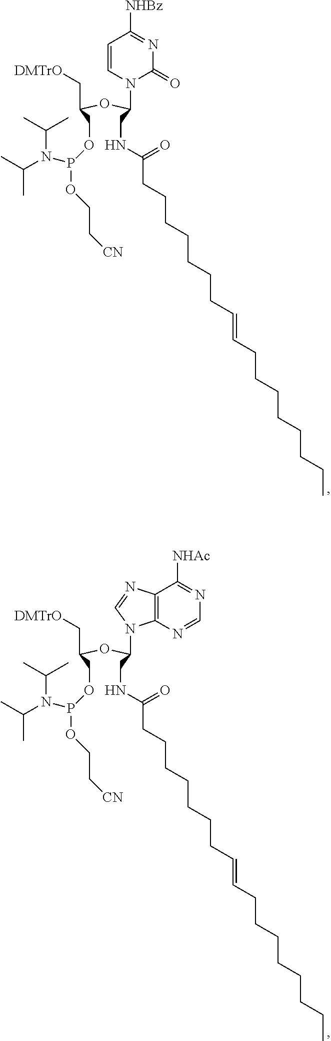

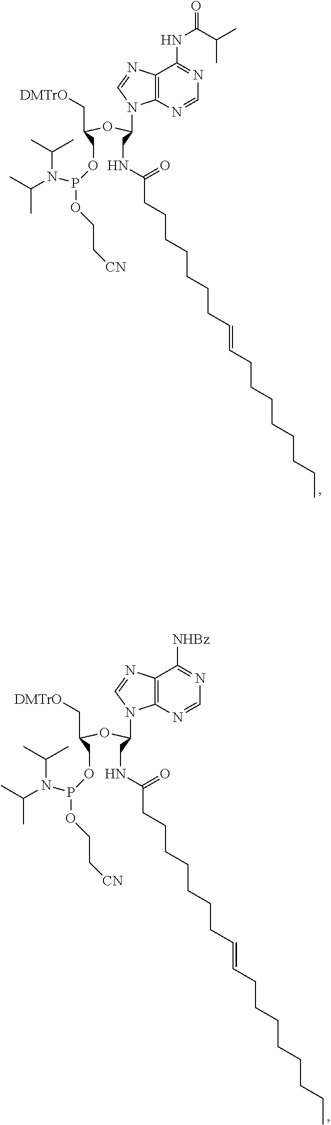

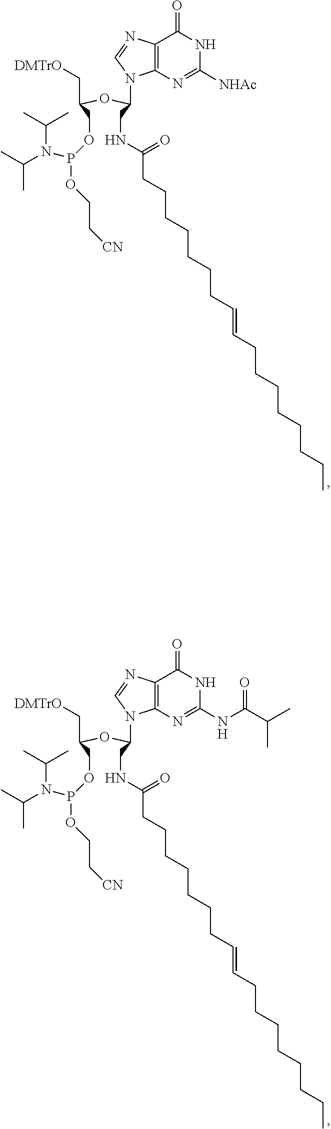

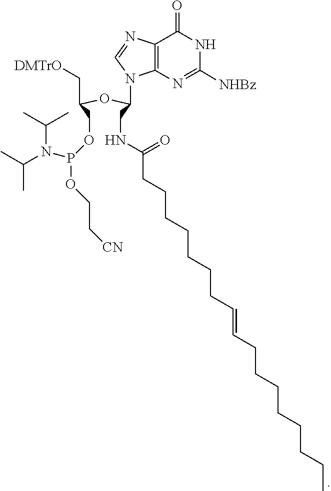

In the second aspect of the present disclosure, the present disclosure provides a nucleoside analog, which is a compound as represented by Formula (Ib), Formula (Ic) or Formula (Id) or a prodrug thereof:

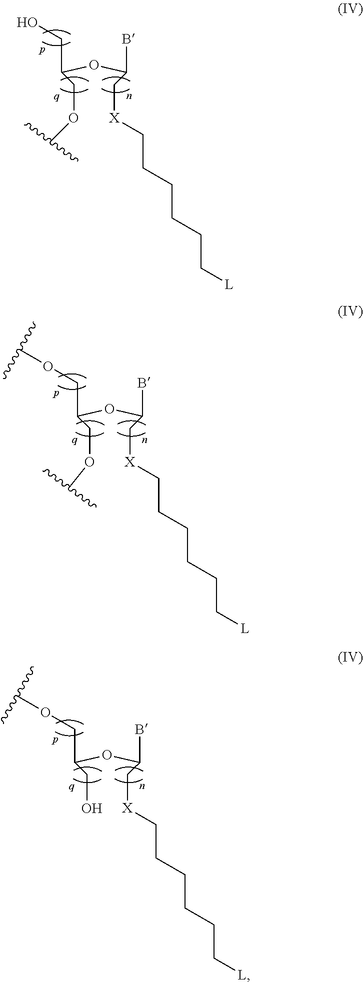

wherein R₁ is selected from the group consisting of H or a hydroxyl protecting group;
R₂ is selected from the group consisting of H or a phosphorus-containing leaving group;
B′ is selected from the group consisting of substituted or unsubstituted nucleobase or substituted or unsubstituted nucleobase analog; each substituent in B′ is independently selected from the group consisting of halogen, C₁-C₃ alkyl, C₁-C₃ alkyloxy, or substituted amide group; and if an amino is present in the B′, the amino is not protected;
X is selected from the group consisting of O, S, NH or —NH—C(═O)—;
L is selected from the group consisting of substituted or unsubstituted C₄-C₁₂ hydrocarbyl, and each substituent in L is independently selected from the group consisting of halogen, C₁-C₃ alkyl or C₁-C₃ alkoxy;
p and q are each independently selected from 1, 2, 3 or 4; and
n is selected from 1, 2 or 3.

In some optional embodiments of the present disclosure, L is selected from the group consisting of substituted or unsubstituted C₄-C₁₂ hydrocarbyl, substituted or unsubstituted C₄-C₁₁ hydrocarbyl, substituted or unsubstituted C₄-C₁₀ hydrocarbyl, substituted or unsubstituted C₅-C₁₂ hydrocarbyl, substituted or unsubstituted C₅-C₁₁ hydrocarbyl, substituted or unsubstituted C₅-C₁₀ hydrocarbyl, substituted or unsubstituted C₆-C₁₂ hydrocarbyl, substituted or unsubstituted C₆-C₁₁ hydrocarbyl, substituted or unsubstituted C₆-C₁₀ hydrocarbyl, substituted or unsubstituted C₇-C₁₂ hydrocarbyl, substituted or unsubstituted C₇-C₁₁ hydrocarbyl, substituted or unsubstituted C₇-C₁₀ hydrocarbyl, substituted or unsubstituted C₈-C₁₂ hydrocarbyl, substituted or unsubstituted C₈-C₁₁ hydrocarbyl, substituted or unsubstituted C₈-C₁₀ hydrocarbyl, substituted or unsubstituted C₉-C₁₂ hydrocarbyl, substituted or unsubstituted C₉-C₁₁ hydrocarbyl, or substituted or unsubstituted C₉-C₁₀ hydrocarbyl; and each substituent in L is independently selected from the group consisting of halogen, C₁-C₃ alkyl or C₁-C₃ alkyloxy.

According to embodiments of the present disclosure, L is selected from the group consisting of substituted or unsubstituted linear or branched C₄-C₁₂ alkenyl, or substituted or unsubstituted linear or branched C₄-C₁₂ alkyl; and each substituent in L is independently selected from the group consisting of halogen, C₁-C₃ alkyl or C₁-C₃ alkyloxy.

According to embodiments of the present disclosure, L is selected from the group consisting of substituted or unsubstituted C₄-C₁₂ linear alkyl or substituted or unsubstituted C₄-C₁₂ linear alkenyl; and each substituent in L is independently selected from the group consisting of halogen, C₁-C₃ alkyl or C₁-C₃ alkyloxy.

In some optional embodiments of the present disclosure, L is selected from the group consisting of substituted or unsubstituted C₄-C₁₂ linear alkyl, substituted or unsubstituted C₄-C₁₁ linear alkyl, substituted or unsubstituted C₄-C₁₀ linear alkyl, substituted or unsubstituted C₅-C₁₂ linear alkyl, substituted or unsubstituted C₅-C₁₁ linear alkyl, substituted or unsubstituted C₅-C₁₀ linear alkyl, substituted or unsubstituted C₆-C₁₂ linear alkyl, substituted or unsubstituted C₆-C₁₁ linear alkyl, substituted or unsubstituted C₆-C₁₀ linear alkyl, substituted or unsubstituted C₇-C₁₂ linear alkyl, substituted or unsubstituted C₇-C₁₁ linear alkyl, substituted or unsubstituted C₇-C₁₀ linear alkyl, substituted or unsubstituted C₈-C₁₂ linear alkyl, substituted or unsubstituted C₈-C₁₁ linear alkyl, substituted or unsubstituted C₈-C₁₀ linear alkyl, substituted or unsubstituted C₉-C₁₂ linear alkyl, substituted or unsubstituted C₉-C₁₁ linear alkyl or substituted or unsubstituted C₉-C₁₀ linear alkyl; and each substituent in L is independently selected from the group consisting of halogen, C₁-C₃ alkyl or C₁-C₃ alkyloxy.

In some optional embodiments of the present disclosure, L is selected from the group consisting of substituted or unsubstituted C₄-C₁₂ linear alkenyl, substituted or unsubstituted C₄-C₁₁ linear alkenyl, substituted or unsubstituted C₄-C₁₀ linear alkenyl, substituted or unsubstituted C₅-C₁₂ linear alkenyl, substituted or unsubstituted C₅-C₁₁ linear alkenyl, substituted or unsubstituted C₅-C₁₀ linear alkenyl, substituted or unsubstituted C₆-C₁₂ linear alkenyl, substituted or unsubstituted C₆-C₁₁ linear alkenyl, substituted or unsubstituted C₆-C₁₀ linear alkenyl, substituted or unsubstituted C₇-C₁₂ linear alkenyl, substituted or unsubstituted C₇-C₁₁ linear alkenyl, substituted or unsubstituted C₇-C₁₀ linear alkenyl, substituted or unsubstituted C₈-C₁₂ linear alkenyl, substituted or unsubstituted C₈-C₁₁ linear alkenyl, substituted or unsubstituted C₈-C₁₀ linear alkenyl, substituted or unsubstituted C₉-C₁₂ linear alkenyl, substituted or unsubstituted C₉-C₁₁ linear alkenyl, or substituted or unsubstituted C₉-C₁₀ linear alkenyl; and each substituent in L is independently selected from the group consisting of halogen, C₁-C₃ alkyl or C₁-C₃ alkyloxy.

In some optional embodiments of the present disclosure, L is selected from the group consisting of substituted or unsubstituted linear or branched C₄-C₁₂ alkenyl, or substituted or unsubstituted linear or branched C₄-C₁₂ alkyl, and each substituent in L is independently selected from the group consisting of halogen, C₁-C₃ alkyl or C₁-C₃ alkoxy.

In some optional embodiments of the present disclosure, L is selected from the group consisting of substituted or unsubstituted C₄-C₁₂ linear alkyl, or substituted or unsubstituted C₄-C₁₂ linear alkenyl, and each substituent in L is independently selected from the group consisting of halogen, C₁-C₃ alkyl or C₁-C₃ alkyloxy.

In some optional embodiments of the present disclosure, L is selected from the group consisting of C₄-C₁₂ linear alkyl or C₄-C₁₂ linear alkenyl.

In some optional embodiments of the present disclosure, L is selected from the group consisting of C₉-C₁₂ linear alkyl or C₉-C₁₂ linear alkenyl.

In some optional embodiments of the present disclosure, L is selected from the group consisting of C₉-C₁₂ linear alkyl or C₉-C₁₂ linear alkenyl.

According to embodiments of the present disclosure, L has any structure selected from the group consisting of:

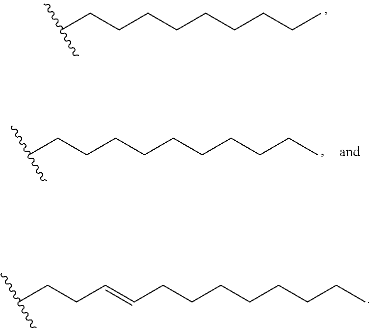

According to embodiments of the present disclosure, L is selected from

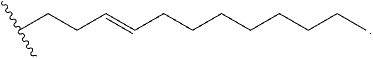 [SMILES: *CC/C=C/CCCCCCCC]

According to embodiments of the present disclosure, L is selected from

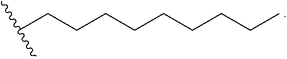 [SMILES: *CCCCCCCCC]

According to embodiments of the present disclosure, L is selected from

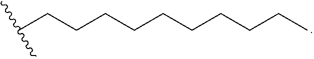 [SMILES: *CCCCCCCCCC]

According to embodiments of the present disclosure, X is selected from the group consisting of O or —NH—C(═O)—.

According to embodiments of the present disclosure, X is selected from O.

According to embodiments of the present disclosure, X is selected from —NH—C(═O)—.

According to embodiments of the present disclosure, X is selected from O, and L is selected from

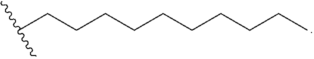 [SMILES: *CCCCCCCCCC]

According to embodiments of the present disclosure, X is selected from —NH—C(═O)—, and L is selected from

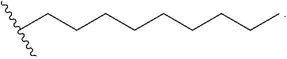 [SMILES: *CCCCCCCCC]

According to embodiments of the present disclosure, X is selected from —NH—C(═O)—, and L is selected from

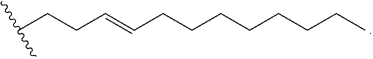 [SMILES: *CC/C=C/CCCCCCCC]

According to embodiments of the present disclosure, n=1.

In some optional embodiments of the present disclosure, p and q are each independently selected from 1 or 2.

According to embodiments of the present disclosure, p=1 and q=1.

According to embodiments of the present disclosure, p=1 and q=2.

According to embodiments of the present disclosure, p=2 and q=1.

According to embodiments of the present disclosure, p=2 and q=2.

In some optional embodiments of the present disclosure, B′ is selected from the group consisting of nucleobase or nucleobase analog, and if an amino is present in the B′, the amino is not protected.

In some optional embodiments of the present disclosure, the nucleobase is selected from the group consisting of cytosine, adenine, guanine, thymine, or uracil.

In some optional embodiments of the present disclosure, the nucleobase analog is selected from the group consisting of hypoxanthine-9-yl, purin-9-yl, 2-aminopurine-9-yl, 2,4-difluoro-5-methylphenyl, 5-nitroindol-1-yl, 3-nitropyrrole-1-yl, 4-fluoro-6-methylbenzimidazole-1-yl, or 4-methylbenzimidazole-1-yl.

According to embodiments of the present disclosure, B′ is selected from nucleobase.

According to embodiments of the present disclosure, B′ is selected from the group consisting of

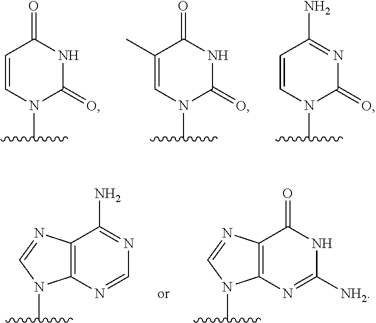

According to embodiments of the present disclosure, B′ is selected from

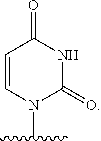 [SMILES: *n1ccc(=O)[nH]c1=O]

According to embodiments of the present disclosure, B′ is selected from

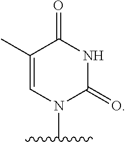 [SMILES: *n1cc(C)c(=O)[nH]c1=O]

According to embodiments of the present disclosure, B′ is selected from

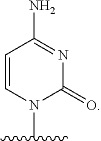 [SMILES: *n1ccc(N)nc1=O]

According to embodiments of the present disclosure, B′ is selected from

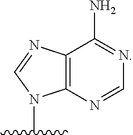 [SMILES: *n1cnc2c(N)ncnc21]

According to embodiments of the present disclosure, B′ is selected from

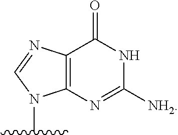 [SMILES: *n1cnc2c(=O)[nH]c(N)nc21]

In some optional embodiments of the present disclosure, the hydroxyl protecting group comprises, but is not limited to, monomethoxytrityl, dimethoxytrityl, trimethoxytrityl, 9-phenylxanthine-9-yl (Pixyl) and 9-(p-methoxyphenyl)xanthine-9-yl (Mox), trityl (Tr group), 4-methoxytrityl (MMTr group), 4,4′-dimethoxytrityl (DMTr group) and 4,4′,4″-trimethoxytriphenyl (TMTr group).

In some optional embodiments of the present disclosure, the hydroxyl protecting group is selected from the group consisting of trityl (Tr group), 4-methoxytrityl (MMTr group), 4,4′-dimethoxytrityl (DMTr group) or 4,4′,4″-trimethoxytriphenyl (TMTr group).

According to embodiments of the present disclosure, the hydroxyl protecting group is selected from 4,4′-dimethoxytrityl.

According to embodiments of the present disclosure, R₁ is selected from 4,4′-dimethoxytrityl.

According to embodiments of the present disclosure, R₁ is selected from H.

In some optional embodiments of the present disclosure, the phosphorus-containing leaving group is selected from the group consisting of

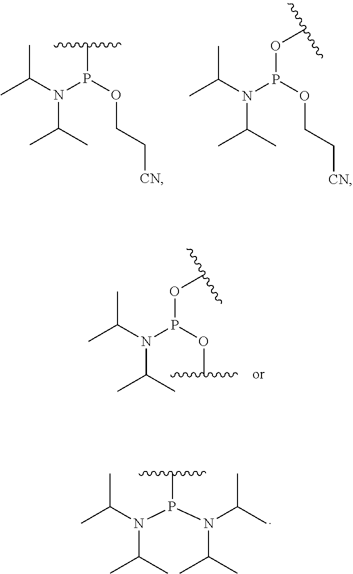 [SMILES: *P(OCCC#N)N(C(C)C)C(C)C ; *OP(OCCC#N)N(C(C)C)C(C)C ; *OP(O*)N(C(C)C)C(C)C ; *P(N(C(C)C)C(C)C)N(C(C)C)C(C)C]

In some optional embodiments of the resent disclosure, the phosphorus-containing leaving group is selected from the group consisting of

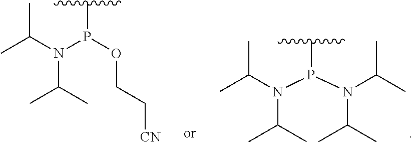 [SMILES: *P(OCCC#N)N(C(C)C)C(C)C ; *P(N(C(C)C)C(C)C)N(C(C)C)C(C)C]

According to embodiments of the present disclosure, the phosphorus-containing leaving group is selected from

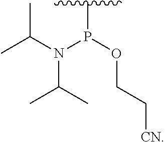 [SMILES: *P(OCCC#N)N(C(C)C)C(C)C]

According to embodiments of the present disclosure, R₂ is selected from

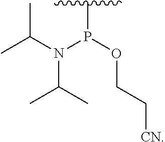 [SMILES: *P(OCCC#N)N(C(C)C)C(C)C]

According to embodiments of the present disclosure, R₂ is selected from H.

In some optional embodiments of the present disclosure, the nucleoside analog is selected from the group consisting of a compound as represented by Formula (IIb), Formula (IIc) or Formula (IId) or prodrugs thereof:

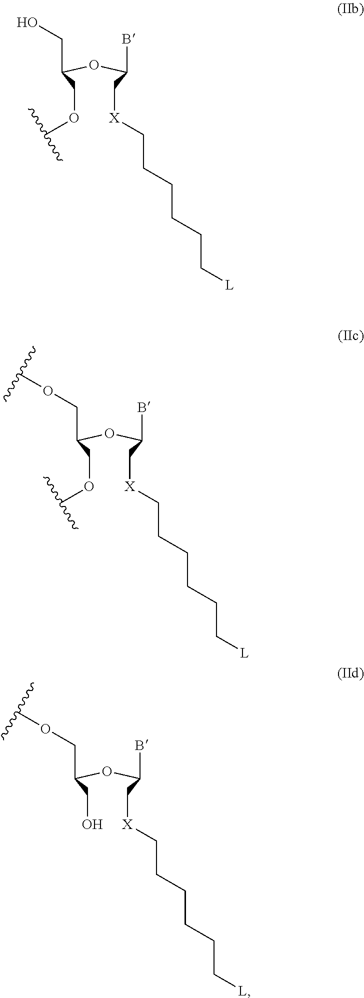

wherein groups of B′, X and L are as shown in the preceding.

In some optional embodiments of the present disclosure, the nucleoside analog is selected from the group consisting of a compound as represented by Formula (IIIb), Formula (IIIc) or Formula (IIId) or prodrugs thereof:

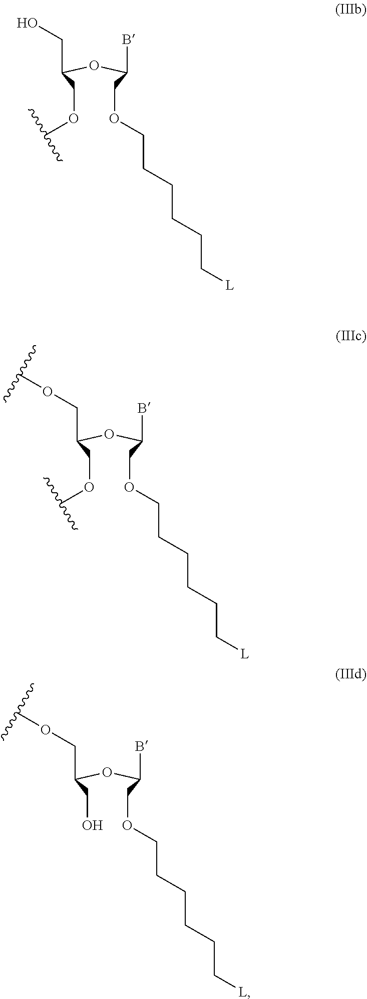

wherein groups of B′ and L are as shown in the preceding.

According to embodiments of the present disclosure, the nucleoside analog has a structure selected from any one of

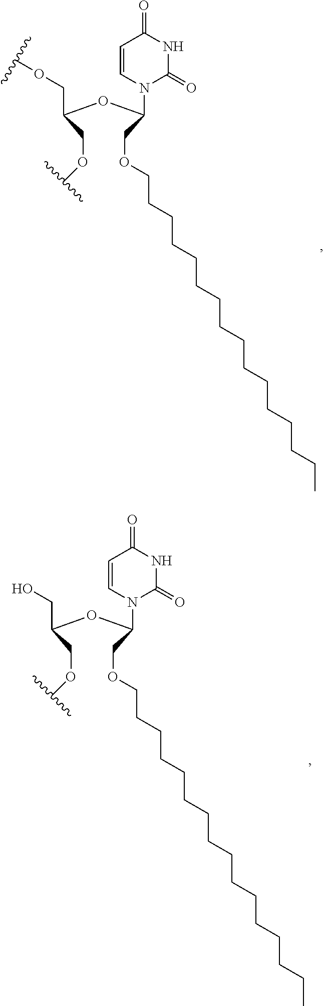

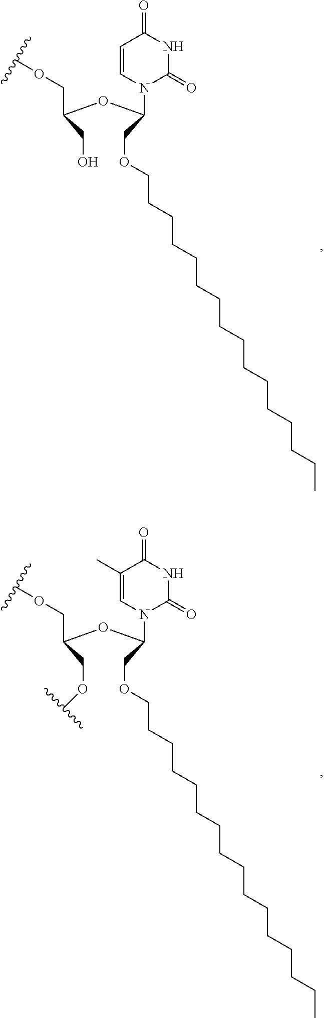

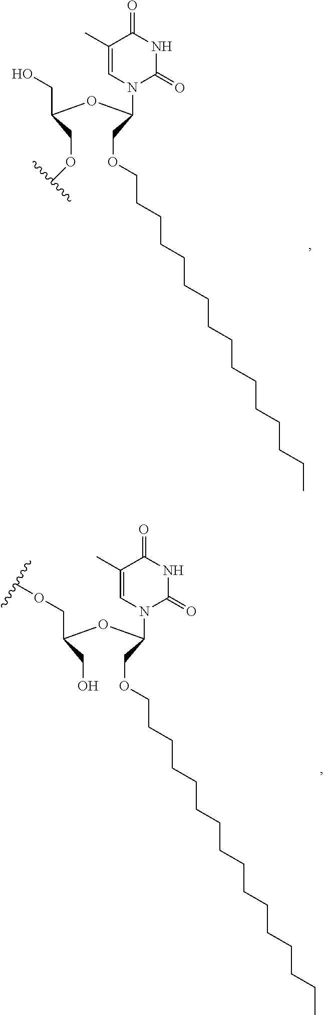 [SMILES: *OC[C@@H](CO)O[C@H](COCCCCCCCCCCCCCCCC)n1cc(C)c(=O)[nH]c1=O ; *OC[C@H](CO)O[C@H](COCCCCCCCCCCCCCCCC)n1cc(C)c(=O)[nH]c1=O]

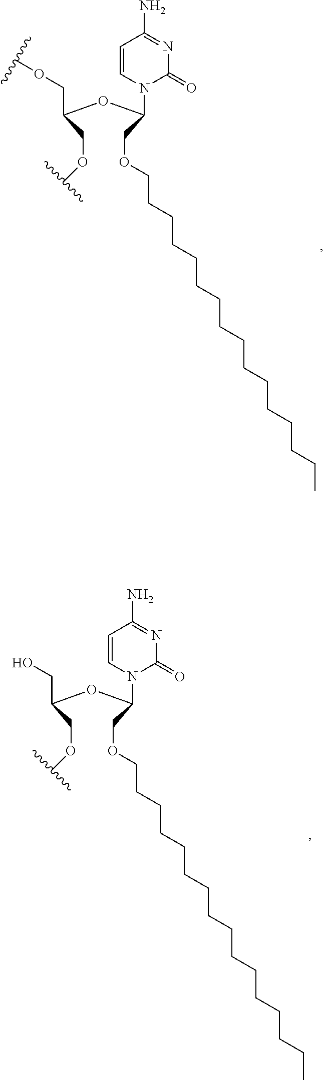

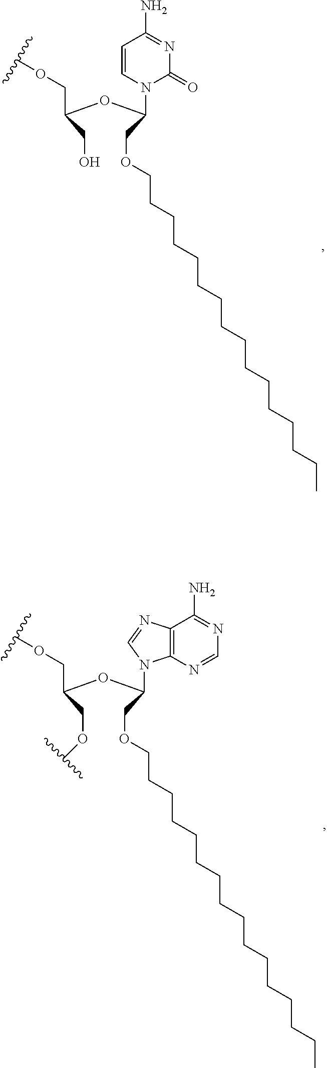

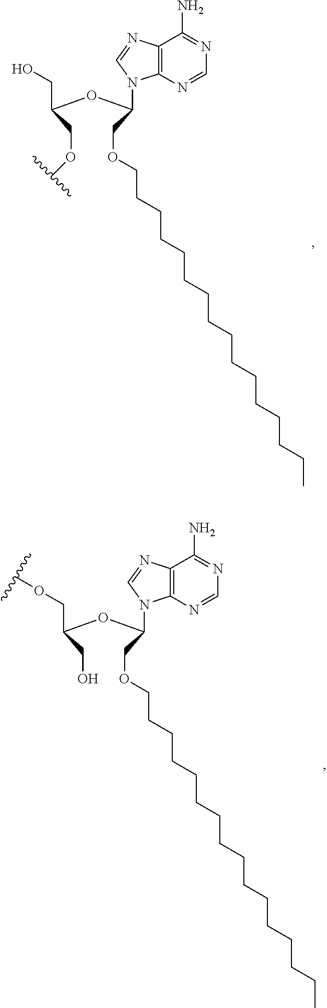 [SMILES: *OC[C@@H](CO)O[C@H](COCCCCCCCCCCCCCCCC)n1cnc2c(N)ncnc21 ; *OC[C@H](CO)O[C@H](COCCCCCCCCCCCCCCCC)n1cnc2c(N)ncnc21]

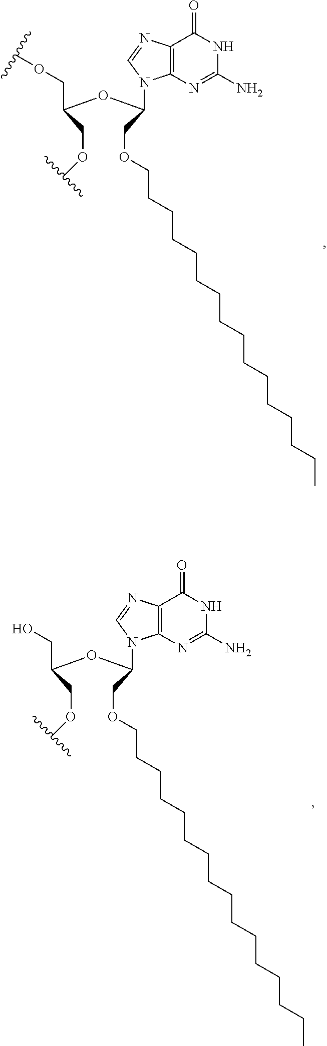

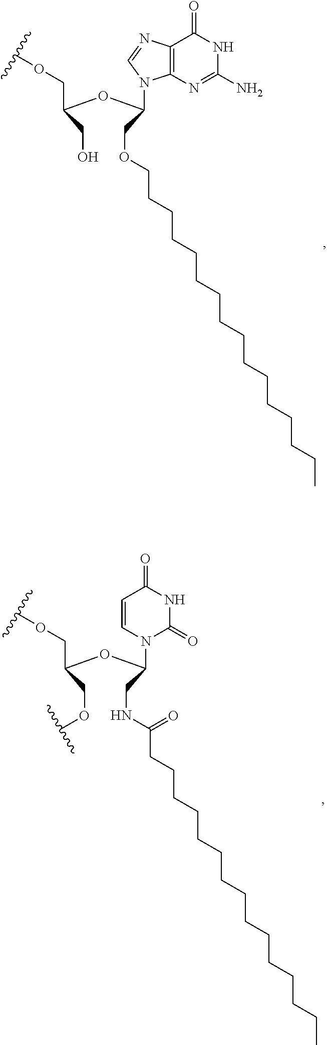

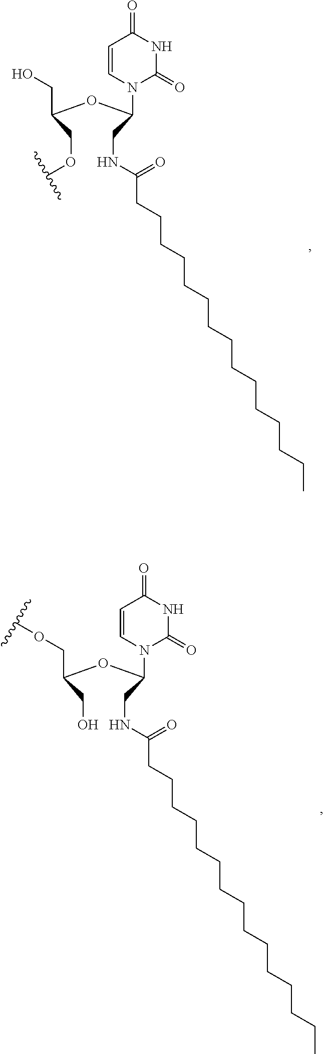 [SMILES: *OC[C@@H](CO)O[C@H](CNC(=O)CCCCCCCCCCCCCCC)n1ccc(=O)[nH]c1=O ; *OC[C@H](CO)O[C@H](CNC(=O)CCCCCCCCCCCCCCC)n1ccc(=O)[nH]c1=O]

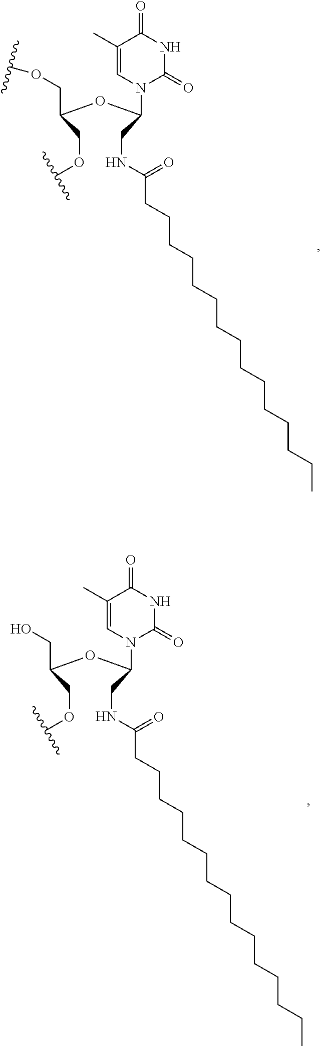

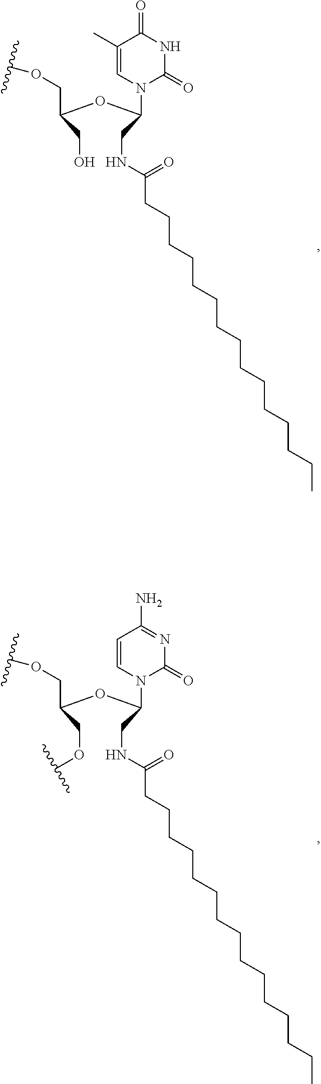

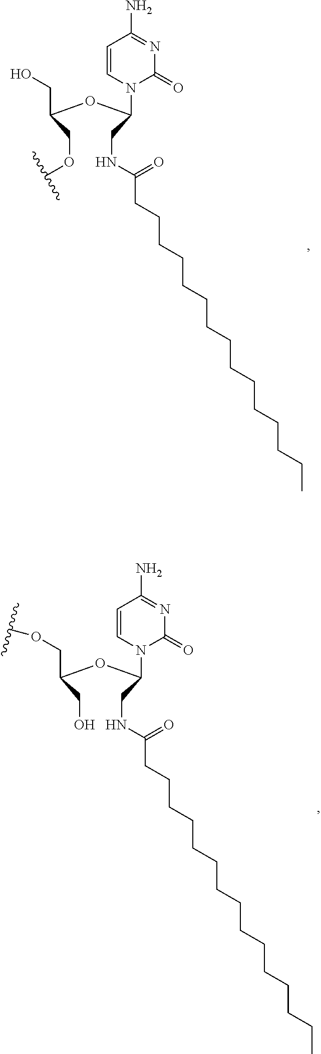 [SMILES: *OC[C@@H](CO)O[C@H](CNC(=O)CCCCCCCCCCCCCCC)n1ccc(N)nc1=O ; *OC[C@H](CO)O[C@H](CNC(=O)CCCCCCCCCCCCCCC)n1ccc(N)nc1=O]

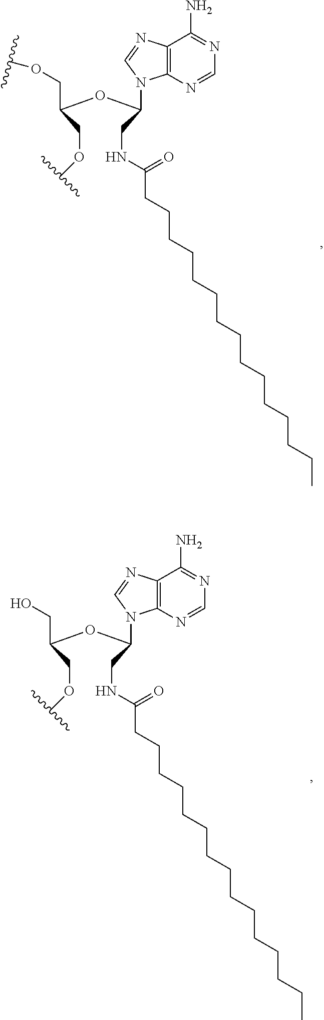

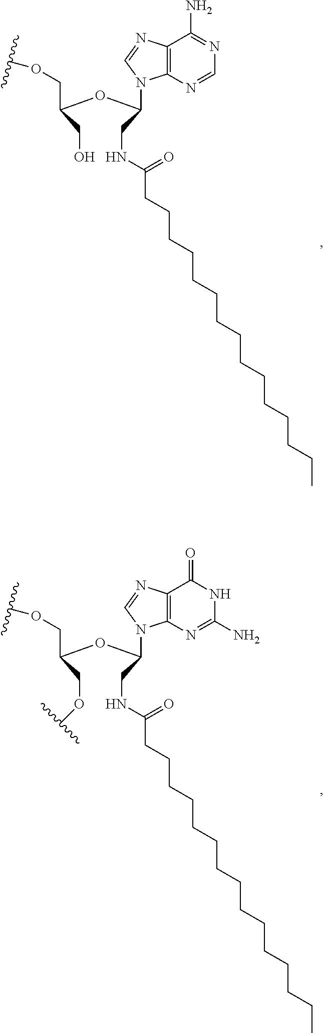

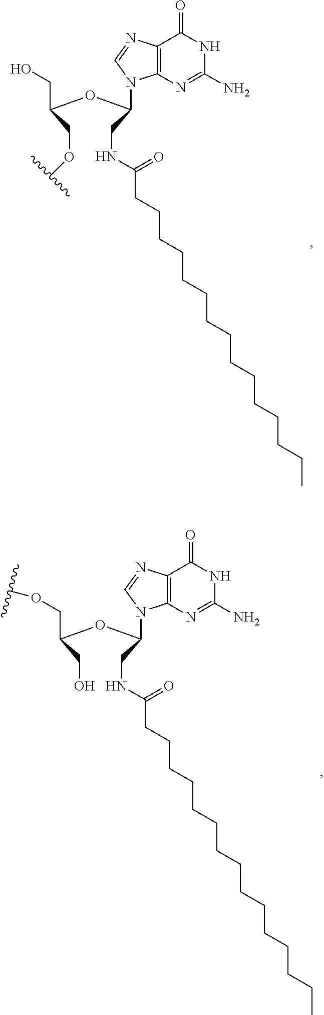 [SMILES: *OC[C@@H](CO)O[C@H](CNC(=O)CCCCCCCCCCCCCCC)n1cnc2c(=O)[nH]c(N)nc21 ; *OC[C@H](CO)O[C@H](CNC(=O)CCCCCCCCCCCCCCC)n1cnc2c(=O)[nH]c(N)nc21]

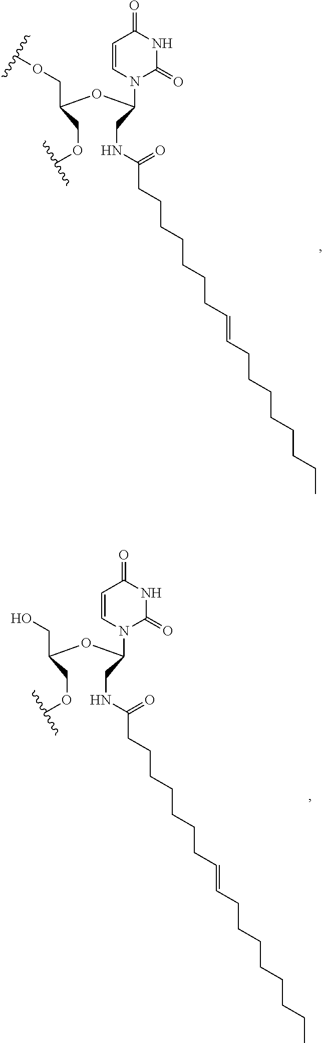

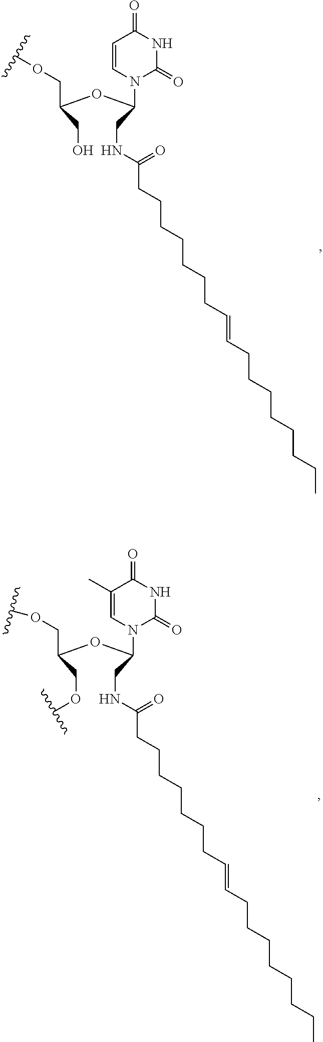

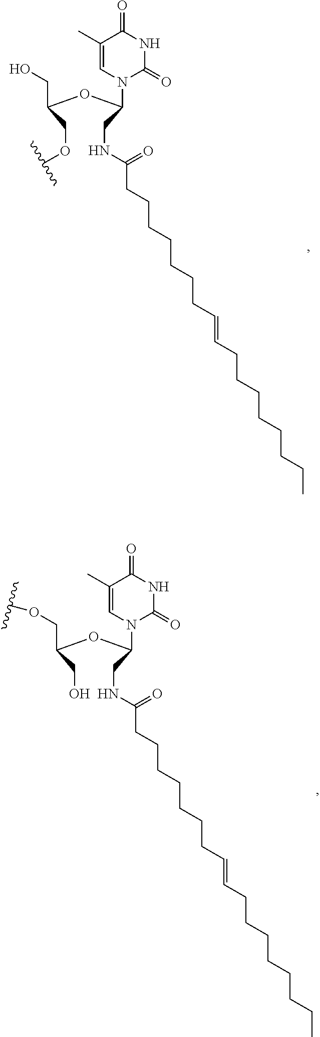 [SMILES: *OC[C@@H](CO)O[C@H](CNC(=O)CCCCCCC/C=C/CCCCCCCC)n1cc(C)c(=O)[nH]c1=O ; *OC[C@H](CO)O[C@H](CNC(=O)CCCCCCC/C=C/CCCCCCCC)n1cc(C)c(=O)[nH]c1=O]

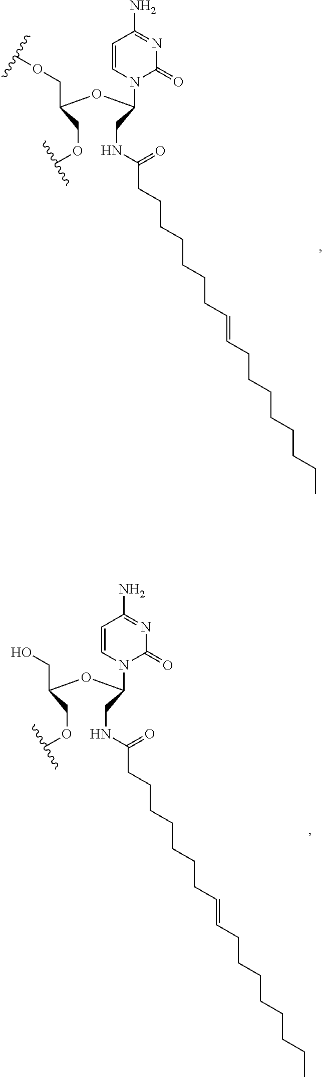

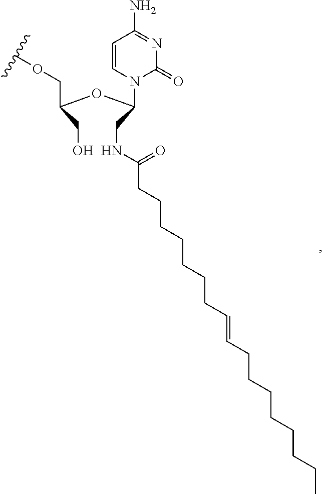 [SMILES: *OC[C@H](CO)O[C@H](CNC(=O)CCCCCCC/C=C/CCCCCCCC)n1ccc(N)nc1=O]

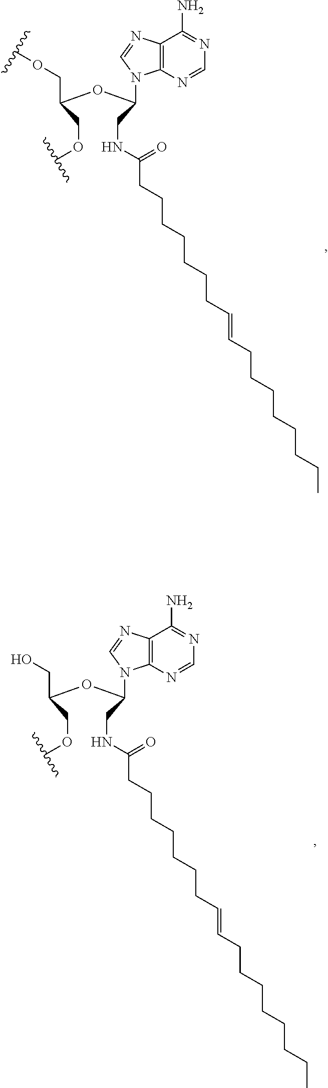

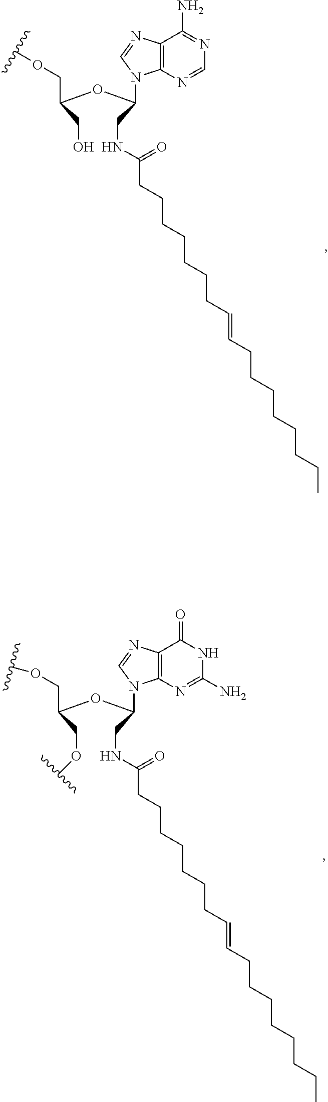

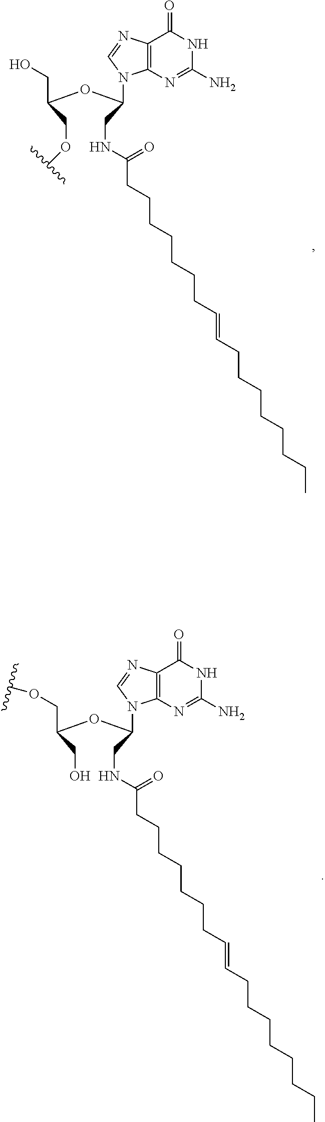 [SMILES: *OC[C@@H](CO)O[C@H](CNC(=O)CCCCCCC/C=C/CCCCCCCC)n1cnc2c(=O)[nH]c(N)nc21 ; *OC[C@H](CO)O[C@H](CNC(=O)CCCCCCC/C=C/CCCCCCCC)n1cnc2c(=O)[nH]c(N)nc21]

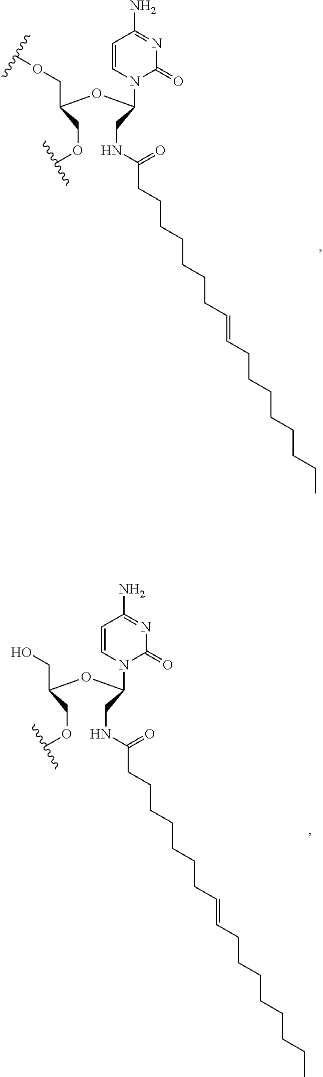

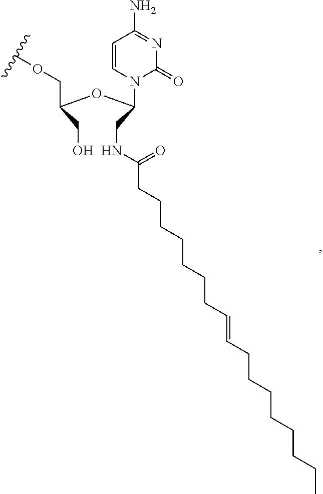 [SMILES: *OC[C@H](CO)O[C@H](CNC(=O)CCCCCCC/C=C/CCCCCCCC)n1ccc(N)nc1=O]

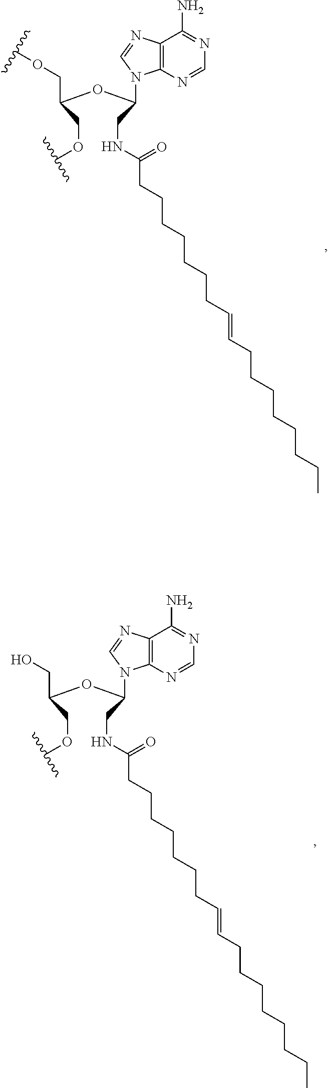

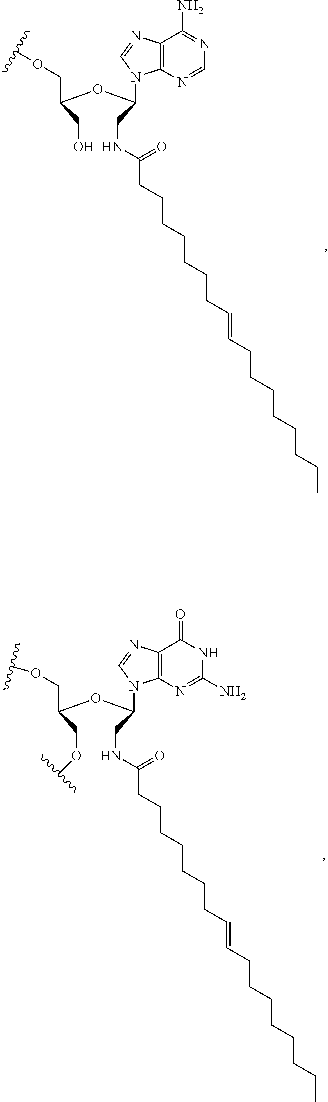

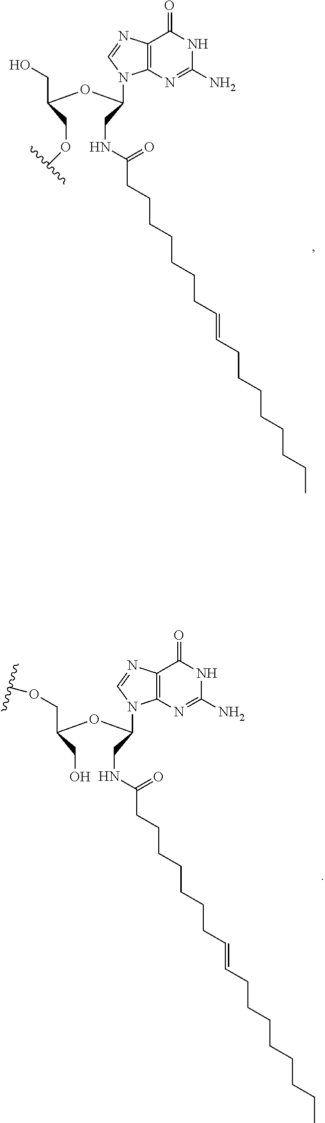 [SMILES: *OC[C@@H](CO)O[C@H](CNC(=O)CCCCCCC/C=C/CCCCCCCC)n1cnc2c(=O)[nH]c(N)nc21 ; *OC[C@H](CO)O[C@H](CNC(=O)CCCCCCC/C=C/CCCCCCCC)n1cnc2c(=O)[nH]c(N)nc21]

In the third aspect of the present disclosure, the present disclosure provides use of the nucleoside analog according to the first aspect, and/or the nucleoside analog according to the second aspect in preparation of oligonucleotides.

The nucleoside analogs according to the first and second aspects can be used to replace nucleotides, such as A, U, C, G or T. Further, the B base group of the nucleoside analog according to the first aspect or the B′ base group of the nucleoside analog according to the second aspect can be selected according to a type of nucleotide to be replaced. For example, a nucleotide replaced with the nucleoside analog is A, correspondingly, the B base group of the nucleoside analog according to the first aspect is protected or unprotected A, and the B′ base group of the nucleoside analog according to the second aspect is unprotected A; a nucleotide replaced with the nucleoside analog is U, correspondingly, the B base group of the nucleoside analog according to the first aspect is protected or unprotected U, and the B′ base group of the nucleoside analog according to the second aspect is unprotected U; a nucleotide replaced with the nucleoside analog is C, correspondingly, the B base group of the nucleoside analog according to the first aspect is protected or unprotected C, and the B′ base group of the nucleoside analog according to the second aspect is unprotected C; a nucleotide replaced with the nucleoside analog is G, correspondingly, the B base group of the nucleoside analog according to the first aspect is protected or unprotected G, and the B′ base group of the nucleoside analog according to the second aspect is unprotected G; and a nucleotide replaced with the nucleoside analog is T, correspondingly, the B base group of the nucleoside analog according to the first aspect is protected or unprotected T, and the B′ base group of the nucleoside analog according to the second aspect is unprotected T.

It should be noted that the preceding nucleoside analogs can be compounds participating in reaction, and after the reaction thereof, the hydroxyl protecting group, and/or the phosphorus-containing leaving group, and/or the amino protecting group (for example, benzoyl (Bz), acetyl (Ac), isobutyryl) on the B base group are removed. For example, for the nucleoside analog according to the first aspect, R₁, and/or R₂, and/or the amino protecting group on the B base group are removed after reaction, and it can be also a compound after reaction, which is located in an oligonucleotide or double-stranded oligonucleotide, and is linked to other nucleotides, for instance, the nucleoside analog according to the second aspect.

According to embodiments of the present disclosure, the oligonucleotide is selected from the group consisting of single-stranded oligonucleotide or double-stranded oligonucleotide.

According to embodiments of the present disclosure, the single-stranded oligonucleotide is selected from the group consisting of antisense oligonucleotide, nucleic acid aptamer, ribozyme, deoxyribozyme, circular RNA, sense strand of siRNA or antisense strand of siRNA.

According to embodiments of the present disclosure, the oligonucleotide is selected from antisense oligonucleotide (ASO).

According to embodiments of the present disclosure, the double-stranded oligonucleotide is selected from the group consisting of small interfering RNA, double-stranded RNA, miRNA, small guide RNA, small activating RNA or short hairpin RNA.

According to embodiments of the present disclosure, the oligonucleotide is selected from small interfering RNA (siRNA).

In the fourth aspect of the present disclosure, the present disclosure provides a double-stranded oligonucleotide, wherein the double-stranded oligonucleotide comprises a sense strand and an antisense strand, the antisense strand and the sense strand have a complementary region with bases at least partially paired, and the sense strand and/or the antisense strand contain at least one nucleoside analog according to the second aspect.

According to embodiments of the present disclosure, each nucleotide in the double-stranded oligonucleotide other than the nucleoside analog is independently a modified or unmodified nucleotide. It should be noted that, in the double-stranded oligonucleotide of the present disclosure, the nucleoside analog described in the second aspect of the present disclosure can be used to replace one or more modified or unmodified nucleotides (A, U, C, G or T) in the nucleotide sequence of the double-stranded oligonucleotide; that is, the nucleobase in the double-stranded oligonucleotide replaced with the nucleoside analog according to the second aspect is the same as the B′ nucleobase of the nucleoside analog according to the second aspect. For example, the nucleobase in the double-stranded oligonucleotide replaced with the nucleoside analog is A, and correspondingly, the B′ base group of the nucleoside analog according to the second aspect is A; the nucleobase in the double-stranded oligonucleotide replaced with the nucleoside analog is U, and correspondingly, the B′ base group of the nucleoside analog according to the second aspect is U; the nucleobase in the double-stranded oligonucleotide replaced with the nucleoside analog is C, and correspondingly, the B′ base group of the nucleoside analog according to the second aspect is C; the nucleobase in the double-stranded oligonucleotide replaced with the nucleoside analog is G, and correspondingly, the B′ base group of the nucleoside analog according to the second aspect is G; and the nucleobase in the double-stranded oligonucleotide replaced with the nucleoside analog is T, and correspondingly, the B′ base group of the nucleoside analog according to the second aspect is T.

According to embodiments of the present disclosure, modification of each of the nucleotides in the double-stranded oligonucleotide each is at least one independently optionally selected from the the group consisting of:

2′-deoxy modification, 2′-fluoro modification, 2′-amino modification, 2′-O-allyl modification, 2′-O-methyl modification, 2′-methoxyethyl modification, 2′-allyl modification, abasic modification, tetrahydropyran modification, 1,5′-anhydrohexitol modification, cyclohexenyl modification, PEG modification, 5′-phosphoramidate modification, 5′-phosphorothioate modification, 5′-methylphosphonate modification, 5′-phosphate mimic modification and 5′-methylated cytosine modification.

According to embodiments of the present disclosure, modification of each of the nucleotides is at least one independently optionally selected from the group consisting of: 2′-deoxy modification, 2′-fluoro modification, 2′-O-methyl modification and 2′-O-allyl modification.

According to embodiments of the present disclosure, the double-stranded oligonucleotide is selected from the group consisting of a small interfering RNA, double-stranded RNA, miRNA, small guide RNA, small activating RNA and short hairpin RNA.

According to embodiments of the present disclosure, the double-stranded oligonucleotide is selected from siRNA.

According to embodiments of the present disclosure, the sense strand and the antisense strand each independently have a length of 15-25 nucleotides, for example, 15-24, 15-23, 15-22, 15-21, 15-20, 15-19, 16-25, 16-24, 16-23, 16-22, 16-21, 16-20, 16-19, 17-25, 17-24, 17-23, 17-22, 17-21, 17-20, 17-19, 18-25, 18-24, 18-23, 18-22, 18-21, 18-20, 18-19, 19-25, 19-24, 19-23, 19-22, 19-21 or 19-20.

According to embodiments of the present disclosure, the sense strand and/or the antisense strand contain one of the nucleoside analogs.

According to embodiments of the present disclosure, a structure the sense strand or the antisense strand is any structure of the following:
the nucleoside analog is located at 5′-end of the sense strand or the antisense strand, and the sense strand or the antisense strand has a structural formula as follows:

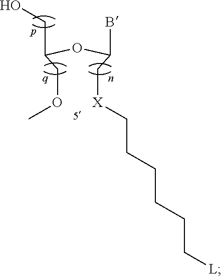

or
the nucleoside analog is located at 3′-end of the sense strand or the antisense strand, and the sense strand or the antisense strand has a structural formula as follows:

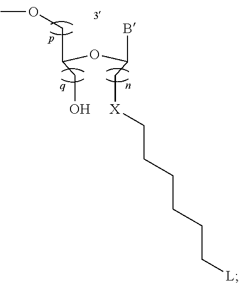

the nucleoside analog is located at non-5′-end and non-3′-end of the sense strand or the antisense strand, and the sense strand or the antisense strand has a structural formula as follows:

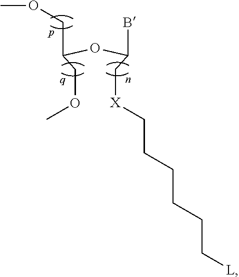

wherein [symbol] represents the sense strand or at least part thereof, or the antisense strand or at least part thereof; and
p, q, n, B′, X and L are respectively the same as p, q, n, B′, X and L as defined for the nucleoside analog according to the second aspect.

According to some embodiments of the present disclosure, the antisense strand does not contain the nucleoside analog, and the sense strand contains one of the nucleoside analogs.

According to embodiments of the present disclosure, the nucleoside analog is located at Positions 1-8 of the sense strand counting from the 5′-end.

According to embodiments of the present disclosure, the nucleoside analog is located at Positions 2-8 of the sense strand counting from the 5′-end.

According to embodiments of the present disclosure, the nucleoside analog is located at Positions 3-7 of the sense strand counting from the 5′-end.

According to some embodiments of the present disclosure, the nucleoside analog is located at Positions 4-6 of the sense strand counting from the 5′-end.

According to some embodiments of the present disclosure, the nucleoside analog is located at Position 4 of the sense strand counting from the 5′-end.

According to some embodiments of the present disclosure, the nucleoside analog is located at Position 5 of the sense strand counting from the 5′-end.

According to some embodiments of the present disclosure, the nucleoside analog is located at Position 6 of the sense strand counting from the 5′-end.

In the fifth aspect of the present disclosure, the present disclosure provides a composition. According to embodiments of the present disclosure, the composition comprises the double-stranded oligonucleotide according to the fourth aspect.

According to embodiments of the present disclosure, the composition further comprises optionally one or more pharmaceutically acceptable carriers or adjuvants.

In the sixth aspect of the present disclosure, the present disclosure provides use of the nucleoside analog according to the first aspect, the nucleoside analog according to the second aspect, the double-stranded oligonucleotide according to the fourth aspect or the composition according to the fifth aspect in preparation of medicaments for treating and/or preventing pathological conditions or diseases caused by abnormal expression of a specific gene in target cells.

The term “abnormal expression” herein refers to a case wherein an expression level of a gene in cells is higher than a normal expression level, for example, gene overexpression, or an expression level of a gene which is not present occurs.

According to embodiments of the present disclosure, the target cells are selected from nervous system cells.

In the seventh aspect of the present disclosure, the present disclosure provides a method for inhibiting expression of a specific gene in target cells, wherein the method comprises: contacting the double-stranded oligonucleotide according to the fourth aspect or the composition according to the fifth aspect with the target cells.

In the eighth aspect of the present disclosure, the present disclosure provides a method for preventing and/or treating pathological conditions or diseases caused by abnormal expression of a specific gene in target cells, wherein the method comprises: administering to a subject a pharmaceutically acceptable dose of the double-stranded oligonucleotide according to the fourth aspect or the composition according to the fifth aspect.

According to embodiments of the present disclosure, the target cells are selected from nervous system (CNS) cells.

According to embodiments of the present disclosure, the nervous system cells comprise neuronal cells and neuroglial cells.

According to embodiments of the present disclosure, the specific gene is at least one selected from the group consisting of APP, ATXN2, HTT, SNCA, FUS, PRNP, SOD1, DMPK and TTR.

An effective amount of the double-stranded oligonucleotide or composition according to the present disclosure may vary depending on a mode of administration, severity of a disease being treated, and the like. The choice of a preferred effective amount can be determined by those of ordinary skill in the art according to various factors (for example, through clinical trials). The factors comprise, but are not limited to: pharmacokinetic parameters of an active ingredient, such as bioavailability, metabolism, and half-life, the severity of the disease of a patient being treated, weight of the patient, immune status of the patient, route of administration, etc. For example, depending on exigencies of therapeutic situation, several divided doses can be administered monthly, quarterly or annually, for example, in doses of four times a month, three times a month, twice a month, once a month, once every two months, once every three months, once every half year or once every year.

Any suitable route known in the art can be used for administration to the subject, including, but is not limited to, oral or parenteral route, including intravenous administration, intramuscular administration, subcutaneous administration, transdermal administration, endotracheal administration (aerosol), pulmonary administration, nasal administration, intracerebroventricular administration, intrathecal administration, rectal administration and topical administration (including buccal administration and sublingual administration), preferably intravenous injection administration.

It should be noted that the nucleotide sequence of the double-stranded oligonucleotide herein can be adjusted and designed according to the target gene, as long as it can have a complementary region with bases at least partially paired with mRNA of the target gene, so as to ensure that at least one strand of the double-stranded oligonucleotide can be complementarily paired with the mRNA of the target gene, and a specific sequence thereof is not limited.

In the ninth aspect of the present disclosure, the present disclosure provides a nucleotide analog as represented by (100), or a tautomer thereof, or a stereoisomer thereof, or a pharmaceutically acceptable salt thereof:

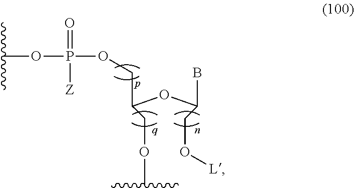

wherein [symbol] represents a covalently linking position of the nucleotide analog;
B is selected from the group consisting of substituted or unsubstituted nucleobase, or substituted or unsubstituted nucleobase analog; and if the B contains a substituent, the substituent is independently selected from the group consisting of halogen, C₁-C₃ alkyl, C₁-C₃ alkyloxy, or amide group;
L′ is selected from the group consisting of substituted or unsubstituted C₁₆-C₂₄ hydrocarbyl; and if the L′ contains a substituent, the substituent is independently selected from the group consisting of halogen, C₁-C₃ alkyl or C₁-C₃ alkoxy;
p is selected from 1, 2, 3 or 4;
q is selected from 1, 2, 3 or 4;
n is selected from 1, 2 or 3; and
Z is selected from the group consisting of hydroxyl or thiol.

In some embodiments of the present disclosure, the C₁₆-C₂₄ hydrocarbyl is selected from the group consisting of saturated or unsaturated C₁₆-C₂₄ hydrocarbon chain, the hydrocarbon chain being linear hydrocarbyl or branched hydrocarbyl, such as linear C₁₆-C₂₄ alkyl or alkenyl, branched C₁₆-C₂₄ alkyl or alkenyl.

In some embodiments of the present disclosure, the nucleotide analog is selected from the group consisting of a structure as represented by Formula (200), or a tautomer thereof, or a stereoisomer thereof, or a pharmaceutically acceptable salt thereof:

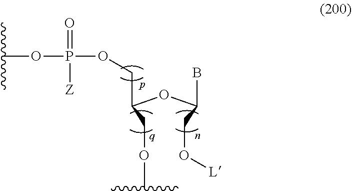

In some optional embodiments of the present disclosure, B is selected from nucleobases A, U, G C, or T, or analogs of the nucleobases.

In some optional embodiments of the present disclosure, L′ is selected from the group consisting of saturated or unsaturated C₁₈-C₂₂ hydrocarbyl.

In some optional embodiments of the present disclosure, p is selected from 1 or 2.

In some optional embodiments of the present disclosure, q is selected from 1 or 2.

In some optional embodiments of the present disclosure, n is selected from 1 or 2.

In some optional embodiments of the present disclosure, L′ is selected from the group consisting of C₁₈-C₂₂ alkyl, such as C₁₈-C₂₁ alkyl, C₁₈-C₂₀ alkyl, C₁₉-C₂₂ alkyl, C₁₉-C₂₁ alkyl, C₁₉-C₂₀ alkyl or C₂₀ alkyl.

In some optional embodiments of the present disclosure, L′ is selected from the group consisting of C₁₈-C₂₂ linear alkyl, such as C₁₈-C₂₁ linear alkyl, C₁₈-C₂₀ linear alkyl, C₁₉-C₂₂ linear alkyl, C₁₉-C₂₁ linear alkyl, C₁₉-C₂₀ linear alkyl or C₂₀ linear alkyl.

In some optional embodiments of the present disclosure, L′ is selected from the group consisting of C₁₈ linear alkyl, C₁₉ linear alkyl, C₂₀ linear alkyl, C₂₁ linear alkyl or C₂₂ linear alkyl.

In some optional embodiments of the present disclosure, n is selected from 1.

In some optional embodiments of the present disclosure, the nucleotide analog has a structure selected from any one of

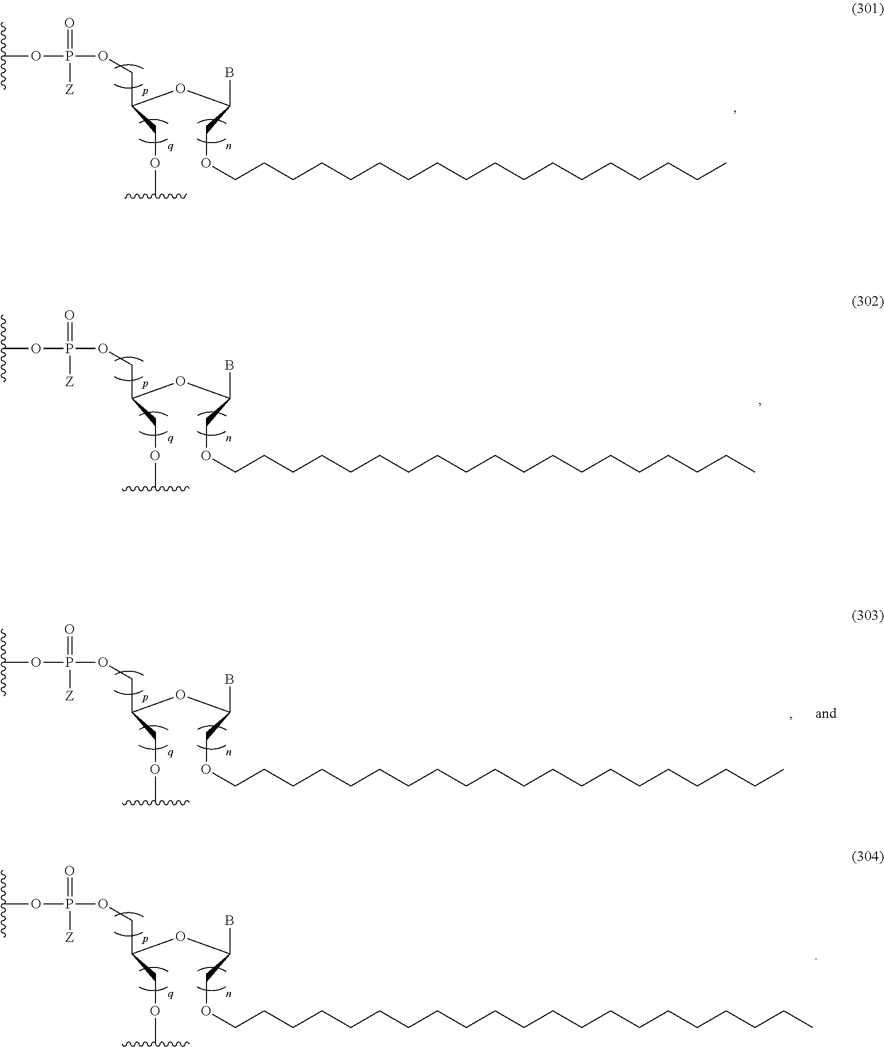

In the tenth aspect of the present disclosure, the present disclosure provides a double-stranded oligonucleotide, wherein the double-stranded oligonucleotide comprises a sense strand and an antisense strand, each strand has 17-25 modified and/or unmodified nucleotides, the antisense strand and the sense strand are complementary to form a duplex region; and the sense strand and/or the antisense strand contains at least one nucleotide analog according to the ninth aspect of the present disclosure.

The nucleotide analog has a structure as represented by Formula (100), or a tautomer thereof, or a stereoisomer thereof, or a pharmaceutically acceptable salt thereof:

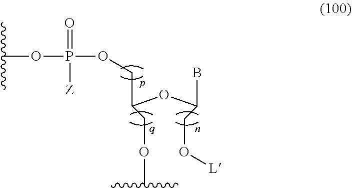

wherein [symbol] a covalently linking position between the nucleotide analog and a neighboring nucleotide;
B is selected from the group consisting of substituted or unsubstituted nucleobase, or substituted or unsubstituted nucleobase analog; and if the B contains a substituent, the substituent is independently selected from the group consisting of halogen, C₁-C₃ alkyl, C₁-C₃ alkyloxy, or amide group;
L′ is selected from the group consisting of substituted or unsubstituted C₁₆-C₂₄ hydrocarbyl; and if the L′ contains a substituent, the substituent is independently selected from the group consisting of halogen, C₁-C₃ alkyl or C₁-C₃ alkoxy;
p is selected from 1, 2, 3 or 4;
q is selected from 1, 2, 3 or 4;
n is selected from 1, 2 or 3; and
Z is selected from the group consisting of hydroxyl or thiol.

In some optional embodiments of the present disclosure, the sense stand and/or the antisense strand contains at least one nucleotide analog as represented by Formula (200), or a tautomer thereof, or a pharmaceutically acceptable salt thereof:

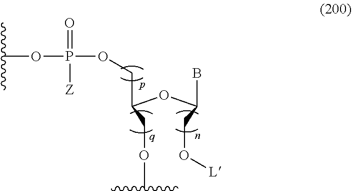

wherein definitions of substituents are the same as those in Formula (100).

In some optional embodiments of the present disclosure, B is selected from nucleobases A, U, G, C or T, or analogs of the nucleobases.

In some optional embodiments of the present disclosure, L′ is selected from saturated or unsaturated C₁₈-C₂₂ hydrocarbyl.
p is selected from 1 or 2;
q is selected from 1 or 2;
n is selected from 1 or 2; and
Z is selected from hydroxyl or thiol.

In some optional embodiments of the present disclosure, L′ is selected from the group consisting of C₁₈-C₂₂ alkyl, such as C₁₈-C₂₁ alkyl, C₁₈-C₂₀ alkyl, C₁₉-C₂₂ alkyl, C₁₉-C₂₁ alkyl, C₁₉-C₂₀ alkyl or C₂₀ alkyl.

In some optional embodiments of the present disclosure, L′ is selected from the group consisting of C₁₈-C₂₂ linear alkyl, such as C₁₈-C₂₁ linear alkyl, C₁₈-C₂₀ linear alkyl, C₁₉-C₂₂ linear alkyl, C₁₉-C₂₁ linear alkyl, C₁₉-C₂₀ linear alkyl or C₂₀ linear alkyl.

In some optional embodiments of the present disclosure, L′ is selected from the group consisting of C₁₈ linear alkyl, C₁₉ linear alkyl, C₂₀ linear alkyl, C₂₁ linear alkyl or C₂₂ linear alkyl.

In some optional embodiments of the present disclosure, n is selected from 1.

In some optional embodiments of the present disclosure, the nucleotide analog is a compound selected from any one with following structures, or tautomers thereof, or pharmaceutically acceptable salts thereof:

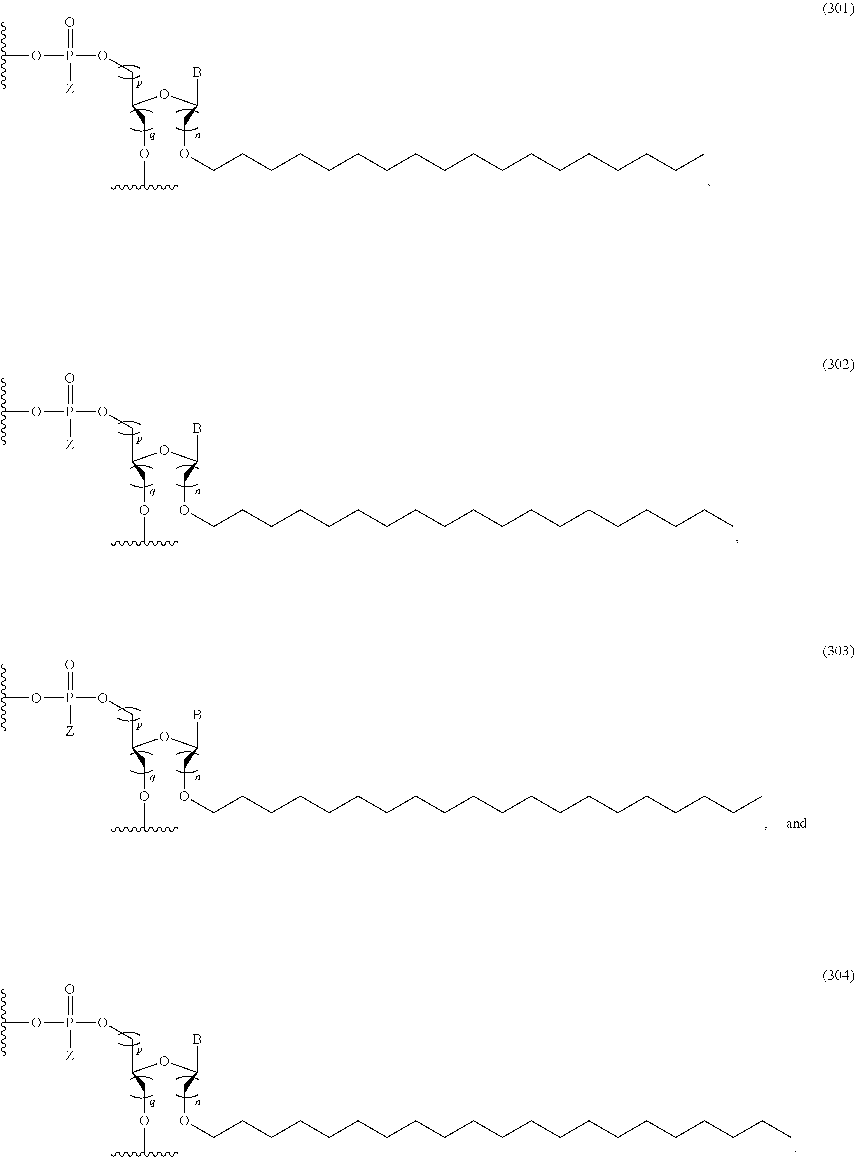

In some embodiments of the present disclosure, the sense stand contains one or two of the nucleotide analogs.

In some embodiments of the present disclosure, the sense stand contains one of the nucleotide analogs. In some embodiments of the present disclosure, the nucleotide analog can be located at 5′-end of the sense strand, or at 3′-end of the sense strand, or at any position between the 5′-end and the 3′-end of the sense strand.

In some embodiments of the present disclosure, the nucleotide analog is located at the 5′-end or the 3′-end of the sense strand.

In some embodiments of the present disclosure, the nucleotide analog is located at any position in Positions 2-19 of the sense strand counting from the 5′-end.

In some embodiments of the present disclosure, the nucleotide analog is located at any position in Positions 2, 3, 4, 5, 6, 7, 8, 9, 10, 11, 12, 13, 14, 15, 16, 17, 18 and 19 of the sense strand counting from the 5′-end.

In some embodiments of the present disclosure, the nucleoside analog is located at any position in Positions 2-8 of the sense strand counting from the 5′-end.

In some embodiments of the present disclosure, the nucleoside analog is located at any position in Positions 4-6 of the sense strand counting from the 5′-end.

In some embodiments of the present disclosure, the sense strand and the antisense strand each independently have a length of 19-23 nucleotides. In some embodiments, the sense strand has a length of 21 nucleotides. In some embodiments, the antisense strand has a length of 19 nucleotides.

In some embodiments of the present disclosure, the antisense strand comprises 1, 2 or 3 phosphorothioate diester bonds located at the 5′-end and 1, 2 or 3 phosphorothioate diester bonds located at the 3′-end; and/or the sense strand comprises 1, 2 or 3 phosphorothioate diester bonds located at the 5′-end.

In some embodiments of the present disclosure, the 5′-end of the antisense strand contains a phosphate or phosphate mimic. In an embodiment, the phosphate mimic is selected from 5′-vinylphosphonates (VP).

In some embodiments of the present disclosure, the 5′-end of the antisense strand is free of 5′-vinylphosphonates (VP).

In some embodiments of the present disclosure, the antisense strand does not contain the nucleotide analog, and the antisense strand is basically reversely complementary, substantially reversely complementary or fully reversely complementary to a nucleotide sequence in an mRNA expressed by the target gene.

In some optional embodiments of the present disclosure, nucleotides of the double-stranded oligonucleotide other than the nucleotide replaced with the nucleotide analog are all modified, and are each independently selected from the following modified nucleotides: 2′-O-methoxy modified nucleotides, 2′-fluoro modified nucleotides and 2′-O-methoxyethyl (2′-O-MOE) modified nucleotides.

In some embodiments of the present disclosure, in a direction from the 5′-end to the 3′-end, nucleotides at Position 2, Position 6, Position 14, and Position 16 of the antisense strand are selected from 2′-fluoro modified nucleotides; and/or nucleotides at Position 7 and Position 9 of the sense strand are selected from 2′-fluoro modified nucleotides.

In some embodiments of the present disclosure, in the direction from the 5′-end to the 3′-end, at least three nucleotides at Positions 7-10 of the sense strand are selected from 2′-fluoro modified nucleotides; and/or at least four nucleotides at Positions 2, 6, 9, 12, 14 and 16 of the antisense strand are selected from 2′-fluoro modified nucleotides, and a nucleotide at Position 15 is selected from 2′-O-methoxyethyl or 2′-O-methyl modified nucleotide.

In some embodiments of the present disclosure, in the direction from the 5′-end to the 3′-end, at least three nucleotides at Positions 7-10 of the sense strand are selected from 2′-fluoro modified nucleotides, and one nucleotide at Positions 2-8 is selected from the nucleotide analog; and/or at least four nucleotides at Positions 2, 6, 9, 12, 14 and 16 of the antisense strand are selected from 2′-fluoro modified nucleotides, and a nucleotide at Position 15 is selected from 2′-O-methoxyethyl modified nucleotide or 2′-O-methyl modified nucleotide; and nucleotides at remaining positions are selected from 2′-O-methyl modified nucleotides.

In some embodiments of the present disclosure, in the direction from the 5′-end to the 3′-end, nucleotides at Positions 7-10 of the sense strand are selected from 2′-fluoro modified nucleotides, and one nucleotide at Positions 4-6 is selected from the nucleotide analog; and/or at least five nucleotides at Positions 2, 6, 9, 12, 14 and 16 of the antisense strand are selected from 2′-fluoro modified nucleotides, and a nucleotide at Position 15 is selected from 2′-O-methoxyethyl modified nucleotide; and nucleotides at remaining positions are selected from 2′-O-methyl modified nucleotides.

In some embodiments of the present disclosure, the double-stranded oligonucleotide further contains a targeting ligand, wherein the targeting ligand is targeted, mediated and delivered to a receptor of specific CNS tissues.

In some embodiments of the present disclosure, the double-stranded oligonucleotide containing the ligand has a structure as represented by Formula (400), or a tautomer thereof, or a stereoisomer thereof, or a pharmaceutically acceptable salt thereof:

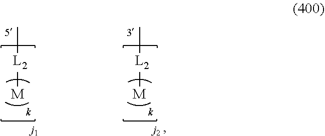

wherein [symbol] represents the double-stranded oligonucleotide;
SS represents the sense strand of the double-stranded oligonucleotide;
AS represents the antisense strand of the double-stranded oligonucleotide;
j₁ and j₂ are each independently selected from 0 or 1;
each k is independently selected from 1 or 2;
each M is independently selected from the targeting ligand; and
each L₂ is independently selected from a covalent linking group.

In some embodiments of the present disclosure, the j₁ is selected from 0, and the j₂ is selected from 0.

In some embodiments of the present disclosure, the j₁ is selected from 1, and the j₂ is selected from 0. In the above, one

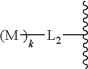

is conjugated to the 5′-end of the sense strand.

In some embodiments of the present disclosure, the j₁ is selected from 0, and the j₂ is selected from 1. In the above, one

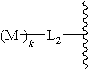

is conjugated to the 3′-end of the sense strand.

In some embodiments of the present disclosure, the j₁ is selected from 1, and the j₂ is selected from 1. In the above, the two

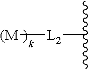

are conjugated to the 5′-end and the 3′-end of the sense strand, respectively.

In some embodiments of the present disclosure, each M is independently any structure selected from the group consisting of following structures, or tautomers thereof, or stereoisomers thereof, or pharmaceutically acceptable salts thereof:

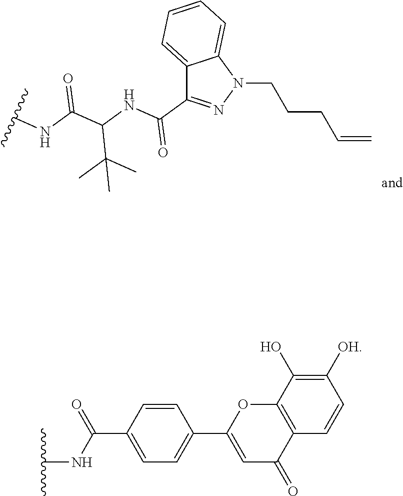 [SMILES: *NC(=O)C(NC(=O)c1nn(CCCC=C)c2ccccc12)C(C)(C)C ; *NC(=O)c1ccc(-c2cc(=O)c3ccc(O)c(O)c3o2)cc1]

In some embodiments of the present disclosure, each L₂ is independently any structure selected from the group consisting of following structures, or tautomers thereof, or stereoisomers thereof, or pharmaceutically acceptable salts thereof:

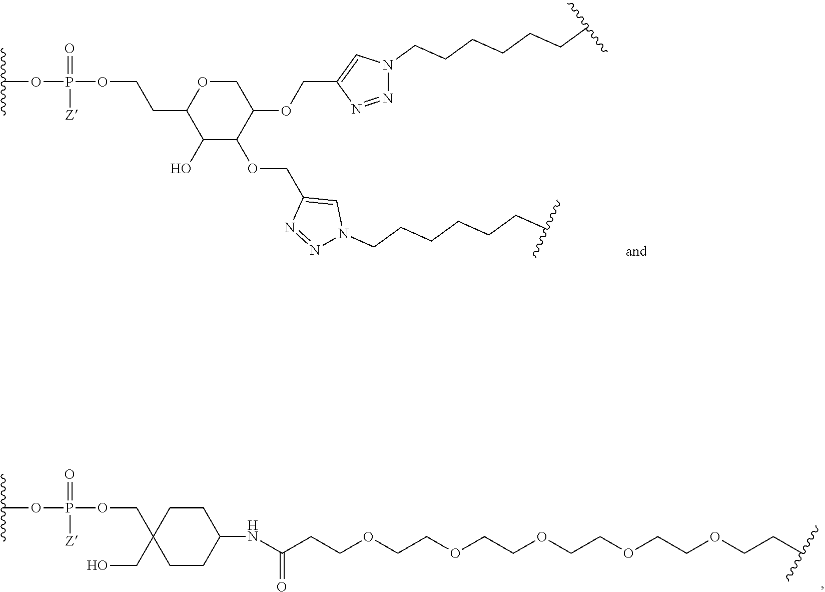

wherein Z′ is independently selected from hydroxyl or thiol.

In the above, when k is selected from 1, the L₂ is selected from the group consisting of following structure, or a tautomer thereof, or a stereoisomer thereof, or a pharmaceutically acceptable salt thereof:

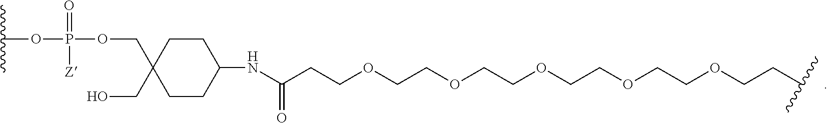

When k is selected from 2, the L₂ is selected from the group consisting of following structure, or a tautomer thereof, or a stereoisomer thereof, or a pharmaceutically acceptable salt thereof:

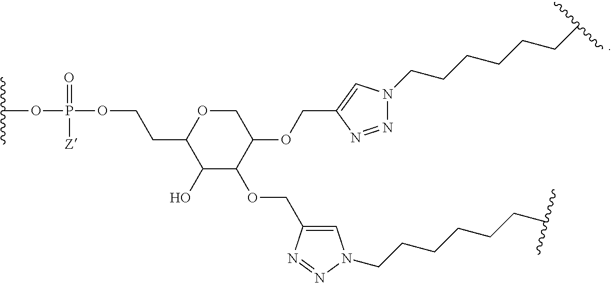

In some embodiments of the present disclosure, each

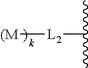

is independently any structure selected from the group consisting of following structures, or tautomers thereof, or stereoisomers thereof, or pharmaceutically acceptable salts thereof:

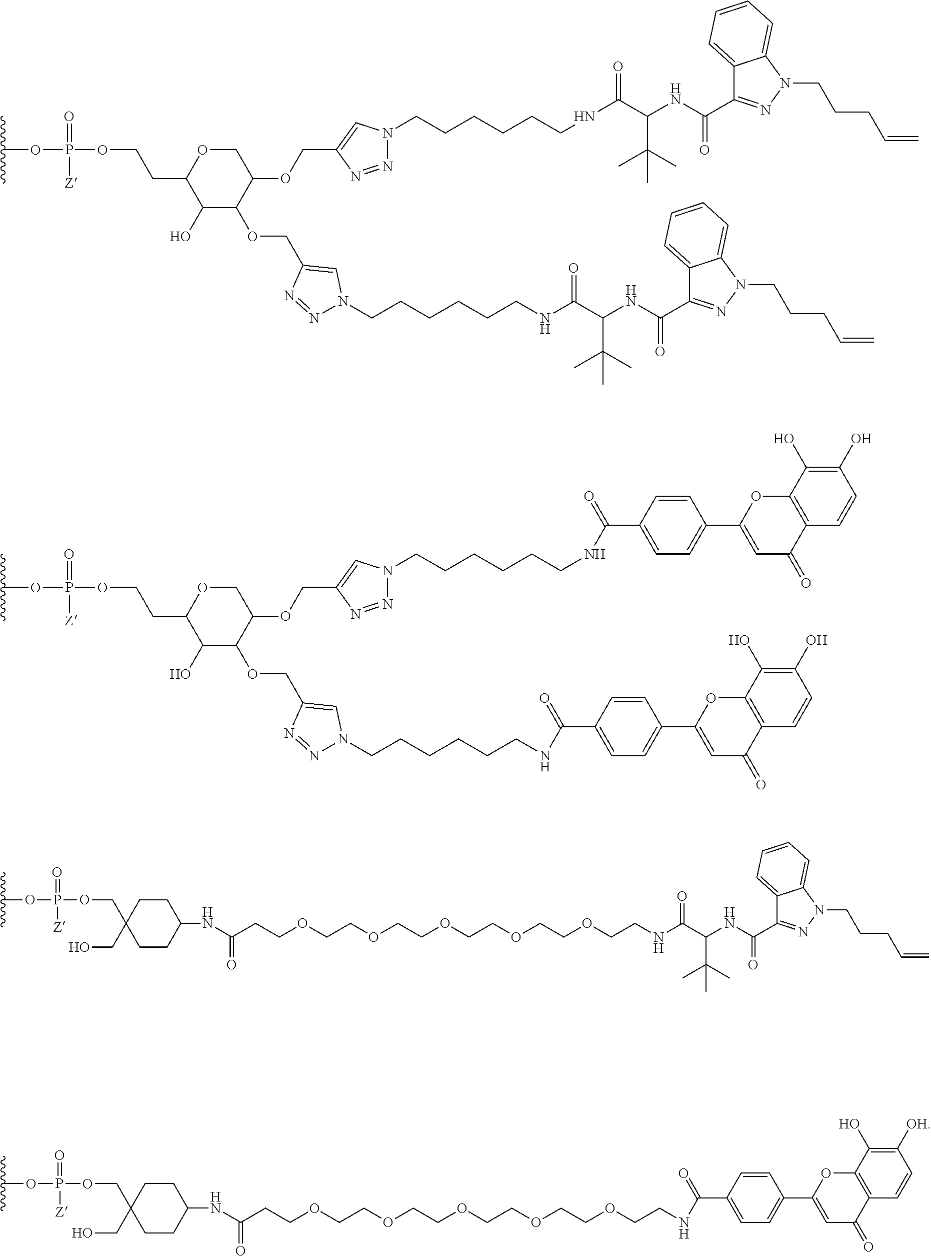

In some embodiments of the present disclosure, the double-stranded oligonucleotide has any structure selected from the group consisting of following structures, or tautomers thereof, or stereoisomers thereof, or pharmaceutically acceptable salts thereof:

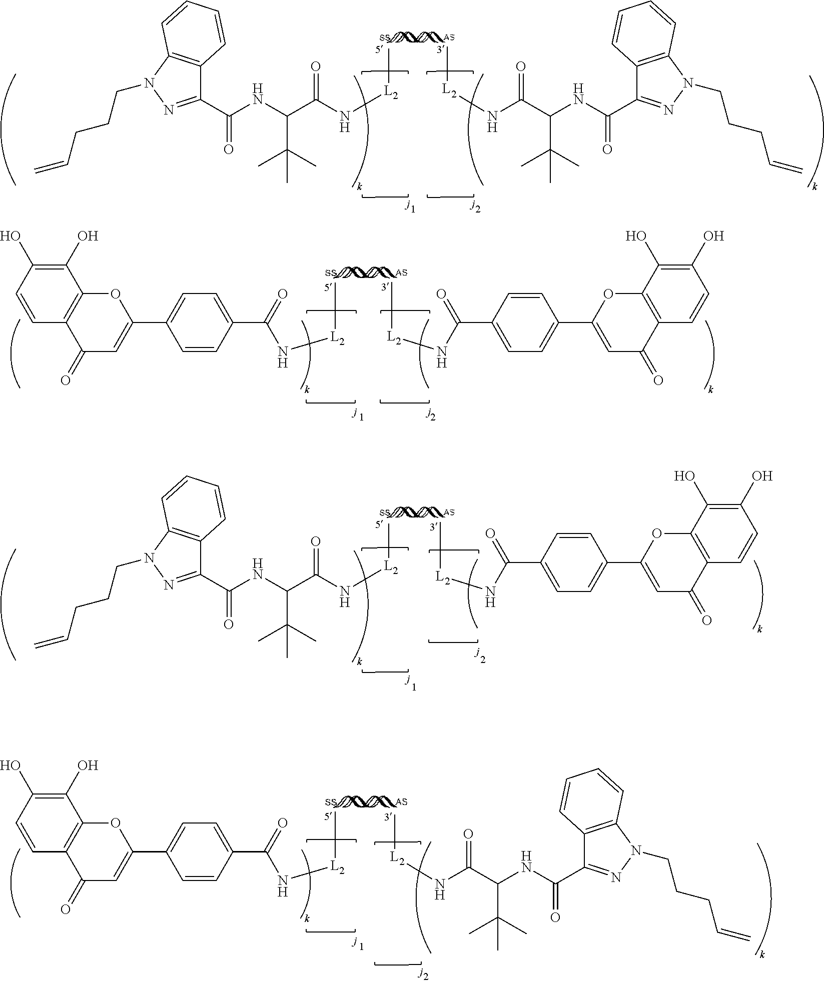

In some embodiments of the present disclosure, the double-stranded oligonucleotide has any structure selected from the group consisting of following structures, or tautomers thereof, or stereoisomers thereof, or pharmaceutically acceptable salts thereof:

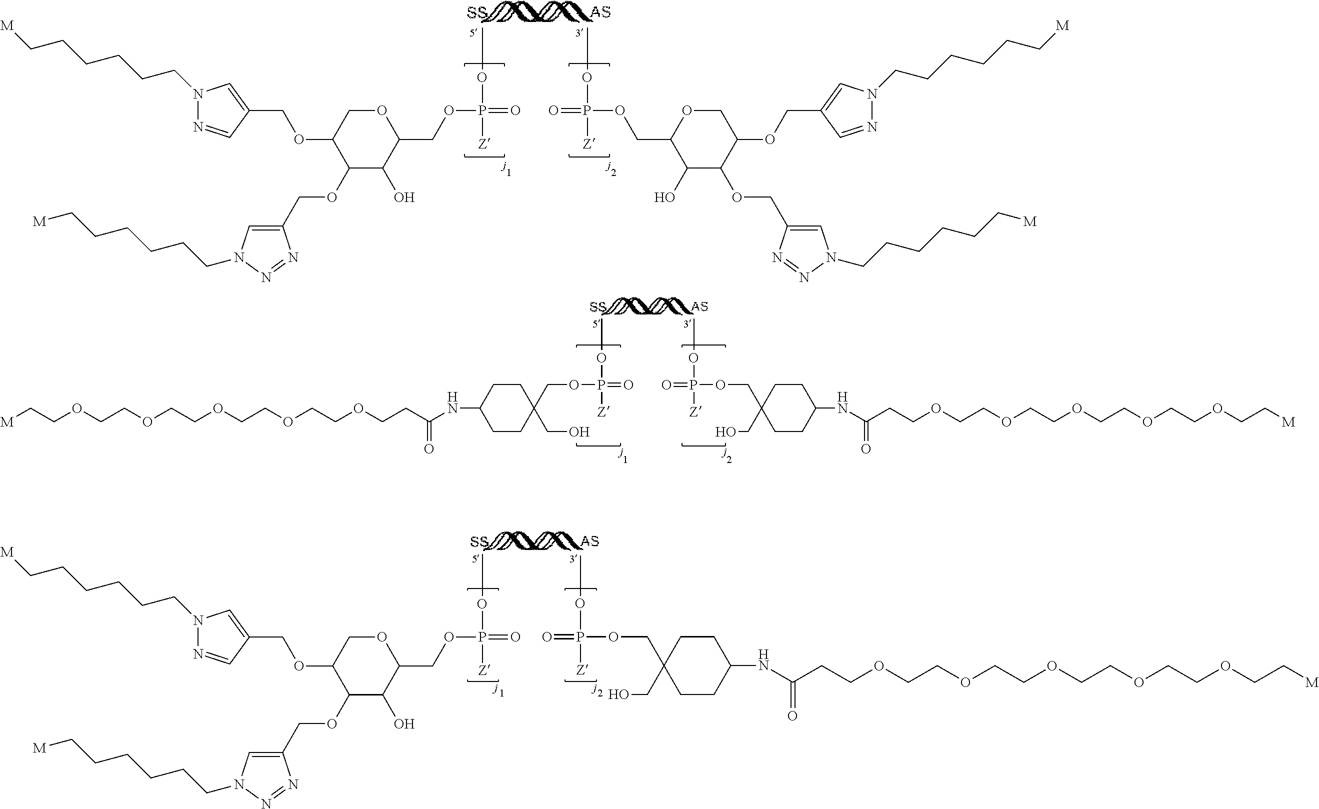

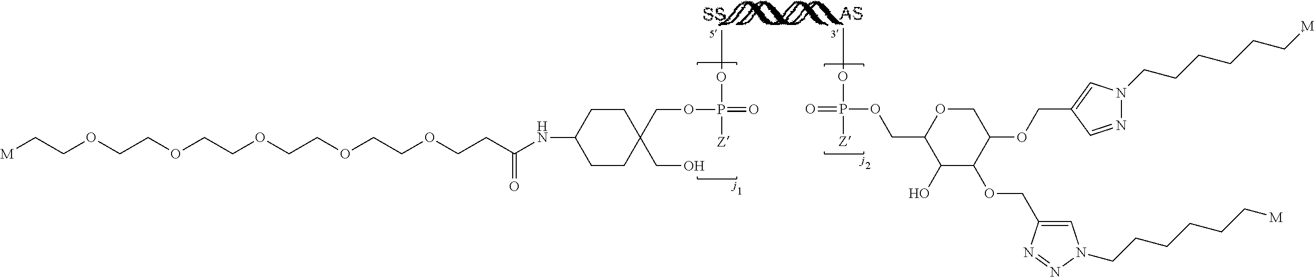

In an optional embodiment, the targeting ligand is selected from the group consisting of Angiopep-2, lipoprotein receptor-related protein (LRP) ligand, bEnd.3 cell binding ligand, transferrin receptor (TfR) ligand, mannose receptor ligand, glucose transporter and LDL receptor ligand.

In the eleventh aspect of the present disclosure, the present disclosure provides a composition, which comprises the double-stranded oligonucleotide according to the tenth aspect of the present disclosure.

In the twelfth aspect of the present disclosure, the present disclosure provides use of the nucleotide analog according to the ninth aspect of the present disclosure, and/or the double-stranded oligonucleotide according to the tenth aspect of the present disclosure, and/or the composition according to the eleventh aspect of the present disclosure in preparation of medicaments for treating and/or preventing pathological conditions or diseases caused by abnormal expression of a specific gene in nervous system cells.

In the thirteenth aspect of the present disclosure, the present disclosure further provides a pharmaceutical composition, which comprises the double-stranded oligonucleotide according to the tenth aspect of the present disclosure, and/or the composition according to the eleventh aspect of the present disclosure.

In some embodiments of the present disclosure, the composition further contains optionally one or more pharmaceutically acceptable carriers or adjuvants.

In the fourteenth aspect of the present disclosure, the present disclosure further provides a pharmaceutical composition, which comprises the double-stranded oligonucleotide according to the tenth aspect of the present disclosure, and/or the composition according to the eleventh aspect of the present disclosure, and/or the pharmaceutical composition according to the thirteenth aspect of the present disclosure.

In the fifteenth aspect of the present disclosure, the present disclosure further provides a method for reducing expression of a gene in target cells, wherein the method comprises: contacting the double-stranded oligonucleotide according to the tenth aspect of the present disclosure, and/or the composition according to the eleventh aspect of the present disclosure, and/or the pharmaceutical composition according to the thirteenth aspect of the present disclosure, and/or the medicament according to the fourteenth aspect of the present disclosure with the target cells.

In some embodiments of the present disclosure, the target cells are selected from nervous system cells.

In the sixteenth aspect of the present disclosure, the present disclosure further provides a method for reducing expression of a target gene in a subject, wherein the method comprises: administering to the subject the double-stranded oligonucleotide according to the tenth aspect of the present disclosure, and/or the composition according to the eleventh aspect of the present disclosure, and/or the pharmaceutical composition according to the thirteenth aspect of the present disclosure, and/or the medicament according to the fourteenth aspect of the present disclosure.

In some embodiments of the present disclosure, the method comprises intrathecal administration.

In some embodiments of the present disclosure, the method reduces the expression of the target gene in brain or spine tissues.

In some embodiments of the present disclosure, the brain or spine tissues comprise, but are not limited to, the group consisting of cortex, cerebellum, cervical spine, lumbar spine and thoracic spine.

In some embodiments of the present disclosure, an exemplary target gene is selected from the group consisting of APP, ATXN2, C9orf72, TARDBP, MAPT (Tau), HTT, SNCA, FUS, ATXN3, ATXN1, SCA1, SCA7, SCA8, MeCP2, PRNP, SOD1, DMPK or TTR.

In the seventeenth aspect of the present disclosure, the present disclosure further provides a method for preventing and/or treating pathological conditions or diseases caused by abnormal expression of a specific gene in target cells, wherein the method comprises: administering to the subject a pharmaceutically acceptable dose of the double-stranded oligonucleotide according to the tenth aspect of the present disclosure, and/or the composition according to the eleventh aspect of the present disclosure, and/or the pharmaceutical composition according to the thirteenth aspect of the present disclosure, and/or the medicament according to the fourteenth aspect of the present disclosure.

In some embodiments of the present disclosure, the target cells are selected from nervous system cells.

In the eighteenth aspect of the present disclosure, the present disclosure further provides a method for treating a subject suffering from CNS disorder-associated diseases or conditions, wherein the method comprises:
administering to the subject a therapeutically effective amount of the double-stranded oligonucleotide according to the tenth aspect of the present disclosure, and/or the composition according to the eleventh aspect of the present disclosure, and/or the pharmaceutical composition according to the thirteenth aspect of the present disclosure, and/or the medicament according to the fourteenth aspect of the present disclosure, so as to treat the CNS disorder-associated diseases or conditions of the subject.

In some embodiments of the present disclosure, the CNS disorder is selected from the group consisting of: Parkinson's disease, amyotrophic lateral sclerosis (ALS), diffuse Lewy body disease, Parkinson's disease dementia, Huntington's disease, multiple system atrophy, Alzheimer's disease and other neurodegenerative diseases.

In some embodiments of the present disclosure, the subject is selected from humans.

In the nineteenth aspect of the present disclosure, the present disclosure provides a kit, wherein the kit comprises the nucleotide analog according to the ninth aspect of the present disclosure, and/or the double-stranded oligonucleotide according to the tenth aspect of the present disclosure, and/or the composition according to the eleventh aspect of the present disclosure, and/or the pharmaceutical composition according to the thirteenth aspect of the present disclosure, and/or the medicament according to the fourteenth aspect of the present disclosure.

In the twentieth aspect of the present disclosure, the present disclosure further provides a method for inhibiting expression of a target gene in skeletal muscle cells, ocular cells, myocardial cells or adipose cells, wherein the method comprises: administering to the subject a therapeutically effective amount of the double-stranded oligonucleotide according to the tenth aspect of the present disclosure, and/or the composition according to the eleventh aspect of the present disclosure, and/or the pharmaceutical composition according to the thirteenth aspect of the present disclosure.

In the twenty-first aspect of the present disclosure, the present disclosure further provides a method for preventing and/or treating diseases or conditions associated with skeletal muscles, cardiac muscles, eyes or adipose tissues, wherein the method comprises: administering to the subject a therapeutically effective amount of the double-stranded oligonucleotide according to the tenth aspect of the present disclosure, and/or the composition according to the eleventh aspect of the present disclosure, and/or the pharmaceutical composition according to the thirteenth aspect of the present disclosure.

In some embodiments of the present disclosure, the subject is a human.

In some embodiments of the present disclosure, the diseases or conditions associated with skeletal muscles are muscular atrophies.

In some embodiments of the present disclosure, muscular atrophies are selected from the group consisting of Duchenne muscular atrophy, myotonic muscular atrophy, Becker muscular atrophy, limb-girdle type muscular atrophy, facioscapulohumeral muscular atrophy, congenital muscular atrophy, oculopharyngeal muscular atrophy, distal muscular atrophy and Emery-Dreifuss muscular atrophy, myostatin-related muscular hypertrophy, congenital myasthenic syndrome and facioscapulohumeral muscular dystrophy (FSHD).

In some embodiments of the present disclosure, the diseases or conditions associated with cardiac muscles consist of: hypertrophic obstructive cardiomyopathy (HOCM); familial hypertrophic cardiomyopathy (FHC); heart failure with preserved ejection fraction (HFPEF); atrial fibrillation (AFIB); ventricular fibrillation (VFIB); angina pectoris; myocardial infarction (MI); heart failure or heart failure with reduced ejection fraction (HFREF); supra ventricular tachycardia (SVT); hypertrophic cardiomyopathy (HCM), dilated cardiomyopathy (DCM), arrhythmia and congestive heart failure (CHF).

In some embodiments of the present disclosure, the diseases or conditions associated with the adipose tissues are metabolic diseases.

In some embodiments of the present disclosure, the metabolic diseases are selected from the group consisting of: lipid metabolism conditions, hypertension, cardiovascular diseases or overweight-related conditions.

In some embodiments of the present disclosure, the diseases or conditions associated with the eyes are selected from the group consisting of: glaucoma (including but not limited to primary open-angle glaucoma, secondary glaucoma, pigmentary glaucoma, pseudo-exfoliative glaucoma, traumatic glaucoma, neovascular glaucoma, uveitic glaucoma, ocular conditions of angle-closure glaucoma, normal-tension glaucoma, juvenile open-angle glaucoma, primary open-angle glaucoma), iridocorneal endothelial syndrome, macular degeneration, cataracts, diabetic retinopathy, dry eye, night vision, strabismus, ocular tremor, color blindness, uveitis, ocular inflammation, presbyopia, retinal diseases, corneal diseases, diabetic macular edema, ocular hypertension, astigmatism, diabetic eye diseases, hypermetropia, myopia, and macular edema.

In some embodiments of the present disclosure, a double-stranded oligonucleotide agent is administered to the subject by subcutaneous administration, intramuscular administration, intravenous administration, intraperitoneal administration or intravitreal administration.

In some embodiments of the present disclosure, administration of the double-stranded oligonucleotide agent results in that expression of a target gene is no more than 40%, no more than 50%, no more than 60% or no more than 70% of that without administration.

Solutions of the present disclosure will be explained below in conjunction with examples. Those skilled in the art would understand that the following examples are merely used for illustrating the present disclosure, but should not be considered as limitation to the scope of the present disclosure. Where specific techniques or conditions are not specified in the examples, they are carried out according to techniques or conditions described in documents in the art or according to product specifications. If manufacturers of reagents or apparatuses used are not specified, all of them are conventional products commercially available.

1-[(2R,3R,4S,5R)-5-{[bis(4-methoxyphenyl)(phenyl)methoxy]methyl}-3,4-dihydroxyoxolan-2-yl]-3H-pyrimidin-2,4-dione, sodium periodate, sodium borohydride, lipase TLIM, benzoyl chloride, tert-butyldimethylsilyl chloride, 1-bromohexadecane, tetrabutylammonium fluoride, 3-{[bis(diisopropylamino)phosphoryl]oxy}propanenitrile, 1H-imidazole-4,5-dinitrile and similar reagents used in preparation examples of the present disclosure are all purchased from Beijing Ouhe Technology Co., Ltd.

Unless otherwise stated, meanings of base composition and modifications used in the present disclosure are as follows: capital letters A, U, G, C, and T represent base composition of nucleotides; lowercase letter m means that a nucleotide adjacent to the left of the letter m is a 2′-O-methyl modified (also called: 2′-methoxy modified) nucleotide; lowercase letter f means that a nucleotide adjacent to the left of the letter f is a 2′-fluoro modified nucleotide; (moe) means that a nucleotide adjacent to the left of combined identity (moe) is a 2′-O-methoxyethyl (i,e., 2′-O-MOE) modified nucleotide; and lowercase letter s means that two nucleotides adjacent to the left and right of the letter s are linked by a phosphorothioate diester bond. In the context of the present disclosure, unless otherwise stated, synthesis of siRNA sequences used in the present disclosure was entrusted to be completed by Suzhou Biosyntech Co., Ltd.; synthesis of PCR primers used in the present disclosure was entrusted to be completed by Beijing Tsinigke iotech Co., Ltd.; and experimental animals C57BL/6J mice used in the present disclosure were all purchased from SPF (Beijing) Biotechnology Co., Ltd.

In the context of the present disclosure, unless otherwise stated, Real-time PCR detection data in in vivo activity experiments involved in the present disclosure are all used for relative quantitative calculation of gene mRNA of interest in various test groups by a ΔΔCt method, and a calculation method is summarized as follows:

$$Δ Ct ( test group ) = Ct ( gene of interest in test group ) - Ct ( internal reference gene in test group ) ;$$

$$Δ Ct ( control group ) = Ct ( gene of interest in control group ) - Ct ( internal reference gene in control group ) ;$$

$$Δ Δ Ct ( test group ) = Δ Ct ( test group ) - Δ Ct ( control group mean ) ; and$$

$$Δ Δ Ct ( control group ) = Δ Ct ( control group ) - Δ Ct ( control group mean ) .$$

Taking the control group as reference, expression level of the gene mRNA of interest in the test group is normalized, and remaining expression level of the gene mRNA of interest in the control group is defined as 100%.

$$Relative remaining expression level of gene mRNA of interest in test group = {2}^{- Δ Δ Ct} ( test group ) \times 100 \%$$

$$Inhibition rate of gene mRNA of interest in test group = 100 \% - relative expression level of gene mRNA of interest in test group$$

In the context of the present disclosure, unless otherwise stated, in vivo activity experimental data are all expressed by X̅±SD or X̅±STDEV, and experimental data are all plotted and analyzed using GraphPad prism 8.0 software.

In the context of the present disclosure, proportions of reagents in various examples of the present disclosure are calculated as volume ratio (v/v), unless otherwise specified.

### Preparation Example 1: Synthesis of Compound NM031

A synthesis process of compound NM031 was as follows:

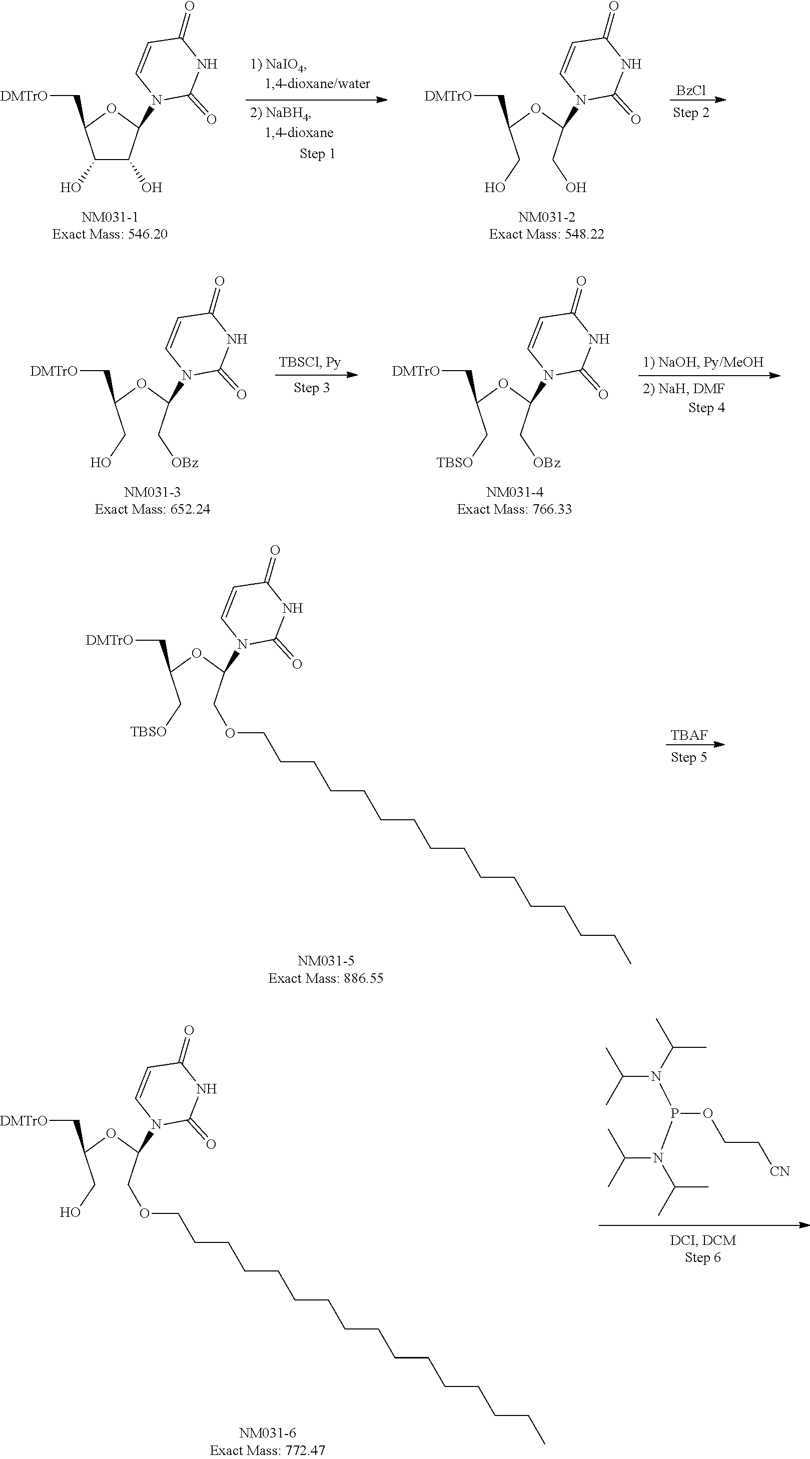

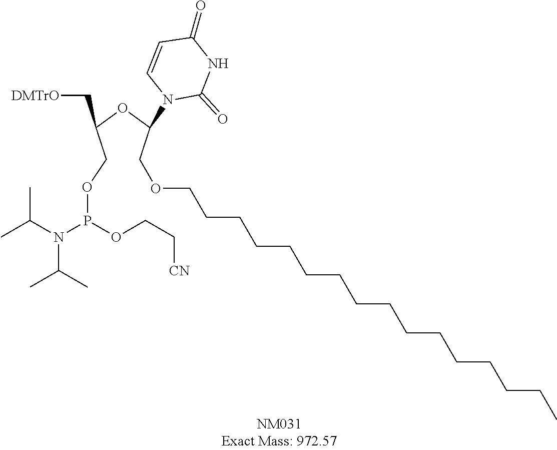

### (1-1) Synthesis of Compound NM031-2 (1-[(1R)-1-{[(2S)-1-[bis(4-methoxyphenyl)(phenyl)methoxy]-3-hydroxypropan-2-yl]oxy}-2-hydroxyethyl]-3H-pyrimidin-2,4-dione)

Compound NM031-1 (1-[(2R,3R,4S,5R)-5-{[bis(4-methoxyphenyl)(phenyl)methoxy]methyl}-3,4-dihydroxyoxolan-2-yl]-3H-pyrimidin-2,4-dione, 20 g, 36.592 mmol) was dissolved in a mixed solution of dioxane (160 mL) and water (40 mL), and added with sodium periodate (7.82 g, 36.592 mmol). In an argon atmosphere, reaction solution was stirred under a condition of 25° C. for 2 h. After reaction was ended, the reaction solution was concentrated to remove solvent, diluted by adding water (300 mL), and extracted with dichloromethane five times (5×200 mL). Organic phases were combined, washed with a saturated aqueous sodium bicarbonate solution twice (2×100 mL), washed with saturated brine twice (2×100 mL), dried over anhydrous sodium sulfate, filtered, and concentrated, to provide a white solid (16.2 g).

At 25° C., the white solid (16.2 g) was dissolved in dioxane (160 mL), and sodium borohydride (2.08 g, 54.888 mmol) was added portion-wise. In the argon atmosphere, reaction solution was stirred at 25° C. for 2 h, and quenched by adding a saturated aqueous ammonium chloride solution (120 mL). After reaction was ended, the reaction solution was extracted with dichloromethane four times (4×150 mL). Organic phases were combined, washed with saturated brine twice (2×100 mL), dried over anhydrous sodium sulfate, filtered, and concentrated, to provide white solid compound NM031-2 (13.5 g, yield 67.5%).

$$M S E S I ( m / z ) = {547.15 [ M - H ]}^{-} .$$

¹H NMR (400 MHz, DMSO-d6) δ 11.34 (br s, 1H), 7.64 (d, J=8.0 Hz, 1H), 7.40-7.24 (m, 5H), 7.23-7.11 (m, 5H), 6.89-6.82 (m, 4H), 5.81 (t, J=5.6 Hz, 1H), 5.52 (dd, J=8.0, 2.0 Hz, 1H), 5.13 (t, J=6.4 Hz, 1H), 4.74 (t, J=5.2 Hz, 1H), 3.73 (s, 6H), 3.71-3.60 (m, 2H), 3.40 (t, J=5.2 Hz, 2H), 3.07-2.88 (m, 2H).

### (1-2) Synthesis of Compound NM031-3 ((2R)-2-{[(2S)-1-[bis(4-methoxyphenyl)(phenyl)methoxy]-3-hydroxypropan-2-yl]oxy}-2-(2,4-dioxo-3H-pyrimidin-1-yl)ethyl benzoate)

At 25° C., compound NM031-2 (13 g, 23.696 mmol) was dissolved in toluene (120 mL), and added with lipase TLIM (0.5 g) and benzoyl chloride (3.33 g, 23.696 mmol). In a nitrogen atmosphere, reaction solution was stirred under a condition of 25° C. for 6 h, and quenched by adding a saturated aqueous ammonium chloride solution (150 mL). After reaction was ended, resultant was extracted with dichloromethane (3×120 mL). Organic phases were combined, washed with a saturated aqueous sodium bicarbonate solution (2×200 mL) and saturated brine (2×100 mL), dried over anhydrous sodium sulfate, filtered, concentrated, and subjected to reversed phase chromatography column purification (chromatography column: C18 silica gel, and elution gradient: acetonitrile/water=5/95, v/v), to provide yellow solid compound NM031-3 (10.2 g, yield 65.9%).

$$M S E S I ( m / z ) = {651.2 [ M - H ]}^{-} .$$

¹H NMR (400 MHz, Chloroform-d) δ 8.00-7.91 (m, 2H), 7.63-7.53 (m, 1H), 7.50-7.37 (m, 3H), 7.30-7.09 (m, 12H), 6.88-6.74 (m, 4H), 6.30-6.07 (m, 1H), 5.61 (dd, J=8.0, 2.0 Hz, 1H), 4.62-4.43 (m, 2H), 3.84-3.61 (m, 8H), 3.22-3.20 (m, 2H).

### (1-3) Synthesis of Compound NM031-4 ((2R)-2-{[(2R)-1-[bis(4-methoxyphenyl)(phenyl)methoxy]-3-[(tert-butyldimethylsilyl)oxy]propan-2-yl]oxy}-2-(2,4-dioxo-3H-pyrimidin-1-yl)ethyl benzoate)

At 25° C., compound NM031-3 (10.1 g, 15.474 mmol) was dissolved in pyridine (100 mL), and added with tert-butyldimethylsilyl chloride (2.80 g, 18.569 mmol). In a nitrogen atmosphere, reaction solution was stirred under a condition of 25° C. for 16 h. After reaction was ended, the reaction solution was spin-dried to remove solvent, diluted by adding water (400 mL), and extracted with dichloromethane (4×150 mL). Organic phases were combined, washed with a saturated aqueous sodium bicarbonate solution (2×80 mL) and saturated brine (100 mL), dried over anhydrous sodium sulfate, filtered, concentrated, and subjected to reversed phase chromatography column purification (chromatography column: C18 silica gel, and elution gradient: acetonitrile/water=5/95, v/v), to provide yellow solid compound NM031-4 (8.5 g, yield 71.6%).

$$M S E S I ( m / z ) = {765.3 [ M - H ]}^{-} .$$

¹H NMR (400 MHz, Chloroform-d) δ 7.96 (d, J=8.4 Hz, 2H), 7.60-7.51 (m, 2H), 7.45-7.33 (m, 4H), 7.29-7.19 (m, 8H), 6.84-6.73 (m, 4H), 6.36 (t, J=5.2 Hz, 1H), 5.58 (dd, J=8.0, 2.0 Hz, 1H), 4.59-4.33 (m, 2H), 3.77 (s, 6H), 3.72-3.58 (m, 3H), 3.17 (d, J=4.0 Hz, 2H), 0.84 (s, 9H), −0.01˜−0.03 (m, 6H).

### (1-4) Synthesis of Compound NM031-5 (1-((6R,8R)-6-((bis(4-methoxyphenyl)(phenyl)methoxy)methyl)-2,2,3,3-tetramethyl-4,7,10-trioxa-3-silahexane-8-yl)pyrimidin-2,4(1H,3H)-dione)

At 25° C., compound NM031-4 (7.5 g, 9.779 mmol) was dissolved in a mixed solution of pyridine (20 mL) and methanol (20 mL), cooled to 0° C. by an ice bath, and added with sodium hydroxide (0.23 g, 5.867 mmol). In a nitrogen atmosphere, reaction solution was stirred under a condition of 0° C. for 6 h. After reaction was ended, the reaction solution was spin-dried to remove solvent, diluted by adding water (150 mL), and extracted with dichloromethane (3×200 mL). Organic phases were combined, washed with a saturated aqueous ammonium chloride solution (2×100 mL) and saturated brine (2×100 mL), dried over anhydrous sodium sulfate, filtered and concentrated, to provide a white solid (3.84 g), which was directly used in the next step.

The white solid (3.84 g) was dissolved in N,N-dimethylformamide (50 mL), cooled to 0° C. by an ice bath, and added with sodium hydride (0.70 g, 60%, 29.337 mmol). In the nitrogen atmosphere, reaction solution was stirred under a condition of 0° C. for 20 min, added with 1-bromohexadecane (4.48 g, 14.668 mmol), slowly warmed to 25° C., stirred under a condition of 25° C. for 16 h, and quenched by adding a saturated aqueous ammonium chloride solution (150 mL). After reaction was ended, resultant was extracted with dichloromethane (3×150 mL). Organic phases were combined, washed with a saturated aqueous sodium bicarbonate solution (2×80 mL) and saturated brine (100 mL), dried over anhydrous sodium sulfate, filtered, concentrated, and subjected to normal phase chromatography column purification (elution gradient: petroleum ether/ethyl acetate=1/1, v/v), to provide yellow solid compound NM031-5 (1.82 g, yield 21.6%).

$$M S E S I ( m / z ) = {885.5 [ M - H ]}^{-} .$$

### (1-5) Synthesis of Compound NM031-6 (1-[(1R)-1-{[(2S)-1-[bis(4-methoxyphenyl)(phenyl)methoxy]-3-hydroxypropan-2-yl]oxy}-2-(pentaalkoxy)ethyl]-3H-pyrimidin-2,4-dione)

At 25° C., compound NM031-5 (1.8 g, 2.029 mmol) was cooled to 0° C. by an ice bath, and added with tetrabutylammonium fluoride (0.79 g, 3.044 mmol) dropwise. In a nitrogen atmosphere, reaction solution was stirred under a condition of 0° C. for 4 h. After reaction was ended, reaction solution was diluted by adding water (50 mL), and extracted with dichloromethane (3×50 mL). Organic phases were combined, washed with a saturated aqueous sodium bicarbonate solution (2×30 mL) and saturated brine (40 mL), dried over anhydrous sodium sulfate, filtered, concentrated, and subjected to normal phase chromatography column purification (elution gradient: petroleum ether/ethyl acetate=3/7, v/v), to provide yellow solid compound NM031-6 (1.05 g, yield 67.3%).

$$M S E S I ( m / z ) = {771.45 [ M - H ]}^{-} .$$

¹H NMR (300 MHz, DMSO-d6) δ 11.38 (d, J=2.1 Hz, 1H), 7.67 (d, J=8.1 Hz, 1H), 7.38-7.10 (m, 9H), 6.86 (d, J=9.0, 2.1 Hz, 4H), 5.98 (t, J=6.0 Hz, 1H), 5.53 (dd, J=8.1, 2.1 Hz, 1H), 4.87-4.67 (m, 1H), 3.73 (s, 6H), 3.66 (d, J=6.0 Hz, 2H), 3.43 (d, J=5.7 Hz, 2H), 3.10-2.82 (m, 2H), 1.43 (s, 2H), 1.23-1.20 (m, 30H), 0.87-0.83 (m, 3H).

### (1-6) Synthesis of Compound NM031 ((8R,10R)-8-{[bis(4-methoxyphenyl)(phenyl)methoxy]methyl}-5-(diisopropylamino)-10-(2,4-dioxo-3H-pyrimidin-1-yl)-4,6,9,12-tetraoxo-5-phosphoheterocyclic octane nitrile)

Compound NM031-6 (1 g, 1.294 mmol) was repeatedly dried with acetonitrile (3×20 mL), then dissolved in dichloromethane (15 mL), added with a dichloromethane (15 mL) solution of 3-{[bis(diisopropylamino)phosphoryl]oxy}propanenitrile (584.88 mg, 1.941 mmol) dried with acetonitrile (3×10 mL), and added with 1H-imidazole-4,5-dinitrile (106.94 mg, 0.906 mmol). Argon replacement was performed three times. In an argon atmosphere, reaction solution was stirred at 25° C. for 1 h. After reaction was completed, the reaction solution was diluted by adding a saturated aqueous sodium bicarbonate solution (100 mL), and extracted with dichloromethane (3×100 mL). Organic phases were combined, washed with saturated brine (2×50 mL), dried over anhydrous sodium sulfate, filtered, concentrated, and subjected to reversed phase chromatography column purification (chromatography column: C18 silica gel, and elution gradient: acetonitrile/water=30/70, v/v), to provide yellow oily compound NM031 (605 mg, yield 47.6%).

$$M S E S I ( m / z ) = {971.5 [ M - H ]}^{-} .$$

¹H NMR (400 MHz, Acetonitrile-d3) δ 9.00 (s, 1H), 7.38 (dd, J=8.0, 3.2 Hz, 1H), 7.32-7.24 (m, 2H), 7.22-7.09 (m, 7H), 6.79-6.69 (m, 4H), 5.96-5.83 (m, 1H), 5.39 (dd, J=8.0, 1.2 Hz, 1H), 3.70-3.58 (m, 10H), 3.58-3.41 (m, 5H), 3.33 (dd, J=6.4, 4.0 Hz, 2H), 3.12-2.89 (m, 2H), 2.50 (d, J=7.6 Hz, 2H), 1.37 (q, J=6.4 Hz, 2H), 1.16 (d, J=10.4 Hz, 26H), 1.08-0.94 (m, 12H), 0.78 (t, J=6.8 Hz, 3H).

### Preparation Example 2: Synthesis of Compound NM058

A synthesis process of compound NM058 was as follows:

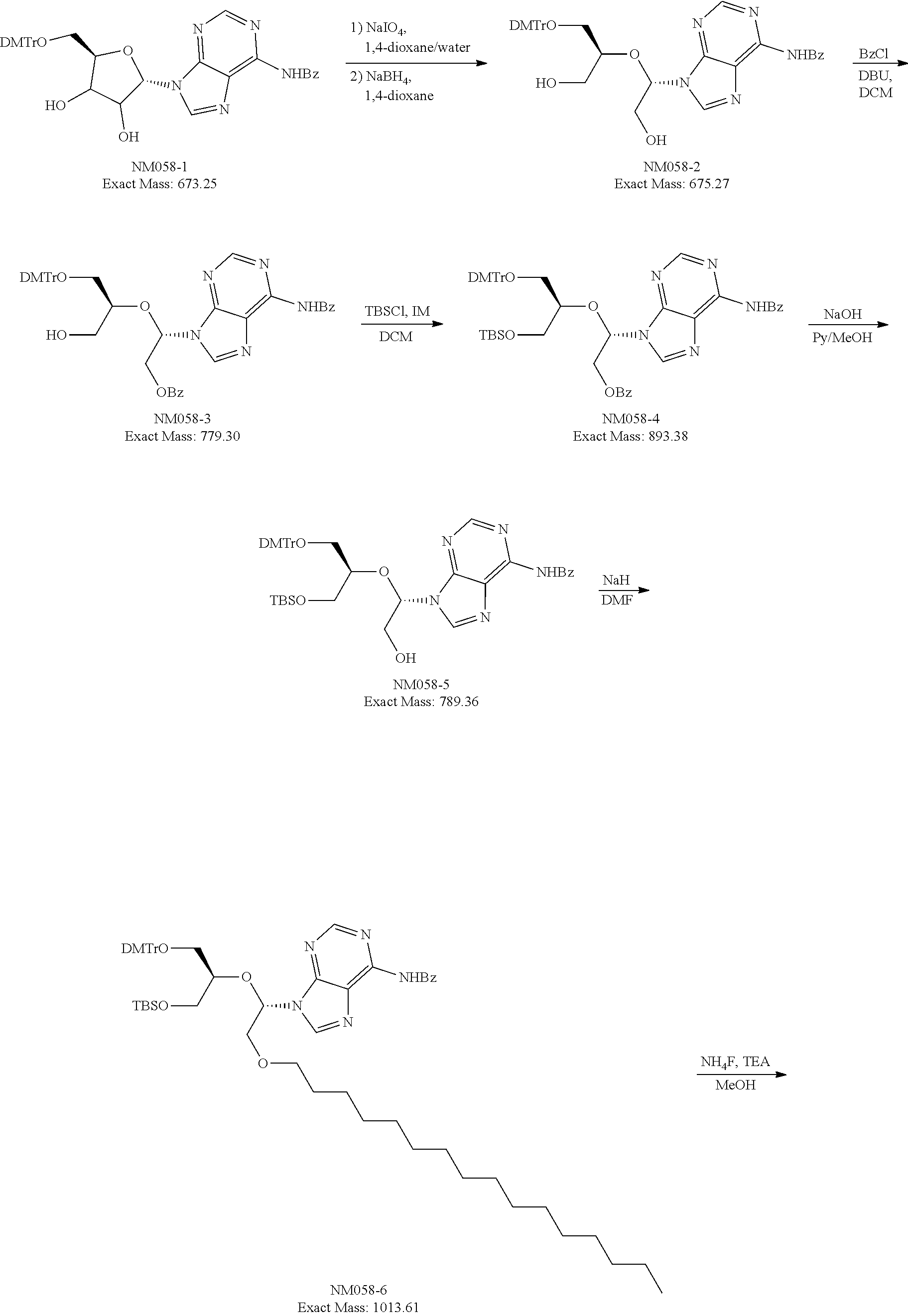

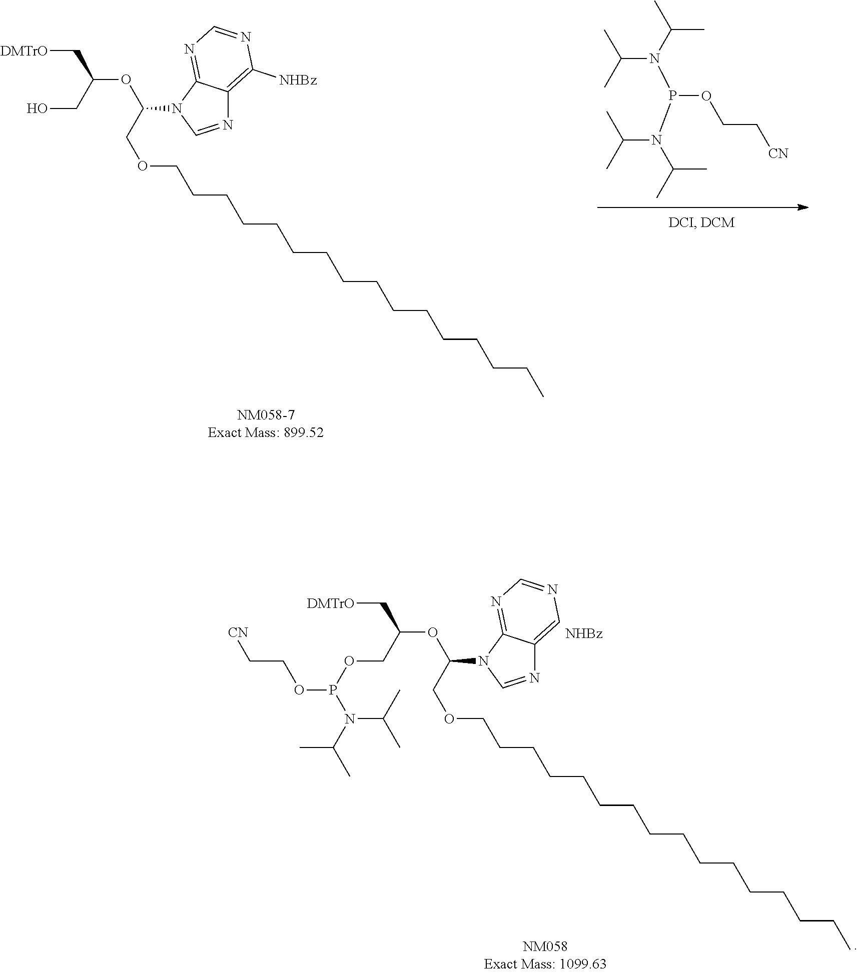

### (2-1) Synthesis of Compound NM058-2 (N-(9-[(S)-1-{[(S)-1-[bis(4-methoxyphenyl)(phenyl)methoxy]-3-hydroxypropan-2-yl]oxy}-2-hydroxyethyl]-9H-purin-6-yl)benzamide)

Compound NM058-1 (1N-(9-[(2S,5R)-5-{[bis(4-methoxyphenyl)(phenyl)methoxy]methyl}-3,4-dihydroxytetrahydrofuran-2-yl]-9H-purin-6-yl)benzamide, 22.6 g, 33.56 mmol) was dissolved in a mixed solution of dioxane (345 mL) and water (115 mL), and added with sodium periodate (8.04 g, 36.92 mmol). In an argon atmosphere, reaction solution was stirred at 25° C. for 2 h. After reaction was ended, the reaction solution was concentrated to remove solvent, diluted by adding water (300 mL), and extracted with dichloromethane five times (5×200 mL). Organic phases were combined, washed with a saturated aqueous sodium bicarbonate solution twice (2×100 mL), washed with saturated brine twice (2×100 mL), dried over anhydrous sodium sulfate, filtered, and concentrated, to provide a white solid (22.6 g), which was directly used in the next step.

At 25° C., the white solid (22.6 g) was dissolved in dioxane (350 mL), and added with sodium borohydride (1.4 g, 36.92 mmol) portion-wise. In the argon atmosphere, reaction solution was stirred under a condition of 25° C. for 2 h, and quenched by adding a saturated aqueous ammonium chloride solution (120 mL). After reaction was ended, the reaction solution was extracted with dichloromethane four times (4×150 mL). Organic phases were combined, washed with saturated brine twice (2×100 mL), dried over anhydrous sodium sulfate, filtered, and concentrated, to provide white solid compound

NM058-2 (20.4 g, yield 98.7%).

$$M S E S I ( m / z ) = {676.27 [ M + H ]}^{-} .$$

### (2-2) Synthesis of Compound NM058-3 ((S)-2-(6-benzoylamino-9H-purin-9-yl)-2-{[(S)-1-[bis(4-methoxyphenyl)(phenyl)methoxy]-3-hydroxypropyl-2-yl]oxy}ethyl benzoate)

At 25° C., compound NM058-2 (20 g, 31.079 mmol) was dissolved in dichloromethane (200 mL), and added with 1,8-Diazabicyclo[5.4.0]undec-7-ene (9.456 g, 62.158 mmol). Nitrogen replacement was performed three times, and in a nitrogen atmosphere, reaction solution was cooled to −78° C. Benzoyl chloride (4.805 g, 34.187 mmol) was added dropwise at −78° C. The reaction solution was stirred at −78° C. for 1 h, and quenched by adding a saturated aqueous ammonium chloride solution (150 mL). After reaction was ended, resultant was extracted with dichloromethane three times (3×120 mL). Organic phases were combined, washed with a saturated aqueous sodium bicarbonate solution twice (2×200 mL), washed with saturated brine twice (2×100 mL), dried over anhydrous sodium sulfate, filtered, and concentrated, to provide yellow solid crude product compound NM058-3 (25 g).

$$M S E S I ( m / z ) = {780.3 [ M + H ]}^{-} .$$

### (2-3) Synthesis of Compound NM058-4 ((S)-2-(6-benzoylamino-9H-purin-9-yl)-2-{[(R)-1-[bis(4-methoxyphenyl)(phenyl)methoxy]-3-[(tert-butyldimethylsilyloxy)oxy]propan-2-yl]oxy}ethyl benzoate)

At 25° C., compound NM058-3 (24.7 g, 31.695 mmol) was dissolved in dichloromethane (250 mL), and added with imidazole (3.23 g, 47.543 mmol) and tert-butyldimethylsilyl chloride (6.21 g, 41.203 mmol). In a nitrogen atmosphere, reaction solution was stirred at 25° C. for 16 h. After reaction was ended, resultant was spin-dried to remove solvent, diluted by adding water (400 mL), and extracted with dichloromethane four times (4×150 mL). Organic phases were combined, washed with a saturated aqueous sodium bicarbonate solution twice (2×80 mL), washed with saturated brine once (1×100 mL), dried over anhydrous sodium sulfate, filtered, and concentrated, to provide yellow crude product compound NM058-4 (28.6 g).

$$M S E S I ( m / z ) = {894.38 [ M + H ]}^{-} .$$

### (2-4) Synthesis of Compound NM058-5 (N-(9-[(S)-1-{[(R)-1-[bis(4-methoxyphenyl)(phenyl)methoxy]-3-[(tert-butyldimethylsilyloxy)oxy]propan-2-yl]oxy}-2-hydroxyethyl]-9H-purin-6-yl)benzamide)

At 25° C., compound NM058-4 (28.3 g, 31.689 mmol) was dissolved in a mixed solution of pyridine (170 mL) and methanol (68 mL), cooled to 0° C. by an ice bath, and added with a sodium hydroxide aqueous solution (31.6 mL, 1 M). In the nitrogen atmosphere, reaction solution was stirred under a condition of 0° C. for 0.5 h. After reaction was ended, the reaction solution was spin-dried to remove solvent, diluted by adding water (150 mL), and extracted with dichloromethane three times (3×200 mL). Organic phases were combined, washed with a saturated aqueous ammonium chloride solution twice (2×100 mL), washed with saturated brine twice (2×100 mL), dried over anhydrous sodium sulfate, filtered, concentrated, and subjected to normal phase chromatography column purification (elution gradient: petroleum ether/ethyl acetate=1/1, v/v), to provide white solid compound NM058-5 (16.21 g, yield 64.8%).

$$M S E S I ( m / z ) = {790.36 [ M + H ]}^{-} .$$

### (2-5) Synthesis of Compound NM058-6 (N-(9-[(6R,8S)-6-{[bis(4-methoxyphenyl)(phenyl)methoxy]methyl}-2,2,3,3-tetramethyl-4,7,10-trioxa-3-silaxane-8-yl]-9H-purin-6-yl)benzamide)

In an ice bath, N,N-dimethylformamide (30 mL) was cooled to 0° C., added first with sodium hydrogen (501.7 mg, 11.4 mmol), and then added with compound NM058-5 (3 g, 3.8 mmol). In a nitrogen atmosphere, reaction solution was stirred under a condition of 0° C. for 30 min, added with 1-bromohexadecane (1.74 g, 5.7 mmol) and potassium iodide (946.33 mg, 5.7 mmol), slowly warmed to 25° C., stirred under a condition of 25° C. for 16 h, and quenched by adding a saturated aqueous ammonium chloride solution (150 mL). After reaction was ended, the reaction solution was extracted with ethyl acetate three times (3×150 mL). Organic phases were combined, washed with a saturated aqueous sodium bicarbonate solution twice (2×80 mL), washed with saturated brine once (1×100 mL), dried over anhydrous sodium sulfate, filtered, and concentrated, to provide yellow solid crude product compound NM058-6 (3.9 g).

$$M S E S I ( m / z ) = {1014.61 [ M + H ]}^{-} .$$

### (2-6) NM058-7 (N-(9-[(S)-1-{[(S)-1-[bis((4-methoxyphenyl)(phenyl)methoxy]-3-hydroxypropan-2-yl]oxy}-2-(hexadecyloxy)ethyl]-9H-purin-6-yl)benzamide)

At 25° C., compound NM058-6 (3.9 g, 3.847 mmol) was dissolved in methanol (24 mL), and added with ammonium fluoride (984.28 mg, 26.933 mmol) and triethylamine (767.66 mg, 7.694 mmol). In a nitrogen atmosphere, reaction solution was stirred under a condition of 40° C. for 3 h. After reaction was ended, the reaction solution was directly concentrated, and subjected to normal phase chromatography column purification (eluent gradient: petroleum ether/ethyl acetate=1/1, v/v), to provide white solid compound NM058-7 (1.34 g, yield 39.2%).

$$M S E S I ( m / z ) = {900.52 [ M + H ]}^{-} .$$

### (2-7) Synthesis of Compound NM058 ((R)-2-[(R)-1-(6-benzoylamino-9H-purin-9-yl)-2-(hexadecyloxy)ethoxy]-3-[bis(4-methoxyphenyl)(phenyl)methoxy]propyl(2-cyanoethyl)diisopropylphosphoramidite)

Compound NM058-7 (1 g, 1.11 mmol) was first dried with acetonitrile three times (3×20 mL), then dissolved in dichloromethane (15 mL), added with a dichloromethane (15 mL) solution of 3-{[bis(diisopropylamino)phosphoryl]oxy}propanenitrile (402.8 mg, 1.332 mmol) (3-{[bis(diisopropylamino)phosphoryl]oxy}propanenitrile was first dried with acetonitrile three times (3×10 mL), and then dissolved in dichloromethane), and then added with 1H-imidazole-4,5-dinitrile (104.92 mg, 0.888 mmol). Nitrogen replacement was performed three times. In a nitrogen atmosphere, reaction solution was stirred under a condition of 25° C. for 1 h. After reaction was ended, resultant was diluted by adding a saturated aqueous sodium bicarbonate solution (30 mL), and extracted with dichloromethane three times (3×20 mL). Organic phases were combined, washed with saturated brine twice (2×30 mL), dried over anhydrous sodium sulfate, filtered, concentrated, and subjected to normal phase chromatography column purification (elution gradient: petroleum ether/ethyl acetate=2/1, v/v), to provide white solid compound NM058 (750 mg, yield 61.4%).

$$M S E S I ( m / z ) = {1100.63 [ M + H ]}^{-} .$$

¹H NMR (400 MHz, DMSO-d6) δ 8.71 (d, J=17.9 Hz, 2H), 8.06 (d, J=7.5 Hz, 2H), 7.55 (t, J=7.6 Hz, 2H), 7.24-7.03 (m, 5H), 7.01-6.91 (m, 4H), 6.80-6.74 (m, 4H), 4.00 (dd, J=10.6, 4.9 Hz, 2H), 3.68 (s, 9H), 3.49 (ddt, J=30.2, 14.3, 7.1 Hz, 4H), 2.74 (dt, J=11.8, 5.9 Hz, 2H), 1.35-0.88 (m, 39H), 0.85 (t, J=6.7 Hz, 3H).

### Preparation Example 3: Synthesis of Compound NM073

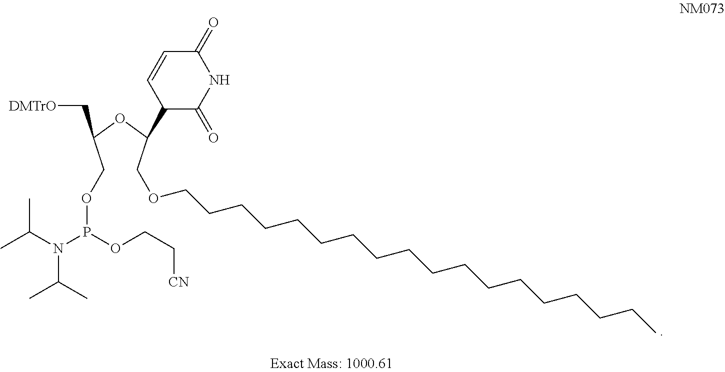

In the present preparation example, a synthesis route of compound NM073 was as follows:

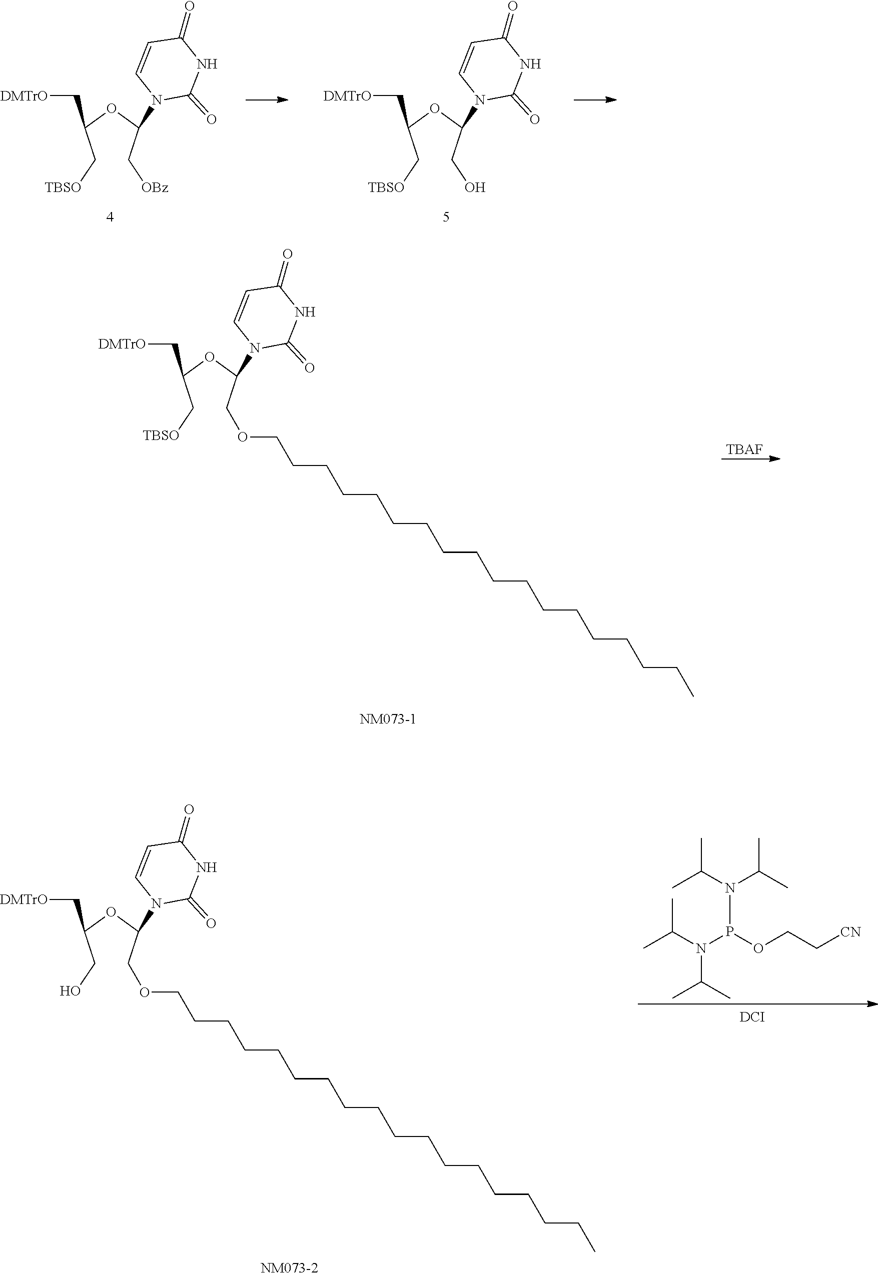

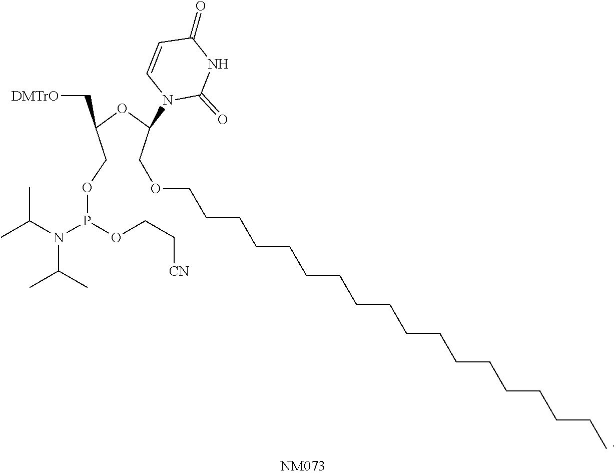

### (3-1) Synthesis of Compound 5

At 25° C., compound 4 (17.1 g, 22.3 mmol, 1 eq) was dissolved in a mixed solution of pyrimidine (102 ml) and methanol (41 ml). A temperature of reaction system was reduced to 0° C. by an ice bath. An aqueous sodium hydroxide solution (16.7 mL, 1 M, 1.2 eq) was added at 0° C. Nitrogen replacement was performed three times. In a nitrogen atmosphere, reaction solution was stirred at 0° C. for 1 h. After reaction was completed, the reaction solution was spin-dried to remove solvent, diluted by adding water (150 mL), and extracted with dichloromethane (3×200 mL). Organic phases were combined, washed with a saturated aqueous ammonium chloride solution (2×100 mL), washed with a saturated aqueous sodium chloride solution (2×100 mL), dried over anhydrous sodium sulfate, filtered, concentrated, and subjected to normal phase chromatography column purification (eluent: petroleum ether/ethyl acetate=1/1, v/v), to provide white solid compound 5 (13.1 g, yield 88.6%). MS ESI (m/z)=663.3 [M+H]⁺.

### (3-2) Synthesis of Compound NM073-1

Compound 5 (4 g, 6.04 mmol, 1 eq) was dissolved in N,N-dimethylformamide (40 ml). A temperature of reaction system was reduced to 0° C. by an ice bath. An N,N-dimethylformamide solution of sodium hydride (0.72 g, 18.11 mmol, 3 eq) with mass percentage concentration of 60% was added at 0° C. Nitrogen replacement was performed three times. In a nitrogen atmosphere, reaction solution was stirred at 0° C. for 30 min, added with 1-bromooctadecane (3.02 g, 9.06 mmol, 1.5 eq, CAS No.: 112-89-0), and slowly warmed to 25° C. and stirred at 25° C. for 16 h. After reaction was completed, the reaction solution was quenched by adding a saturated aqueous ammonium chloride solution (200 ml), and extracted with ethyl acetate (3×100 ml). Organic phases were combined, washed with a saturated aqueous sodium bicarbonate solution (2×80 ml) and a saturated aqueous sodium chloride solution (100 ml), dried over anhydrous sodium sulfate, filtered, concentrated, and subjected to normal phase chromatography column purification (eluent: petroleum ether/ethyl acetate=3/1, v/v), to provide yellow solid compound NM073-1 (1.97 g, yield 35.7%).

$$M S E S I ( m / z ) = {915.5 [ M + H ]}^{+} .$$

### (3-3) Synthesis of Compound NM073-2

At 25° C., compound NM073-1 (1.97 g, 2.15 mmol, 1 eq) was dissolved in tetrahydrofuran (20 ml), and added with a tetrahydrofuran solution (4.3 mL, 1 M, 2 eq) of tetrabutylammonium fluoride dropwise. Nitrogen replacement was performed three times, and reaction solution was stirred at 0° C. for 4 h in a nitrogen atmosphere. After reaction was completed, the reaction solution was directly concentrated to remove solvent, and subjected to normal phase chromatography column purification (eluent: petroleum ether/ethyl acetate=1/1, v/v), to provide colorless oily compound NM073-2 (930 mg, yield 53.9%). MS ESI (m/z)=801.45 [M+H]⁺.

### (3-4) Synthesis of Compound NM073

Compound NM073-2 (930 mg, 1.16 mmol, 1 eq) dried with acetonitrile (3×10 mL) was dissolved in dichloromethane (10 mL), added with a dichloromethane (10 mL) solution of bis(diisopropylamino)(2-cyanoethoxy)phosphine (526.3 mg, 1.74 mmol, 1.5 eq) dried with acetonitrile (3×10 mL), and added with 1H-imidazole-4,5-dinitrile (109.67 mg, 0.93 mmol, 0.8 eq, CAS No.: 1122-28-7, abbreviated as DCI). Argon replacement was performed three times, and reaction solution was stirred at 25° C. for 1 h in an argon atmosphere. After reaction was completed, the reaction solution was diluted by adding a saturated aqueous sodium bicarbonate solution (50 ml), and extracted with dichloromethane (3×20 ml). Organic phases were combined, washed with a saturated aqueous sodium chloride solution (2×50 ml), dried over anhydrous sodium sulfate, filtered, concentrated, and subjected to normal phase chromatography column purification (eluent: petroleum ether/ethyl acetate=2/1, v/v), to provide colorless oily compound NM073 (1 g, yield 86.0%). MS ESI (m/z)=1001.50 [M+H]⁺.

¹H NMR (400 MHz, DMSO-d6) δ 11.37 (s, 1H), 7.65 (d, J=8.1 Hz, 1H), 7.34-7.24 (m, 4H), 7.18 (ddd, J=8.9, 4.0, 2.4 Hz, 5H), 6.93-6.74 (m, 4H), 5.96 (q, J=5.9 Hz, 1H), 5.53 (d, J=8.0 Hz, 1H), 3.73 (s, 7H), 3.65 (dd, J=8.4, 5.3 Hz, 5H), 3.60-3.35 (m, 5H), 3.10-2.93 (m, 2H), 2.70 (dt, J=11.8, 5.9 Hz, 2H), 1.42 (q, J=6.5 Hz, 2H), 1.22 (d, J=10.5 Hz, 30H), 1.10 (dd, J=6.8, 3.1 Hz, 6H), 1.04 (dd, J=12.1, 6.7 Hz, 6H), 0.85 (t, J=6.7 Hz, 3H).

### Preparation Example 4: Synthesis of Compound NM074

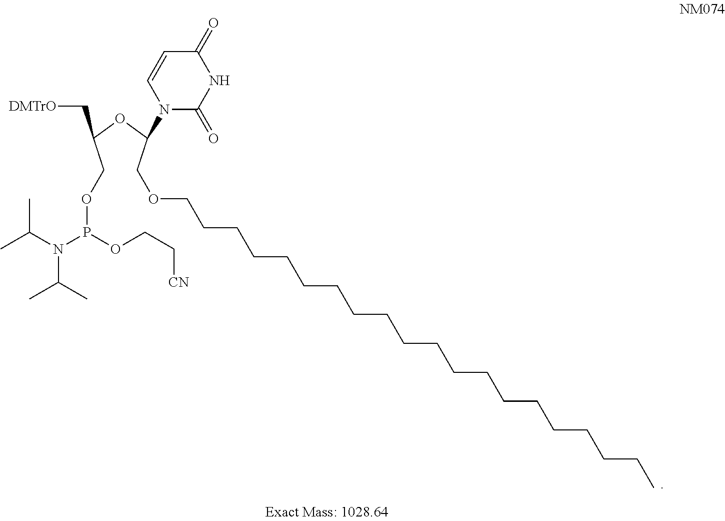

In the present preparation example, a synthesis route of compound NM074 was as follows:

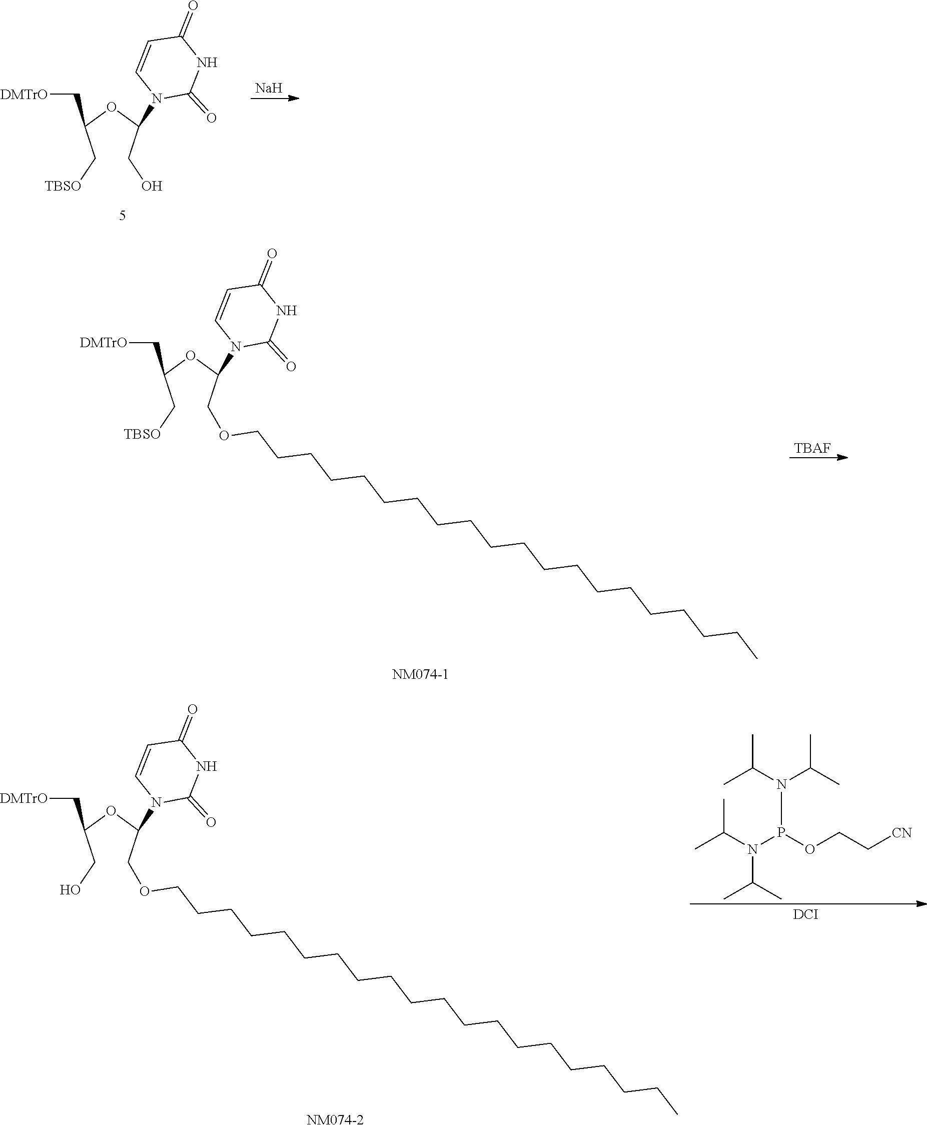

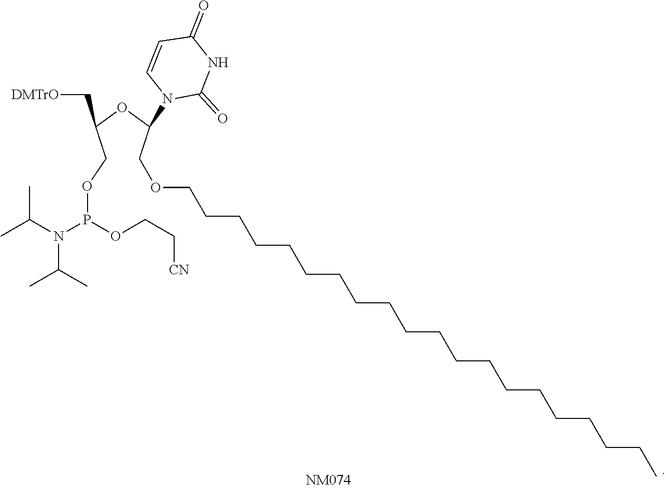

### (4-1) Synthesis of Compound NM074-1

Compound 5 (4 g, 6.04 mmol, 1 eq) was dissolved in N,N-dimethylformamide (40 ml). Reaction system was reduced to a temperature of 0° C. by an ice bath, and added with an N,N-dimethylformamide solution of sodium hydride (0.72 g, 18.11 mmol, 3 eq) with mass percentage concentration of 60% at 0° C. Nitrogen replacement was performed three times, and reaction solution was stirred at 0° C. for 30 min in a nitrogen atmosphere, added with 1-bromoicosane (3.27 g, 9.06 mmol, 1.5 eq, CAS No.: 4276-49-7), slowly warmed to 25° C. and stirred at 25° C. for 16 h. After reaction was completed, the reaction solution was quenched by adding a saturated aqueous ammonium chloride solution (200 ml), and extracted with ethyl acetate (3×100 ml). Organic phases were combined, washed with a saturated aqueous sodium bicarbonate solution (2×80 ml) and a saturated aqueous sodium chloride solution (100 ml), dried over anhydrous sodium sulfate, filtered, concentrated, and subjected to normal phase chromatography column purification (eluent: petroleum ether/ethyl acetate=3/1, v/v), to provide yellow solid compound NM074-1 (2.08 g, yield 36.6%).

$$M S E S I ( m / z ) = {943.6 [ M + H ]}^{+} .$$

### (4-2) Synthesis of Compound NM074-2

At 25° C., compound NM074-1 (2.08 g, 2.21 mmol, 1 eq) was dissolved in tetrahydrofuran (20 ml), and added with a tetrahydrofuran solution of tetrabutylammonium fluoride (4.4 mL, 1 M, 2 eq) dropwise. Nitrogen replacement was performed three times, and reaction solution was stirred at 0° C. for 4 h in a nitrogen atmosphere. After reaction was completed, the reaction solution was directly concentrated to remove solvent, and subjected to normal phase chromatography column purification (eluent: petroleum ether/ethyl acetate=1/1, v/v), to provide colorless oily compound NM074-2 (970 mg, yield 53%). MS ESI (m/z)=829.55 [M+H]⁺.

### (4-3) Synthesis of Compound NM074

Compound NM074-2 (970 mg, 1.17 mmol, 1 eq) dried with acetonitrile (3×10 mL) was dissolved in dichloromethane (10 mL), added with a dichloromethane (10 mL) solution of bis(diisopropylamino)(2-cyanoethoxy)phosphine (530.3 mg, 1.76 mmol, 1.5 eq) dried with acetonitrile (3×10 mL), and added with 1H-imidazole-4,5-dinitrile (110.5 mg, 0.94 mmol, 0.8 eq, CAS No.: 1122-28-7, abbreviated as DCI). Argon replacement was performed three times, and reaction solution was stirred at 25° C. for 1 h in an argon atmosphere. After reaction was completed, the reaction solution was diluted by adding a saturated aqueous sodium bicarbonate solution (50 ml), and extracted with dichloromethane (3×20 ml). Organic phases were combined, washed with a saturated aqueous sodium chloride solution (2×50 ml), dried over anhydrous sodium sulfate, filtered, concentrated, and subjected to normal phase chromatography column purification (eluent: petroleum ether/ethyl acetate=2/1, v/v), to provide colorless oily compound NM074 (1 g, yield 83.0%). MS ESI (m/z)=1029.64 [M+H]+.

¹H NMR (400 MHz, DMSO-d6) δ 11.37 (s, 1H), 7.65 (d, J=8.1 Hz, 1H), 7.34-7.23 (m, 4H), 7.18 (ddd, J=8.9, 4.1, 2.5 Hz, 5H), 6.89-6.80 (m, 4H), 5.96 (q, J=5.8 Hz, 1H), 5.53 (d, J=8.0 Hz, 1H), 3.73 (s, 7H), 3.71-3.36 (m, 10H), 3.12-2.92 (m, 2H), 2.70 (dt, J=11.7, 5.9 Hz, 2H), 1.43 (s, 2H), 1.22 (d, J=10.4 Hz, 34H), 1.10 (dd, J=6.8, 3.0 Hz, 6H), 1.04 (dd, J=12.1, 6.7 Hz, 6H), 0.85 (t, J=6.6 Hz, 3H).

### Preparation Example 5: Synthesis of Compound NM201

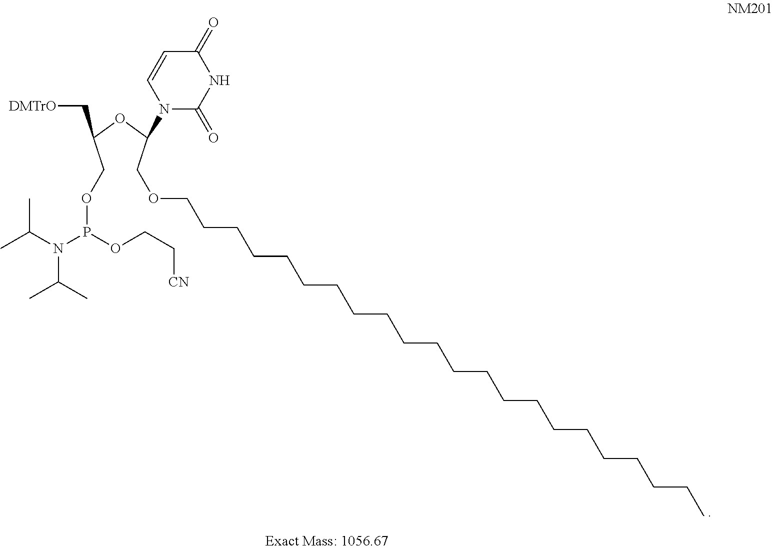

In the present preparation example, a synthesis route of compound NM201 was as follows:

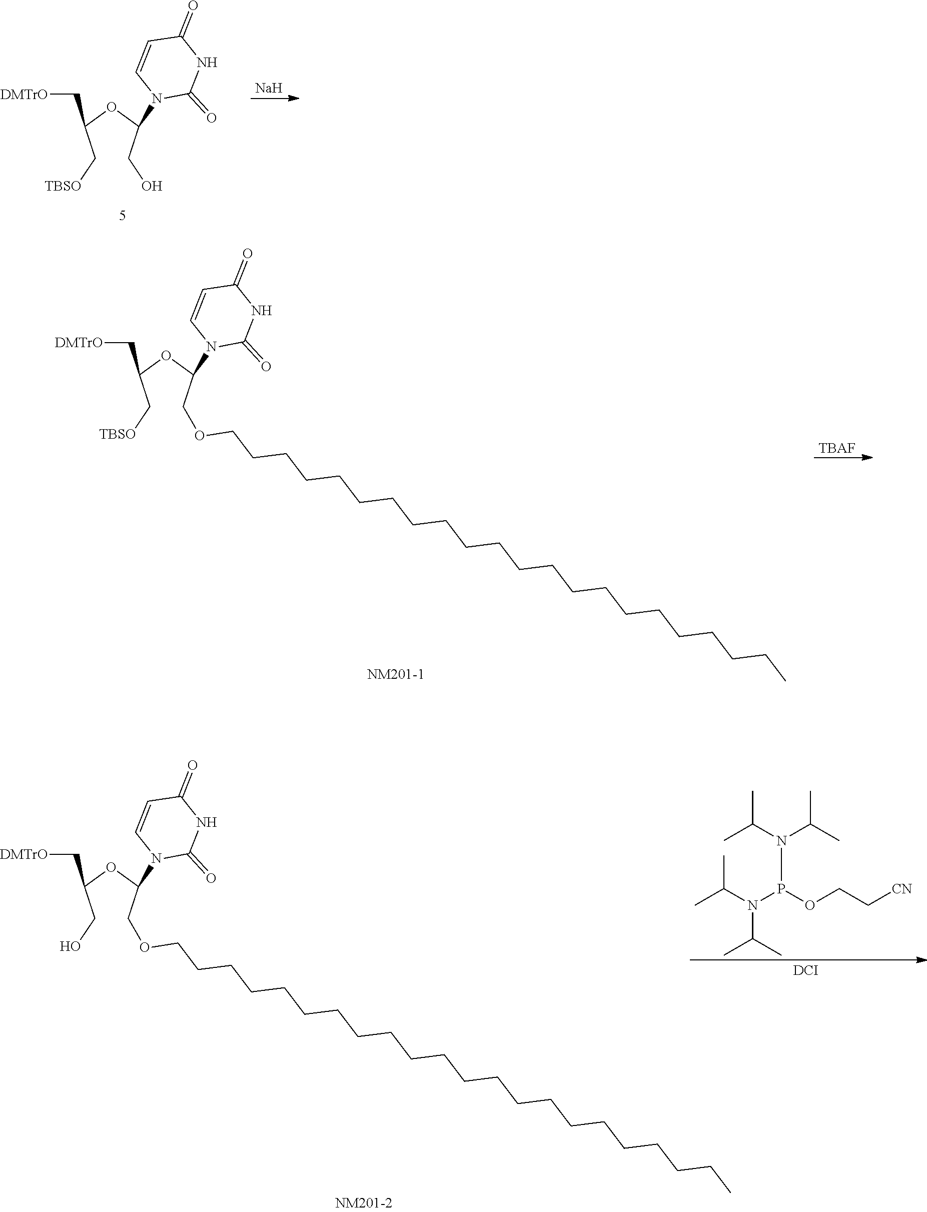

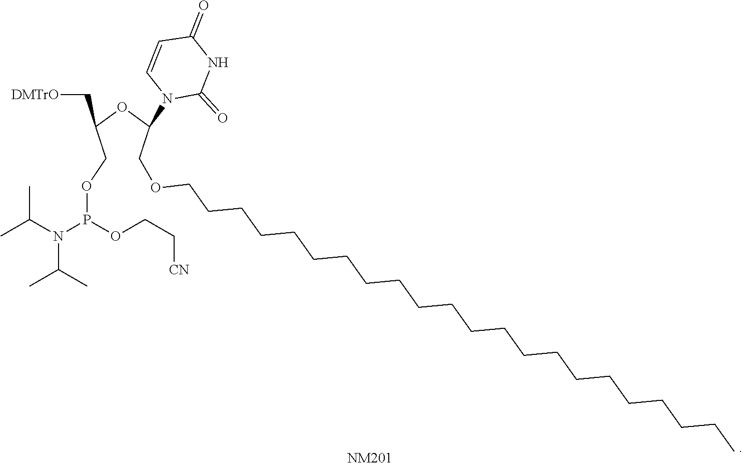

### (5-1) Synthesis of Compound NM201-1

Compound 5 (12.4 g, 18.7 mmol, 1 eq) was dissolved in N,N-dimethylformamide (120 ml). Reaction system was reduced to a temperature of 0° C. by an ice bath, and added with an N,N-dimethylformamide solution of sodium hydride (2.25 g, 60%, 56.1 mmol, 3 eq) with mass percentage concentration of 60%. Nitrogen replacement was performed three times, and reaction solution was stirred at 0° C. for 30 min in a nitrogen atmosphere, added with 1-bromodocosane (10.9 g, 28.1 mmol, 1.5 eq, CAS No.: 6938-66-5), and slowly warmed to 25° C., and stirred at 25° C. for 16 h. After reaction was completed, the reaction solution was quenched by adding a saturated aqueous ammonium chloride solution (600 ml), and extracted with ethyl acetate (3×200 ml). Organic phases were combined, washed with a saturated aqueous sodium bicarbonate solution (2×100 ml) and a saturated aqueous sodium chloride solution (100 ml), dried over anhydrous sodium sulfate, filtered, concentrated, and subjected to normal phase chromatography column purification (eluent: petroleum ether/ethyl acetate=3/1, v/v), to provide yellow solid compound NM201-1 (8.2 g, yield 45.1%). MS ESI (m/z)=971.66 [M+H]⁺.

### (5-2) Synthesis of Compound NM201-2

At 25° C., compound NM201-1 (8.2 g, 8.44 mmol, 1 eq) was dissolved in tetrahydrofuran (80 ml), and added with a tetrahydrofuran solution (12.7 mL, 1 M, 1.5 eq) of tetrabutylammonium fluoride dropwise. Nitrogen replacement was performed three times, and reaction solution was stirred at 0° C. for 4 h in a nitrogen atmosphere. After reaction was completed, the reaction solution was directly concentrated to remove solvent, and subjected to normal phase chromatography column purification (eluent: petroleum ether/ethyl acetate=1/1, v/v), to provide colorless oily compound NM201-2 (3.21 g, yield 44.4%). MS ESI (m/z)=857.55 [M+H]⁺.

### (5-3) Synthesis of Compound NM201

Compound NM201-2 (3 g, 3.50 mmol, 1 eq) dried with acetonitrile (3×10 mL) was dissolved in dichloromethane (30 mL), added with a dichloromethane (10 mL) solution of bis(diisopropylamino)(2-cyanoethoxy)phosphine (1.59 g, 5.25 mmol, 1.5 eq) dried with acetonitrile (3×10 mL), and added with 1H-imidazole-4,5-dinitrile (330.6 mg, 2.80 mmol, 0.8 eq). Argon replacement was performed three times, and reaction solution was stirred at 25° C. for 1 h in an argon atmosphere. After reaction was completed, the reaction solution was diluted by adding a saturated aqueous sodium bicarbonate solution (50 ml), and extracted with dichloromethane (3×30 ml). Organic phases were combined, washed with a saturated aqueous sodium chloride solution (2×50 ml), dried over anhydrous sodium sulfate, filtered, concentrated, and subjected to normal phase chromatography column purification (eluent: petroleum ether/ethyl acetate=2/1, v/v), to provide colorless oily compound NM201 (2.83 g, yield 76.5%). MS ESI (m/z)=1056.56 [M+H]⁺.

¹H NMR (400 MHz, Chloroform-d) δ 7.64 (d, J=8.2 Hz, 1H), 7.29 (s, 4H), 7.18 (d, J=9.4 Hz, 5H), 6.84 (d, J=8.4 Hz, 4H), 5.96 (s, 1H), 5.56-5.49 (m, 1H), 3.72 (d, J=2.6 Hz, 7H), 3.64 (s, 6H), 3.48 (s, 2H), 3.40 (d, J=6.3 Hz, 2H), 3.01 (d, J=28.8 Hz, 2H), 2.68 (d, J=6.2 Hz, 2H), 1.34-1.14 (m, 40H), 1.10 (dt, J=6.5, 2.8 Hz, 5H), 1.07-1.00 (m, 5H), 0.84 (dd, J=7.4, 4.8 Hz, 3H).

### Preparation Example 6: Synthesis of Compound NM087

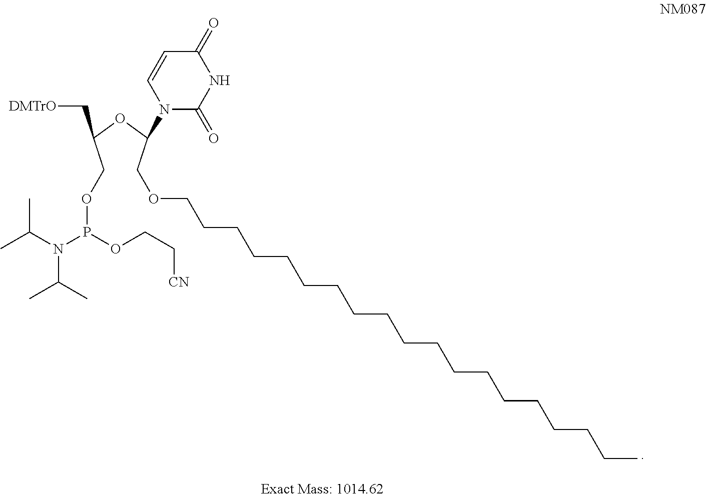

In the present preparation example, a synthesis route of compound NM087 was as follows:

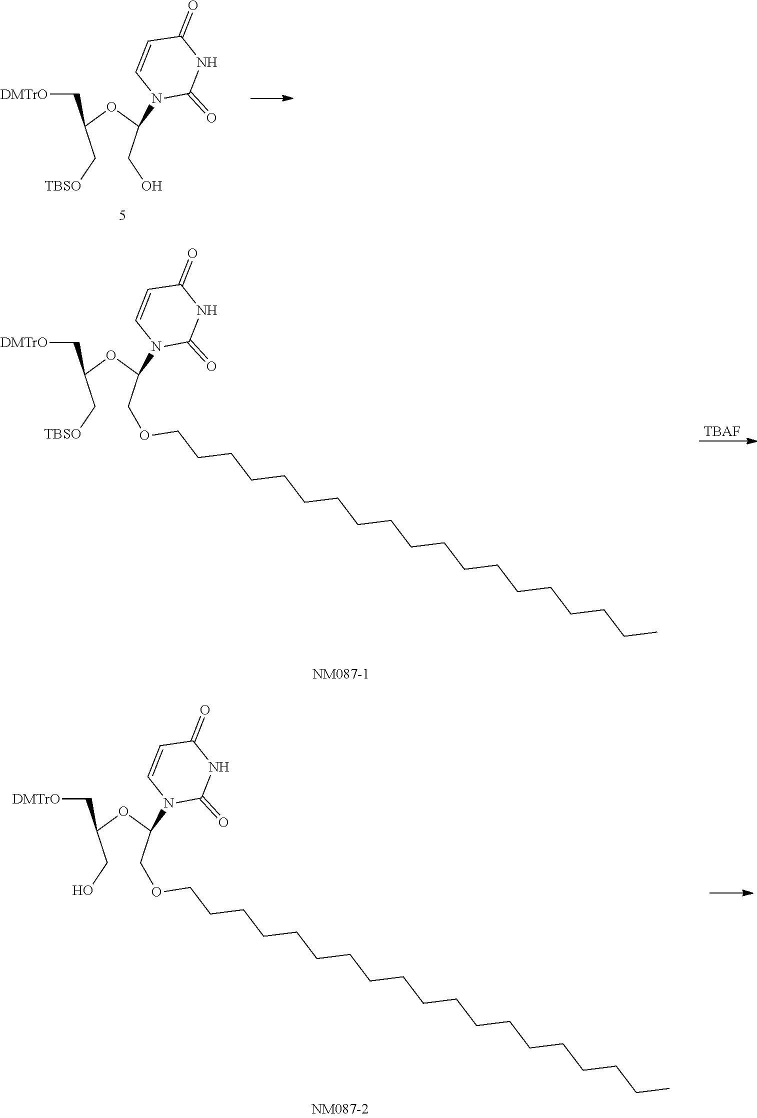

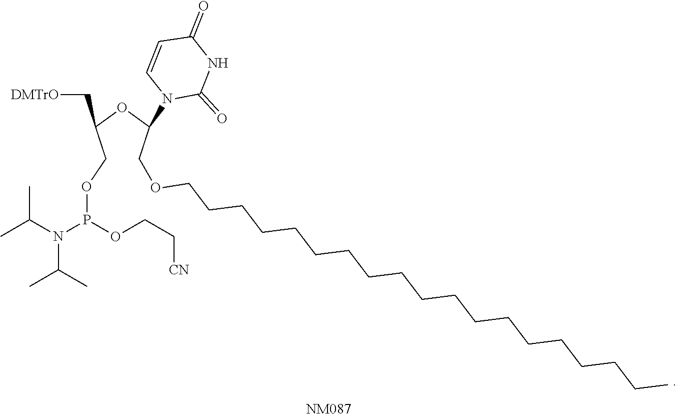

### (6-1) Synthesis of Compound NM087-1

At 25° C., compound 5 (5.92 g, 8.94 mmol, 1 eq) was dissolved in N,N-dimethylformamide (60 ml). Reaction system was reduced to a temperature of 0° C. by an ice bath, and added with an N,N-dimethylformamide solution of sodium hydride (893.9 mg, 22.35 mmol, 2.5 eq) with mass percentage concentration of 60% at 0° C. Nitrogen replacement was performed three times, and the reaction system was stirred at 0° C. for 30 min in a nitrogen atmosphere, added with 1-bromononadecane (4.658 g, 13.41 mmol, 1.5 eq, CAS No.: 4434-66-6), slowly warmed to 25° C., and stirred at 25° C. for 16 h. After reaction was completed, the reaction solution was quenched by adding a saturated aqueous ammonium chloride solution (200 ml), and extracted with ethyl acetate (3×100 ml). Organic phases were combined, washed with a saturated aqueous sodium bicarbonate solution (2×50 ml) and a saturated aqueous sodium chloride solution (100 ml), dried over anhydrous sodium sulfate, filtered, concentrated, and subjected to column chromatography normal phase purification (eluent: petroleum ether/ethyl acetate=3/1, v/v), to provide yellow solid compound NM087-1 (2.386 g, 34.46% yield). MS ESI (m/z)=929.54 [M+H]⁺.

### (6-2) Synthesis of Compound NM087-2

At 25° C., compound NM087-1 (2.386 g, 2.57 mmol, 1 eq) was dissolved in tetrahydrofuran (30 ml), and added with a tetrahydrofuran solution of tetrabutylammonium fluoride (5.2 mL, 1 M, 2 eq) dropwise. Nitrogen replacement was performed three times, and reaction system was stirred at 25° C. for 16 h in a nitrogen atmosphere. After reaction was completed, reaction solution was concentrated to remove solvent, and subjected to column chromatography normal phase purification (eluent: petroleum ether/ethyl acetate=2/1, v/v), to provide colorless oily compound NM087-2 (690 mg, yield 32.97%). MS ESI (m/z)=816.62 [M+H]⁺.

### (6-3) Synthesis of Compound NM087

At 25° C., NM087-2 (690 mg, 0.847 mmol, 1 eq) dried with acetonitrile (3×5 mL) three times was dissolved in dichloromethane (10 mL), added with a dichloromethane (10 mL) solution of bis(diisopropylamino)(2-cyanoethoxy)phosphine (383.76 g, 1.27 mmol, 1.5 eq) dried with acetonitrile (3×5 mL) three times, and added with 1H-imidazole-4,5-dinitrile (79.97 mg, 0.678 mmol, 0.8 eq). Argon replacement was performed three times, and reaction system was stirred at 25° C. for 1 h in an argon atmosphere. After reaction was completed, reaction solution was diluted by adding a saturated aqueous sodium bicarbonate solution (30 ml), and extracted with dichloromethane (3×20 ml). Organic phases were combined, washed with a saturated aqueous sodium chloride solution (2×20 ml), dried over anhydrous sodium sulfate, filtered, concentrated, and subjected to column chromatography normal phase purification (eluent: petroleum ether/ethyl acetate=3/1, v/v), to provide colorless oily compound NM087 (5.0 mg, yield 76.5%). MS ESI (m/z)=1015.43 [M+H]⁺.

¹H NMR (400 MHz, DMSO-d6) δ 11.40 (s, 1H), 7.67 (d, J=8.0 Hz, 1H), 7.35-7.26 (m, 4H), 7.20 (ddd, J=9.0, 4.2, 2.6 Hz, 5H), 6.87 (dd, J=8.7, 1.8 Hz, 4H), 5.99 (t, J=5.7 Hz, 1H), 5.59-5.52 (m, 1H), 3.75 (s, 6H), 3.67 (d, J=5.9 Hz, 4H), 3.56-3.39 (m, 4H), 3.13-2.92 (m, 2H), 2.73 (dd, J=11.9, 5.9 Hz, 2H), 1.45 (s, 3H), 1.24 (d, J=10.6 Hz, 36H), 1.12 (dd, J=6.8, 3.0 Hz, 5H), 1.06 (dd, J=12.2, 6.8 Hz, 5H), 0.87 (t, J=6.7 Hz, 3H).

### Preparation Example 7: Synthesis of Reference Compound NM200

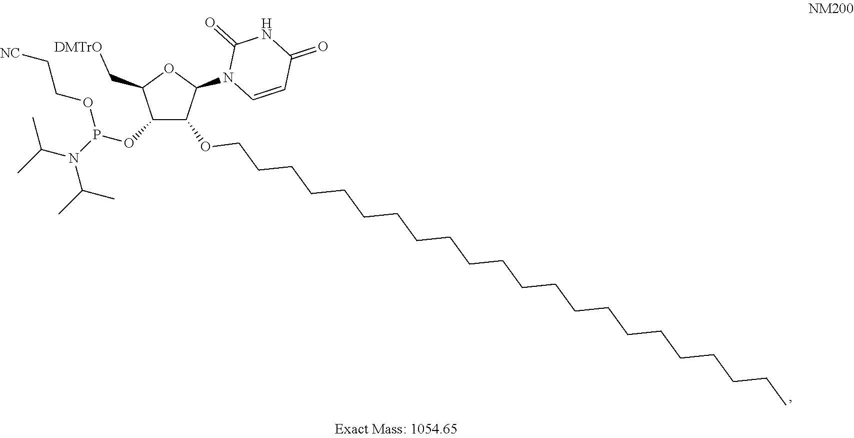

In the present preparation example, a synthesis route of reference compound NM200 was as follows:

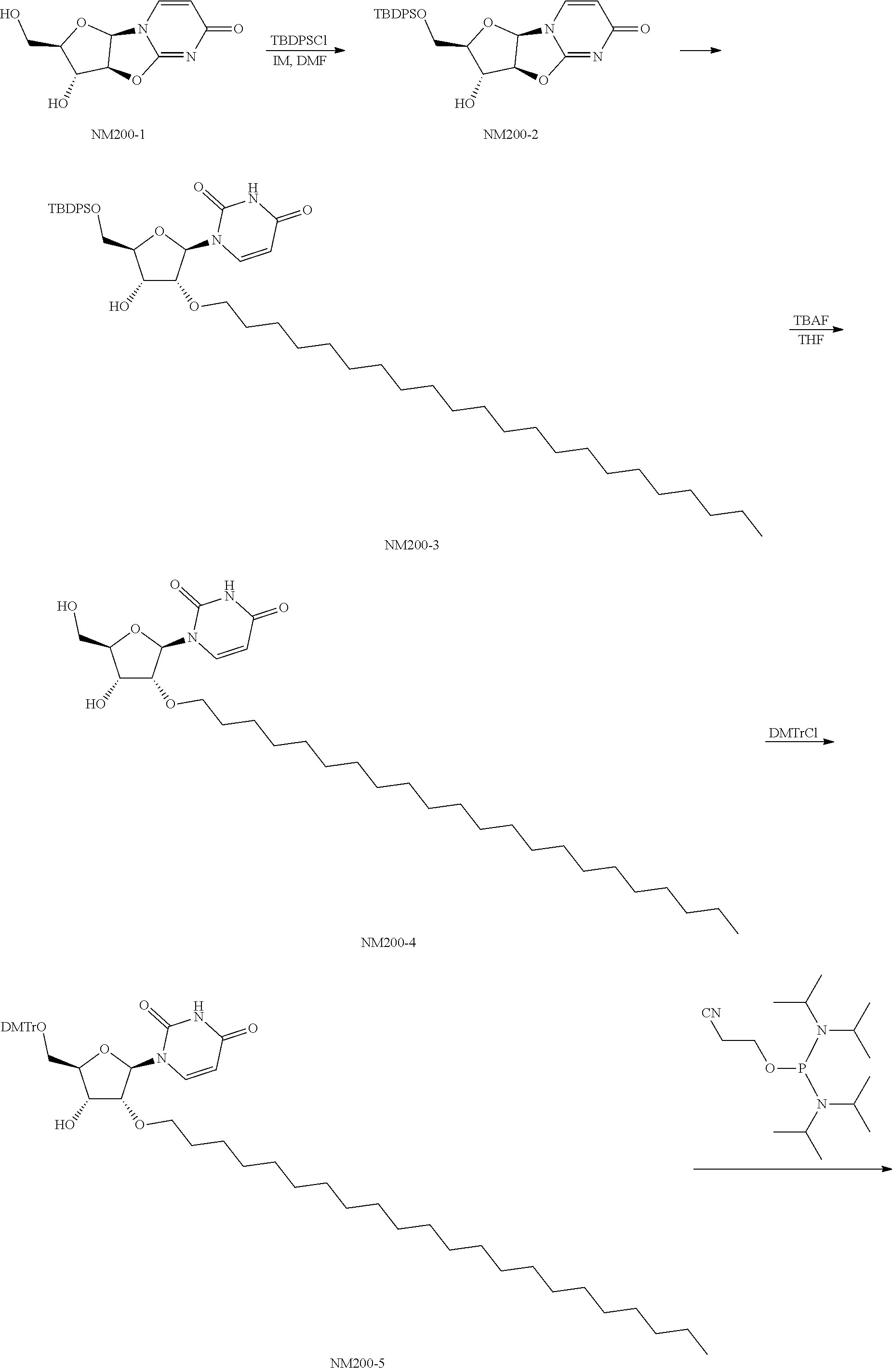

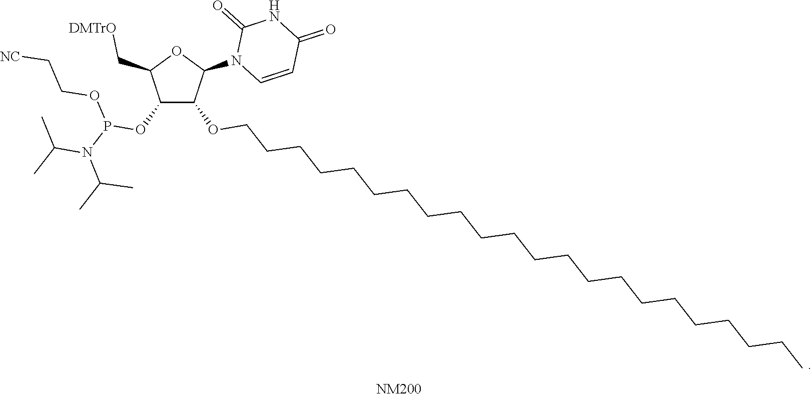

### (7-1) Synthesis of Compound NM200-2

Compound NM200-1 (10 g, 44.24 mmol, 1 eq, CAS No.: 3736-77-4) was dissolved in N,N-dimethylformamide (100 ml, alternative DMF), added with imidazole (6.02 g, 88.48 mmol, 2 eq, CAS No.: 288-32-4, alternative IM) and tert-butylchlorodiphenylsilane (13.38 g, 48.66 mmol, 1.1 eq, CAS No.: 58479-61-1, alternative TBDPSCl), and stirred at 25° C. for 16 h. After reaction was completed, reaction system was added with a saturated aqueous ammonium chloride solution (300 ml), and extracted with ethyl acetate (3×200 ml). Organic phases were combined, washed with a saturated aqueous sodium chloride solution (2×100 ml), dried over anhydrous sodium sulfate, filtered, concentrated, and subjected to normal phase chromatography column purification (eluent: dichloromethane/methanol=20/1, v/v), to provide white solid compound NM200-2 (17.08 g, yield 83.2%). MS ESI (m/z)=465.2 [M+H]⁺.

### (7-2) Synthesis of Compound NM200-3

At 25° C., 1-docosanol (90.16 g, 275.96 mmol, 7.5 eq, CAS No.: 661-19-8) was dissolved in diethylene glycol dimethyl ether (170 ml, CAS No.: 111-96-6). Nitrogen replacement was performed three times, a diethylene glycol dimethyl ether solution of trimethylaluminum (46 mL, 91.99 mmol, 2.5 eq, CAS No.: 75-24-1) was added in a nitrogen atmosphere, mixture was warmed to 100° C. and stirred at 100° C. for 30 min, and cooled to 60° C. and added with compound NM200-2 (17.08 g, 36.79 mmol, 1 eq) at 60° C. Reaction system was stirred at 145° C. for 16 h in the nitrogen atmosphere. After reaction was completed, the reaction system was added with a saturated aqueous ammonium chloride solution (300 ml), and extracted with ethyl acetate (3×200 ml). Organic phases were combined, washed with a saturated aqueous sodium chloride solution (2×100 ml), dried over anhydrous sodium sulfate, filtered, concentrated, and subjected to normal phase chromatography column purification (eluent: petroleum ether/ethyl acetate=1/1, v/v), to provide white solid compound NM200-3 (10.8 g, yield 63.46%). MS ESI (m/z)=791.6 [M+H]⁺.

### (7-3) Synthesis of Compound NM200-4

At 25° C., compound NM200-3 (10.8 g, 13.66 mmol, 1 eq) was dissolved in tetrahydrofuran (100 ml), and added with a tetrahydrofuran solution (20.5 mL, 1 M, 20.49 mmol, 1.5 eq) of tetrabutylammonium fluoride. Reaction solution was stirred at 25° C. for 3 h. After reaction was completed, the reaction solution was spin-dried to remove solvent, and subjected to reversed phase chromatography column purification (eluent: water/acetonitrile=5/95, v/v), to provide white solid compound NM200-4 (3.74 g, yield 49.56%). MS ESI (m/z)=553.4 [M+H]⁺.

### (7-4) Synthesis of Compound NM200-5

At 25° C., compound NM200-4 (3.26 g, 5.9 mmol, 1 eq) was dissolved in pyridine (30 ml), and added with triethylamine (1.19 g, 9.8 mmol, 2 eq). Reaction system was cooled to 0° C. by an ice bath, added with 4,4′-dimethoxy triphenylchloromethane (4 g, 9.8 mmol, 2 eq, CAS No.: 40615-36-9, alternative DMTrCl) portion-wise at 0° C., and stirred at 25° C. for 16 h. After reaction was completed, reaction solution was quenched by adding methanol, spin-dried to remove solvent, diluted by adding ethyl acetate (100 ml), washed with a saturated aqueous ammonium chloride solution (2×50 ml) and a saturated aqueous sodium chloride solution (2×30 ml), dried over anhydrous sodium sulfate, filtered, concentrated, and subjected to normal phase chromatography column purification (eluent: petroleum ether/ethyl acetate=3/1, v/v), to provide yellow solid compound NM200-5 (5 g, yield 99.2%). MS ESI (m/z)=855.4 [M+H]⁺.

### (7-5) Synthesis of Compound NM200

Compound NM200-5 (4.73 g, 5.54 mmol, 1 eq) dried with acetonitrile (3×30 mL) was dissolved in dichloromethane (25 mL), added with a dichloromethane (25 mL) solution of bis(diisopropylamino)(2-cyanoethoxy)phosphine (2.515 g, 8.30 mmol, 1.5 eq) dried with acetonitrile (3×10 mL), and added with 1H-imidazole-4,5-dinitrile (524.2 mg, 4.43 mmol, 0.8 eq). Nitrogen replacement was performed three times, and reaction solution was stirred at 25° C. for 1 h in a nitrogen atmosphere. After reaction was completed, the reaction solution was diluted by adding a saturated aqueous sodium bicarbonate solution (50 ml), and extracted with dichloromethane (3×30 ml). Organic phases were combined, washed with a saturated aqueous sodium chloride solution (2×30 ml), dried over anhydrous sodium sulfate, filtered, concentrated, and subjected to normal phase chromatography column purification (eluent: petroleum ether/ethyl acetate=2/1, v/v), to provide white solid compound NM-200 (4.87 g, yield 83.43%). MS ESI (m/z)=1055.3 [M+H]⁺.

¹H NMR (400 MHz, Chloroform-d) δ 7.78 (dd, J=13.2, 8.1 Hz, 1H), 7.43-7.15 (m, 9H), 6.89 (ddd, J=8.6, 6.2, 2.1 Hz, 4H), 5.80 (t, J=3.5 Hz, 1H), 5.26 (ddd, J=14.2, 8.1, 1.8 Hz, 1H), 4.37 (ddt, J=24.1, 10.7, 5.5 Hz, 1H), 4.18-3.99 (m, 2H), 3.74 (d, J=2.5 Hz, 8H), 3.56 (tdd, J=13.1, 10.2, 5.2 Hz, 5H), 3.39-3.33 (m, 1H), 2.80-2.73 (m, 1H), 2.61 (td, J=6.0, 3.5 Hz, 1H), 1.63-1.43 (m, 3H), 1.22 (d, J=3.3 Hz, 40H), 1.16-1.01 (m, 8H), 0.98 (d, J=6.7 Hz, 2H), 0.84 (t, J=6.6 Hz, 3H).

### Preparation Example 8: Synthesis of Compound NM202

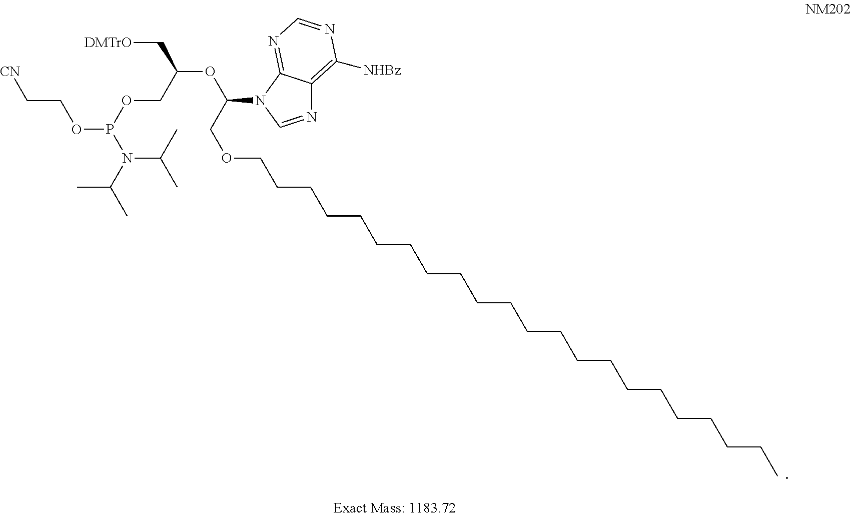

In the present preparation example, a synthesis route of compound NM202 was as follows:

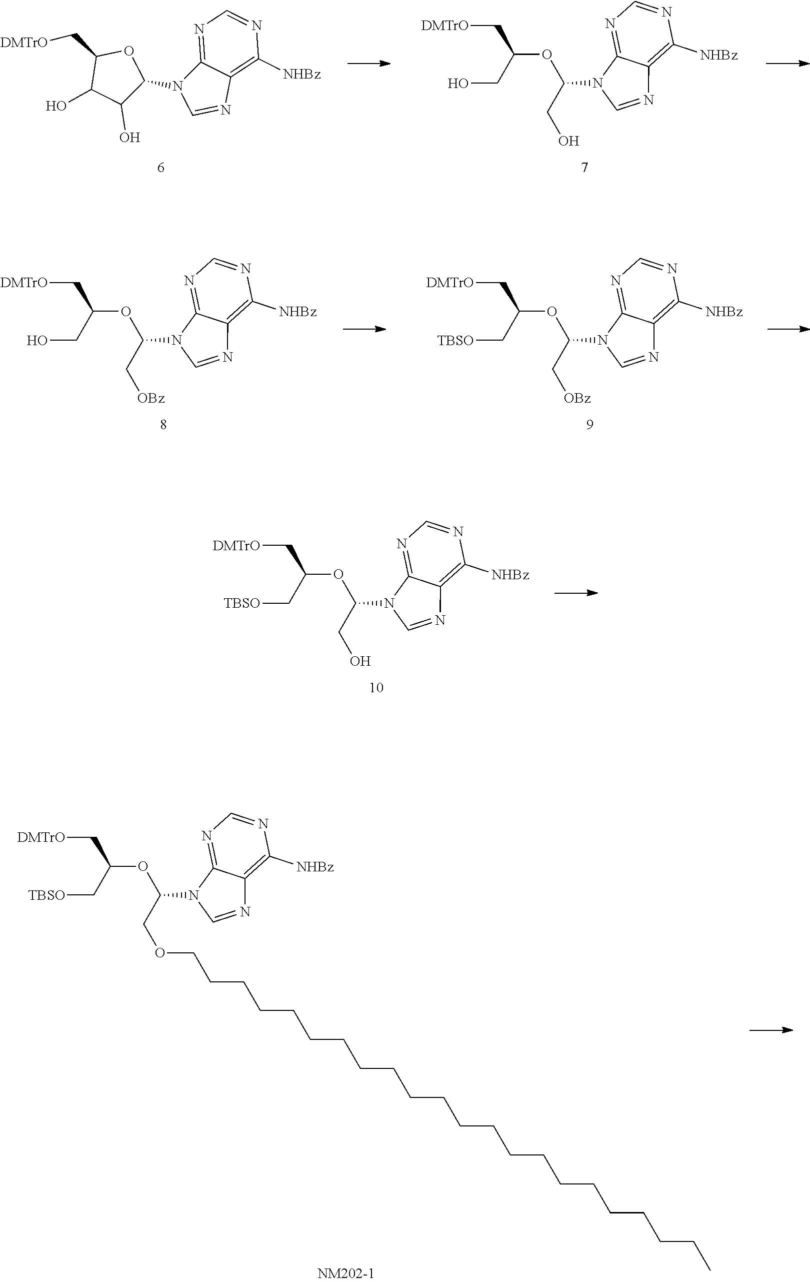

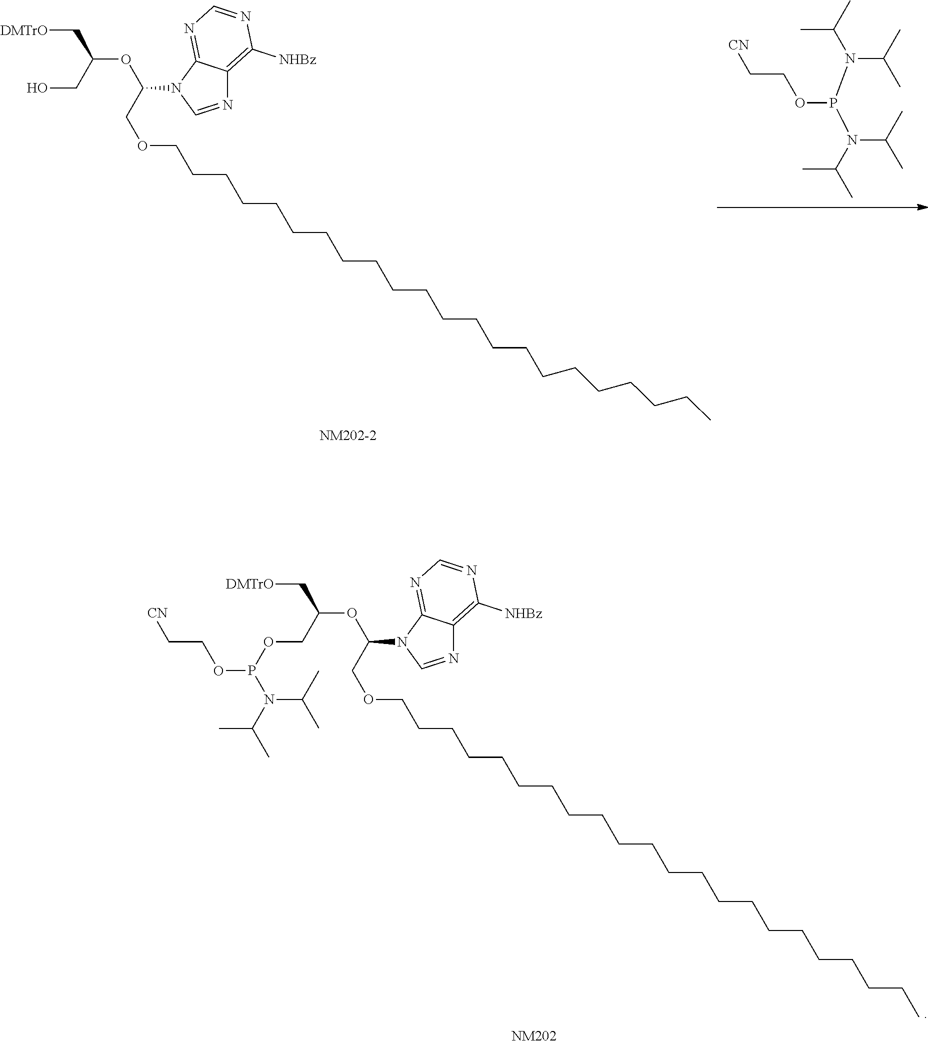

### (8-1) Synthesis of Compound 7

Compound 6 (22.6 g, 33.56 mmol, 1 eq, CAS No.: 81246-82-4) was dissolved in a mixed solution of 1,4-dioxane (345 ml) and water (115 ml), and added with sodium periodate (8.04 g, 36.92 mmol, 1.1 eq). Nitrogen replacement was performed three times, and reaction solution was stirred at 25° C. for 2 h in a nitrogen atmosphere. After reaction was completed, the reaction solution was concentrated to remove solvent, diluted by adding water (300 ml), and extracted with dichloromethane (5×200 ml). Organic phases were combined, washed with a saturated aqueous sodium bicarbonate solution (2×100 ml) and a saturated aqueous sodium chloride solution (2×100 ml), dried over anhydrous sodium sulfate, filtered, and concentrated, to provide a white solid (22.6 g), which was directly used in the next step without purification.

At 25° C., the white solid (22.6 g) in this step was dissolved in 1,4-dioxane (350 ml), and added with sodium borohydride (1.4 g, 36.92 mmol, 1.1 eq, CAS No.: 16940-66-2) portion-wise. Argon replacement was performed three times, and reaction solution was stirred at 25° C. for 2 h in an argon atmosphere. After reaction was completed, the reaction solution was quenched by adding a saturated aqueous ammonium chloride solution (120 ml), and extracted with dichloromethane (4×150 ml). Organic phases were combined, washed with a saturated aqueous sodium chloride solution (2×100 mL), dried over anhydrous sodium sulfate, filtered, and concentrated, to provide white solid compound 7 (20.4 g, yield 98.7%). MS ESI (m/z)=676.27 [M+H]⁺.

### (8-2) Synthesis of Compound 8

At 25° C., compound 7 (20 g, 31.079 mmol, 1 eq) was dissolved in dichloromethane (200 ml), and added with 1,8-diazabicyclo[5.4.0]undeca-7-ene (9.456 g, 62.158 mmol, 2 eq, CAS No.: 6674-22-2). Nitrogen replacement was performed three times, and reaction solution was cooled to −78° C. in a nitrogen atmosphere, and added with benzoyl chloride (4.805 g, 34.187 mmol, 1.1 eq, CAS No.: 98-88-4) dropwise at −78° C. After the dropwise addition was completed, the reaction solution was stirred at −78° C. for 1 h. After reaction was completed, the reaction solution was quenched by adding a saturated aqueous ammonium chloride solution (150 ml), and extracted with dichloromethane (3×120 ml). Organic phases were combined, washed with a saturated aqueous sodium bicarbonate solution (2×200 mL) and a saturated aqueous sodium chloride solution (2×100 mL), dried over anhydrous sodium sulfate, filtered, and concentrated, to provide yellow solid compound 8 (25 g). MS ESI (m/z)=780.30 [M+H]⁺.

### (8-3) Synthesis of Compound 9

At 25° C., compound 8 (24.7 g, 31.695 mmol, 1 eq) was dissolved in dichloromethane (250 ml), and added with imidazole (3.23 g, 47.543 mmol, 1.5 eq, CAS No: 288-32-4) and tert-butyldimethylsilyl chloride (6.21 g, 41.203 mmol, 1.3 eq, CAS No.: 18162-48-6). Nitrogen replacement was performed three times, and reaction solution was stirred at 25° C. for 16 h in a nitrogen atmosphere. After reaction was completed, the reaction solution was spin-dried to remove solvent, diluted by adding water (400 ml), and extracted with dichloromethane (4×150 ml). Organic phases were combined, washed with a saturated aqueous sodium bicarbonate solution (2×80 ml) and a saturated aqueous sodium chloride solution (100 ml), dried over anhydrous sodium sulfate, filtered, and concentrated, to provide yellow solid compound 9 (28.6 g), which was directly used in the next step without purification. MS ESI (m/z)=894.38 [M+H]⁺.

### (8-4) Synthesis of Compound 10

At 25° C., compound 9 (28.3 g, 31.689 mmol, 1 eq) was dissolved in a mixed solution of pyrimidine (170 ml) and methanol (68 ml). Reaction system was cooled to a temperature of 0° C. by an ice bath, and added with an aqueous sodium hydroxide solution (31.6 mL, 1 M, 1 eq) at 0° C. Nitrogen replacement was performed three times, and reaction solution was stirred at 0° C. for 0.5 h in a nitrogen atmosphere. After reaction was completed, the reaction solution was spin-dried to remove solvent, diluted by adding water (150 mL), and extracted with dichloromethane (3×200 mL). Organic phases were combined, washed with a saturated aqueous ammonium chloride solution (2×100 mL) and a saturated aqueous sodium chloride solution (2×100 mL), dried over anhydrous sodium sulfate, filtered, concentrated, and subjected to normal phase chromatography column purification (eluent: petroleum ether/ethyl acetate=1/1, v/v), to provide white solid compound 10 (16.21 g, yield 64.8%). MS ESI (m/z)=790.36 [M+H]⁺.

### (8-5) Synthesis of Compound NM202-1

N,N-dimethylformamide (30 ml) was cooled to 0° C. by an ice bath, added with sodium hydride (668.8 mg, 16.7 mmol, 3.3 eq), and added with compound 10 (4 g, 5.1 mmol, 1 eq). Nitrogen replacement was performed three times, and reaction solution was stirred at 0° C. for 30 min in a nitrogen atmosphere, added with 1-bromodocosane (2.96 g, 7.7 mmol, 1.5 eq, CAS No.: 6938-66-5) and potassium iodide (1.26 g, 7.7 mmol, 1.5 eq), slowly warmed to 25° C. and stirred at 25° C. for 16 h. After reaction was completed, the reaction solution was quenched by adding a saturated aqueous ammonium chloride solution (150 ml), and extracted with ethyl acetate (3×150 ml). Organic phases were combined, washed with a saturated aqueous sodium bicarbonate solution (2×80 ml) and a saturated aqueous sodium chloride solution (100 ml), dried over anhydrous sodium sulfate, filtered, concentrated, and subjected to normal phase chromatography column purification (eluent: petroleum ether/ethyl acetate=3/1), to provide yellow solid compound NM202-1 (3.65 g, yield 65.6%). MS ESI (m/z)=1098.61 [M+H]⁺.

### (8-6) Synthesis of Compound NM202-2

At 25° C., compound NM202-1 (3.65 g, 3.3 mmol, 1 eq) was dissolved in tetrahydrofuran (40 ml), and added with a tetrahydrofuran solution (5 mL, 1 M, 5 mmol, 1.5 eq) of tetrabutylammonium fluoride. Nitrogen replacement was performed three times, and reaction solution was stirred at 25° C. for 3 h in a nitrogen atmosphere. After reaction was completed, the reaction solution was directly concentrated, and subjected to normal phase chromatography column purification (eluent: petroleum ether/ethyl acetate=1/1, v/v), to provide white solid compound NM202-2 (1.3 g, yield 39.8%). MS ESI (m/z)=984.52 [M+H]⁺.

### (8-7) Synthesis of Compound NM202

Compound NM202-7 (1.3 g, 1.32 mmol) dried with acetonitrile (3×20 mL) was dissolved in dichloromethane (15 mL), added with a dichloromethane (15 mL) solution of bis(diisopropylamino)(2-cyanoethoxy)phosphine (598.9 mg, 1.98 mmol, 1.5 eq) dried with acetonitrile (3×10 mL), and added with 1H-imidazole-4,5-dinitrile (124.8 mg, 1.06 mmol, 0.8 eq). Nitrogen replacement was performed three times, and reaction solution was stirred at 25° C. for 1 h in a nitrogen atmosphere. After reaction was completed, the reaction solution was diluted by adding a saturated aqueous sodium bicarbonate solution (30 ml), and extracted with dichloromethane (3×20 ml). Organic phases were combined, washed with a saturated aqueous sodium chloride solution (2×30 ml), dried over anhydrous sodium sulfate, filtered, concentrated, and subjected to normal phase chromatography column purification (eluent: petroleum ether/ethyl acetate=2/1, v/v), to provide white solid compound NM202 (1 g, yield 58.9%). MS ESI (m/z)=1100.63 [M+H]⁺.

¹H NMR (400 MHz, DMSO-d6) δ 8.73 (s, 1H), 8.68 (d, J=1.6 Hz, 1H), 8.06 (d, J=7.6 Hz, 2H), 7.64 (t, J=7.4 Hz, 1H), 7.55 (t, J=7.6 Hz, 2H), 7.22-7.03 (m, 5H), 7.01-6.91 (m, 4H), 6.81-6.74 (m, 4H), 6.21 (s, 1H), 4.09-3.91 (m, 2H), 3.69 (d, J=1.7 Hz, 9H), 3.61-3.40 (m, 4H), 3.03-2.89 (m, 1H), 2.86-2.79 (m, 1H), 2.73 (dt, J=11.8, 5.9 Hz, 2H), 1.39 (t, J=6.7 Hz, 2H), 1.30-1.10 (m, 52H), 0.85 (d, J=6.4 Hz, 3H).

### Reference Compound Uhd

Compound Uhd has a structural formula as follows:

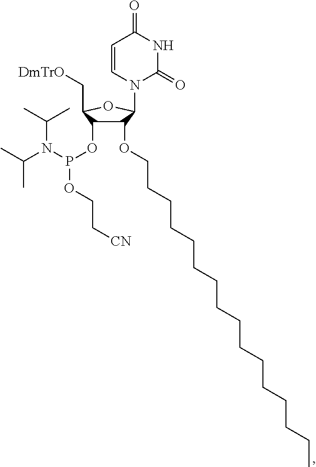

purchased from Shanghai Hongene Biotech Corporation.

### Example 1: Synthesis of Double-Stranded Oligonucleotide siRNA

### (1) Synthesis of Sense Strand (SS) and Antisense Strand (AS)

According to a standard oligonucleotide solid phase synthesis scheme, solid phase vector CPG was used to start a cycle, and nucleoside monomers or vector (i.e. nucleoside analog vectors prepared in Preparation Examples or vector Uhd purchased from Shanghai Hongene Biotech Corporation.) were linked one by one in a direction of 3′-5′ of nucleotide sequences of TBLE 1.

Each nucleoside monomer was linked through four-step reactions of deprotection, coupling, capping, oxidation or sulfurization. Synthesis conditions were given as follows:

The nucleoside monomer was formulated into an acetonitrile solution of nucleoside monomer at a concentration of 0.1 M.

Conditions of deprotection reaction in each step were the same. The conditions of the deprotection reaction were as follows: a temperature was 25° C., reaction duration was 70 s, deprotection reagent was a dichloromethane solution (3 vol %) of dichloroacetic acid, and a molar ratio of dichloroacetic acid to 4,4′-dimethoxytrityl protecting group on the solid phase vector was 5:1.

Conditions of coupling reaction in each step were the same. The conditions of the coupling reaction were as follows: a temperature was 25° C., a molar ratio of nucleic acid sequences linked to the solid phase vector to nucleoside monomers was 1:10, a molar ratio of nucleic acid sequences linked to the solid phase vector to a coupling reagent was 1:65, reaction duration was 600 s, a coupling reagent was an acetonitrile solution of 5-ethylthio-1H-tetrazole at a concentration of 0.5 M, and a thionating reagent was an acetonitrile/pyridine mixed solution of hydroxanthogen at a concentration of 0.2 M (a volume ratio of acetonitrile to pyridine was 1:1).

Conditions of capping reaction in each step were the same. The conditions of the capping reaction were as follows: a temperature was 25° C.; reaction duration was 2 min; a capping reagent solution was a mixed solution of Cap1 and Cap2 in a molar ratio of 1:1, Cap1 was a pyridine/acetonitrile mixed solution of N-methylimidazole at a concentration of 20 vol %, a volume ratio of pyridine to acetonitrile was 3:5, and Cap2 was an acetonitrile solution of acetic anhydride at a concentration of 20 vol %; a molar ratio of N-methyl imidazole in Cap1 capping reagent, acetic anhydride in Cap2 capping reagent to nucleic acid sequences linked to the solid phase vector was 1:1:1.

Conditions of oxidation reaction in each step were the same. The conditions of the oxidation reaction were as follows: a temperature was 25° C.; reaction duration was 3 s; an oxidation reagent was iodine solution at a concentration of 0.05 M, a molar ratio of iodine to nucleic acid sequences linked to the solid phase vector in the coupling reaction was 30:1; and the oxidation reaction was carried out in a water/pyridine mixed solvent (a volume ratio of water to pyridine was 1:9). Conditions of sulfurization were as follows: a temperature was 25° C.; reaction duration was 360 s; a thionating reagent was pyridine solution of hydrogenated xanthogen at a concentration of 0.2 M, a molar ratio of the thionating reagent to nucleic acid sequences linked to the solid phase vector in the coupling reaction was 4:1; and a thionation reaction was carried out in a water/pyridine mixed solvent (a volume ratio of water to pyridine was 1:9).

After the last nucleoside monomer was linked, the nucleic acid sequences linked on the solid phase vector were cleaved, deprotected, purified, and desalted in turn, and subsequently lyophilized to provide the sense strand.

Conditions of cleavage and deprotection were as follows: the synthesized nucleotide sequences linked with the solid phase vector were added into ammonia water at a concentration of 25 mass % to react at 55° C. for 16 h, an amount of the ammonia water being 0.5 ml/μmol, solvent was removed, and resultant was concentrated in vacuum to dryness. After treatment with the ammonia water, product was dissolved with 0.4 ml/μmol N-methylpyrrolidone relative to an amount of single-stranded nucleic acid, subsequently, 0.3 ml/μmol trimethylamine and 0.6 ml/μmol triethylamine trihydrofluoride were added, to remove 2′-O-TBDMS protection on ribose. Conditions of purification and desalination were as follows: purification of nucleic acid was completed by gradient elution of NaCl using a preparative ion chromatography purification column (Source 15Q). Specifically, eluent 1 was 20 mM sodium phosphate (pH=8.1), and solvent was a water/acetonitrile mixed solution (a volume ratio of water to acetonitrile was 9:1); eluent 2 was 1.5 M sodium chloride, 20 mM sodium phosphate (pH=8.1), and solvent was a water/acetonitrile mixed solution (a volume ratio of water to acetonitrile was 9:1); and elution gradient was eluent 1:eluent 2=(100:0)−(50:50). Product eluates were collected, combined, and desalted using a reversed phase chromatography purification column, wherein desalination conditions comprised desalination using a dextran gel column, dextran gel G25 as a filler, and elution with deionized water.

Detection: purity was detected using ion exchange chromatography (IEX-HPLC); and the molecular weights of the sense strand and antisense strand were detected using liquid chromatography-mass spectrometry (LC-MS). An actually measured value and a theoretical value of the molecular weight were compared, and if the actually measured value was approximately equal to the theoretical value, it demonstrated that the sense strand and antisense strand of the siRNA were obtained. Detection results are as shown in TABLE 2.

(2) Synthesis of siRNA

The sense strand and the antisense strand synthesized in step (1) were mixed in an equal molar ratio, dissolved in water for injection, heated to 95° C., slowly cooled to 25° C. and kept at 25° C. for 10 min, to make the sense strand and the antisense strand form a double-stranded structure via a hydrogen bond, so as to provide the siRNAs having the sense strand and the antisense strand as shown in TABLE 1.

The siRNAs prepared in Examples of the present disclosure were all obtained by nucleotide replacement and/or modification of the following nucleotide sequences:

Sense strand (5′-3′):
(SEQ ID NO: 11)
UUUUAAUCCUCACUCUAAA
Antisense strand (5′-3′):
(SEQ ID NO: 12)
UUUAGAGUGAGGAUUAAAAUG

Modified nucleotide sequence information of the siRNAs prepared in Examples of the present disclosure is as shown in the following table:

TABLE 1
Sense Strand and Antisense Strand Information of Various siRNAs

| SIRNA No. | Sense strand sequence (5′-3′) | Antisense strand sequence (5′-3′) |
|---|---|---|
| RX899001 | UmsUmsUmUmAmAmUfCfCfUmCmAmCm | UmsUfsUmAmGmAfGmUmGmAmGmGmAm |
| | UmCmUmAmAmAm(SEQ ID NO: 1) | UfUmAfAmAmAmsUmsGm(SEQ ID NO: 2) |
| RZ899008 | UmsUmsUm(NM031)AmAmUfCfCfUmCmA | UmsUfsUmAmGmAfGmUmGmAmGmGmAm |
| | mCmUmCmUmAmAmAm(SEQ ID NO: 3) | UfUmAfAmAmAmsUmsGm(SEQ ID NO: 2) |
| RZ899001 | UmsUmsUm(Uhd)AmAmUfCfCfUmCmAmC | UmsUfsUmAmGmAfGmUmGmAmGmGmAm |
| | mUmCmUmAmAmAm(SEQ ID NO: 4) | UfUmAfAmAmAmsUmsGm(SEQ ID NO: 2) |
| RZ899052 | UmsUmsUmUm(NM058)AmUfCfCfUmCmA | VPUmsUfsUmAmGmAfGmUmGmAmGmGm |
| | mCmUmCmUmAmAmAm(SEQ ID NO: 5) | AmUfUmAfAmAmAmsUmsGm (SEQ ID |
| | | NO: 22) |
| RZ899053 | UmsUmsUmUmAm(NM058) UfCfCfUmCmA | VPUmsUfsUmAmGmAfGmUmGmAmGmGm |
| | mCmUmCmUmAmAmAm(SEQ ID NO: 6) | AmUfUmAfAmAmAmsUmsGm (SEQ ID |
| | | NO: 22) |
| RZ899056 | UmsUmsUmUmAmAmUfCfCfUmCmAmCm | VPUmsUfsUmAmGmAfGmUmGmAmGmGm |
| | UmCmUmAmAmAm (SEQ ID NO: 1) | AmUfUmAfAmAmAmsUmsGm (SEQ ID |
| | | NO: 22) |
| RZ899055 | UmsUmsUm(Uhd)AmAmUfCfCfUmCmAmC | VPUmsUfsUmAmGmAfGmUmGmAmGmGm |
| | mUmCmUmAmAmAm (SEQ ID NO: 4) | AmUfUmAfAmAmAmsUmsGm (SEQ ID |
| | | NO: 22) |
| RZ899060 | UmsUmsUm(NM031)AmAmUfCfCfUmCmA | VPUmsUfsUmAmGmAfGmUmGmAmGmGm |
| | mCmUmCmUmAmAmAm (SEQ ID NO: 3) | AmUfUmAfAmAmAmsUmsGm (SEQ ID |
| | | NO: 22) |
| RZ899093 | UmsUmsUm(Uhd)AmAmUfCfCfUfCmAmC | VPUmsUfsUmAmGmAfGmUmGmAmGmGfA |
| | mUmCmUmAmAmAm (SEQ ID NO: 13) | mUfU(moe)AfAmAmAmsUmsGm (SEQ ID |
| | | NO: 23) |
| RZ899092 | UmsUmsUm(NM031)AmAmUfCfCfUfCmA | VPUmsUfsUmAmGmAfGmUmGmAmGmGfA |
| | mCmUmCmUmAmAmAm (SEQ ID NO: 14) | mUfU(moe)AfAmAmAmsUmsGm (SEQ ID |
| | | NO: 23) |
| RZ899090 | UmsUmsUm(NM073)AmAmUfCfCfUfCmA | VPUmsUfsUmAmGmAfGmUmGmAmGmGfA |
| | mCmUmCmUmAmAmAm (SEQ ID NO: 15) | mUfU(moe)AfAmAmAmsUmsGm (SEQ ID |
| | | NO: 23) |
| RZ899091 | UmsUmsUm(NM074)AmAmUfCfCfUfCmA | VPUmsUfsUmAmGmAfGmUmGmAmGmGfA |
| | mCmUmCmUmAmAmAm (SEQ ID NO: 16) | mUfU(moe)AfAmAmAmsUmsGm (SEQ ID |
| | | NO: 23) |
| RX599002 | UmsUmsUmUmAmAmUfCfCfUfCmAmCmU | VPUmsUfsUmAmGmAfGmUmGfAmGmGmA |
| | mCmUmAmAmAm (SEQ ID NO: 17) | mUfU(moe)AfAmAmAmsUmsGm (SEQ ID |
| | | NO: 24) |
| RZ899076 | UmsUmsUm(NM200)AmAmUfCfCfUfCmA | VPUmsUfsUmAmGmAfGmUmGfAmGmGmA |
| | mCmUmCmUmAmAmAm (SEQ ID NO: 18) | mUfU(moe)AfAmAmAmsUmsGm (SEQ ID |
| | | NO: 24) |
| RZ899077 | UmsUmsUm(NM201)AmAmUfCfCfUfCmA | VPUmsUfsUmAmGmAfGmUmGfAmGmGmA |
| | mCmUmCmUmAmAmAm (SEQ ID NO: 19) | mUfU(moe)AfAmAmAmsUmsGm (SEQ ID |
| | | NO: 24) |
| RZ899078 | UmsUmsUmUmAm(NM202)UfCfCfUfCmA | VPUmsUfsUmAmGmAfGmUmGfAmGmGmA |
| | mCmUmCmUmAmAmAm (SEQ ID NO: 20) | mUfU(moe)AfAmAmAmsUmsGm (SEQ ID |
| | | NO: 24) |
| RZ899122 | UmsUmsUm(NM087)AmAmUfCfCfUfCmA | VPUmsUfsUmAmGmAfGmUmGfAmGmGmA |
| | mCmUmCmUmAmAmAm (SEQ ID NO: 21) | mUfU(moe)AfAmAmAmsUmsGm (SEQ ID |
| | | NO: 24) |
| RX899121 | UmsUmsUmUmAmAmUfCfCfUfCmAmCmU | VPUmsUfsUmAmGmAfGmUmGmAmGmGfA |
| | mCmUmAmAmAm (SEQ ID NO: 25) | mUfU(moe)AfAmAmAmsUmsGm (SEQ ID |
| | | NO: 23) |

Unless otherwise stated, meanings of base composition and modifications used in the present disclosure are as follows: capital letters A, U, G, C, and T represent base composition of nucleotides; lowercase letter m means that a nucleotide adjacent to the left of the letter e is a 2′-O-methyl modified (also called: 2′-methoxy modified) nucleotide; lowercase letter f means that a nucleotide adjacent to the left of the letter f is a 2′-fluoro modified nucleotide; (moe) means that a nucleotide adjacent to the left of combined identity (moe) is a 2′-O-methoxyethyl (i.e., 2′-O-MOE) modified nucleotide; and lowercase letter s means that two nucleotides adjacent to the left and right of the letter s are linked by a phosphorothioate diester bond.

Detection: molecular weight of siRNA was detected using liquid chromatography-mass spectrometry (LC-MS). An actually measured value and a theoretical value of the molecular weight were compared, and if the actually measured value was approximately equal to the theoretical value, it indicated that the siRNA was obtained. Detection results are as shown in TABLE 2.

TABLE 2
Detection Results of Sense Strands and Antisense Strands of Various siRNAs

| | Sense strand | | | Antisense strand | | | siRNA | | |
|---|---|---|---|---|---|---|---|---|---|
| | Theoretical | Actual | | Theoretical | Actual | | Theoretical | Actual | |
| siRNA | molecular | molecular | Purity | molecular | molecular | Purity | molecular | molecular | Purity |
| No. | weight | weight | % | weight | weight | % | weight | weight | % |
| RX899001 | 6151.11 | 6152.7 | 96.1% | 7096.83 | 7098.3 | 95.8% | 13247.94 | 13251.00 | 94.60 |
| RZ899008 | 6361.5 | 6361.6 | 98.8% | 7096.83 | 7097.1 | 96.8% | 13458.33 | 13458.70 | 93.60 |
| RZ899001 | 6363.2 | 6364.0 | 98.6% | 7096.83 | 7097.1 | 96.8% | 13460.03 | 13461.10 | 92.10 |
| RZ899052 | 6363.19 | 6364.40 | 99.00 | 7172.77 | 7173.90 | 93.40 | 13536.00 | 13537.20 | 92.20 |
| RZ899053 | 6363.19 | 6364.10 | 99.10 | 7172.77 | 7173.90 | 93.40 | 13536.00 | 13536.90 | 93.50 |
| RZ899056 | 6151.10 | 6151.80 | 97.70 | 7172.77 | 7173.90 | 93.40 | 13323.90 | 13324.57 | 91.90 |
| RZ899055 | 6361.50 | 6362.50 | 99.30 | 7172.77 | 7173.90 | 93.40 | 13534.30 | 13535.27 | 92.70 |
| RZ899060 | 6363.10 | 6364.60 | 97.10 | 7172.76 | 7173.30 | 91.80 | 13535.90 | 13537.40 | 94.20 |
| RZ899093 | 6349.46 | 6350.10 | 98.50 | 7204.68 | 7205.20 | 98.50 | 13554.10 | 13554.80 | 96.00 |
| RZ899092 | 6351.16 | 6352.30 | 97.10 | 7204.68 | 7205.20 | 98.50 | 13555.80 | 13557.00 | 90.90 |
| RZ899090 | 6379.16 | 6380.50 | 99.00 | 7204.68 | 7205.20 | 98.50 | 13583.80 | 13585.20 | 96.10 |
| RZ899091 | 6407.16 | 6408.30 | 98.20 | 7204.68 | 7205.20 | 98.50 | 13611.80 | 13613.00 | 96.30 |
| RX599002 | 6139.06 | 6139.90 | 95.40 | 7204.68 | 7205.30 | 98.00 | 13343.70 | 13344.60 | 91.50 |
| RZ899076 | 6433.27 | 6434.40 | 97.50 | 7204.68 | 7205.30 | 98.00 | 13638.00 | 13639.10 | 92.60 |
| RZ899077 | 6435.28 | 6436.40 | 97.60 | 7204.68 | 7205.30 | 98.00 | 13640.00 | 13641.10 | 93.50 |
| RZ899078 | 6435.26 | 6436.40 | 97.30 | 7204.68 | 7205.30 | 98.00 | 13639.90 | 13641.10 | 92.40 |
| RX899121 | 6139.03 | 6135.40 | 98.50 | 7204.70 | 7201.70 | 94.70 | 13343.70 | 13337.10 | 96.20 |

It can be seen from the data in TABLE 2 that double helix structure siRNAs consisting of a sense strand and an antisense strand were obtained, and the siRNAs had higher purity.

Structural formulas of compound NM031 and compound UHd after being linked with nucleotide are as shown below:

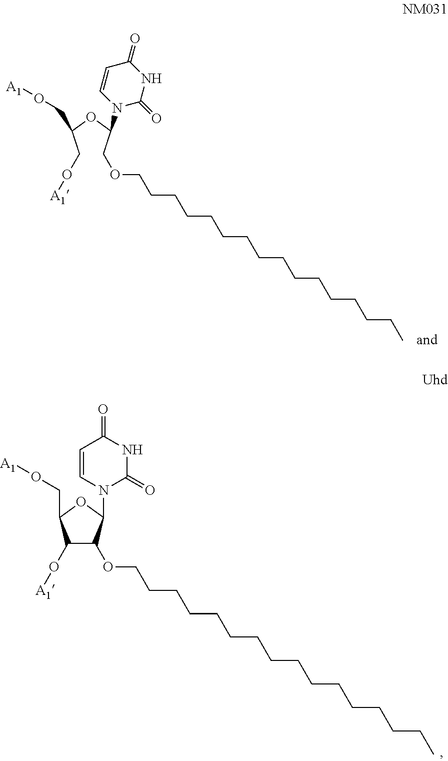

wherein A₁ is UmsUmsUm, and A₁′ is AmAmUfCfCfUmCmAmCmUmCmUmAmAmAm or AmAmUfCfCfUfCmAmCmUmCmUmAmAmAm.

Structural formula of compound NM058 after being linked with nucleotide is as shown below:

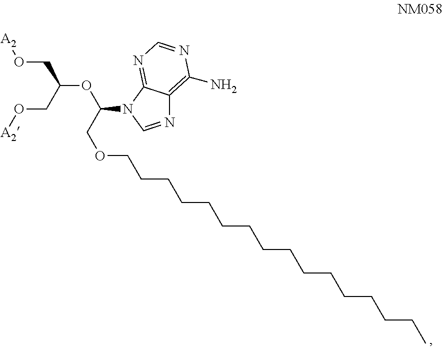

wherein A₂ is UmsUmsUmUm, and A₂′ is AmUfCfCfUmCmAmCmUmCmUmAmAmAm;
or

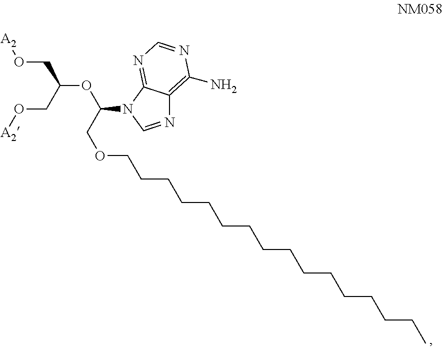

wherein A₃ is UmsUmsUmUmAm, and A₃′ is UfCfCfUmCmAmCmUmCmUmAmAmAm.

(Uhd) represents nucleotide at this site being replaced with nucleotide analog (Uhd); (NM031) represents nucleotide at this site being replaced with nucleotide analog (NM031); (NM058) represents nucleotide at this site being replaced with nucleotide analog (NM058); (NM073) represents nucleotide at this site being replaced with nucleotide analog (NM073); (NM074) represents nucleotide at this site being replaced with nucleotide analog (NM074); (NM087) represents nucleotide at this site being replaced with nucleotide analog (NM087); (NM200) represents nucleotide at this site being replaced with nucleotide analog (NM200); (NM201) represents nucleotide at this site being replaced with nucleotide analog (NM201); and (NM202) represents nucleotide at this site being replaced with nucleotide analog (NM202).

2′-O-methyl modified nucleotide had a structural formula

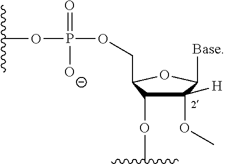

2′-fluoro modified nucleotide had a structural formula

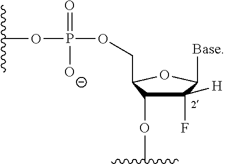

2′-O-MOE modified nucleotide had a structural formula

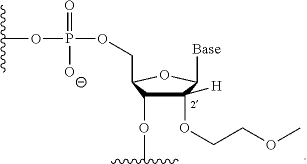

The nucleotide analog (Uhd) had a structural formula

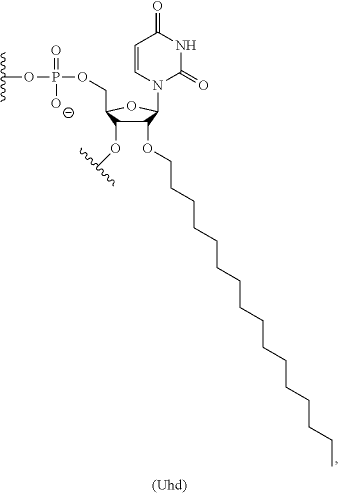

which was formed by the nucleoside analog Uhd participating in siRNA synthesis. Herein, the nucleoside analog Uhd had a structural formula

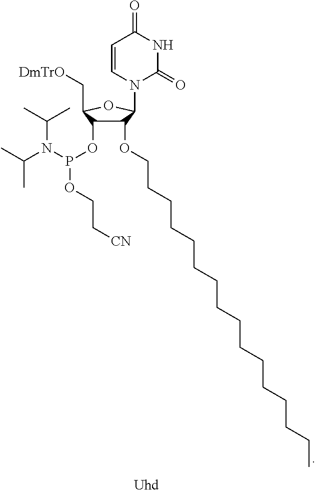

Nucleotide analog (NM031) had a structural formula

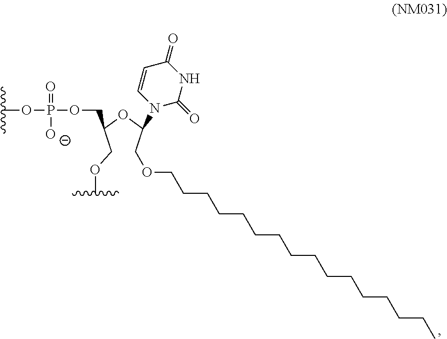 [SMILES: *OC[C@@H](COP(=O)([O-])O*)O[C@H](COCCCCCCCCCCCCCCCC)n1ccc(=O)[nH]c1=O]

which was formed by the nucleoside analog NM031 participating in siRNA synthesis.

Nucleotide analog (NM058) had a structural formula

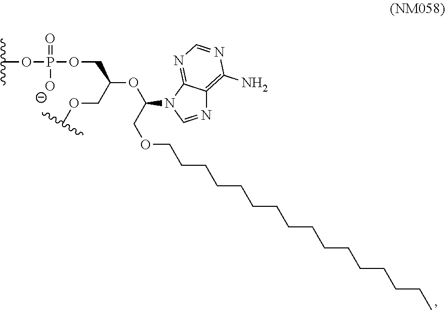 [SMILES: *OC[C@@H](COP(=O)([O-])O*)O[C@H](COCCCCCCCCCCCCCCCC)n1cnc2c(N)ncnc21]

which was formed by the nucleoside analog NM058 participating in siRNA synthesis. Herein, the nucleoside analog NM058 had a structural formula

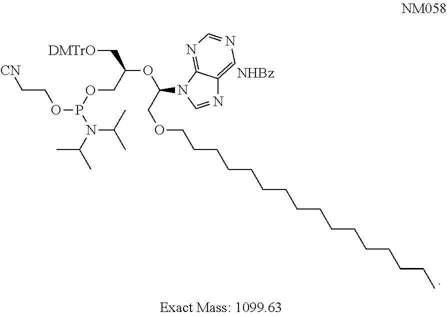

Nucleotide analog (NM073) had a structural formula

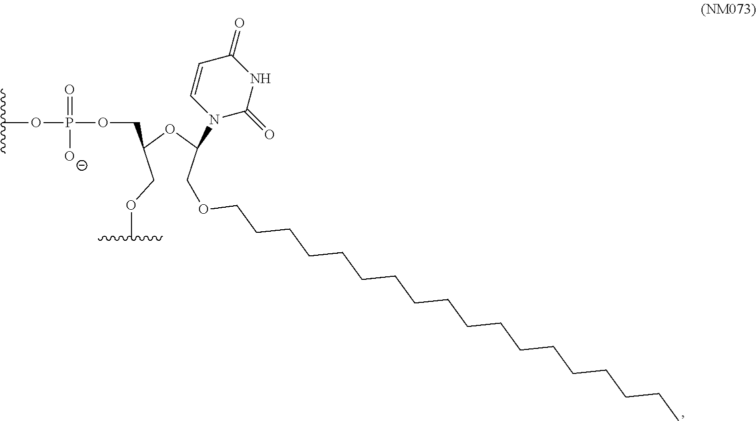 [SMILES: *OC[C@@H](COP(=O)([O-])O*)O[C@H](COCCCCCCCCCCCCCCCCCC)n1ccc(=O)[nH]c1=O]

which was formed by the nucleoside analog NM073 participating in siRNA synthesis.

Nucleotide analog (NM074) had a structural formula

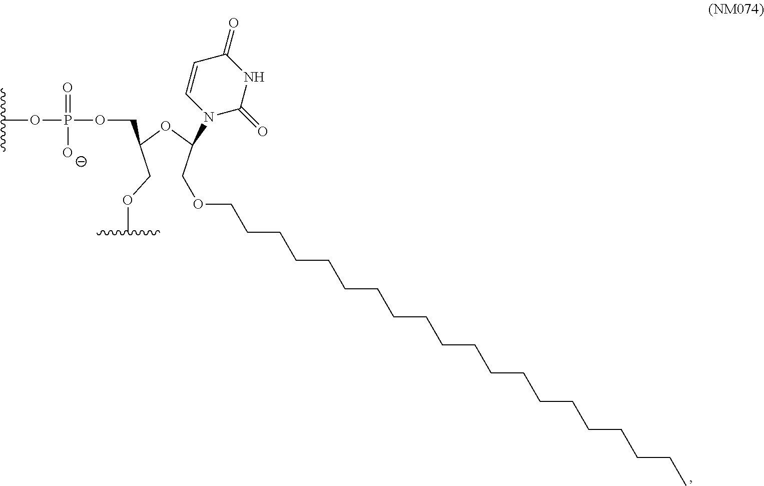 [SMILES: *OC[C@@H](COP(=O)([O-])O*)O[C@H](COCCCCCCCCCCCCCCCCCCCC)n1ccc(=O)[nH]c1=O]

which was formed by the nucleoside analog NM074 participating in siRNA synthesis.

Nucleotide analog (NM200) had a structural formula

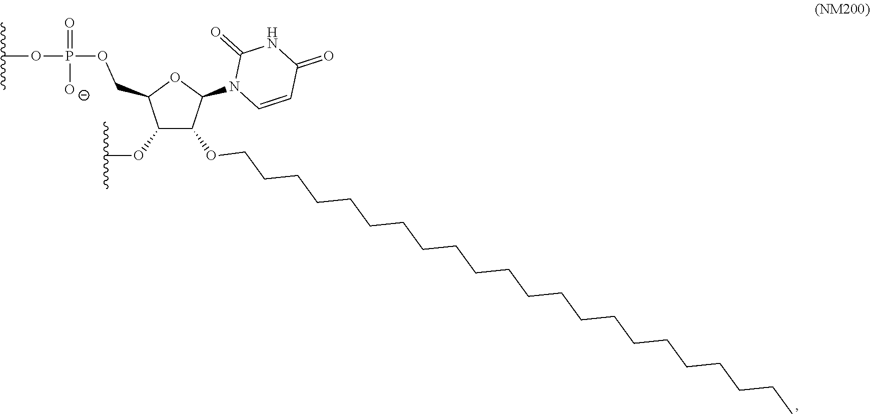 [SMILES: *O[C@H]1[C@@H](OCCCCCCCCCCCCCCCCCCCCCC)[C@H](n2ccc(=O)[nH]c2=O)O[C@@H]1COP(=O)([O-])O*]

which was formed by the nucleoside analog NM200 participating in siRNA synthesis.

Nucleotide analog (NM201) had a structural formula

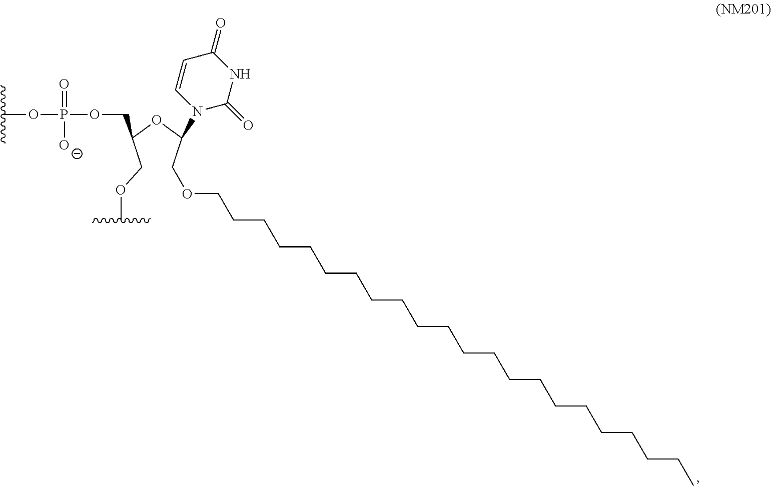 [SMILES: *OC[C@@H](COP(=O)([O-])O*)O[C@H](COCCCCCCCCCCCCCCCCCCCCCC)n1ccc(=O)[nH]c1=O]

which was formed by the nucleoside analog NM201 participating in siRNA synthesis.

Nucleotide analog (NM202) had a structural formula

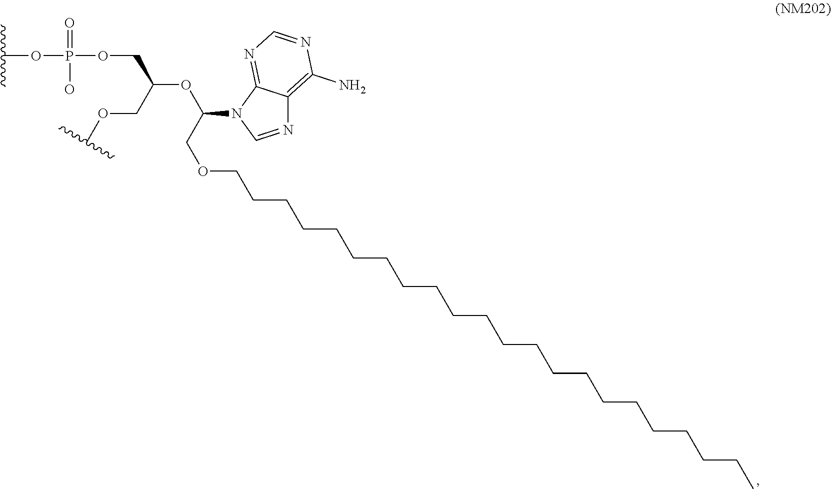 [SMILES: *OC[C@@H](COP(=O)(O)O*)O[C@H](COCCCCCCCCCCCCCCCCCCCCCC)n1cnc2c(N)ncnc21]

which was formed by the nucleoside analog NM202 participating in siRNA synthesis.

Nucleotide analog (NM087) had a structural formula

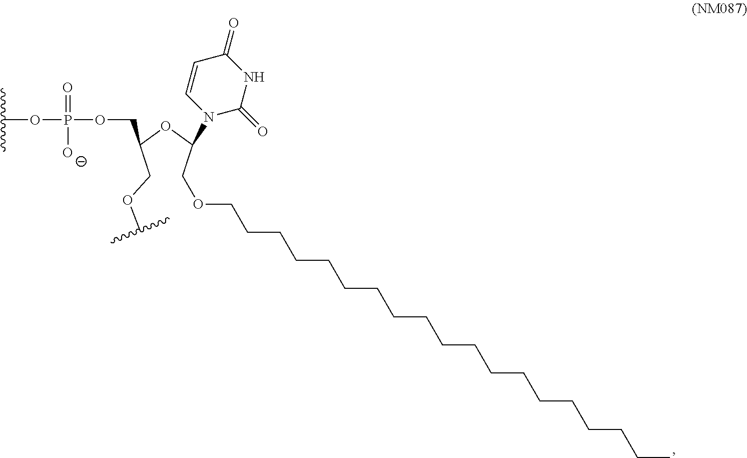 [SMILES: *OC[C@@H](COP(=O)([O-])O*)O[C@H](COCCCCCCCCCCCCCCCCCCC)n1ccc(=O)[nH]c1=O]

which was formed by the nucleoside analog NM087 participating in siRNA synthesis.

In the above, Base represents base of nucleotide, such as uracil U, thymine T, cytosine C, adenine A or guanine G.

### Biological Detection Experiments

Unless otherwise stated, experimental animals C57BL/6J mice and SD rats used in the present disclosure were all purchased from SPF (Beijing) Biotechnology Co., Ltd.

Unless otherwise stated, synthesis of PCR primers used in the present disclosure was entrusted to be completed by Beijing Tsinigke iotech Co., Ltd.

Unless otherwise stated, reagents, consumable materials as well as instrument and equipment used in the present disclosure were all commercially available. Main reagents and consumable materials are as shown in TABLE 3, and main instrument and equipment are as shown in TABLE 4.

TABLE 3
Main Reagents and Consumable Materials

| Name | Manufacturer |
|---|---|
| 1 × PBS | M&C GENE TECHNOLOGY(BEIJING) LTD. |
| Hanwei RNA extraction Kit | Zhejiang Hanwei Science and Technology Co. Ltd. |
| Reverse Transcription System | Promega Corporation |
| SYBR Select Master Mix | ABI |
| RNALater | Thermo Fisher Scientific |

TABLE 4
Main Instrument and Equipment

| Name | Manufacturer |
|---|---|
| Full-automatic nucleic acid extractor | Zhejiang Hanwei Science and |
| | Technology Co. Ltd. |
| High speed refrigerated centrifuge | Eppendorf |
| NANODROP OneC | Thermo Fisher Scientific |
| Gradient PCR amplifier | Eppendorf |
| Fluorescence quantitative PCR instrument | ABI StepOne Plus |
| Gel imager | Shanghai Tanon Science & |
| | Technology Co., Ltd. |
| Electrophoresis apparatus | BEIJING LIUYI INSTRUMENT |
| | PLANT |
| Tissuelyser type II full-automatic | Shanghai Jingxin Industrial |
| tissue homogenizer | Development Co., Ltd. |
| Digital cerebral stereotaxic apparatus | Shanghai Yuyan Instruments Co., Ltd. |

Test of Evaluation of Activity of siRNA on Mice by Lateral Ventricle Administration

6-8 Week-old C57BL/6j mice were randomly grouped by body weight. Mice in each group were administered with siRNA by lateral ventricle administration, with an administration dose per mouse depending on a specific test. Taking Bregma in mouse brain as point 0 (origin), coordinates of right ventricle were found using the cerebral stereotaxic apparatus and position was marked on surface of skull (position of lateral ventricle was AP (anteroposterior, bregma anterior and posterior): −0.58 mm, ML (mediolateral, left and right of middle raphe): −1.2 mm, DV (dorsoventral, up and down cerebral dura mater plane): −2.2 mm). A dental electric drill was used to drill in the skull above a position of interest, and then medicament was injected into the lateral ventricle by a micro-dosing pump, wherein a total injection volume was 5 μL, and injection duration was 10 min. Needle was retained for 5 min after completion of injection, and then slowly withdrawn. Afterwards, needle hole was sealed with biomedical glue, and then head skin was adhered. After animals woke up, the animals were returned back to animal house and further fed. PBS control group was administered with the same volume of siRNA-free PBS solution. The day of administration was recorded as 0 day (D0), and the mice were sacrificed at observation point after the administration. Different parts of brain region were respectively taken and stored in the RNAlater.

Test of Evaluation of Activity of siRNA Conjugate on Rats by Intrathecal Administration

6-8 Week-old SD rats were randomly grouped by body weight. Rats in each group were administered with siRNA by intrathecal administration, with an administration dose of 900 μg per rat. Injection was administered from rat spinal cord L5-L6, with an administration volume of 40 μL. Needle was retained for 2 min after completion of injection, and then slowly withdrawn. PBS control group was administered with the same volume of siRNA conjugate-free PBS solution. The day of administration was recorded as 0 day (D0), and the rats were sacrificed at observation point after administration. Different segments of spinal cord and different regions of brain were respectively taken and stored in the RNAlater.

### RNA Extraction and Detection

RNA extraction: the above tissue samples were taken out from the RNAlater, and smashed for 60 s in Tissuelyser type II full-automatic tissue homogenizer, and total RNAs of various tissue samples were extracted using the full-automatic nucleic acid extraction instrument and the nucleic acid extraction kit from Zhejiang Hanwei Science and Technology Co. Ltd. according to methods described in instructions.

Reverse transcription reaction: 1 μg of the extracted total RNAs of the tissue samples were taken, and configured into 20 μL of reverse transcription system according to a method described in kit instructions by using a reverse transcription kit (Reverse Transcription System, A3500) from Promega Corporation and selecting Oligo (dT)₁₅ reverse transcription primers, and the reverse transcription reaction was completed. After the reaction was ended, 80 μL of RNase-Free water was added into the reverse transcription system to provide cDNA solution for Real-time PCR detection.

Real-time PCR detection: 20 μL of Real-time PCR reaction system per PCR detection well was configured according to a method described in kit instructions using SYBR™ Select Master Mix (Catalog number: 4472908) reagent from ABI company. Each detection system contained 5 μL of cDNA template obtained from the above reverse transcription reaction, 10 μL of SYBR™ Select Master Mix, 0.5 μL of 10 μM upstream primers, 0.5 μL of 10 μM downstream primers, and 4 μL of RNase-Free H₂O. The configured reaction system was subjected to Real-time PCR amplification on ABI StepOnePlus PCR instrument by a three-step method, wherein an amplification procedure was pre-denaturation at 95° C. for 10 min, then denaturation at 95° C. for 30 s, annealing at 60° C. for 30 s, and extension at 72° C. for 30 s; and processes of denaturation, annealing, and extension were repeated for 40 cycles. Gene expression differences were calculated by the ΔΔCt method after completion of the procedure.

In the real-time fluorescence quantitative PCR method, relative quantitative calculation of remaining expression level and inhibition rate of gene mRNA of interest in various test groups was performed by the ΔΔCt method, and the calculation method is as follows:

$$Δ Ct ( test group ) = Ct ( gene of interest in test group ) - Ct ( internal reference gene in test group ) ;$$

$$Δ Ct ( control group ) = Ct ( gene of interest in control group ) - Ct ( internal reference gene in control group ) ;$$

$$Δ Δ Ct ( test group ) = Δ Ct ( test group ) - Δ Ct ( control group mean ) ;$$

$$Δ Δ Ct ( control group ) = Δ Ct ( control group ) - Δ Ct ( control group mean ) .$$

In the above, ΔCt (control group mean) was an arithmetic mean value of ΔCt (control group) of animals sacrificed at the same point of time in the control group. Thus, each animal in the test group and the control group is corresponding to one ΔΔCt value.

$$Relative remaining expression level of gene mRNA of interest in contorl group = {2}^{- Δ Δ Ct ( control group )} \times 100 \%$$

$$Relative remaining expression level of gene mRNA of interest in test group = {2}^{- Δ Δ Ct ( test group )} \times 100 \%$$

Taking a mean value of the relative remaining expression level of the gene mRNA of interest in the control group as reference, the relative remaining expression level of the gene mRNA of interest in the control group was normalized, and the mean value of the relative remaining expression level of the gene mRNA of interest in the control group was defined as 100%.

$$Inhibition rate of expression of gene mRNA of interest in test group = ( 1 - relative remaining expression level of gene mRNA of interest in test group ) \times 100 \%$$

Unless otherwise stated, in vivo activity experimental data are all expressed by X̅±STDEV, and experimental data are all plotted and analyzed using GraphPad prism 8.0 software.

### Test Example 1

Evaluation of Inhibitory Activity of NM031 Conjugated siRNA on Target Gene Superoxide Dismutase 1 (SOD1) by Lateral Ventricular Administration to Mice

### (1) Animal Grouping, Dosing and Tissue Sample Collection

6-8 Week-old C57BL/6j mice were randomly grouped by body weight, 5 per group, 2 groups in total, i.e., PBS group (dosed with siRNA-free PBS solution) and RZ899008 group (dosed with siRNA RZ899008 (the sense strand with the NM031 conjugation at Position 4) prepared in Example 1). Mice in each group were anaesthetized by intraperitoneal injection of 5% chloral hydrate, and were immobilized with the cerebral stereotaxic apparatus after being anaesthetized. Head skin was cut, positioning coordinates of right lateral ventricle were found with the stereotaxic apparatus and position was marked on surface of skull, wherein the positioning coordinates of the lateral ventricle were as follows: in coordinate axes with Bregma point as an origin, ML (mediolateral, left and right of middle raphe, i.e., X-axis) value=1.2 mm, and AP (anteroposterior, bregma anterior and posterior, i.e., Y-axis) value=−0.58 mm, and DV (dorsoventral, down skull (cerebral dura mater) plane, i.e., Z-axis) value=2.2 mm. A dental electric drill was used to drill in the skull above a position of interest, and then sucked drug was injected into the lateral ventricle by a micro-dosing pump. An administration dose to each mouse in the RZ899008 group was 200 μg (dose being measured as siRNA, concentration of siRNA being 20 μg/μL, PBS buffer as a solvent), and a total injection volume was L. PBS group was administered with the same volume of siRNA-free PBS solution. Injection duration was 10 min. Needle was retained for 5 min after completion of injection, and then slowly withdrawn (about 2 min). Afterwards, needle hole was sealed with biomedical glue, and then head skin was adhered. After animals woke up, the animals were returned back to animal feeding room and further fed. The day of administration was recorded as first day (D1), and the mice were sacrificed on the fifth day (D5). Cerebellum, administration-side hippocampus and administration-side parietal cortex were respectively taken and stored in the RNAlater.

### (2) Detection of Inhibitory Activity of Gene of Interest in Various Tissue Samples

RNA extraction: an appropriate amount of cerebellum sample, lateral hippocampus sample and lateral parietal cortex sample were respectively taken out from the RNAlater, and smashed for 60 s in Tissuelyser type II full-automatic tissue homogenizer, and total RNAs of various tissue samples were extracted using the full-automatic nucleic acid extraction instrument and the nucleic acid extraction kit from Zhejiang Hanwei Science and Technology Co. Ltd. according to methods described in instructions.

Reverse transcription reaction: 1 μg of the extracted total RNAs of various tissue samples were taken, and configured into 20 μL of reverse transcription system according to a method described in kit instructions by using a reverse transcription kit (Reverse Transcription System, A3500) from Promega Corporation and selecting Oligo (dT)₁₅ reverse transcription primers, and the reverse transcription reaction was completed. After the reaction was ended, 80 μL of RNase-Free water was added into the reverse transcription system to provide cDNA solution for Real-time PCR detection.

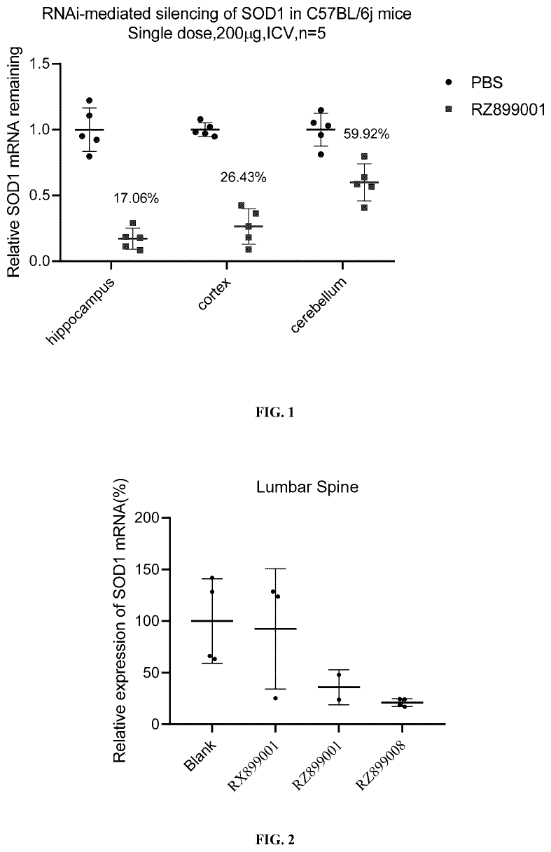

Real-time PCR detection: 20 μL of Real-time PCR reaction system per PCR detection well was configured according to a method described in kit instructions using SYBR™ Select Master Mix (Catalog number: 4472908) reagent from ABI company. Each detection system contained 5 μL of cDNA template obtained from the above reverse transcription reaction, 10 μL of SYBR™ Select Master Mix, 0.5 μL of 10 μM upstream primers, 0.5 μL of 10 μM downstream primers (see TABLE 5 for primer information), and 4 μL of RNase-Free H₂O. The configured reaction system was subjected to Real-time PCR amplification on ABI StepOnePlus PCR instrument by a three-step method, wherein an amplification procedure was pre-denaturation at 95° C. for 10 min, then denaturation at 95° C. for 30 s, annealing at 60° C. for 30 s, and extension at 72° C. for 30 s; and processes of denaturation, annealing, and extension were repeated for 40 cycles. Gene expression differences were calculated by the above ΔΔCt method after completion of the procedure, and results are shown in FIG. 1 and TABLE 6. The results show that on D5, RZ899008 had a good inhibitory action on the target gene in different parts of the brain region, wherein the inhibitory effect on the administration-side hippocampus was the best, with the inhibition rate of 82.94%; the inhibitory effect on the administration-side parietal cortex took the second place, with the inhibition rate of 73.57%; and the inhibitory activity in the cerebellum was 40.08%.

TABLE 5
Table of Primer Sequences of Internal
Reference Gene and Gene of Interest

| | | | Primer | SEQ |
|---|---|---|---|---|
| Target | | Primer | sequence | ID |
| point | | type | (5′-3′) | NO: |
| Gene of | SOD1 | Upstream | GGGTTCCACG | 7 |
| interest | | Primer | TCCATCAGTA | |
| | | Downstream | ACACCGTCCT | 8 |
| | | Primer | TTCCAGCAGT | |
| Internal | GAPDH | Upstream | TGCACCACCA | 9 |
| reference | | Primer | ACTGCTTAG | |
| gene | | Downstream | GGATGCAGGG | 10 |
| | | Primer | ATGATGTTC | |

TABLE 6
Inhibitory Activity on Target Gene of Interest in Various Tissue Samples

| | Hippocampus | | Parietal cortex | | Cerebellum | |
|---|---|---|---|---|---|---|
| | Mean | | Mean | | Mean | |
| | value of | | value of | | value of | |
| | inhibition | ±STDEV | inhibition | ±STDEV | inhibition | ±STDEV |
| Group | rate % | value | rate % | value | rate % | value |
| PBS | 0.00 | 16.60 | 0.00 | 5.16 | 0.00 | 12.49 |
| RZ899008 | 82.94 | 7.99 | 73.57 | 13.52 | 40.08 | 14.10 |

### Test Example 2

Evaluation of Inhibitory Activity of NM031 Conjugated siRNA on SOD1 by Intrathecal Administration

### (1) Animal Grouping, Dosing and Tissue Sample Collection

6-8 Week-old C57BL/6j mice were randomly grouped into 4 groups by body weight. PBS group was dosed with siRNA-free PBS solution, RX899001 group was dosed with siRNA compound RX899001 without vector conjugation, RZ899001 group was dosed with siRNA RZ899001 (the sense strand with the Uhd conjugation at Position 4) prepared in Example 1, and RZ899008 was dosed with siRNA RZ899008 (the sense strand with the NM031 conjugation at Position 4) prepared in Example 1. Mices in each group were anesthetized with isoflurane. After anesthetization, lumbar region thereof was sprayed wet with 75% disinfecting alcohol. 10 μL (concentration 80 μg/L) of siRNA drug containing PBS solvent was sucked with an insulin injection needle, the mouse lumbar region was pinched with the left-hand index finger and thumb, patella crest was propped against, and needle was held by the right hand. The needle was bent by forceps in advance, wherein the needle was first bent in the middle by 80° to 90°, then a front half part of the needle was bent in half, and the whole needle was bent in an are shape of about 60° to 90°. Mouse abdominal region was padded with an article with a thickness of about 2 cm. The needle was inserted from a joint of lumbar spines L5 and L6 of mice, and when inserting the needle, the hand was slightly inclined forwards to insert the needle. After the needle was inserted, if mouse tail suddenly trembled or swayed, it indicated that the needle was inserted correctly, in which case, the needle was rotated forwards or inclined forwards, and after it was fixed, the drug was injected. After completion of the injection, the needle was slowly withdrawn at uniform velocity. The administration dose to each mouse in RX899001 group, RZ899001 group and RZ899008 group was 800 μg, a total volume injected was 10 μL, and the PBS group was administered with the same volume of siRNA-free PBS solution. After the animals woke up, the animals were returned back to the animal feeding room and further fed. The day of administration was recorded as first day (D1), and the mice were sacrificed on ninth day (D9). Lumbar spine, thoracic spine and cerebellum were respectively taken and stored in the RNAlater.

### (2) Detection of Inhibitory Activity of Gene of Interest in Various Tissue Samples

RNA extraction: an appropriate amount of lumbar spine, thoracic spine and cerebellum samples were respectively taken out from the RNAlater, and smashed for 60 s in Tissuelyser type II full-automatic tissue homogenizer, and total RNAs of various tissue samples were extracted using the full-automatic nucleic acid extraction instrument and the nucleic acid extraction kit from Zhejiang Hanwei Science and Technology Co. Ltd. according to methods described in instructions. Reverse transcription reaction: 1 μg of the extracted total RNAs of various tissue samples were respectively taken, and configured into 20 μL of reverse transcription system according to a method described in kit instructions by using a reverse transcription kit (Reverse Transcription System, A3500) from Promega Corporation and selecting Oligo (dT)₁₅ reverse transcription primers, and the reverse transcription reaction was completed. After the reaction was ended, 80 μL of RNase-Free water was added into the reverse transcription system to provide cDNA solution for Real-time PCR detection.

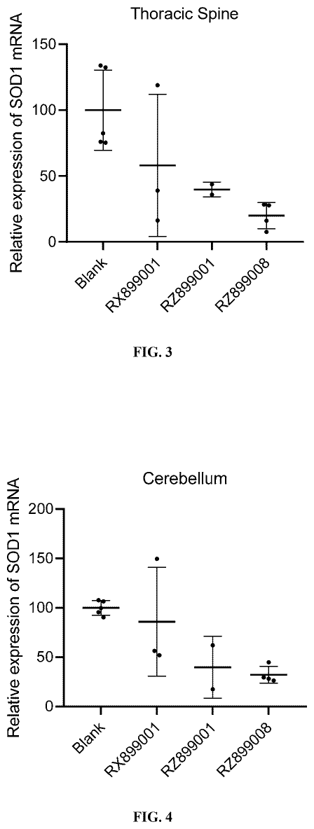

Real-time PCR detection: 20 μL of Real-time PCR reaction system per PCR detection well was configured according to a method described in kit instructions using SYBR™ Select Master Mix (Catalog number: 4472908) reagent from ABI company. Each detection system contained 5 μL of cDNA template obtained from the above reverse transcription reaction, 10 μL of SYBR™ Select Master Mix, 0.5 μL of 10 μM upstream primers, 0.5 μL of 10 μM downstream primers (see TABLE 5 for primer information), and 4 μL of RNase-Free H₂O. The configured reaction system was subjected to Real-time PCR amplification on ABI StepOnePlus PCR instrument by a three-step method, wherein an amplification procedure was pre-denaturation at 95° C. for 10 min, then denaturation at 95° C. for 30 s, annealing at 60° C. for 30 s, and extension at 72° C. for 30 s; and processes of denaturation, annealing, and extension were repeated for 40 cycles. Gene expression differences were calculated by the above ΔΔCt method after completion of the procedure, and results are shown in FIG. 2 to FIG. 4 and TABLE 7. The results show that on D9, RZ899008 D9 had a good inhibitory action on the target gene in all of the lumbar spine, thoracic spine and cerebellum, and the activity was superior to that of control molecule RZ899001 and molecule RX899001 without vector conjugation. RZ899008 had the best inhibitory effect on the lumbar spine, with the inhibition rate reaching 80.09%; the inhibition rate in the thoracic spine was 78.99%; and the inhibition rate in the cerebellum was 67.72%.

TABLE 7
Inhibitory Activity on Target Gene of Interest in Various Tissue Samples

| | Lumbar spine | | Thoracic spine | | Cerebellum | |
|---|---|---|---|---|---|---|
| | Mean | | Mean | | Mean | |
| | value of | | value of | | value of | |
| | inhibition | ±STDEV | inhibition | ±STDEV | inhibition | ±STDEV |
| Group | rate % | value | rate % | value | rate % | value |
| PBS | 0.00 | 41.01 | 0.00 | 27.17 | 0.00 | 7.35 |
| RX899001 | 7.54 | 58.27 | 41.98 | 53.98 | 14.01 | 55.16 |
| RZ899001 | 64.13 | 16.92 | 60.27 | 5.55 | 60.22 | 31.44 |
| RZ899008 | 78.99 | 3.75 | 80.09 | 9.94 | 67.72 | 8.48 |

### Test Example 3

Evaluation of Inhibitory Activity of Lipid Conjugated siRNA Sequences on SOD1 by Lateral Ventricle Administration to Mice

The present example evaluated, by an evaluation method for inhibitory activity on a target gene by lateral ventricular administration to mice, the inhibitory activity of lipid conjugates RZ899060 (the sense strand with the NM031 conjugation at Position 4), RZ899052 (the sense strand with the NM058 conjugation at Position 5) and RZ899053 (the sense strand with the NM058 conjugation at Position 6) with the same carbon chain length, and different base sites on the target gene SOD1 of interest in different regions in mouse brain.

6-8 Week-old C57BL/6j mice were randomly grouped by body weight, 5 per group, 4 groups in total. Mice in each group were dosed with the above siRNA conjugates by lateral ventricle administration, with an administration dose of 200 μg per mouse. PBS control group was administered with the same volume of siRNA conjugate-free PBS solution. The day of administration was recorded as 0 day (D0), and the mice were sacrificed on D5 after the administration. The hippocampus, cortex and cerebellum were respectively taken and stored in the RNAlater. RNA extraction, reverse transcription, and real-time fluorescence quantitative detection were performed on various tissues according to the above method, and gene expression differences were calculated by the above ΔΔCt method.

In the above, PCR primers used in Test Example 3 were as shown in TABLE 5 of Test Example 1.

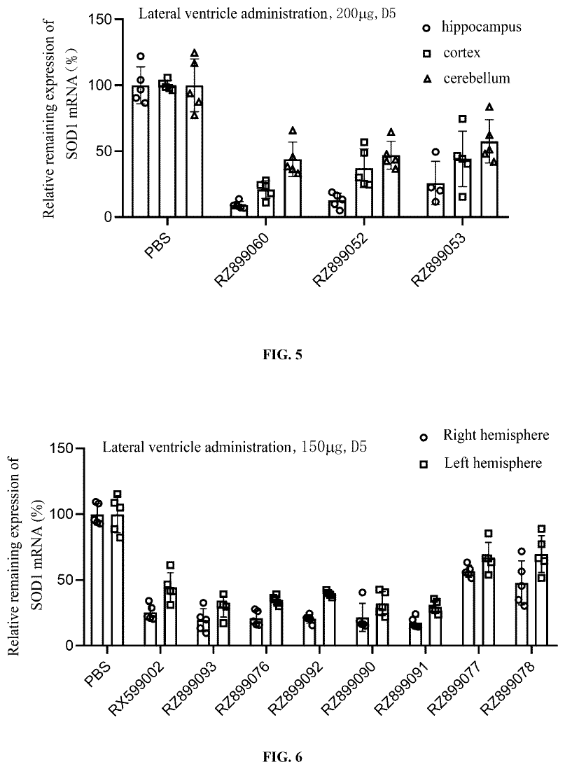

Experimental results of Test Example 3 were shown in FIG. 5 and TABLE 8. The results show that after the administration, on D5, RZ899060 with the sense strand with the NM031 lipid conjugate at Position 4, RZ899052 and RZ899053 with the NM058 lipid conjugate at Position 5 and Position 6 all had a certain inhibitory action on the target gene in hippocampus, cortex and cerebellum. The inhibitory activity in the hippocampus can reach 70% or above, the inhibitory activity in the cortex was 50% or above, and the inhibitory activity in the cerebellum was 40% or above, wherein the NM031 lipid conjugate at Position 4 of the sense strand had the best inhibitory activity, reaching 90% or above in the hippocampus, and still maintained the inhibitory activity of 56.11% in the cerebellum.

TABLE 8
Inhibitory Activity on Target Gene of Interest in Mouse Brain
after Administration of siRNAs of Present Test Example

| | Hippocampus | | Cortex | | Cerebellum | |
|---|---|---|---|---|---|---|
| | Mean | | Mean | | Mean | |
| | value of | | value of | | value of | |
| | inhibition | | inhibition | | inhibition | |
| Group | rate % | ±STDEV | rate % | ±STDEV | rate % | ±STDEV |
| PBS | 0.00 | 14.03 | 0.00 | 3.73 | 0.00 | 20.01 |
| RZ899060 | 90.98 | 2.76 | 79.05 | 6.59 | 56.11 | 13.06 |
| RZ899052 | 87.35 | 5.43 | 62.93 | 14.78 | 53.02 | 10.62 |
| RZ899053 | 74.13 | 16.41 | 55.87 | 21.04 | 42.55 | 16.45 |

### Test Example 4

Evaluation of Inhibitory Activity of Lipid Conjugated siRNA Sequences with Different Carbon Chain Lengths on SOD1 in Mouse Brain by Lateral Ventricle Administration

The present example evaluated, by an evaluation method for inhibitory activity on a target gene by lateral ventricular administration to mice, the inhibitory activity of siRNA sequence RZ899092 with the sense strand with the NM031 (C16) conjugation at Position 4, siRNA sequence RZ899090 with the sense strand with the NM073 (C18) conjugation at Position 4, siRNA sequence RZ899091 with the sense strand with the NM074 (C20) conjugation at Position 4, siRNA sequence RZ899077 with the sense strand with the NM201 (C22) conjugation at Position 4; and siRNA sequence RZ899078 with the sense strand with the NM202 (C22) conjugation at Position 6, vector-free siRNA sequence RX599002, siRNA reference sequence RZ899093 with the sense strand with the Uhd (C16) conjugation at Position 4, and siRNA reference sequence RZ899076 with the sense strand with the NM200 (C22) conjugation at Position 4 on the target gene SOD1 of interest in mouse brain.

6-8 Week-old C57BL/6j mice were randomly grouped by body weight, 5 per group, 9 groups in total. Mice in each group were dosed with the above siRNA conjugates by lateral ventricle administration, with an administration dose of 150 μg per mouse. PBS control group was administered with the same volume of siRNA conjugate-free PBS solution. The day of administration was recorded as 0 day (D0), and the mice were sacrificed on D5 after administration. Left and right brains were respectively taken and stored in the RNAlater. RNA extraction, reverse transcription, and real-time fluorescence quantitative detection were performed on various tissues according to the above method, and gene expression differences were calculated by the above ΔΔCt method.

In the above, PCR primers used in Test Example 4 were as shown in TABLE 5 of Test Example 1.

Experimental results of Test Example 4 are shown in FIG. 6 and TABLE 9. The results show that, on D5, NM031 (C16), NM073 (C18), and NM074 (C20) lipid conjugates all exhibited superior inhibitory activity in left and right hemisphere to the vector-free siRNA sequence RX599002, wherein the NM073 (C18) lipid conjugate RZ899090 and the NM074 (C20) lipid conjugate RZ899091, compared with the Uhd conjugated reference sequence RZ899093 and the NM200 (C22) conjugated reference sequence RZ899076, exhibited substantially comparable or superior activity in the right hemisphere (administration side) and left hemisphere (non-administration side), especially the C20 lipid conjugate RZ899091 exhibited better inhibitory activity in the brain. The C16 lipid conjugate RZ899092 had substantially comparable activity in the right hemisphere compared with the Uhd conjugated reference control sequence RZ899093 and NM200(C22) conjugated reference control sequence RZ899076.

TABLE 9
Inhibitory Activity on Target Gene of Interest in Mice after
Administration of siRNA Conjugates of Present Test Example

| | Right hemisphere | | Left hemisphere | |
|---|---|---|---|---|
| | Mean value of | | Mean value of | |
| Group | inhibition rate | ±STDEV | inhibition rate | ±STDEV |
| PBS | 0.00 | 8.10 | 0.00 | 13.94 |
| RX599002 | 74.62 | 5.92 | 55.55 | 11.01 |
| RZ899093 | 80.47 | 8.84 | 70.23 | 7.95 |
| RZ899076 | 79.02 | 5.69 | 65.25 | 3.40 |
| RZ899092 | 79.45 | 3.20 | 60.45 | 2.33 |
| RZ899090 | 78.49 | 10.74 | 67.87 | 9.16 |
| RZ899091 | 82.32 | 4.22 | 70.41 | 4.91 |
| RZ899077 | 43.33 | 4.51 | 32.81 | 11.43 |
| RZ899078 | 52.15 | 16.83 | 30.24 | 14.08 |

### Test Example 5

Evaluation of Inhibitory Activity of Lipid Conjugated siRNA Sequences on SOD1 by Intrathecal Administration to Rats

The present example evaluated, by an evaluation method for inhibitory activity on a target gene by intrathecal administration to rats, the inhibitory activity of siRNA sequence RZ899060 with the sense strand with the NM031 conjugation at Position 4 and vector-free siRNA sequence RZ899056, and Uhd conjugated siRNA reference sequence RZ899055 on the target gene SOD1 of interest in spinal cord and brain of rats.

6-8 Week-old SD rats were randomly grouped by body weight, 5 per group, 4 groups in total. Rats in each group were dosed with the above siRNA conjugates by intrathecal administration, with an administration dose of 900 μg per rat, and an administration volume of 40 μL. PBS control group was administered with the same volume of siRNA conjugate-free PBS solution. The day of administration was recorded as 0 day (D0), and the rats were sacrificed on D7 after administration. Different spinal cord segments (lumbar segment, thoracic segment, cervical segment) and tissues such as cerebral cortex, hippocampus, and cerebellum were respectively taken and stored in the RNAlater. RNA extraction, reverse transcription, and real-time fluorescence quantitative detection were performed on various tissues according to the above method, and gene expression differences were calculated by the above ΔΔCt method.

TABLE 10
Table of Sequences of PCR
Primers Used in Test Example 5

| Target | Primer | Primer sequence | |
|---|---|---|---|
| point | type | (5′-3′) | |
| SOD1 | Upstream | TTAACTGAAG | SEQ ID |
| | Primer | GCGAGCATG | NO. 26 |
| | Downstream | TGCCCAGGTC | SEQ ID |
| | Primer | TCCAACAT | NO. 27 |
| Actin | Upstream | CCGTGAAAAGA | SEQ ID |
| | Primer | TGACCCAGAT | NO. 28 |
| | Downstream | GCCAGGTCCAG | SEQ ID |
| | Primer | ACGCAGG | NO. 29 |

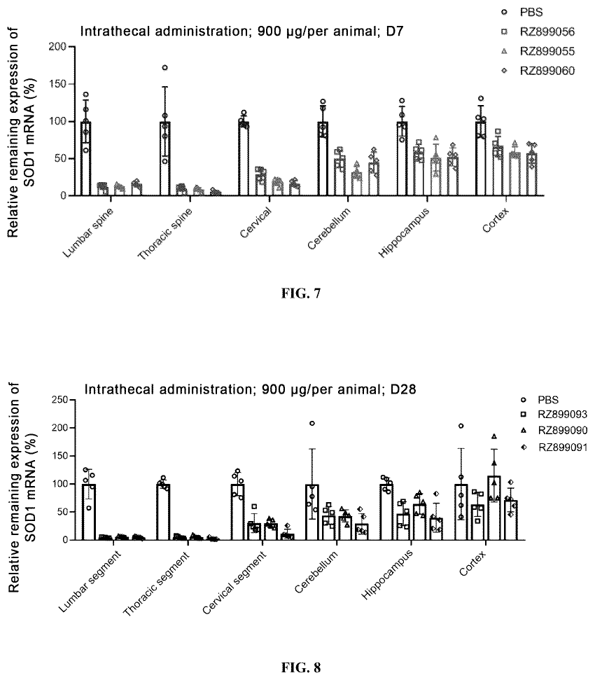

Experimental results of Test Example 5 are shown in FIG. 7 and TABLE 11. The results show that, on D7, the activity of the NM031 conjugate RZ899060 in the thoracic segment and cervical segment in the spinal cord segments was superior to that of the Uhd conjugated reference sequence RZ899055, the activity in the lumbar segment was substantially comparable to that of the reference sequence, and the inhibitory activity was all maintained at 80% or above; the activity in the hippocampus and cortex in the brain region was substantially comparable to that of the reference sequence RZ899055.

TABLE 11
Inhibitory Activity on Target Gene of Interest in Rats after Administration of the siRNA Conjugates in Present Test Example

| | Lumbar | | Thoracic | | Cervical | | | | | | | |
|---|---|---|---|---|---|---|---|---|---|---|---|---|
| | segmen | | segment | | segment | | Cerebellum | | Hippocampus | | Cortex | |
| | Mean | | Mean | | Mean | | Mean | | Mean | | Mean | |
| | value of | | value of | | value of | | value of | | value of | | value of | |
| | inhibi- | | inhibi- | | inhibi- | | inhibi- | | inhibi- | | inhibi- | |
| | tion | ±STDEV | tion | ±STDEV | tion | ±STDEV | tion | ±STDEV | tion | ±STDEV | tion | ±STDEV |
| Group | rate | value | rate | value | rate | value | rate | value | rate | value | rate | value |
| PBS | 0.00 | 28.70 | 0.00 | 46.34 | 0.00 | 7.34 | 0.00 | 20.94 | 0.00 | 19.57 | 0.00 | 21.04 |
| RZ899056 | 87.58 | 3.81 | 89.69 | 3.55 | 70.86 | 7.97 | 50.22 | 11.49 | 41.25 | 10.03 | 33.88 | 13.44 |
| RZ899055 | 87.46 | 2.32 | 91.10 | 2.74 | 81.31 | 4.80 | 68.43 | 7.84 | 48.56 | 17.80 | 41.47 | 7.40 |
| RZ899060 | 84.24 | 3.33 | 94.54 | 1.76 | 83.37 | 4.07 | 55.10 | 14.10 | 47.77 | 12.20 | 43.00 | 13.18 |

### Test Example 6

Evaluation of Inhibitory Activity of NM073, NM074 Conjugated siRNA Sequences on SOD1 by Intrathecal Administration to Rats

The present test example evaluated, by an evaluation method for inhibitory activity on a target gene by intrathecal administration to rats, the inhibitory activity of siRNA sequence RZ899090 with the NM073 conjugation at Position 4, siRNA sequence RZ899091 with the NM074 conjugation at Position 4 and siRNA control sequence RZ899093 with the Uhd conjugation on the target gene SOD1 of interest in spinal cord and brain of rats. The above conjugation vectors were different in that NM073 was C18 aliphatic chain and NM074 was C20 aliphatic chain.

6-8 Week-old SD rats were randomly grouped by body weight, 5 per group, 4 groups in total. Rats in each group were dosed with the above siRNA conjugates by intrathecal administration, with an administration dose of 900 μg per rat, and an administration volume of 40 μL. PBS control group was administered with the same volume of siRNA conjugate-free PBS solution. The day of administration was recorded as D0, and the rats were sacrificed on D28 after administration. Different spinal cord segments (lumbar segment, thoracic segment, cervical segment) and tissues such as cerebral cortex, hippocampus, and cerebellum were respectively taken and stored in the RNAlater. RNA extraction, reverse transcription, and real-time fluorescence quantitative detection were performed on various tissues according to the above method, and gene expression differences were calculated by the above ΔΔCt method. Primers are as shown in TABLE 10 of TEST Example 5.

Results of Test Example 6 show that on D28, the NM074 conjugate (RZ899091) had good inhibitory activity in the spinal cord and brain region of rats, and exhibited a superior level of inhibition on the target gene in the spinal cord segment, cerebellum, and hippocampus to the Uhd control conjugate (RZ899093). The NM073 conjugate (RZ899090) had a comparable level of inhibition in the spinal cord segment and cerebellum to the Uhd control conjugate (FIG. 8, TABLE 12).

TABLE 12
Inhibitory Activity on Target Gene of Interest in Rats after Administration
of the siRNA Conjugates in Present Test Example

| | Lumbar segment | | Thoracic segment | | Cervical segment | |
|---|---|---|---|---|---|---|
| | % Mean | | % Mean | | % Mean | |
| | value of | | value of | | value of | |
| | inhibition | ±STDEV | inhibition | ±STDEV | inhibition | ±STDEV |
| Group | rate | value | rate | value | rate | value |
| PBS | 0.00 | 26.43 | 0.00 | 7.52 | 0.00 | 20.83 |
| RZ899093 | 95.31 | 0.99 | 94.90 | 1.96 | 69.68 | 17.08 |
| RZ899090 | 93.93 | 1.05 | 94.17 | 2.34 | 69.98 | 7.26 |
| RZ899091 | 95.17 | 1.63 | 98.15 | 0.23 | 92.15 | 1.86 |
| | Cerebellum | | Hippocampus | | Cortex | |
| | % Mean | | % Mean | | % Mean | |
| | value of | | value of | | value of | |
| | inhibition | ±STDEV | inhibition | ±STDEV | inhibition | ±STDEV |
| Group | rate | value | rate | value | rate | value |
| PBS | 0.00 | 62.54 | 0.00 | 11.32 | 0.00 | 63.63 |
| RZ899093 | 55.89 | 16.03 | 52.87 | 21.41 | 36.29 | 21.27 |
| RZ899090 | 56.97 | 10.98 | 35.35 | 18.38 | −15.00 | 46.84 |
| RZ899091 | 73.86 | 19.53 | 70.69 | 11.44 | 28.78 | 24.55 |

### Test Example 7

Evaluation of Inhibitory Activity of Lipid Conjugated siRNA Sequences on SOD1 in Mouse Adipose and Muscle Tissues

The present test example evaluated, by an evaluation method for inhibitory activity on a target gene in mice, the inhibitory activity of siRNA sequence RZ899090 with the sense strand with the NM073 conjugation at Position 4, siRNA sequence RZ899091 with the sense strand with the NM074 conjugation at Position 4, siRNA sequence RZ899078 with the sense strand with the NM202 conjugation at Position 6 and siRNA reference sequence RZ899093 with the sense strand with the Uhd conjugation at Position 4, and siRNA reference sequence RZ899076 with the sense strand with the NM200 conjugation at Position 4 on the target gene SOD1 of interest in mice. The above conjugation vectors were different in that NM073 was C18 aliphatic chain, NM074 was C20 aliphatic chain, and NM0202 and NM0200 were C22 aliphatic chains.

6-8 Week-old C57BL/6j mice were randomly grouped by body weight, 15 per group, 6 groups in total. Mice in each group were dosed with the above siRNA conjugates by tail vein injection, with an administration dose of 5 mg/kg per mouse, and an administration volume of 150 μL. PBS control group was administered with the same volume of siRNA conjugate-free PBS solution. The day of administration was recorded as D0, and 5 mice in each group were sacrificed on D14, D28 and D49 after administration. Tissues such as paragonadal fat, left hind leg quadriceps muscle, heart, liver, and kidney were respectively taken and stored in the RNAlater. RNA extraction, reverse transcription, and real-time fluorescence quantitative detection were performed on various tissues according to the above method, and gene expression differences were calculated by the above ΔΔCt method. Primers are as shown in TABLE 5 of Test Example 1.

Results of Test Example 7 show that on D14, D28 and D49, in mouse heart tissues, the NM073 conjugated sequence RZ899090, NM074 conjugated sequence RZ899091, NM202 conjugated sequence RZ899078 all exhibited superior inhibitory activity to the Uhd conjugated reference sequence RZ899093 and NM200 conjugated reference sequence RZ899076 (FIG. 9, TABLE 13).

TABLE 13
Inhibitory Activity on Target Gene of Interest in Mouse Heat Tissues
after Administration of the siRNA Conjugates in Present Test Example

| | D 14 | | D 28 | | D 49 | |
|---|---|---|---|---|---|---|
| | % Mean | | % Mean | | % Mean | |
| | value of | | value of | | value of | |
| | inhibition | ±STDEV | inhibition | ±STDEV | inhibition | ±STDEV |
| Group | rate | value | rate | value | rate | value |
| PBS | 0.00 | 34.93 | 0.00 | 23.28 | 0.00 | 12.44 |
| RZ899093 | 57.83 | 9.34 | 60.42 | 11.94 | 44.18 | 27.66 |
| RZ899076 | 70.04 | 5.02 | 70.45 | 15.65 | 37.03 | 7.55 |
| RZ899090 | 79.58 | 10.58 | 82.12 | 4.00 | 65.43 | 12.96 |
| RZ899091 | 87.44 | 2.10 | 82.78 | 3.15 | 81.89 | 1.91 |
| RZ899078 | 83.53 | 1.66 | 79.52 | 6.68 | 63.42 | 6.79 |

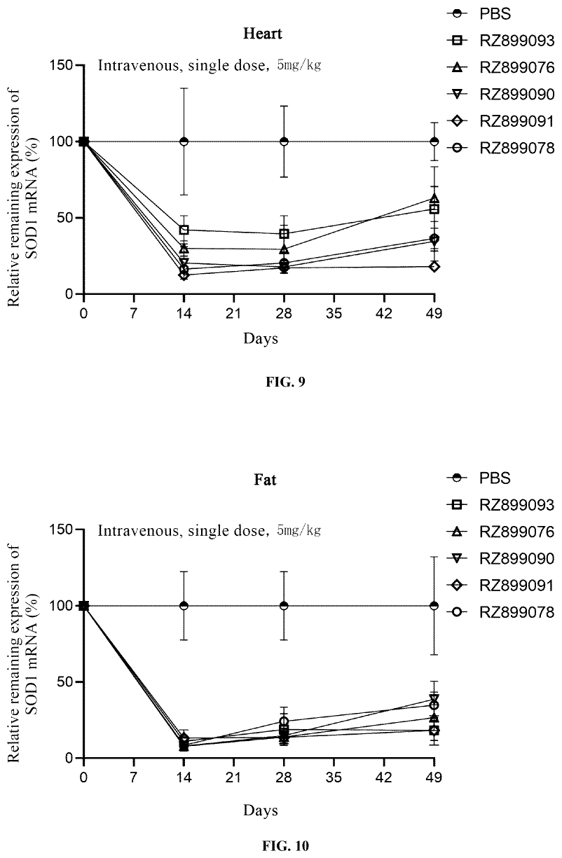

In the mouse adipose tissues, on D14, the NM073 conjugated sequence RZ899090, NM074 conjugated sequence RZ899091, and NM202 conjugated sequence RZ899078 had the inhibitory activity on par with that of the NM200 conjugated reference sequence RZ899076, and on D49, RZ899091 exhibited superior gene inhibitory effect to RZ899076 (FIG. 10, TABLE 14).

TABLE 14
Inhibitory Activity on Target Gene of Interest in Mouse Adipose Tissues
after Administration of the siRNA Conjugates in Present Test Example

| | D 14 | | D 28 | | D 49 | |
|---|---|---|---|---|---|---|
| | % Mean | | % Mean | | % Mean | |
| | value of | | value of | | value of | |
| | inhibition | ±STDEV | inhibition | ±STDEV | inhibition | ±STDEV |
| Group | rate | value | rate | value | rate | value |
| PBS | 0.00 | 22.50 | 0.00 | 22.50 | 0.00 | 32.02 |
| RZ899093 | 88.95 | 4.00 | 81.22 | 10.41 | 81.75 | 6.61 |
| RZ899076 | 92.23 | 3.06 | 86.10 | 5.66 | 73.36 | 8.48 |
| RZ899090 | 92.09 | 2.65 | 85.11 | 4.96 | 61.34 | 11.82 |
| RZ899091 | 86.81 | 5.32 | 86.36 | 4.24 | 81.73 | 9.62 |
| RZ899078 | 91.53 | 2.18 | 75.82 | 9.48 | 65.16 | 8.58 |

In mouse skeletal muscle tissues, the NM073 conjugated sequence RZ899090, NM074 conjugated sequence RZ899091, and NM202 conjugated sequence RZ899078 all had certain inhibitory activity in the tissue sites, wherein the NM202 conjugated sequence RZ899078 had the highest inhibitory activity in muscles, and can still maintain about 57% of the inhibitory activity on D49 (FIG. 11, TABLE 15).

TABLE 15
Inhibitory Activity on Target Gene of Interest of Mouse Skeletal Muscle Tissues
after Administration of the siRNA Conjugates in Present Test Example

| | D 14 | | D 28 | | D 49 | |
|---|---|---|---|---|---|---|
| | % Mean | | % Mean | | % Mean | |
| | value of | | value of | | value of | |
| | inhibition | ±STDEV | inhibition | ±STDEV | inhibition | ±STDEV |
| Group | rate | value | rate | value | rate | value |
| PBS | 0.00 | 24.34 | 0.00 | 32.66 | 0.00 | 29.36 |
| RZ899093 | 68.42 | 11.76 | 64.12 | 8.41 | 44.88 | 14.73 |
| RZ899076 | 82.53 | 6.17 | 81.97 | 14.73 | 66.88 | 14.22 |
| RZ899090 | 57.16 | 19.38 | 51.23 | 25.20 | 42.93 | 7.94 |
| RZ899091 | 69.80 | 7.93 | 72.24 | 14.81 | 41.89 | 21.36 |
| RZ899078 | 71.63 | 11.76 | 69.78 | 14.71 | 57.72 | 9.17 |

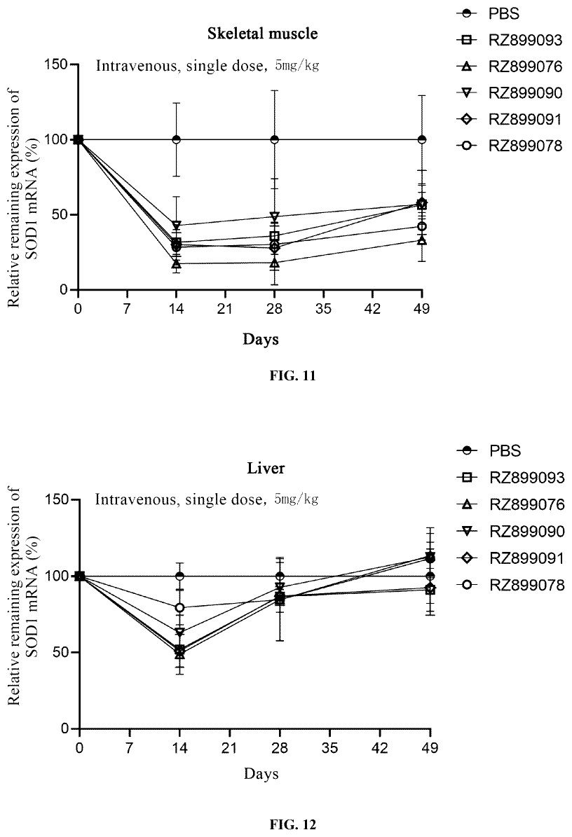

In addition, detection results of inhibition on gene on D28 and D49 show that the NM073 conjugated sequence RZ899090, NM074 conjugated sequence RZ899091, and NM202 conjugated sequence RZ899078 had no inhibitory action on liver and kidney tissues, which reflects the safety of these conjugates in non-target tissues to some extent (FIG. 12, FIG. 13, and TABLE 16).

TABLE 16
Relative Expression Levels of Target Gene of Interest in Mouse Liver and Kidney
Tissues after Administration of the siRNA Conjugates in Present Test Example

| | D 14 | | D 28 | | D 49 | |
|---|---|---|---|---|---|---|
| | % Mean | | % Mean | | % Mean | |
| | value of | | value of | | value of | |
| | inhibition | ±STDEV | inhibition | ±STDEV | inhibition | ±STDEV |
| Group | rate | value | rate | value | rate | value |
| | Liver | | | | | |
| PBS | 0.00 | 8.68 | 0.00 | 12.28 | 0.00 | 17.82 |
| RZ899093 | 48.04 | 11.43 | 13.41 | 4.64 | 13.02 | 13.99 |
| RZ899076 | 51.15 | 13.22 | 15.46 | 26.85 | −13.46 | 14.51 |
| RZ899090 | 36.99 | 11.48 | 7.32 | 16.30 | −12.46 | 19.24 |
| RZ899091 | 49.04 | 10.77 | 13.03 | 6.23 | 7.59 | 18.07 |
| RZ899078 | 20.63 | 11.17 | 15.46 | 26.85 | −11.54 | 10.71 |
| | Kidney | | | | | |
| PBS | 0.00 | 31.07 | 0.00 | 18.27 | 0.00 | 12.86 |
| RZ899093 | 42.87 | 21.12 | 19.28 | 6.53 | 18.90 | 12.30 |
| RZ899076 | 9.13 | 30.25 | 9.82 | 20.74 | 7.06 | 9.75 |
| RZ899090 | 31.38 | 19.89 | 15.90 | 27.29 | 7.12 | 7.34 |
| RZ899091 | 31.71 | 17.92 | 3.50 | 5.29 | 9.97 | 13.42 |
| RZ899078 | −7.92 | 20.59 | 1.46 | 19.16 | 2.03 | 11.14 |

### Test Example 8

Evaluation of Inhibitory Activity of Lipid Conjugated siRNA Sequences on Target Gene SOD1 in Adipose Tissues in Different Parts of Mice

The present test example evaluated, by an evaluation method for inhibitory activity on a target gene in mice, the inhibitory activity of siRNA sequence RZ899090 with the sense strand with the NM073 conjugation at Position 4, siRNA sequence RZ899091 with the sense strand with the NM074 conjugation at Position 4, siRNA sequence RZ899078 with the sense strand with the NM202 conjugation at Position 6 and siRNA reference sequence RZ899093 with the sense strand with the Uhd conjugation at Position 4, siRNA reference sequence RZ899076 with the sense strand with the NM200 conjugation at Position 4 on the target gene SOD1 in adipose tissues of interest in mice.

6-8 Week-old C57BL/6j mice were randomly grouped by body weight, 5 per group, 6 groups in total. Mice in each group were dosed with the above siRNA conjugates by tail vein injection, with an administration dose of 5 mg/kg per mouse, and an administration volume of 150 μL. PBS control group was administered with the same volume of siRNA conjugate-free PBS solution. The day of administration was recorded as D0, and 5 mice in each group were sacrificed on D14 after administration. Paragonadal fat, abdominal subcutaneous fat, and brown fat on back were respectively taken. RNA extraction, reverse transcription, and real-time fluorescence quantitative detection were performed on various tissues according to the above method, and gene expression differences were calculated by the above ΔΔCt method. Primers are as shown in TABLE 5 of Test Example 1.

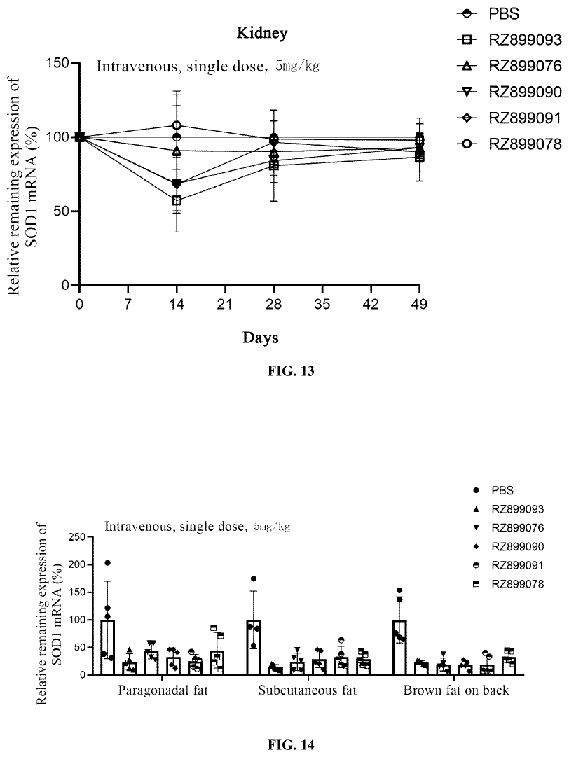

Results of Test Example 8 show that the NM073 conjugated sequence RZ899090, and the NM074 conjugated sequence RZ899091 both exhibited equivalent or superior inhibitory activity in the paragonadal fat and brown fat on back to the NM200 conjugated reference sequence RZ899076, and the inhibitory action on the target gene in the subcutaneous fat was substantially comparable (FIG. 14, TABLE 17).

TABLE 17
Relative Expression Levels of Target Gene of Interest in Mouse Adipose Tissues
after Administration of the siRNA Conjugates in Present Test Example

| | Paragonadal fat | | Subcutaneous fat | | Brown fat on back | |
|---|---|---|---|---|---|---|
| | % Mean | | % Mean | | % Mean | |
| | value of | | value of | | value of | |
| | inhibition | ±STDEV | inhibition | ±STDEV | inhibition | ±STDEV |
| Group | rate | value | rate | value | rate | value |
| PBS | 0.00 | 70.37 | 0.00 | 52.21 | 0.00 | 41.89 |
| RZ899093 | 76.20 | 14.75 | 85.92 | 5.09 | 77.13 | 3.94 |
| RZ899076 | 56.52 | 14.03 | 76.03 | 15.92 | 80.80 | 11.76 |
| RZ899090 | 67.09 | 16.24 | 71.07 | 15.55 | 81.60 | 7.63 |
| RZ899091 | 75.20 | 12.38 | 67.17 | 19.19 | 80.97 | 16.57 |
| RZ899078 | 55.23 | 32.30 | 71.42 | 11.39 | 67.27 | 10.72 |

### Test Example 9

Evaluation of Inhibitory Activity of Lipid Conjugated siRNA Sequences on SOD1 in Mouse Muscle Tissues

The present test example evaluated, by an evaluation method for inhibitory activity on a target gene in mice, the inhibitory activity of siRNA sequence RZ899090 with the sense strand with the NM073 conjugation at Position 4, siRNA sequence RZ899091 with the sense strand with the NM074 conjugation at Position 4, siRNA sequence RZ899078 with the sense strand with the NM202 conjugation at Position 6 and siRNA reference sequence RZ899093 with the sense strand with the Uhd conjugation at Position 4, siRNA reference sequence RZ899076 with the sense strand with the NM200 conjugation at Position 4 on the target gene SOD1 of interest in muscle tissues in different parts of mice.

6-8 Week-old C57BL/6j mice were randomly grouped by body weight, 5 per group, 6 groups in total. Mice in each group were dosed with the above siRNA conjugates by tail vein injection, with an administration dose of 5 mg/kg per mouse, and an administration volume of 150 μL. PBS control group was administered with the same volume of siRNA conjugate-free PBS solution. The day of administration was recorded as D0, and 5 mice in each group were sacrificed on D14 after administration. Tissues such as left hind leg quadriceps muscle, diaphragm, and heart tissues were respectively taken and stored in the RNAlater. RNA extraction, reverse transcription, and real-time fluorescence quantitative detection were performed on various tissues according to the above method, and gene expression differences were calculated by the above ΔΔCt method. Primers are as shown in TABLE 5 of Test Example 1.

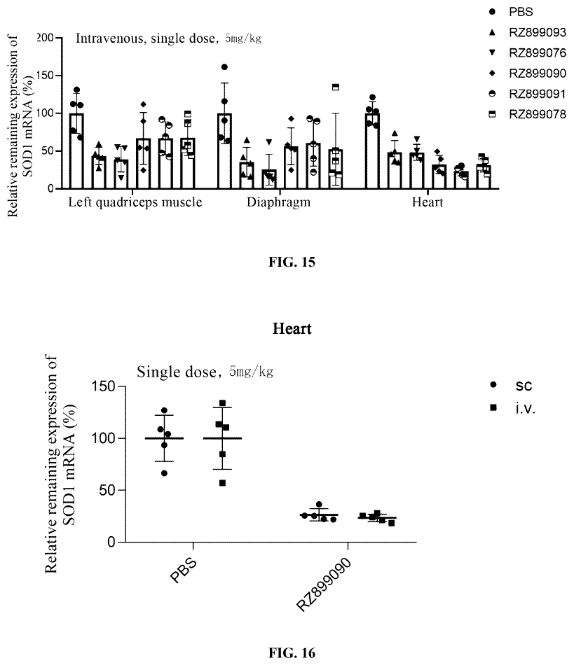

Results of Test Example 9 show that, in the heart tissues, the NM073 conjugated sequence RZ899090, NM074 conjugated sequence RZ899091, NM202 conjugated siRNA sequence RZ899078 all exhibited superior inhibitory activity to the NM200 conjugated reference sequence RZ899076; and all had certain inhibitory action on the left quadriceps muscle and diaphragm (FIG. 15, TABLE 18).

TABLE 18
Relative Expression Levels of Target Gene of Interest in Mice after
Administration of the siRNA Conjugates in Present Test Example

| | Left quadriceps muscle | | Diaphragm | | Heart | |
|---|---|---|---|---|---|---|
| | % Mean | | % Mean | | % Mean | |
| | value of | | value of | | value of | |
| | inhibition | ±STDEV | inhibition | ±STDEV | inhibition | ±STDEV |
| Group | rate | value | rate | value | rate | value |
| PBS | 0.00 | 26.50 | 0.00 | 40.23 | 0.00 | 15.28 |
| RZ899093 | 56.55 | 11.50 | 64.76 | 19.62 | 51.52 | 15.57 |
| RZ899076 | 60.97 | 16.67 | 74.58 | 20.44 | 51.69 | 10.58 |
| RZ899090 | 33.16 | 34.20 | 43.77 | 24.32 | 67.88 | 12.11 |
| RZ899091 | 33.02 | 21.79 | 39.22 | 30.90 | 76.73 | 6.20 |
| RZ899078 | 32.10 | 23.69 | 47.67 | 47.82 | 68.69 | 8.85 |

### Test Example 10

Evaluation of Inhibitory Activity of siRNA Conjugates on Target Gene SOD1 in Mouse Adipose and Heart Tissues by Different Routes of Administration

The present test example evaluated, by an evaluation method for inhibitory activity on a target gene in mice, the inhibitory activity of siRNA sequence RZ899090 with the sense strand with the NM073 conjugation at Position 4 on the target gene SOD1 of interest in mice.

6-8 Week-old C57BL/6j mice were randomly grouped by body weight, 5 per group, 4 groups in total. Mice in one of the groups were dosed with the above siRNA conjugate by tail vein injection, with an administration dose of 5 mg/kg, and an administration volume of 150 μL. One group was dosed with the above siRNA conjugate by subcutaneous administration, with an administration dose of 5 mg/kg, and an administration volume of 5 ml/kg. The other two groups were PBS control groups, and were respectively administered with the same volume of siRNA conjugate-free PBS solution by tail vein injection and subcutaneous administration. The day of administration was recorded as D0, and the mice were sacrificed on D14 after administration. Paragonadal fat and heart tissues were respectively taken and stored in the RNAlater. RNA extraction, reverse transcription, and real-time fluorescence quantitative detection were performed on various tissues according to the above method, and gene expression differences were calculated by the above ΔΔCt method. Primers are as shown in TABLE 5 of Test Example 1. Results of Test Example 10 show that on D14, in the heart tissues of the mice, administration of the same dose of the NM073 conjugated sequence RZ899090 by different routes of administration provided comparable inhibitory activity in heart (FIG. 16, TABLE 19).

TABLE 19
Inhibitory Activity on Target Gene of Interest in Mouse Heat Tissues
after Administration of the siRNA Conjugate in Present Test Example

| | Subcutaneous | | Intravenous | |
|---|---|---|---|---|
| | % Mean value of | | % Mean value of | |
| Group | inhibition rate | ±STDEV value | inhibition rate | ±STDEV value |
| PBS | 0.00 | 22.28 | 0.00 | 29.70 |
| RZ899090 | 73.71 | 5.93 | 76.57 | 3.68 |

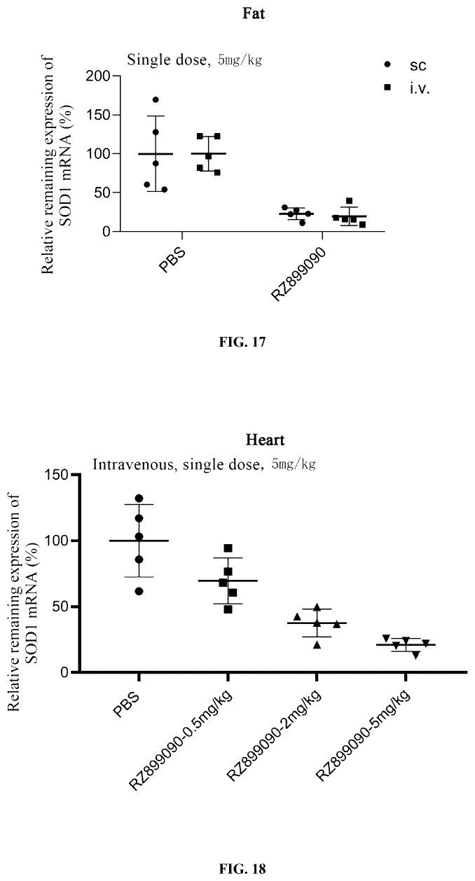

On D14, in the mouse adipose tissues, the NM073 conjugated sequence RZ899090 administered with the same dose by different routes of administration provided comparable inhibitory activity in the fat (FIG. 17, TABLE 20).

TABLE 20
Inhibitory Activity on Target Gene of Interest in Mouse Adipose Tissues
after Administration of the siRNA Conjugate in Present Test Example

| | Subcutaneous | | Intravenous | |
|---|---|---|---|---|
| | % Mean value of | | % Mean value of | |
| Group | inhibition rate | ±STDEV value | inhibition rate | ±STDEV value |
| PBS | 0.00 | 48.58 | 0.00 | 22.06 |
| RZ899090 | 77.05 | 7.52 | 80.33 | 11.70 |

### Test Example 11

Evaluation of Inhibitory Activity of siRNA Conjugates at Different Administration Doses on Target Gene SOD1 in Mouse Adipose and Heart Tissues

The present test example evaluated, by an evaluation method for inhibitory activity on a target gene in mice, the inhibitory activity of siRNA sequence RZ899090 with the sense strand with the NM073 conjugation at Position 4 at different administration doses on the target gene SOD1 of interest in mice.

6-8 Week-old C57BL/6j mice were randomly grouped by body weight, 5 per group, 4 groups in total. Mice were administered with the above siRNA conjugate by tail vein injection, wherein mice in one group were administered with the above siRNA conjugate at an administration dose of 5 mg/kg, mice in one group were administered with the above siRNA conjugate at an administration dose of 2 mg/kg, and one group was administered with the above siRNA conjugate at an administration dose of 0.5 mg/kg, all administration volumes being 150 μL. The remaining group acted as PBS control group and was administered with the same volume of siRNA conjugate-free PBS solution. The day of administration was recorded as D0, and the mice were sacrificed on D14 after administration. Paragonadal fat and heart tissues were respectively taken and stored in the RNAlater. RNA extraction, reverse transcription, and real-time fluorescence quantitative detection were performed on various tissues according to the above method, and gene expression differences were calculated by the above ΔΔCt method. Primers are as shown in TABLE 5 of Test Example 1.

Results of Test Example 11 show that on D14, in the mouse heart tissues, the NM073 conjugated sequence RZ899090 exhibited certain inhibitory activity at different administration doses and had distinct dose dependency (FIG. N, TABLE N).

TABLE 21
Inhibitory Activity on Target Gene of Interest in Mouse Heart Tissues
after Administration of the siRNA Conjugate in Present Test Example

| | 0.5 mg/kg | | 2 mg/kg | | 5 mg/kg | |
|---|---|---|---|---|---|---|
| | % Mean | | % Mean | | % Mean | |
| | value of | | value of | | value of | |
| | inhibition | ±STDEV | inhibition | ±STDEV | inhibition | ±STDEV |
| Group | rate | value | rate | value | rate | value |
| PBS | 0.00 | 27.43 | 0.00 | 27.43 | 0.00 | 27.43 |
| RZ899090 | 30.45 | 17.38 | 62.38 | 10.51 | 79.04 | 5.00 |

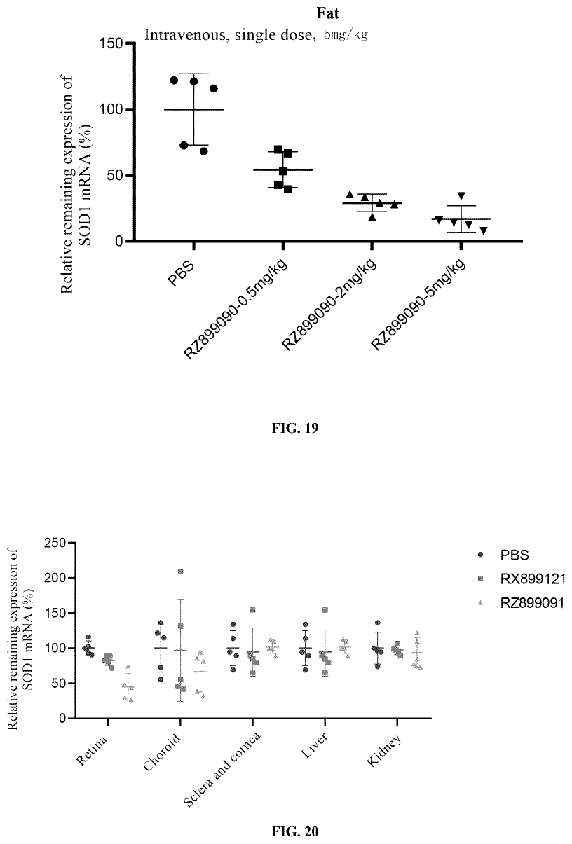

On D14, in the mouse adipose tissues, the NM073 conjugated sequence RZ899090 exhibited certain inhibitory activity at different administration doses, and had distinct dose dependency (FIG. 19, TABLE 22).

TABLE 22
Inhibitory Activity on Target Gene of Interest in Mouse Adipose Tissues
after Administration of the siRNA Conjugate in Present Test Example

| | 0.5 mg/kg | | 2 mg/kg | | 5 mg/kg | |
|---|---|---|---|---|---|---|
| | % Mean | | % Mean | | % Mean | |
| | value of | | value of | | value of | |
| | inhibition | ±STDEV | inhibition | ±STDEV | inhibition | ±STDEV |
| Group | rate | value | rate | value | rate | value |
| PBS | 0.00 | 27.10 | 0.00 | 27.10 | 0.00 | 27.10 |
| RZ899090 | 45.65 | 13.59 | 70.90 | 6.67 | 83.09 | 10.13 |

### Test Example 12

Evaluation of Inhibitory Activity of Lipid Conjugated siRNA Sequence on SOD1 in Mouse Eyes

The present test example evaluated, by an evaluation method for inhibitory activity on a target gene in mice, the inhibitory activity of siRNA sequence RZ899091 with the sense strand with the NM074 conjugation at Position 4 and siRNA reference sequence RX899121 without vector conjugation on the target gene SOD1 of interest in mice.

6-8 Week-old C57BL/6J mice were randomly grouped by body weight, 5 in each group and 3 groups in total. Mice in each group were administered with the above siRNA conjugate by intravitreal administration to right eyes of the mice, with an administration dose of 7.5 μg per mouse, and an administration volume of 2 μL. PBS control group was administered with the same volume of siRNA conjugate-free PBS solution. The day of administration was recorded as D0, and the mice were sacrificed at D14 after administration. Administration-side ocular retina, choroid, cornea, sclera and other tissues were respectively taken and stored in the RNAlater. RNA extraction, reverse transcription, and real-time fluorescence quantitative detection were performed on various tissues according to the above method, and gene expression differences were calculated by the above ΔΔCt method. Primers are as shown in TABLE 5 of Test Example 1.

Results of Test Example 12 show that on D14, in the mouse retina and choroid, the NM074 conjugated sequence RZ899091 exhibited superior inhibitory activity to the reference sequence RX899121 without vector (FIG. 20, TABLE 23).

TABLE 23
Inhibitory Activity on Target Gene of Interest in Mouse Adipose Tissues
after Administration of the siRNA Conjugate in the Present Test Example

| | PBS | | RX899121 | | RZ899091 | |
|---|---|---|---|---|---|---|
| | % Mean | | % Mean | | % Mean | |
| | value of | | value of | | value of | |
| | inhibition | ±STDEV | inhibition | ±STDEV | inhibition | ±STDEV |
| Group | rate | value | rate | value | rate | value |
| Retina | 0.00 | 10.05 | 17.47 | 7.26 | 54.98 | 19.03 |
| Choroid | 0.00 | 34.37 | 3.16 | 72.82 | 33.54 | 28.72 |
| Cornea and | 0.00 | 24.81 | 5.35 | 34.45 | −2.05 | 9.53 |
| sclera | | | | | | |
| Liver | 0.00 | 24.81 | 5.35 | 34.45 | −2.05 | 9.53 |
| Kidney | 0.00 | 22.57 | 2.89 | 6.60 | 6.51 | 21.43 |

In the description of the present description, description with reference to the terms such as “an embodiment”, “some embodiments”, “example”, “specific example”, or “some examples” mean that specific features, structures, materials or characteristics described in conjunction with this embodiment or example are comprised in at least one embodiment or example of the present disclose. In the present description, exemplary expressions of the above terms do not necessarily refer to the same embodiment or example. Moreover, specific features, structures, materials or characteristics described can be combined in any appropriate manner in any one or more embodiments or examples. Furthermore, those skilled in the art could integrate and combine different embodiments or examples as well as features of the different embodiments or examples described in the description, without conflict.

Although the embodiments of the present disclosure have been shown and described in the above, it could be understood that the above embodiments are exemplary, and should not be construed as limitation to the present disclosure. Those ordinarily skilled in the art could change, modify, substitute and vary the above embodiments within the scope of the present disclosure.

### INDUSTRIAL APPLICABILITY

The present disclosure provides a modified nucleoside analog and a nucleotide analog-containing oligonucleotide, wherein the nucleoside analog and oligonucleotide can improve the effect of targeted delivery of drugs on the nervous system cells, so as to improve the inhibition rate of the drugs on expression of a specific gene in the nervous system cells, and realize effective treatment and/or prevention of pathological conditions or diseases caused by abnormal expression of the specific gene in the nervous system cells.

1. A nucleoside analog, wherein the nucleoside analog is a compound as represented by Formula (Ia) or a prodrug thereof: 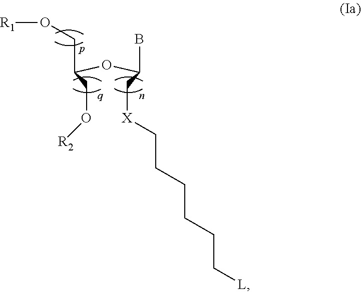
wherein R₁ is selected from H or a hydroxyl protecting group;
R₂ is selected from H or a phosphorus-containing leaving group;
B is selected from substituted or unsubstituted nucleobase, or substituted or unsubstituted nucleobase analog, each substituent in B is independently halogen, C₁-C₃ alkyl, C₁-C₃ alkyloxy, or substituted amide group; and if an amino is present in the B, the amino is protected with an amino protecting group;
X is selected from O, S, NH or —NH—C(═O)—;
L is selected from substituted or unsubstituted C₄-C₁₂ hydrocarbyl, and each substituent in L is independently halogen, C₁-C₃ alkyl or C₁-C₃ alkoxy;
p and q are each independently selected from 1, 2, 3 or 4; and
n is selected from 1, 2 or 3.

2. The nucleoside analog according to claim 1, wherein L is selected from substituted or unsubstituted linear or branched C₄-C₁₂ alkenyl, or substituted or unsubstituted linear or branched C₄-C₁₂ alkyl, and each substituent in L is independently selected from halogen, C₁-C₃ alkyl or C₁-C₃ alkoxy;
optionally, L is selected from substituted or unsubstituted C₄-C₁₂ linear alkyl, or substituted or unsubstituted C₄-C₁₂ linear alkenyl, and each substituent in L is independently selected from halogen, C₁-C₃ alkyl or C₁-C₃ alkyloxy;
optionally, L is selected from C₄-C₁₂ linear alkyl or C₄-C₁₂ linear alkenyl;
optionally, L is selected from C₉-C₁₂ linear alkyl or C₉-C₁₂ linear alkenyl;
optionally, L is selected from C₉-C₁₀ linear alkyl or C₁₂ linear alkenyl;
optionally, L has a structure selected from any one of 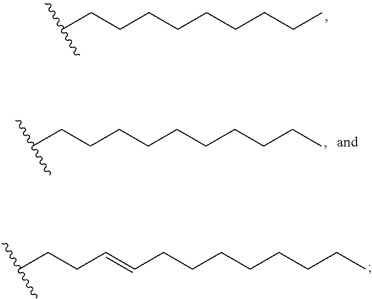
optionally, L is selected from 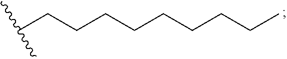 [SMILES: *CCCCCCCCC]
optionally, L is selected from 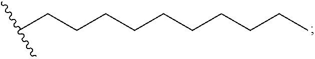 [SMILES: *CCCCCCCCCC]
optionally, L is selected from 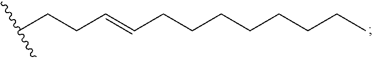 [SMILES: *CC/C=C/CCCCCCCC]
optionally, X is selected from O or —NH—C(═O)—;
optionally, X is selected from O;
optionally, X is selected from —NH—C(═O)—;
optionally, X is selected from O, and L is selected from 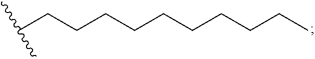 [SMILES: *CCCCCCCCCC]
optionally, X is selected from —NH—C(═O)—, and L is selected from 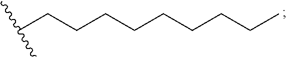 [SMILES: *CCCCCCCCC]
optionally, X is selected from —NH—C(═O)—, and L is selected from 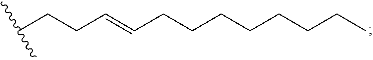 [SMILES: *CC/C=C/CCCCCCCC]
optionally, n=1;
optionally, p and q are each independently selected from 1 or 2;
optionally, p=1 and q=1;
optionally, B is selected from nucleobase or nucleobase analog, and if an amino is present in the B, the amino is protected with an amino protecting group;
optionally, the nucleobase is selected from cytosine, adenine, guanine, thymine, or uracil;
optionally, the nucleobase analog is selected from hypoxanthine-9-yl, purin-9-yl, 2-aminopurine-9-yl, 2,4-difluoro-5-methylphenyl, 5-nitroindol-1-yl, 3-nitropyrrole-1-yl, 4-fluoro-6-methylbenzimidazole-1-yl, or 4-methylbenzimidazole-1-yl;
optionally, the amino protecting group is selected from an alkoxycarbonyl amino protecting group, an acyl amino protecting group or an alkyl amino protecting group;
optionally, the amino protecting group is selected from an acyl amino protecting group;
optionally, the acyl amino protecting group is selected from phthaloyl (Pht), p-toluenesulfonyl (Tos), trifluoroacetyl (Tfa), o- (or p-)nitrobenzenesulfonyl (Ns), pivaloyl (Piv), acetyl (Ac) or benzoyl (Bz);
optionally, the amino protecting group is selected from benzoyl;
optionally, B has a structure selected from any one of 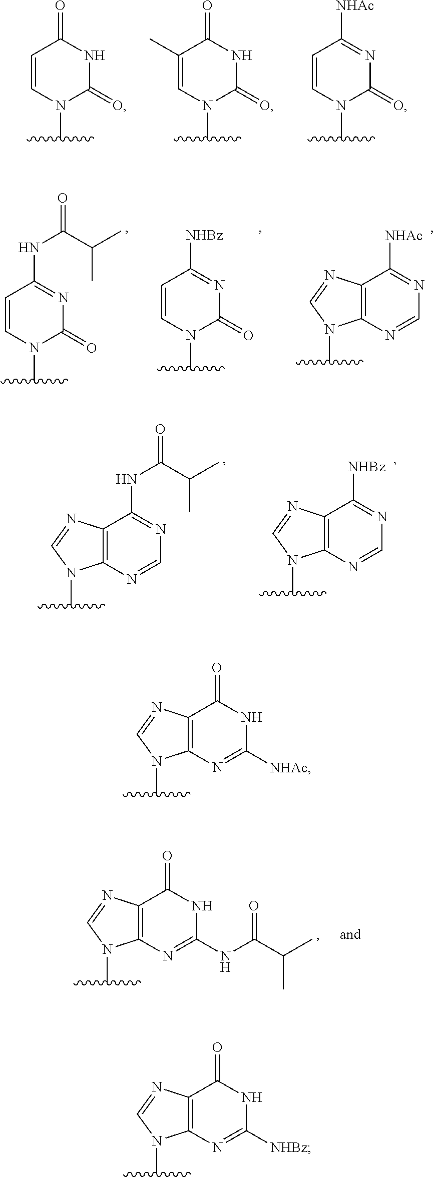
optionally, B is selected from 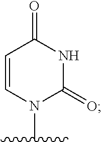 [SMILES: *n1ccc(=O)[nH]c1=O]
optionally, B is selected from 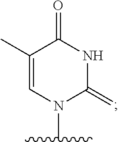
optionally, B is selected from 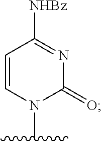
optionally, B is selected from 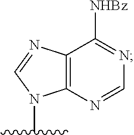
optionally, B is selected from 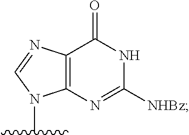
optionally, the hydroxyl protecting group is selected from trityl, 4-methoxytrityl, 4,4′-dimethoxytrityl or 4,4′,4″-trimethoxytriphenyl;
optionally, the hydroxyl protecting group is selected from 4,4′-dimethoxytrityl;
optionally, the phosphorus-containing leaving group is selected from 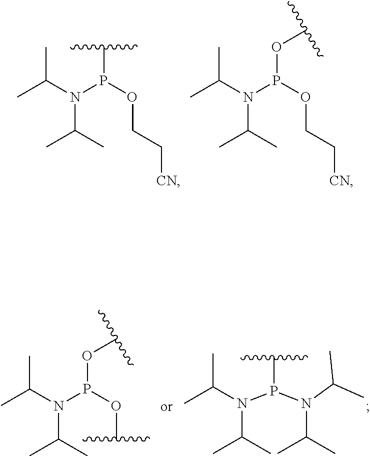 [SMILES: *P(OCCC#N)N(C(C)C)C(C)C ; *OP(OCCC#N)N(C(C)C)C(C)C ; *OP(O*)N(C(C)C)C(C)C ; *P(N(C(C)C)C(C)C)N(C(C)C)C(C)C]
optionally, the phosphorus-containing leaving group is selected from 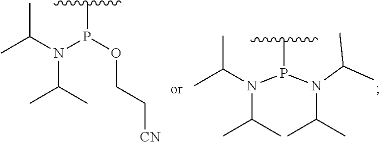 [SMILES: *P(OCCC#N)N(C(C)C)C(C)C ; *P(N(C(C)C)C(C)C)N(C(C)C)C(C)C]
and
optionally, the phosphorus-containing leaving group is selected from

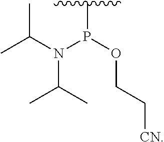 [SMILES: *P(OCCC#N)N(C(C)C)C(C)C]

3. The nucleoside analog according to any one of claims 1-2, wherein the nucleoside analog is a compound as represented by Formula (IIa) or a prodrug thereof:

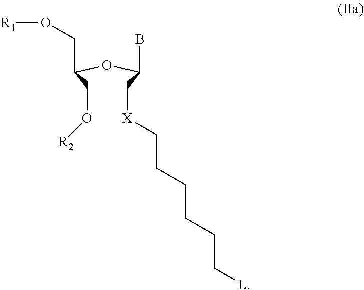

4. The nucleoside analog according to any one of claims 1-2, wherein the nucleoside analog is a compound as represented by Formula (IIIa) or a prodrug thereof:

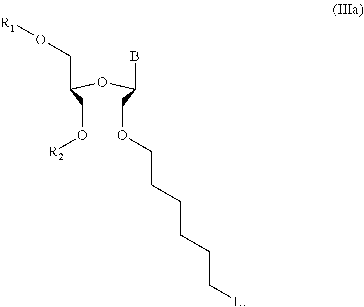

5. The nucleoside analog according to any one of claims 1-2, wherein the nucleoside analog is a compound as represented by Formula (IVa) or a prodrug thereof:

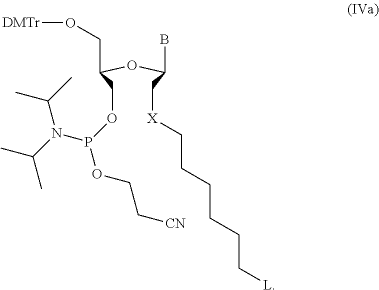

6. The nucleoside analog according to claim 1, wherein the nucleoside analog is a compound having a structure selected from any one of

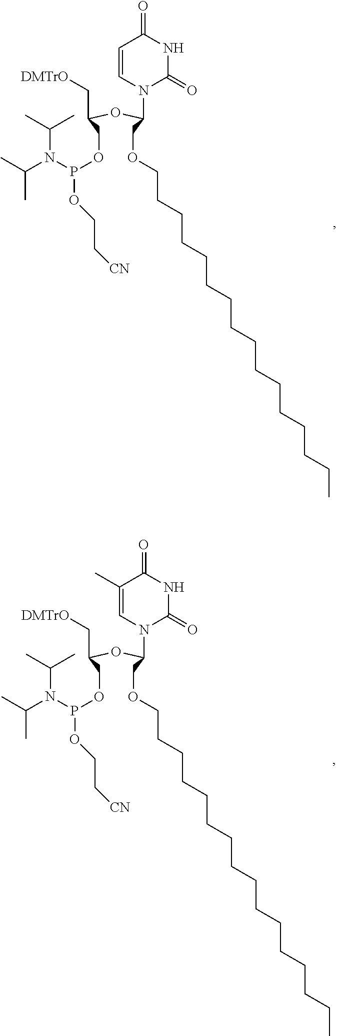

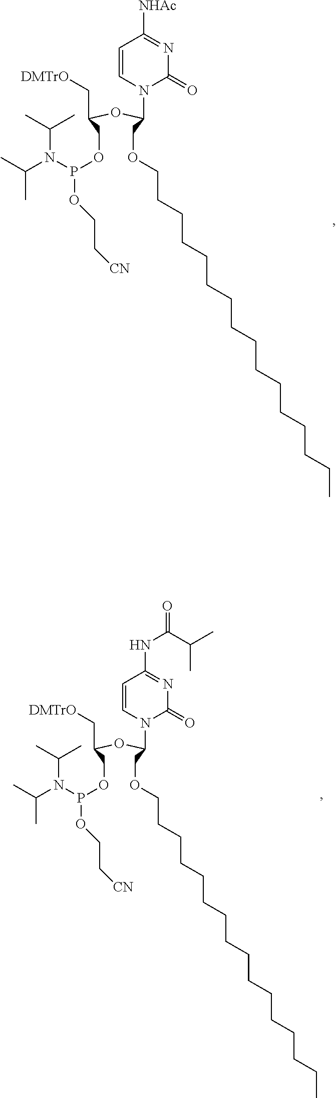

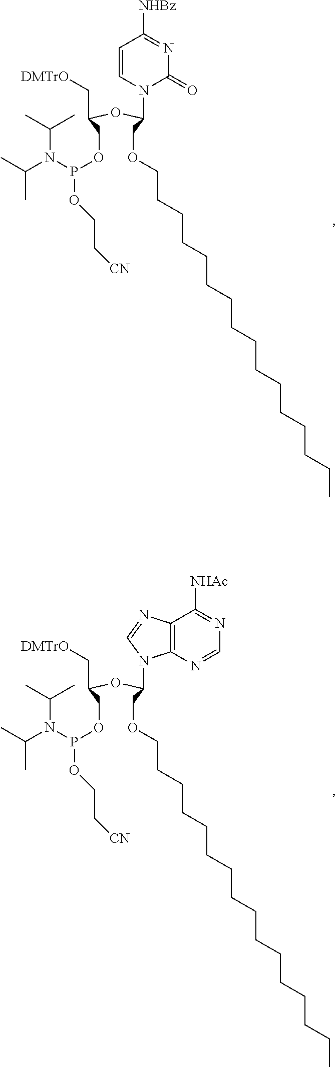

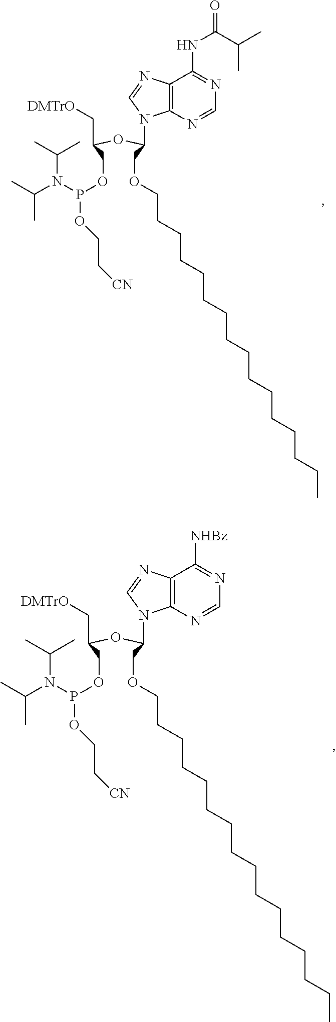

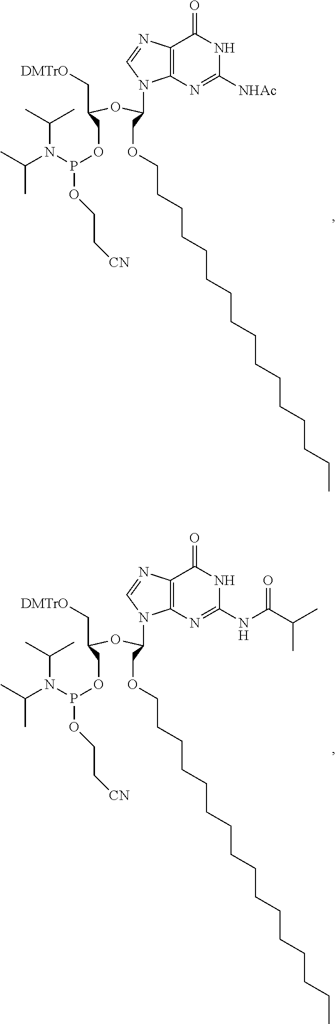

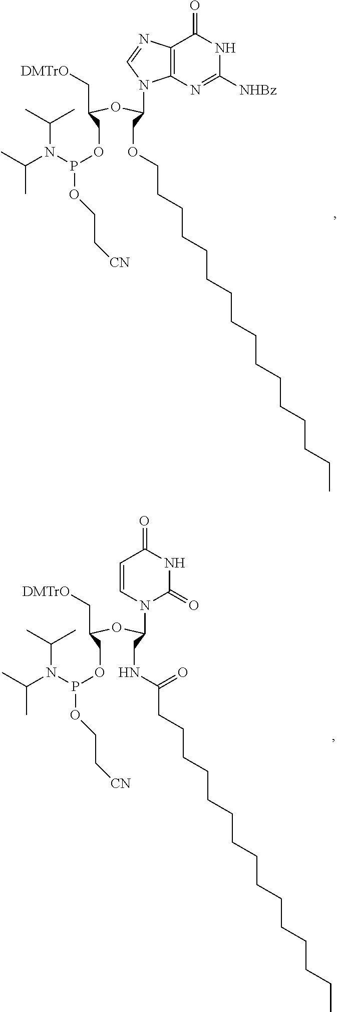

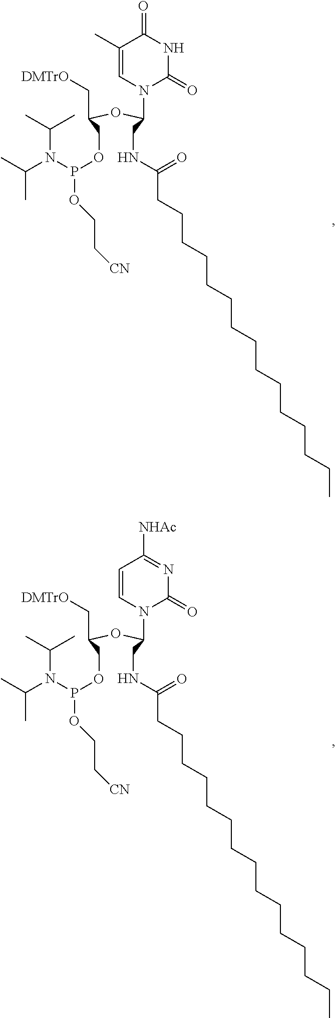

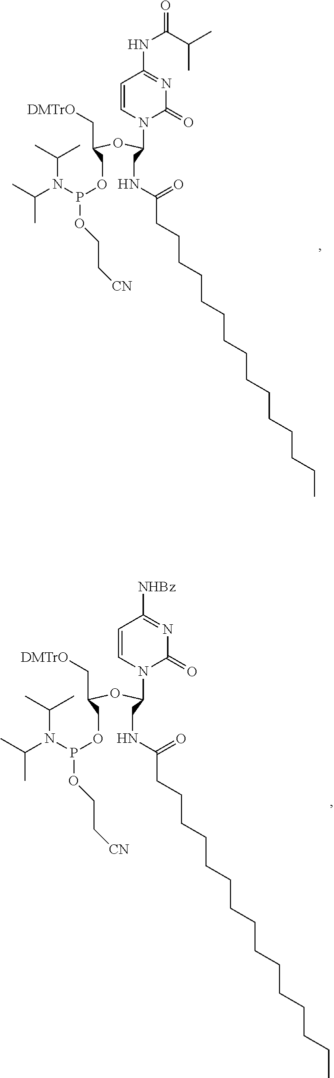

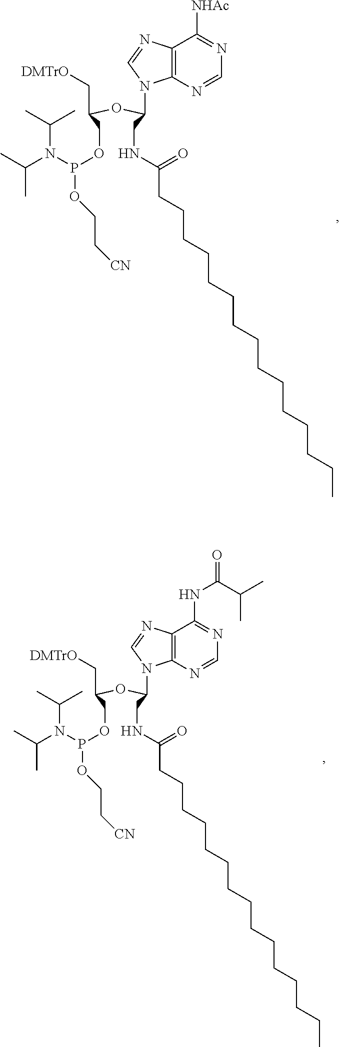

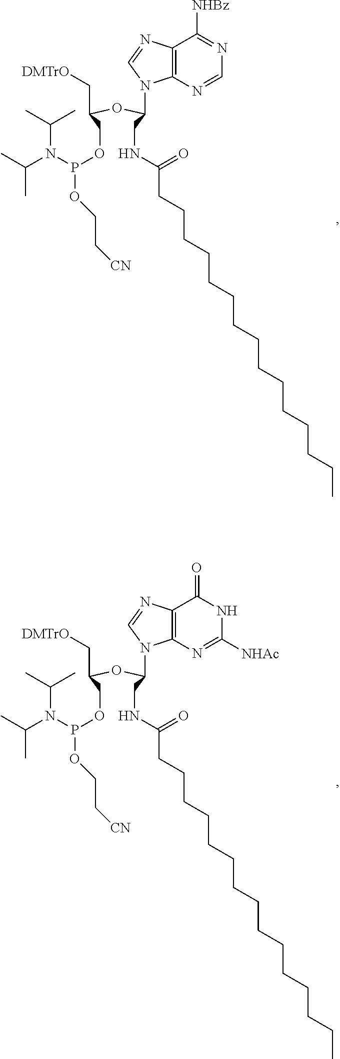

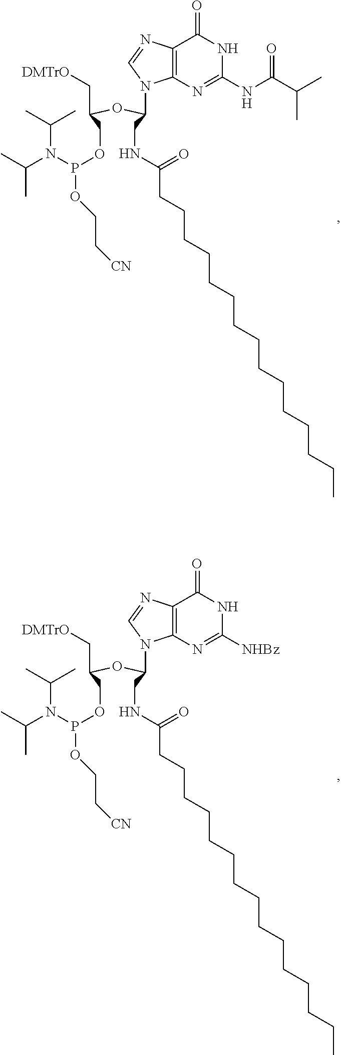

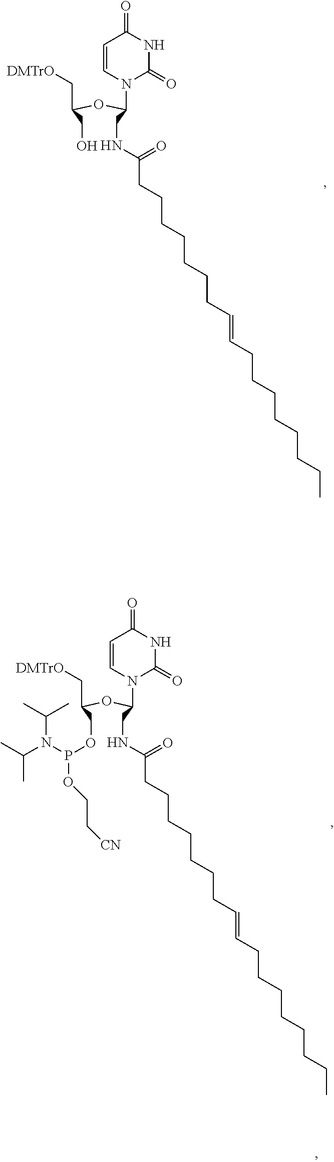

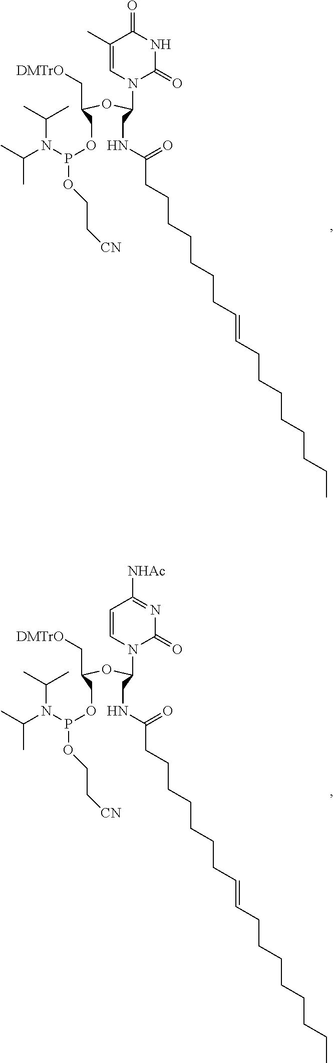

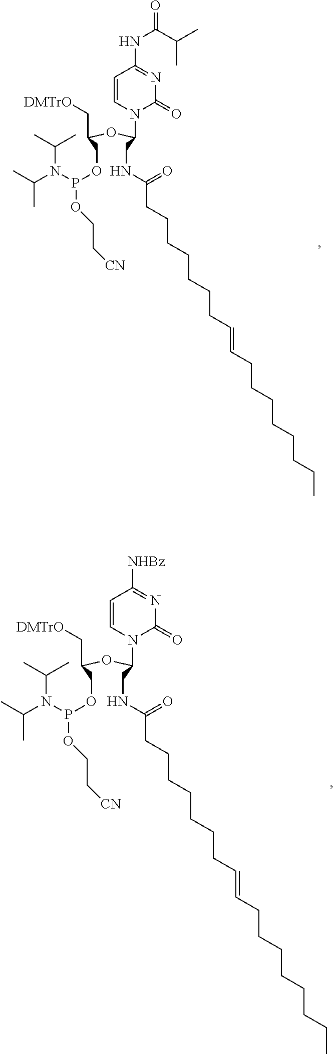

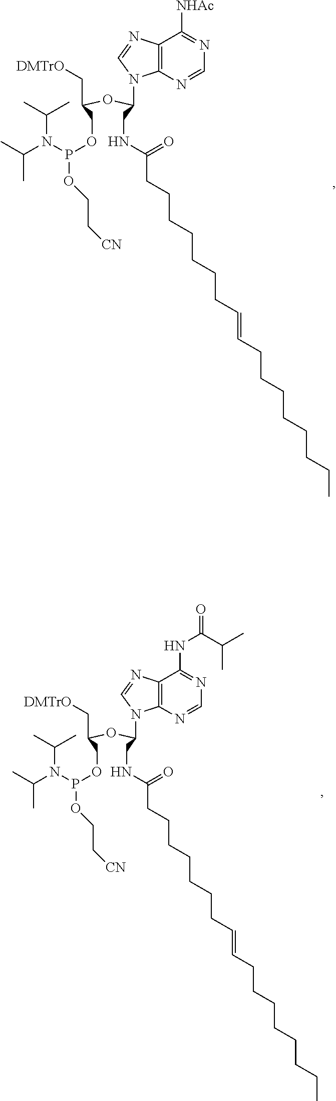

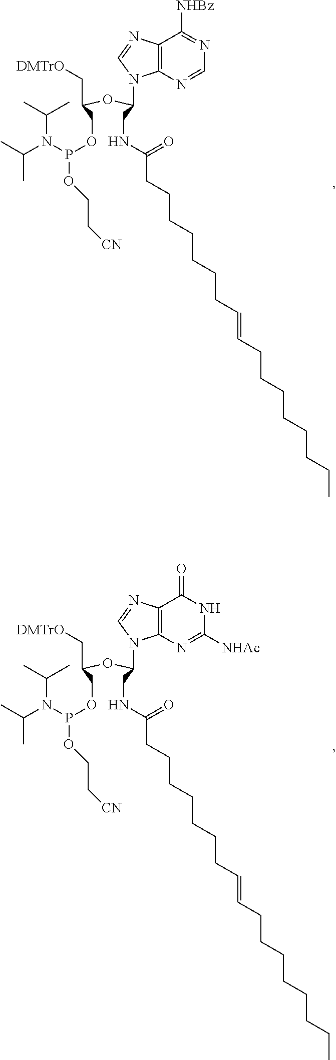

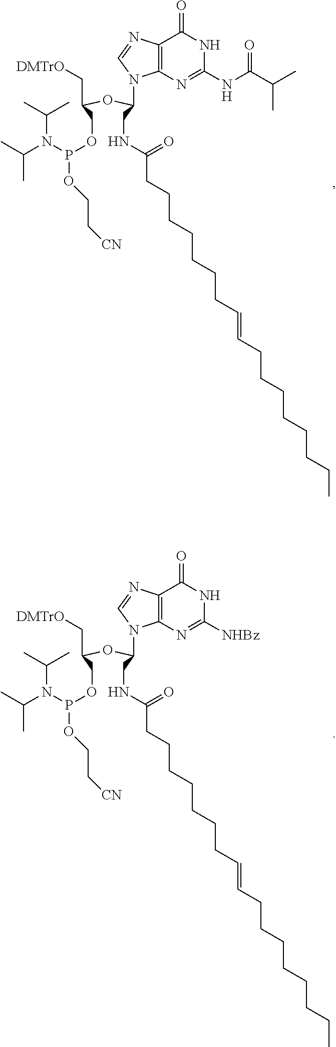

7. A nucleoside analog, wherein the nucleoside analog is a compound as represented by Formula (Ib), Formula (Ic) or Formula (Id) or a prodrug thereof: 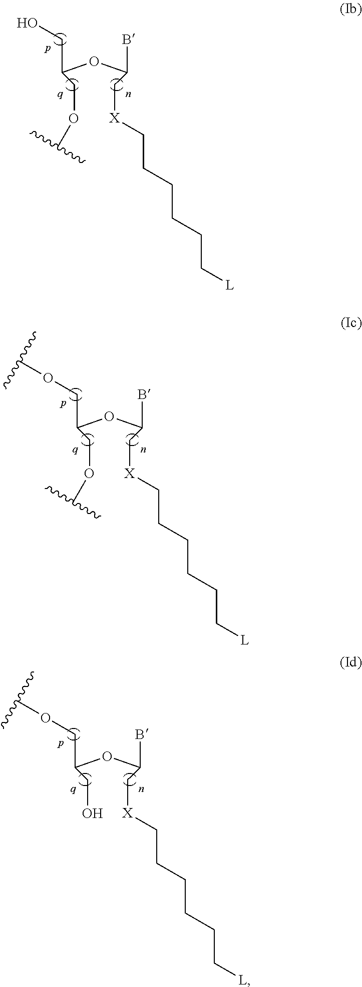
wherein B′ is selected from substituted or unsubstituted nucleobase or substituted or unsubstituted nucleobase analog; each substituent in B′ is independently selected from halogen, C₁-C₃ alkyl, C₁-C₃ alkyloxy, or substituted amide group; and if an amino is present in the B′, the amino is not protected; and
p, q, n, X and L are respectively identical to the p, q, n, X and L defined for the nucleoside analog according to any one of claims 1-6.

8. The nucleoside analog according to claim 7, wherein B′ is selected from nucleobase;
optionally, B′ is selected from 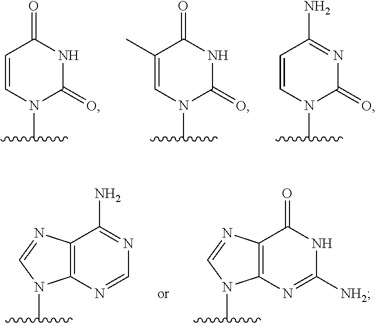
optionally, B′ is selected from 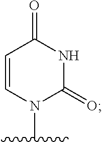 [SMILES: *n1ccc(=O)[nH]c1=O]
optionally, B′ is selected from 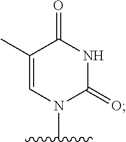 [SMILES: *n1cc(C)c(=O)[nH]c1=O]
optionally, B′ is selected from 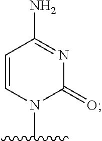 [SMILES: *n1ccc(N)nc1=O]
optionally, B′ is selected from 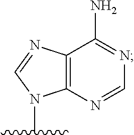 [SMILES: *n1cnc2c(N)ncnc21]
and
optionally, B′ is selected from

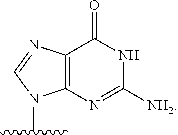 [SMILES: *n1cnc2c(=O)[nH]c(N)nc21]

9. The nucleoside analog according to any one of claims 7-8, wherein the nucleoside analog is selected from a compound as represented by Formula (IIb), Formula (IIc) or Formula (IId) or prodrugs thereof:

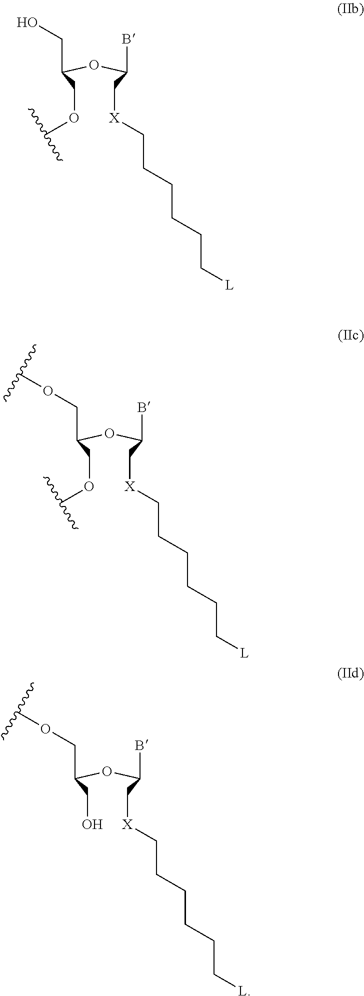

10. The nucleoside analog according to any one of claims 7-8, wherein the nucleoside analog is selected from a compound as represented by Formula (IIIb), Formula (IIIc) or Formula (IIId) or prodrugs thereof:

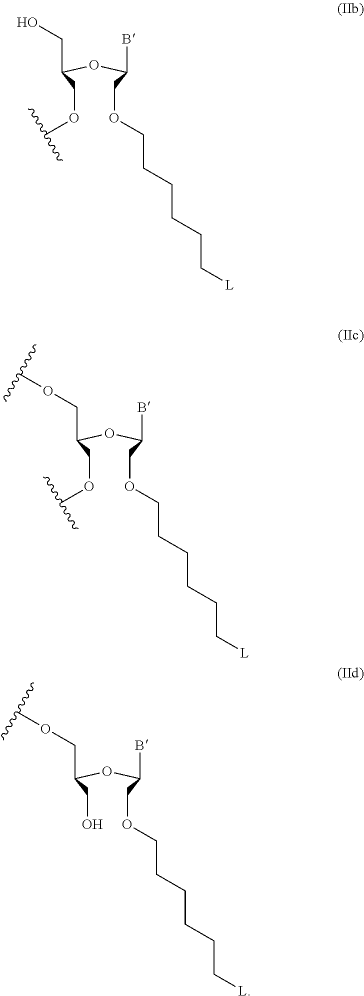

11. The nucleoside analog according to claim 7, wherein the nucleoside analog has a structure selected from any one of

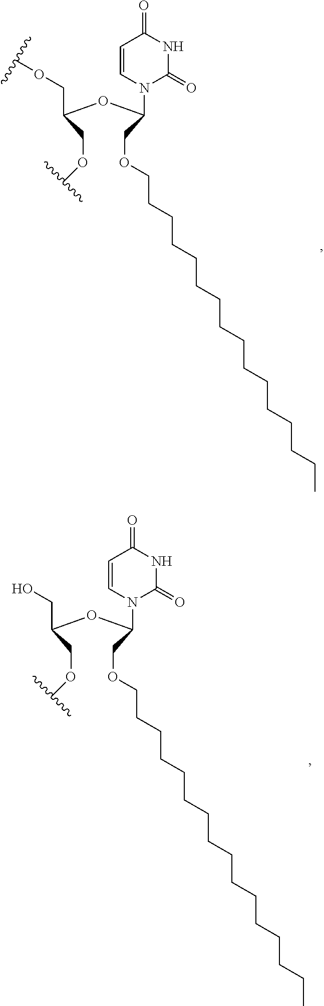

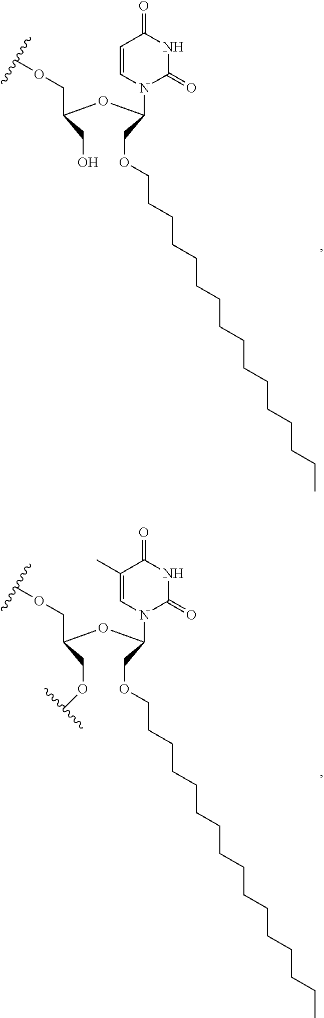

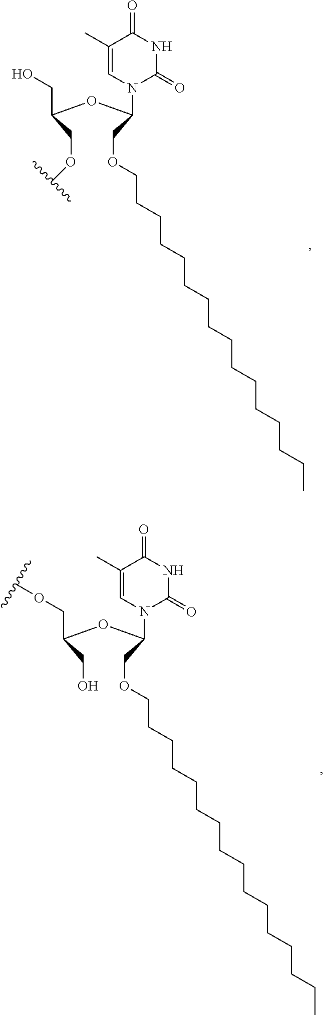 [SMILES: *OC[C@@H](CO)O[C@H](COCCCCCCCCCCCCCCCC)n1cc(C)c(=O)[nH]c1=O ; *OC[C@H](CO)O[C@H](COCCCCCCCCCCCCCCCC)n1cc(C)c(=O)[nH]c1=O]

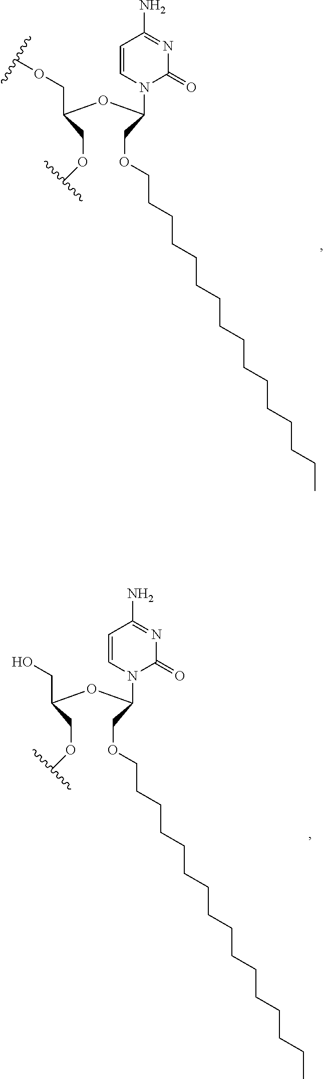

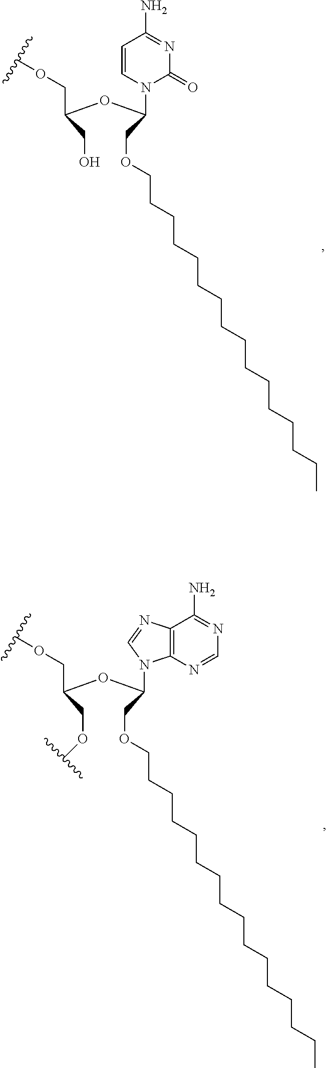

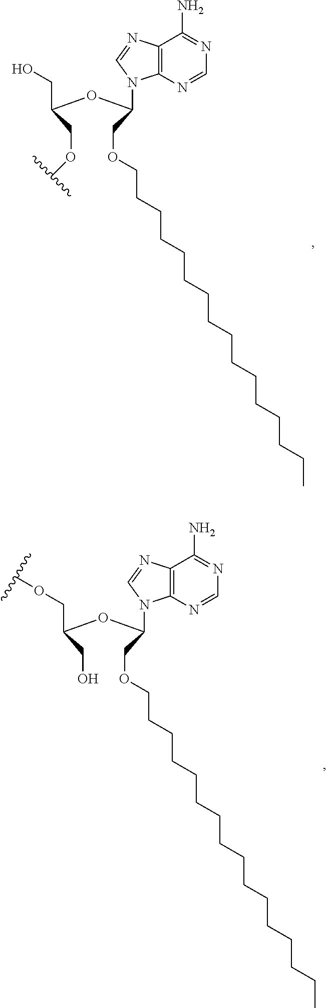 [SMILES: *OC[C@@H](CO)O[C@H](COCCCCCCCCCCCCCCCC)n1cnc2c(N)ncnc21 ; *OC[C@H](CO)O[C@H](COCCCCCCCCCCCCCCCC)n1cnc2c(N)ncnc21]

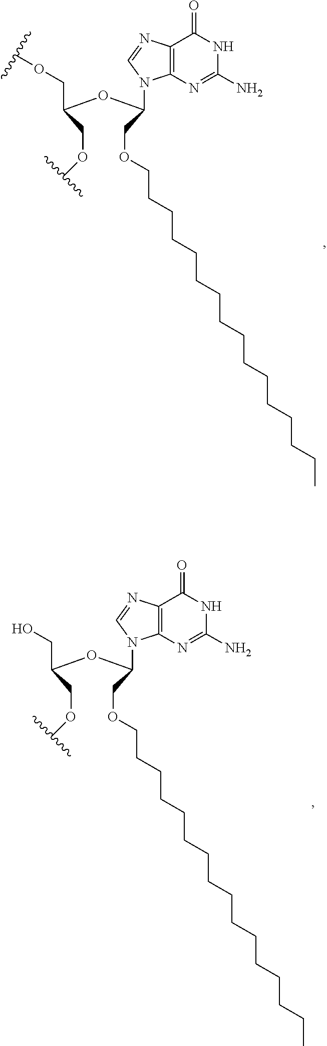

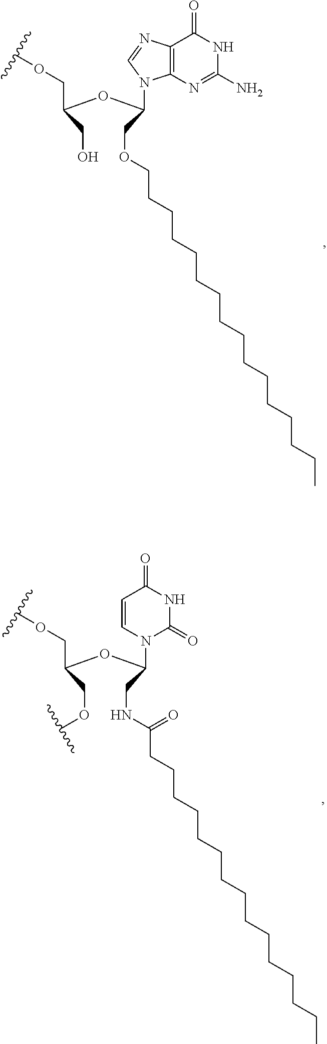

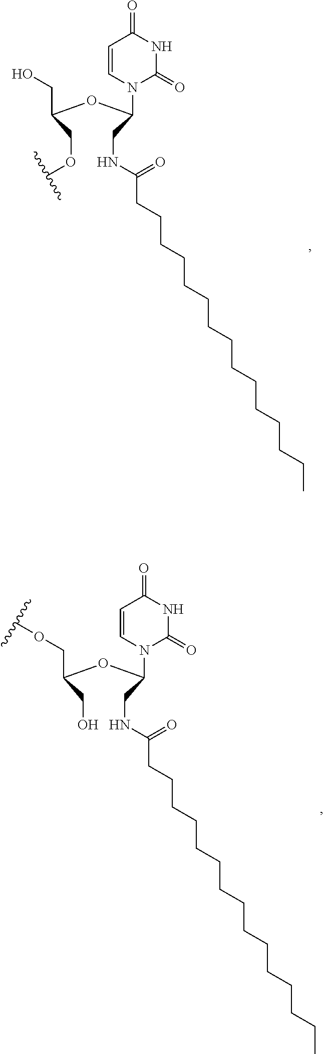 [SMILES: *OC[C@@H](CO)O[C@H](CNC(=O)CCCCCCCCCCCCCCC)n1ccc(=O)[nH]c1=O ; *OC[C@H](CO)O[C@H](CNC(=O)CCCCCCCCCCCCCCC)n1ccc(=O)[nH]c1=O]

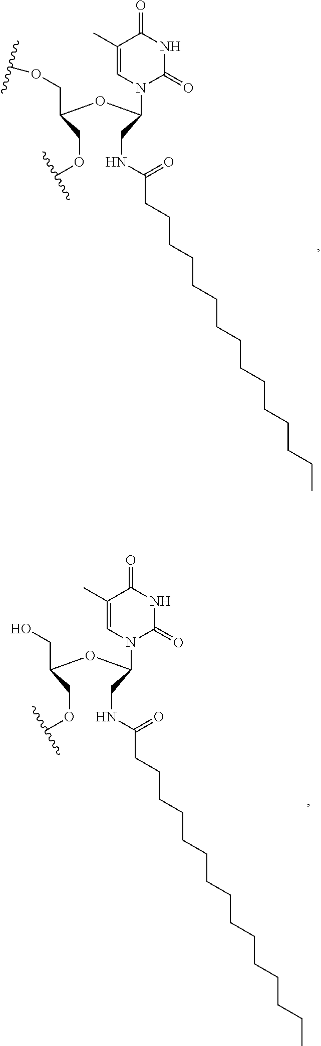

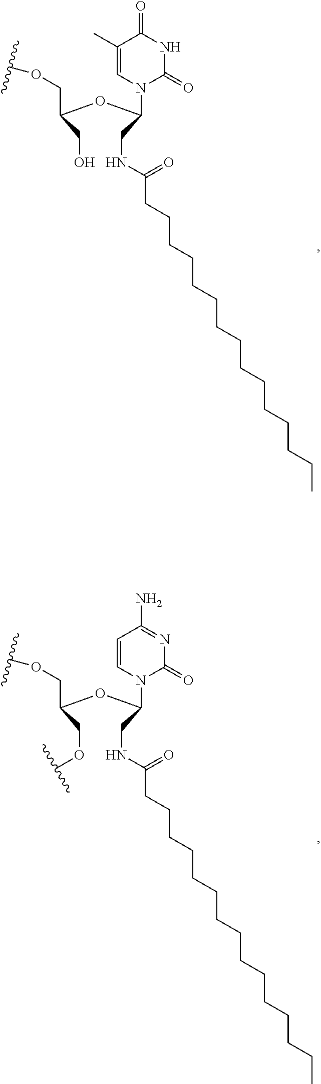

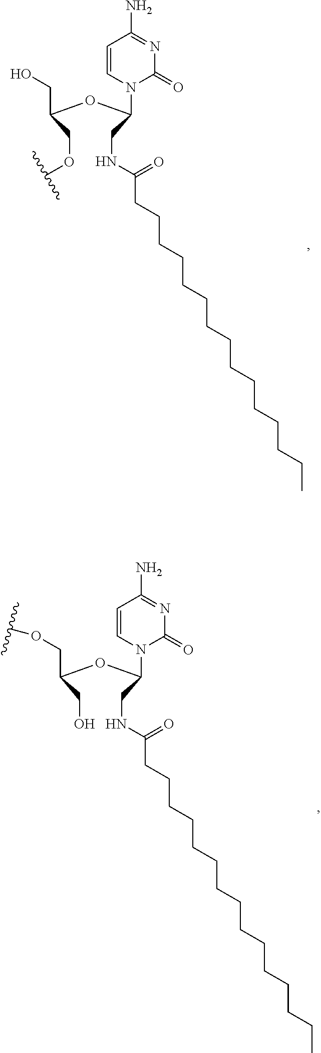 [SMILES: *OC[C@@H](CO)O[C@H](CNC(=O)CCCCCCCCCCCCCCC)n1ccc(N)nc1=O ; *OC[C@H](CO)O[C@H](CNC(=O)CCCCCCCCCCCCCCC)n1ccc(N)nc1=O]

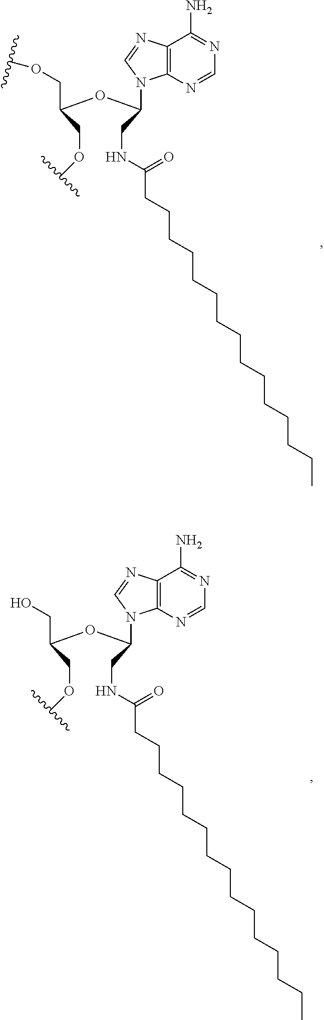

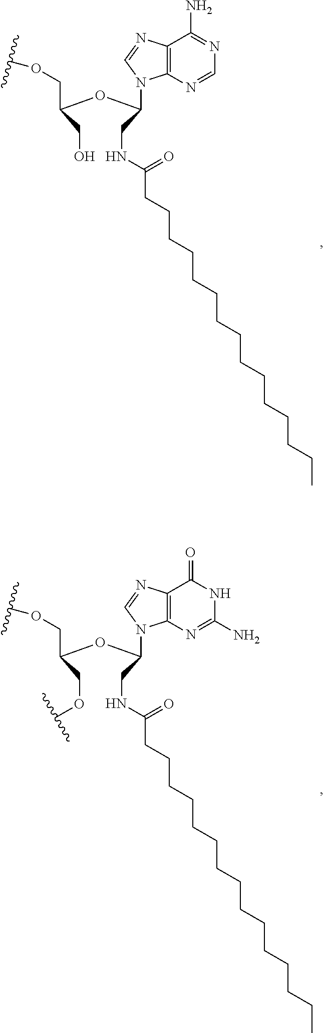

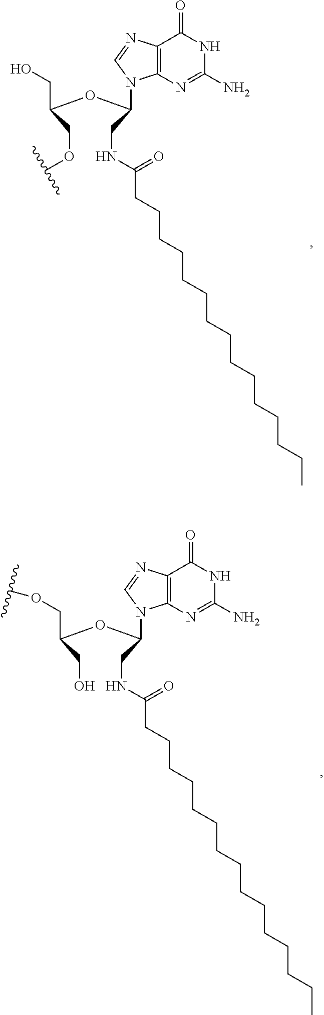 [SMILES: *OC[C@@H](CO)O[C@H](CNC(=O)CCCCCCCCCCCCCCC)n1cnc2c(=O)[nH]c(N)nc21 ; *OC[C@H](CO)O[C@H](CNC(=O)CCCCCCCCCCCCCCC)n1cnc2c(=O)[nH]c(N)nc21]

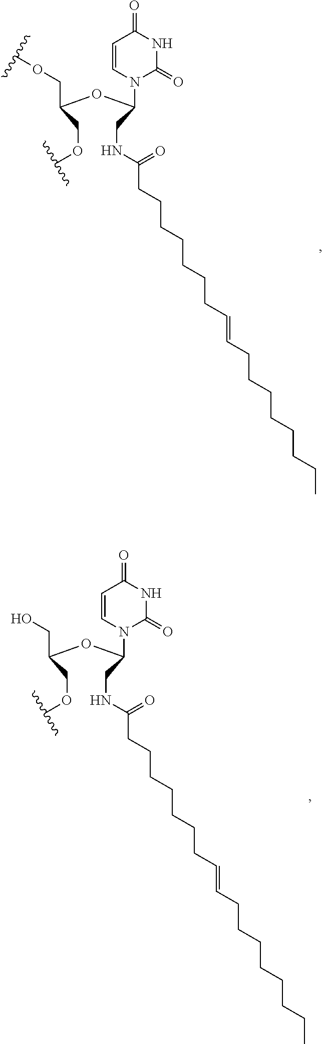

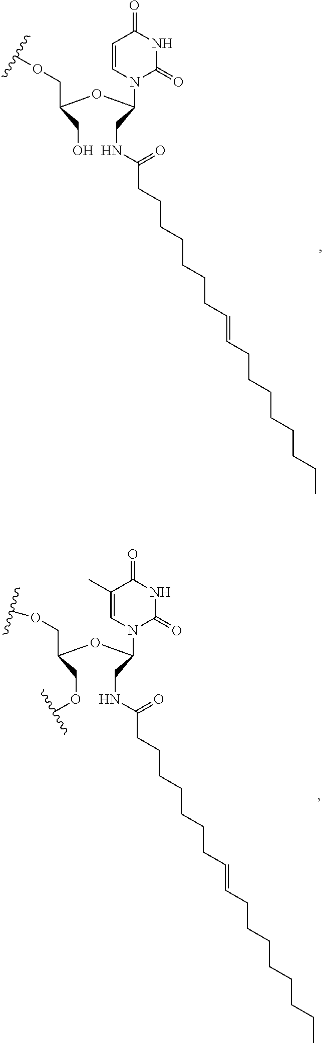

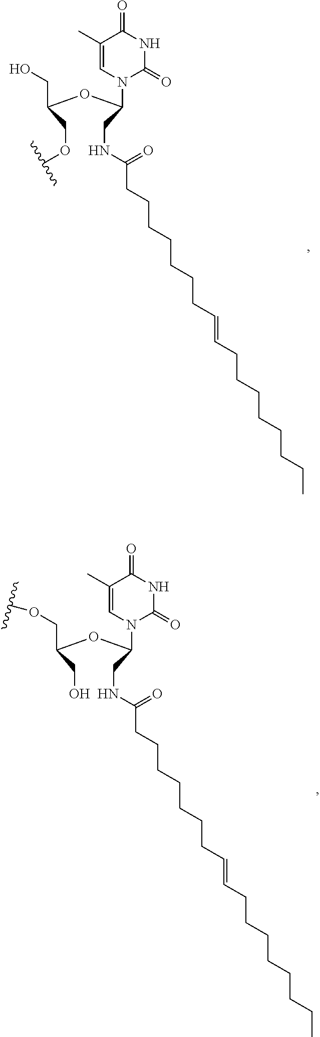 [SMILES: *OC[C@@H](CO)O[C@H](CNC(=O)CCCCCCC/C=C/CCCCCCCC)n1cc(C)c(=O)[nH]c1=O ; *OC[C@H](CO)O[C@H](CNC(=O)CCCCCCC/C=C/CCCCCCCC)n1cc(C)c(=O)[nH]c1=O]

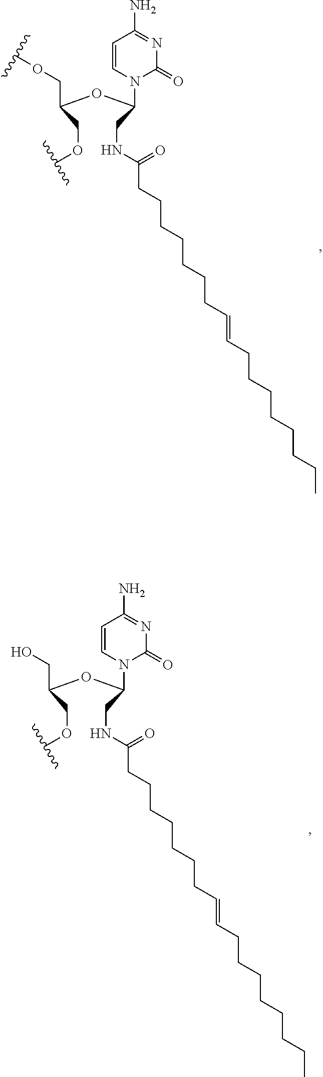

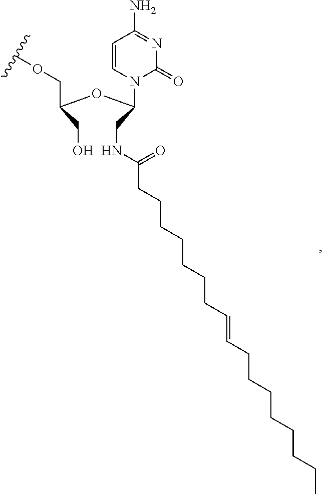 [SMILES: *OC[C@H](CO)O[C@H](CNC(=O)CCCCCCC/C=C/CCCCCCCC)n1ccc(N)nc1=O]

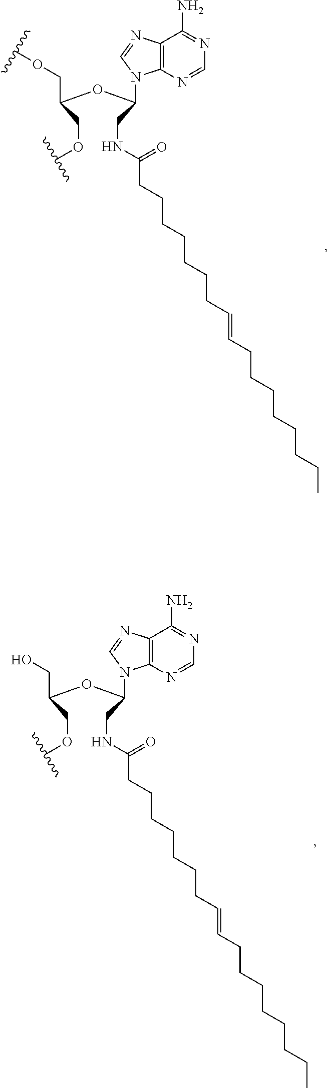

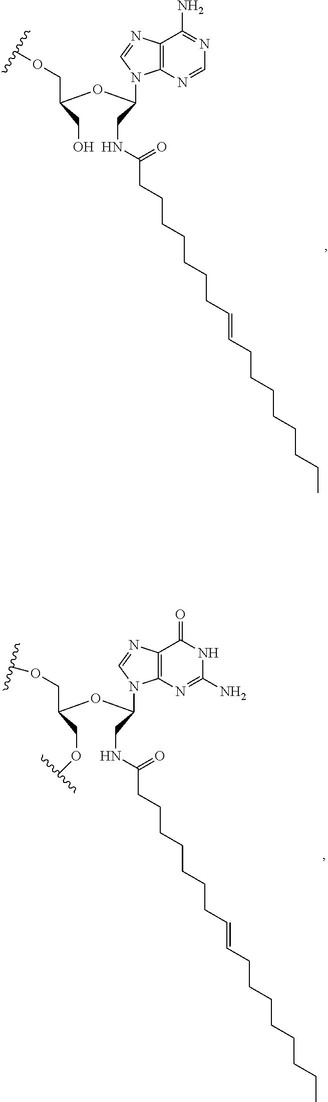

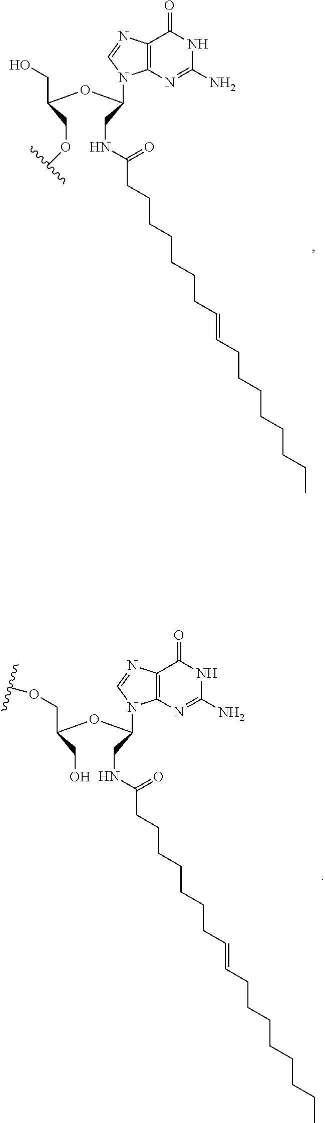 [SMILES: *OC[C@@H](CO)O[C@H](CNC(=O)CCCCCCC/C=C/CCCCCCCC)n1cnc2c(=O)[nH]c(N)nc21 ; *OC[C@H](CO)O[C@H](CNC(=O)CCCCCCC/C=C/CCCCCCCC)n1cnc2c(=O)[nH]c(N)nc21]

12. Use of the nucleoside analog according to any one of claims 1-11 in preparation of oligonucleotide, wherein
optionally, the oligonucleotide is selected from single-stranded oligonucleotide or double-stranded oligonucleotide;
optionally, the oligonucleotide is selected from single-stranded oligonucleotide;
optionally, the oligonucleotide is selected from double-stranded oligonucleotide;
optionally, the single-stranded oligonucleotide is antisense oligonucleotide, nucleic acid aptamer, ribozyme, deoxyribozyme, circular RNA, sense strand of siRNA or antisense strand of siRNA;
optionally, the double-stranded oligonucleotide is small interfering RNA, double-stranded RNA, miRNA, small guide RNA, small activating RNA and or short hairpin RNA; and
optionally, the oligonucleotide is selected from siRNA.

13. A double-stranded oligonucleotide, comprising a sense strand and an antisense strand, the antisense strand and the sense strand have a complementary region with bases at least partially paired, and the sense strand and/or the antisense strand comprises at least one nucleoside analog according to any one of claims 7-11;
optionally, the sense strand and the antisense strand each independently have a length of 15-25 nucleotides;
optionally, the antisense strand is free of the nucleoside analog, and the sense strand comprises one of the nucleoside analogs;
optionally, the nucleoside analog is located at Positions 2-8 of the sense strand counting from 5′-end;
optionally, the nucleoside analog is located at Positions 3-7 of the sense strand counting from the 5′-end;
optionally, the nucleoside analog is located at Positions 4-6 of the sense strand counting from the 5′-end;
optionally, the nucleoside analog is located at Position 4 of the sense strand counting from the 5′-end;
optionally, the nucleoside analog is located at Position 5 of the sense strand counting from the 5′-end;
optionally, the nucleoside analog is located at Position 6 of the sense strand counting from the 5′-end; and
optionally, the double-stranded oligonucleotide is selected from siRNA.

14. A composition, comprising the double-stranded oligonucleotide according to claim 13; and
optionally, the composition further comprises optionally one or more pharmaceutically acceptable carriers or adjuvants.

15. Use of the nucleoside analog according to any one of claims 1-11, the double-stranded oligonucleotide according to claim 13 or the composition according to claim 14 in preparation of medicaments for treating and/or preventing pathological conditions or diseases caused by abnormal expression of a specific gene in nervous system cells.

16. A method for inhibiting expression of a specific gene in target cells, comprising:
contacting the double-stranded oligonucleotide according to claim 13 or the composition according to claim 14 with the target cells; and
optionally, the target cells are selected from nervous system cells.

17. A method for preventing and/or treating pathological conditions or diseases caused by abnormal expression of a specific gene in target cells, comprising: administering to a subject a pharmaceutically acceptable dose of the double-stranded oligonucleotide according to claim 13 or the composition according to claim 14; and
optionally, the target cells are selected from nervous system cells

18. A double-stranded oligonucleotide, comprising a sense strand and an antisense strand, each strand has 17-25 modified and/or unmodified nucleotides, the antisense strand and the sense strand are complementary to form a duplex region; the sense strand and/or the antisense strand comprises at least one nucleotide analog as represented by Formula (100), or a tautomer, or a stereoisomer, or a pharmaceutically acceptable salt thereof: 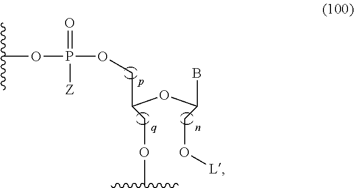
wherein [symbol] represents a covalently linking position between the nucleotide analog and a neighboring nucleotide;
B is selected from unsubstituted or substituted nucleobase, or unsubstituted or substituted nucleobase analog; and if the B contains a substituent, the substituent is independently selected from halogen, C₁-C₃ alkyl, C₁-C₃ alkyloxy, or amide group;
L′ is selected from substituted or unsubstituted C₁₆-C₂₄ hydrocarbyl; and if the L′ contains a substituent, the substituent is independently selected from halogen, C₁-C₃ alkyl or C₁-C₃ alkoxy;
p is selected from 1, 2, 3 or 4;
q is selected from 1, 2, 3 or 4;
n is selected from 1, 2 or 3; and
Z is selected from hydroxyl or thiol.

19. The double-stranded oligonucleotide according to claim 18, wherein the sense strand and/or the antisense strand comprises at least one nucleotide analog as represented by Formula (200), or a tautomer thereof, or a pharmaceutically acceptable salt thereof: 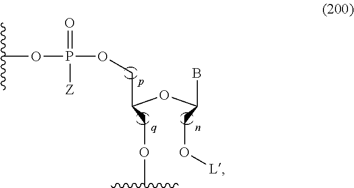
wherein definitions of substituents are identical to those in Formula (100).

20. The double-stranded oligonucleotide according to claim 18 or 19, wherein B is selected from nucleobases A, U, G, C or T, or analogs of the nucleobases, wherein
L′ is selected from saturated or unsaturated C₁₈-C₂₂ hydrocarbyl; p is selected from 1 or 2; q is selected from 1 or 2; n is selected from 1 or 2; and Z is selected from hydroxyl or thiol;
optionally, L′ is selected from C₁₈-C₂₂ alkyl;
optionally, L′ is selected from saturated C₁₈, C₁₉, C₂₀,C₂₁ or C₂₂ linear alkyl; and
optionally, n is selected from 1.

21. The double-stranded oligonucleotide according to claim 20, wherein the nucleotide analog is a compound selected from any one with following structures, or tautomers thereof, or pharmaceutically acceptable salts thereof:

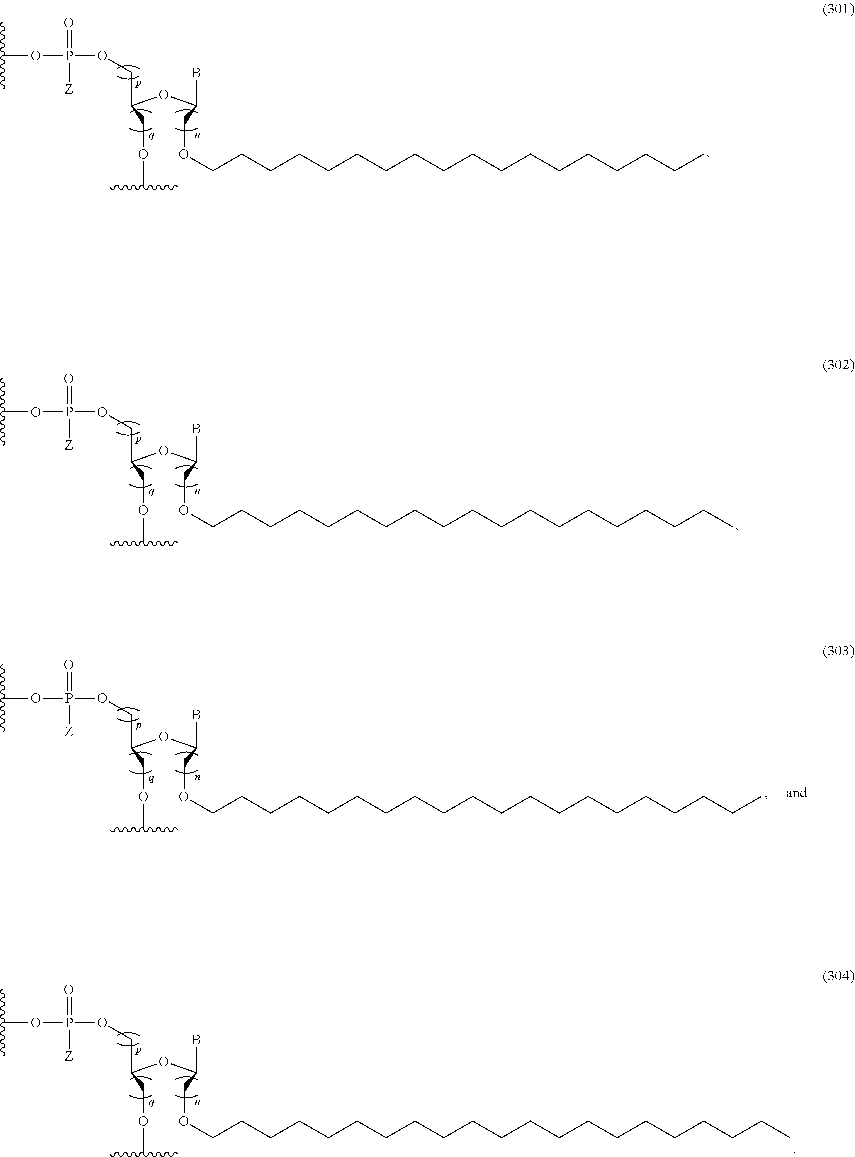

22. The double-stranded oligonucleotide according to any one of claims 18-21, wherein the sense stand comprises one or two of the nucleotide analogs; optionally, the sense stand comprises one of the nucleotide analogs;
optionally, the nucleotide analog is located at 5′-end of the sense strand, or at 3′-end of the sense strand, or at an internal position between the 5′-end and the 3′-end of the sense strand;
optionally, the nucleotide analog is located at any position in Positions 2-19 of the sense strand counting from the 5′-end;
optionally, the nucleotide analog is located at the 5′-end or the 3′-end of the sense strand;
optionally, the nucleotide analog is located at any position in Positions 2-8 of the sense strand counting from the 5′-end;
optionally, the nucleotide analog is located at any position in Positions 4-6 of the sense strand counting from the 5′-end;
optionally, the sense strand and the antisense strand each independently have a length of 19-23 nucleotides;
optionally, the antisense strand comprises 1-3 phosphorothioate diester bonds located at the 5′-end and 1-3 phosphorothioate diester bonds located at the 3′-end; and/or the sense strand comprises 1-3 phosphorothioate diester bonds located at the 5′-end; and
optionally, the antisense strand is free of the nucleotide analog, and the antisense strand is basically reversely complementary, substantially reversely complementary or fully reversely complementary to a nucleotide sequence in an mRNA expressed by the target gene.

23. The double-stranded oligonucleotide according to any one of claims 18-22, wherein nucleotides of the double-stranded oligonucleotide other than the nucleotide replaced with the nucleotide analog are all modified, and are each independently selected from the following modified nucleotides: 2′-O-methyl modified nucleotides, 2′-fluoro modified nucleotides, or 2′-O-methoxyethyl (2′-O-MOE) modified nucleotides;
optionally, in a direction from 5′-end to 3′-end, nucleotides at Position 2, Position 6, Position 14, and Position 16 of the antisense strand are selected from 2′-fluoro modified nucleotides; and/or nucleotides at Position 7 and Position 9 of the sense strand are selected from 2′-fluoro modified nucleotides;
optionally, in the direction from the 5′-end to the 3′-end, at least three nucleotides at Positions 7-10 of the sense strand are selected from 2′-fluoro modified nucleotides; and/or at least four nucleotides at Positions 2, 6, 9, 12, 14 and 16 of the antisense strand are selected from 2′-fluoro modified nucleotides, and a nucleotide at Position 15 is selected from 2′-O-methoxyethyl or 2′-O-methyl modified nucleotide;
optionally, in the direction from the 5′-end to the 3′-end, at least three nucleotides at Positions 7-10 of the sense strand are selected from 2′-fluoro modified nucleotides, and one nucleotide at Positions 2-8 is selected from the nucleotide analog; and/or at least four nucleotides at Positions 2, 6, 9, 12, 14 and 16 of the antisense strand are selected from 2′-fluoro modified nucleotides, and a nucleotide at Position 15 is selected from 2′-O-methoxyethyl modified nucleotide or 2′-O-methyl modified nucleotide; and nucleotides at remaining positions are selected from 2′-O-methyl modified nucleotides; and
optionally, in the direction from the 5′-end to the 3′-end, nucleotides at Positions 7-10 of the sense strand are selected from 2′-fluoro modified nucleotides, and one nucleotide at Positions 4-6 is selected from the nucleotide analog; and/or at least five nucleotides at Positions 2, 6, 9, 12, 14 and 16 of the antisense strand are selected from 2′-fluoro modified nucleotides, and a nucleotide at Position 15 is selected from 2′-O-methoxyethyl modified nucleotide; and nucleotides at remaining positions are selected from 2′-O-methyl modified nucleotides.

24. A composition, comprising the double-stranded oligonucleotide according to any one of claims 18-23.

25. Use of the double-stranded oligonucleotide according to any one of claims 18-23 or the composition according to claim 24 in preparation of medicaments for treating and/or preventing pathological conditions or diseases caused by abnormal expression of a specific gene in nervous system cells.

26. A pharmaceutical composition, comprising the double-stranded oligonucleotide according to any one of claims 18-23 and/or the composition according to claim 24; and
optionally, the composition further contains optionally one or more pharmaceutically acceptable carriers or adjuvants.

27. A method for reducing expression of a gene in target cells, comprising: contacting the double-stranded oligonucleotide according to any one of claims 18-23 and/or the composition according to claim 24 and/or the pharmaceutical composition according to claim 26 with the target cells; and
optionally, the target cells are selected from nervous system cells.

28. A method for preventing and/or treating pathological conditions or diseases caused by abnormal expression of a specific gene in target cells, comprising: administering to a subject a pharmaceutically acceptable dose of the double-stranded oligonucleotide according to any one of claims 18-23 and/or the composition according to claim 24 and/or the pharmaceutical composition according to claim 26; and
optionally, the target cells are selected from nervous system cells.

29. A method for reducing expression of a target gene in a subject, comprising: administering to the subject the double-stranded oligonucleotide according to any one of claims 18-23 and/or the composition according to claim 24 and/or the pharmaceutical composition according to claim 26;
optionally, the method comprises intrathecal administration;
optionally, the method reduces the expression of the target gene in brain or spine tissues; and
optionally, the brain or spine tissues comprise, but are not limited to, the group consisting of cortex, cerebellum, cervical spine, lumbar spine and thoracic spine.

30. A method for treating a subject suffering from CNS disorder-associated diseases or conditions, comprising:
administering to the subject a therapeutically effective amount of the double-stranded oligonucleotide according to any one of claims 18-23 and/or the composition according to claim 24 and/or the pharmaceutical composition according to claim 26, so as to treat the CNS disorder-associated diseases or conditions of the subject;
optionally, the CNS disorder is selected from the group consisting of Parkinson's disease, amyotrophic lateral sclerosis (ALS), diffuse Lewy body disease, Parkinson's disease dementia, Huntington's disease, multiple system atrophy, Alzheimer's disease and other neurodegenerative diseases; and
optionally, the subject is selected from humans.

31. A kit, comprising the double-stranded oligonucleotide according to any one of claims 18-23 and/or the composition according to claim 24 and/or the pharmaceutical composition according to claim 26.

32. A method for inhibiting expression of a target gene in skeletal muscle cells, ocular cells, myocardial cells or adipose cells, comprising: contacting the cells with the double-stranded oligonucleotide according to any one of claims 18-23 and/or the composition according to claim 24 and/or the pharmaceutical composition according to claim 26.

33. A method for preventing and/or treating diseases or conditions associated with skeletal muscles, cardiac muscles, eyes or adipose tissues, wherein the method comprising: administering to a subject a therapeutically effective amount of the double-stranded oligonucleotide according to any one of claims 18-23 and/or the composition according to claim 24 and/or the pharmaceutical composition according to claim 26, so as to treat the subject; and
optionally, the subject is selected from humans.

34. The method according to claim 33, wherein the diseases or conditions associated with skeletal muscles are muscular atrophies; and
optionally, the muscular atrophies are selected from the group consisting of Duchenne muscular atrophy, myotonic muscular atrophy, Becker muscular atrophy, limb-girdle type muscular atrophy, facioscapulohumeral muscular atrophy, congenital muscular atrophy, oculopharyngeal muscular atrophy, distal muscular atrophy and Emery-Dreifuss muscular atrophy, myostatin-related muscular hypertrophy, congenital myasthenic syndrome and facioscapulohumeral muscular dystrophy (FSHD).

35. The method according to claim 33, wherein the diseases or conditions associated with cardiac muscles are selected from the group consisting of hypertrophic obstructive cardiomyopathy (HOCM); familial hypertrophic cardiomyopathy (FHC); heart failure with preserved ejection fraction (HFPEF); atrial fibrillation (AFIB); ventricular fibrillation (VFIB); angina pectoris; myocardial infarction (MI); heart failure or heart failure with reduced ejection fraction (HFREF); supra ventricular tachycardia (SVT); hypertrophic cardiomyopathy (HCM), dilated cardiomyopathy (DCM), arrhythmia and congestive heart failure (CHF).

36. The method according to claim 33, wherein the diseases or conditions associated with adipose tissues are metabolic diseases; and
optionally, the metabolic diseases are selected from the group consisting of lipid metabolism conditions, hypertension, cardiovascular diseases or overweight-related conditions.

37. The method according to claim 33, wherein the diseases or conditions associated with eyes are selected from the group consisting of glaucoma (comprising but not limited to primary open-angle glaucoma, secondary glaucoma, pigmentary glaucoma, pseudo-exfoliative glaucoma, traumatic glaucoma, neovascular glaucoma, uveitic glaucoma, ocular conditions of angle-closure glaucoma, normal-tension glaucoma, juvenile open-angle glaucoma, primary open-angle glaucoma), iridocorneal endothelial syndrome, macular degeneration, cataracts, diabetic retinopathy, dry eye, night vision, strabismus, ocular tremor, color blindness, uveitis, ocular inflammation, presbyopia, retinal diseases, corneal diseases, diabetic macular edema, ocular hypertension, astigmatism, diabetic eye diseases, hypermetropia, myopia, and macular edema.

38. The method according to any one of claims 32-37, wherein the double-stranded oligonucleotide agent is administered to the subject by subcutaneous administration, intramuscular administration, intravenous administration, intraperitoneal administration or intravitreal administration.

39. The method according to claim 38, wherein administration of the double-stranded oligonucleotide agent results in that expression of the target gene is no more than 40%, no more than 50%, no more than 60% or no more than 70% of that without administration.
